SEQUENCE LISTING (229 sequences)

SEQ ID NO: 1 - DNA, 1301 bp, Ruminococcus gnavus
  misc_feature: HB-40_Ruminococcus_gnavus
tgcagtcgagcgaagcacttttgcggatttcttcggattgaagcaattgtgactgagcggcggacgggtgagtaacgcgtgggtaacctgcctcatacagggggataacagttggaaacggctgctaataccgcataagcgcacagtaccgcatggtaccgtgtgaaaaactccggtggtatgagatggacccgcgtctgattagctagttggtggggtaacggcctaccaaggcgacgatcagtagccgacctgagagggtgaccggccacattgggactgaracacggcccaaactcctacgggaggcagcagtggggaatattgcacaatgggggaaaccctgatgcagcgacgccgcgtgagcgatgaagtatttcggtatgtaaagctctatcagcagggaagaaaatgacggtacctgactaagaagccccggctaactacgtgccagcagccgcggtaatacgtagggggcaagcgttatccggatttactgggtgtaaagggagcgtagacggcatggcaagccagatgtgaaagcccggggctcaaccccgggactgcatttggaactgtcaggctagagtgtcggagaggaaagcggaattcctagtgtagcggtgaaatgcgtagatattaggaggaacaccagtggcgaaggcggctttctggacgatgactgacgttgaggctcgaaagcgtggggagcaaacaggattagataccctggtagtccacgccgtaaacgatgaatactaggtgtcgggtggcaaagccattcggtgccgcagcaaacgcaataagtattccacctggggagtacgttcgcaagaatgaaactcaaaggaattgacggggacccgcacaagcggtggagcatgtggtttaattcgaagcaacgcgaagaaccttacctggtcttgacatccctctgaccgctctttaatcggagctttccttcgggacagaggagacaggtggtgcatggttgtcgtcagctcgtgtcgtgagatgttgggttaagtcccgcaacgagcgcaacccctatctttagtagccagcatttaaggtgggcactctagagagactgccagggataacctggaggaaggtggggatgacgtcaaatcatcatgccccttatgaccagggctacacacgtgctacaatggcgtaaacaaagggaagcgagcccgcgagggggagcaaatcccaaaaataacgtctcagttcggattgtagtctgcaactcgactacatgaagctggaatcgctagtaatcgcgaatcagaatgtcg

SEQ ID NO: 2 - DNA, 1328 bp, Ruminococcus gnavus
  misc_feature: HB-516_Ruminococcus_gnavus
  misc_feature (1297)..(1297): n is a, c, g, or t
gctcaggatgaacgctggcggcgtgcttaacacatgcaagtcgagcgaagcacttttgcggatttcttcggattgaagcaattgtgactgagcggcggacgggtgagtaacgcgtgggtaacctgcctcatacagggggataacagttggaaacggctgctaataccgcataagcgcacagtaccgcatggtacggtgtgaaaaactccggtggtatgagatggacccgcgtctgattaggtagttggtggggtaacggcctaccaagccgacgatcagtagccgacctgagagggtgaccggccacattgggactgagacacggcccaaactcctacgggaggcagcagtggggaatattgcacaatgggggaaaccctgatgcagcgacgccgcgtgagcgatgaagtatttcggtatgtaaagctctatcagcagggaagaaaatgacggtacctgactaagaagccccggctaactacgtgccagcagccgcggtaatacgtagggggcaagcgttatccggatttactgggtgtaaagggagcgtagacggcatggcaagccagatgtgaaagcccggggctcaaccccgggactgcatttggaactgtcaggctagagtgtcggagaggaaagcggaattcctagtgtagcggtgaaatgcgtagatattaggaggaacaccagtggcgaaggcggctttctggacgatgactgacgttgaggctcgaaagcgtggggagcaaacaggattagataccctggtagtccacgccgtaaacgatgaatactaggtgtcgggtggcaaagccattcggtgccgcagcaaacgcaataagtattccacctggggagtacgttcgcaagaatgaaactcaaaggaattgacggggacccgcacaagcggtggagcatgtggtttaattcgaagcaacgcgaagaaccttacctggtcttgacatccctctgaccgctctttaatcggagctttccttcgggacagaggagacaggtggtgcatggttgtcgtcagctcgtgtcgtgagatgttgggttaagtcccgcaacgagcgcaacccctatctttagtagccagcatttaaggtgggcactctagagagactgccagggataacctggaggaaggtggggatgacgtcaaatcatcatgccccttatgaccagggctacacacgtgctacaatggcgtaaacaaagggaagcgagcccgcgagggggagcaaatcccaaaaataacgtctcagttcggattgtagtctgcaactcgactacntgaagctggaatcgctagtaatcgcgaatca

SEQ ID NO: 3 - DNA, 722 bp, Enterococcus durans
  misc_feature: HB-48_Enterococcus_durans
tgcaagtcgtacgcttctttttccaccggagcttgctccaccggaaaaagaagagtggcgaacgggtgagtaacacgtgggtaacctgcccatcagaaggggataacacttggaaacaggtgctaataccgtataacaatcgaaaccgcatggttttgatttgaaaggcgctttcgggtgtcgctgatggatggacccgcggtgcattagctagttggtgaggtaacggctcaccaaggctacgatgcatagccgacctgagagggtgatcggccacattgggactgagacacggcccaaactcctacgggaggcagcagtagggaatcttcggcaatggacgaaagtctgaccgagcaacgccgcgtgagtgaagaaggttttcggatcgtaaaactctgttgttagagaagaacaaggatgagagtaactgttcatcccttgacggtatctaaccagaaagccacggctaactacgtgccagcagccgcggtaatacgtaggtggcaagcgttgtccggatttattgggcgtaaagcgagcgcaggcggtttcttaagtctgatgtgaaagcccccggctcaaccggggagggtcattggaaactgggagacttgagtgcagaagaggagagtggaattccatgtgtagcggtgaaatgcgtagatatatggaggaacaccagtggcgaaggcggctctctggtctgtaactgacgctga

SEQ ID NO: 4 - DNA, 1268 bp, Clostridium lavalense
  misc_feature: HB-452c_Clostridium_lavalense
  misc_feature (1069)..(1070): n is a, c, g, or t
tgcagtcgaacgaagcattttagatgaagttttcggatggattctgagatgactgagtggcggacgggtgagtaacacgtggataacctgcctcacactgggggacaacagttagaaatgactgctaataccgcataagcgcacagtaccgcatggtacggtgtgaaaaactccggtggtgtgagatggatccgcgtctgattagccagttggcggggtaacggcccaccaaagcgacgatcagtagccgacctgagagggtgaccggccacattgggactgagacacggcccaaactcctacgggaggcagcagtggggaatattgcacaatgggcgaaagcctgatgcagcgacgccgcgtgagtgaagaagtatttcggtatgtaaagctctatcagcagggaagataatgacggtacctgactaagaagccccggctaactacgtgccagcagccgcggtaatacgtagggggcaagcgttatccggatttactgggtgtaaagggagcgtagacggcatggcaagtctgaagtgaaaacccagggctcaaccctgggactgctttggaaactgtcaagctagagtgcaggagaggtaagtggaattcctagtgtagcggtgaaatgcgtagatattaggaggaacaccagtggcgaaggcggcttactggactgtaactgacgttgaggctcgaaagcgtggggagcaaacaggattagataccctggtagtccacgccgtaaacgatgagtgctaggtgttggggggcaaagcccttcggtgccgtcgcaaacgcaataagcactccacctggggagtacgttcgcaagaatgaaactcaaaggaattgacggggacccgcacaagcggtggagcatgtggtttaattcgaagcaacgcgaagaaccttaccaagtcttgacatcctcttgaccggcgtgtaacggcgcctttccttcgggacaagagagacaggtggtgcatggttgtcgtcagctcgtgtcgtgagatgttgggttaagtcccgcaacgagcgcaacccttatccttagtagccagcattnngatgggcactctagggagactgccagggacaacctggaggaaggtggggatgacgtcaaatcatcatgccccttatgatttgggctacacacgtgctacaatggcgtaaacaaagggaagcgaccctgcgaaggtgagcaaatctcaaaaataacgtcccagttcggactgtagtctgcaacccgactacacgaagct

SEQ ID NO: 5 - DNA, 627 bp, Hungatella effluvii
  misc_feature: HB-02_Hungatella_effluvii
aagttttcggatggaagaggatttgacttagcggcggacgggtgagtaacgcgtgggtaacctgccttacactgggggataacagttagaaatgactgctaataccgcataagcgcacagggccgcatggtctggtgtgaaaaactccggtggtgtaagatggacccgcgtctgattaggtagttggtggggtaacggcccaccaagccgacgatcagtagccgacctgagagggtgaccggccacattgggactgagacacggcccaaactcctacgggaggcagcagtggggaatattggacaatgggcgaaagcctgatccagcgacgccgcgtgagtgaagaagtatttcggtatgtaaagctctatcagcagggaagaaaatgacggtacctgactaagaagccccggctaactacgtgccagcagccgcggtaatacgtagggggcaagcgttatccggatttactgggtgtaaagggagcgtagacggtttagcaagtctgaagtgaaagcccggggctcaaccccggtactgctttggaaactgttagacttgagtgcaggagaggtaagtggaattcctagtgtagcggtgaaatgcgtagatattaggaggaacacc

SEQ ID NO: 6 - DNA, 1396 bp, Bacteroides caccae
  misc_feature: HB-11_Bacteroides_caccae
catgcaagtcgaggggcatcagtttggtttgcttgcaaaccaaagctggcgaccggcgcacgggtgagtaacacgtatccaacctgcctcatactcggggatagcctttcgaaagaaagattaatatccgatagcatatatttcccgcatgggttttatattaaagaaattcggtatgagatggggatgcgttccattagtttgttgggggggtaacggcccaccaagactacgatggataggggttctgagaggaaggtcccccacattggaactgagacacggtccaaactcctacgggaggcagcagtgaggaatattggtcaatggacgcgagtctgaaccagccaagtagcgtgaaggatgactgccctatgggttgtaaacttcttttatatgggaataaagttgtccacgtgtggatttttgtatgtaccatatgaataaggatcggctaactccgtgccagcagccgcggtaatacggaggatccgagcgttatccggatttattgggtttaaagggagcgtaggcggattgttaagtcagttgtgaaagtttgcggctcaaccgtaaaattgcagttgatactggcagtcttgagtgcagtagaggtgggcggaattcgtggtgtagcggtgaaatgcttagatatcacgaagaactccgattgcgaaggcagctcactggagtgtaactgacgctgatgctcgaaagtgtgggtatcaaacaggattagataccctggtagtccacacagtaaacgatgaatactcgctgtttgcgatatacagtaagcggccaagcgaaagcattaagtattccacctggggagtacgccggcaacggtgaaactcaaaggaattgacgggggcccgcacaagcggaggaacatgtggtttaattcgatgatacgcgaggaaccttacccgggcttaaattgcaaatgaattatggggaaacccatacgccgcaaggcatttgtgaaggtgctgcatggttgtcgtcagctcgtgccgtgaggtgtcggcttaagtgccataacgagcgcaacccttatcttcagttactaacaggtcatgctgaggactctggagagactgccgtcgtaagatgtgaggaaggtggggatgacgtcaaatcagcacggcccttacgtccggggctacacacgtgttacaatggggggtacagaaggccgctacctggtgacaggatgccaatcccaaaaacctctctcagttcggatcgaagtctgcaacccgacttcgtgaagctggattcgctagtaatcgcgcatcagccatggcgcggtgaatacgttcccgggccttgtacacaccgcccgtcaagccatgaaagccgggggtacctgaagtacgtaaccgcaaggagc

SEQ ID NO: 7 - DNA, 1400 bp, Bacteroides dorei
  misc_feature: HB-12_Bacteroides_dorei
gcagtcgaggggcagcatggtcttagcttgctaaggctgatggcgaccggcgcacgggtgagtaacacgtatccaacctgccgtctactcttggccagccttctgaaaggaagattaatccaggatgggatcatgagttcacatgtccgcatgattaaaggtattttccggtagacgatggggatgcgttccattagatagtaggcggggtaacggcccacctagtcaacgatggataggggttctgagaggaaggtcccccacattggaactgagacacggtccaaactcctacgggaggcagcagtgaggaatattggtcaatgggcgatggcctgaaccagccaagtagcgtgaaggatgactgccctatgggttgtaaacttcttttataaaggaataaagtcgggtatgcatacccgtttgcatgtactttatgaataaggatcggctaactccgtgccagcagccgcggtaatacggaggatccgagcgttatccggatttattgggtttaaagggagcgtagatggatgtttaagtcagttgtgaaagtttgcggctcaaccgtaaaattgcagttgatactggatgtcttgagtgcagttgaggcaggcggaattcgtggtgtagcggtgaaatgcttagatatcacgaagaactccgattgcgaaggcagcctgctaagctgcaactgacattgaggctcgaaagtgtgggtatcaaacaggattagataccctggtagtccacacggtaaacgatgaatactcgctgtttgcgatatacggcaagcggccaagcgaaagcgttaagtattccacctggggagtacgccggcaacggtgaaactcaaaggaattgacgggggcccgcacaagcggaggaacatgtggtttaattcgatgatacgcgaggaaccttacccgggcttaaattgcactcgaatgatccggaaacggttcagctagcaatagcgagtgtgaaggtgctgcatggttgtcgtcagctcgtgccgtgaggtgtcggcttaagtgccataacgagcgcaacccttgttgtcagttactaacaggtgatgctgaggactctgacaagactgccatcgtaagatgtgaggaaggtggggatgacgtcaaatcagcacggcccttacgtccggggctacacacgtgttacaatggggggtacagagggccgctaccacgcgagtggatgccaatccctaaaacccctctcagttcggactggagtctgcaacccgactccacgaagctggattcgctagtaatcgcgcatcagccacggcgcggtgaatacgttcccgggccttgtacacaccgcccgtcaagccatgggagccgggggtacctgaagtgcgtaaccgcgaggatcgccct

SEQ ID NO: 8 - DNA, 1386 bp, Clostridium saudiense
  misc_feature: HB-142_Clostridium_saudiense
agtcgagcgagtgaatctccttcgggagtgaagctagcggcggacgggtgagtaacacgtgggcaacctgcctcatagaggggaatagccttccgaaaggaagattaataccgcataagattgtagcttcgcatgaagtagcaattaaaggagcaatccgctatgagatgggcccgcggcgcattagctagttggtgaggtaacggctcaccaaggcgacgatgcgtagccgacctgagagggtgatcggccacattgggactgagacacggcccagactcctacgggaggcagcagtggggaatattgcacaatgggggaaaccctgatgcagcaacgccgcgtgagtgatgacggccttcgggttgtaaagctctgtcttcagggacgataatgacggtacctgaggaggaagccacggctaactacgtgccagcagccgcggtaatacgtaggtggcgagcgttgtccggatttactgggcgtaaagggagcgtaggcggacttttaagtgagatgtgaaatacccgggctcaacttgggtgctgcatttcaaactggaagtctagagtgcaggagaggagaatggaattcctagtgtagcggtgaaatgcgtagagattaggaagaacaccagtggcgaaggcgattctctggactgtaactgacgctgaggctcgaaagcgtggggagcaaacaggattagataccctggtagtccacgccgtaaacgatgaatactaggtgtaggggttgtcatgacctctgtgccgccgctaacgcattaagtattccgcctggggagtacggtcgcaagattaaaactcaaaggaattgacgggggcccgcacaagcagcggagcatgtggtttaattcgaagcaacgcgaagaaccttacctagacttgacatctcctgcattacccttaatcggggaagttccttcgggaacaggaagacaggtggtgcatggttgtcgtcagctcgtgtcgtgagatgttgggttaagtcccgcaacgagcgcaacccttattgttagttgctaccatttagttgagcactctagcgagactgcccgggttaaccgggaggaaggtggggatgacgtcaaatcatcatgccccttatgtctagggctacacacgtgctacaatggcaagtacaaagagaagcaagaccgcgaggtggagcaaaactcaaaaacttgtctcagttcggattgtaggctgaaactcgcctacatgaagctggagttgctagtaatcgcgaatcagcatgtcgcggtgaatacgttcccgggccttgtacacaccgcccgtcacaccatgagagttggcaatacccaaagtgcgtgatctgactcgcaagagaggaagcgccctaagg

SEQ ID NO: 9 - DNA, 1380 bp, Gordonibacter pamelaeae
  misc_feature: HB-15_Gordonibacter_pamelaeae
tgcagtcgaacgattaagacggctccggccgtgaatagagtggcgaacgggtgagtaacacgtgaccaacctgccccctaccccgggataacgcaaggaaacctgcgctaataccggatactccttcccctgctcctgcaggggtcgggaaagcccagacggtaggggatggggtcgcggcccattaggtagacggcggggcaacggcccaccgtgcctgcgatgggtagccgggttgagagaccgaccggccacattgggactgagatacggcccagactcctacgggaggcagcagtggggaattttgcgcaatggggggaaccctgacgcagcaacgccgcgtgcgggatgaaggccttcgggttgtaaaccgctttcagcagggaagatagtgacggtacctgcagaagaagccccggctaactacgtgccagcagccgcggtaatacgtagggggcgagcgttatccggattcattgggcgtaaagcgcgcgtaggcggcccgtcaagcggaacctctaacccgagggctcaacccccggtcgggttccgaactggcaggctcgagtttggtagaggaagatggaattcccggtgtagcggtggaatgcgcagatatcgggaagaacaccgatggcgaaggcagtcttctgggccatcaactgacgctgaggcgcgaaagctgggggagcgaacaggattagataccctggtagtcccagccgtaaacgatgggtgctaggtgtggggggatcatccctccgtgccgcagccaacgcattaagcaccccgcctggggagtacggccgcaaggctaaaactcaaaggaattgacgggggcccgcacaagcagcggagcatgtggcttaattcgaagcaacgcgaagaaccttaccagggcttgacatgctggtgaagccggggaaacccggtggccgagaggagctagcgcaggtggtgcatggctgtcgtcagctcgtgtcgtgagatgttgggttaagtcccgcaacgagcgcaacccctatcgtatgttgccagcattgagttggggactcatacgagactgccggcgtcaagccggaggaaggtggggacgacgtcaagtcatcatgccctttatgccctgggctgcacacgtgctacaatggccggtacaacgggccgcgacctggcgacaggaagcgaatccctcaaagccggccccagttcggatcggaggctgcaacccgcctccgtgaagtcggagttgctagtaatcgcggatcagcatgccgcggtgaatacgttcccgggccttgtacacaccgcccgtcacaccacccgagtcgtctgcacccgaagccgccggccgaacccgcaaggggcggaggcgtcgaa

SEQ ID NO: 10 - DNA, 1341 bp, Clostridium hathewayi
  misc_feature: HB-152_Clostridium_hathewayi
gaagttttcggatggattcttatttaccgagtggcggacgggtgagtaacgcgtgggtaacctgcctcatacagggggataacgattggaaacgattgctaataccgcataagcgcacagtaccacatggtgcagtgtgaaaaactccggtggtatgagatggacccgcgtctgattagctagttggtgaggtaacggcccaccaaggcaacgatcagtagccgacctgagagggtgaccggccacattgggactgagacacggcccagactcctacgggaggcagcagtgggggatattgcacaatggaggaaactctgatgcagcgacgccgcgtgagtgaagaagtatttcggtatgtaaagctctatcagcagggaagaaaatgacggtacctgactaagaagccccggctaactacgtgccagcagccgcggtaatacgtagggggcaagcgttatccggatttactgggtgtaaagggagcgtagacggcgatgcaagtctgaagtgaaatacccgggctcaacctgggaactgctttggaaactgtatggctagagtgctggagaggtaagcggaattcctagtgtagcggtgaaatgcgtagatattaggaagaacaccagtggcgaaggcggcttactggacagtaactgacgttcaggctcgaaagcgtggggagcaaacaggattagataccctggtagtccacgccgtaaacgatgaatactaggtgtcgggggacaaagtccttcggtgccgccgcaaacgcaataagtattccacctgggagtacgttcgcaagaatgaaactcaaaggaattgacggggacccgcacaagcggtggagcatgtggtttaattcgaagcaacgcgaagaaccttaccaaatcttgacatccctctgaaacacttttaatcgagtgcctccttcgggacagaggagacaggtggtgcatggttgtcgtcagctcgtgtcgtgagatgttgggttaagtcccgcaacgagcgcaacccctattgtcagtagccagcaggtaaagctgggcactctgatgagactgccagggataacctggaggaaggtggggatgacgtcaaatcatcatgccccttatgatttgggctacacacgtgctacaatggcgtaaacaaagagaagcgaagctgtgaggcagagcaaatctcaaaaataacgtctcagttcggattgtagtctgcaactcgactacatgaagctggaatcgctagtaatcgcggatcagaatgccgcggtgaatacgttcccgggtcttgtacacaccgcccgtcacaccatgggagtcggaaatgcccgaa

SEQ ID NO: 11 - DNA, 1352 bp, Bifidobacterium faecale
  misc_feature: HB-159_Bifidobacterium_faecale
tcccaggagcttgctcctgggtgagagtggcgaacgggtgagtaatgcgtgaccgacctgccccatacaccggaatagctcctggaaacgggtggtaatgccggatgctccagttgaccgcatggtcctctgggaaagattcatcggtatgggatggggtcgcgtcctatcagcttgatggcggggtaacggcccaccatggcttcgacgggtagccggcctgagagggcgaccggccacattgggactgagatacggcccagactcctacgggaggcagcagtggggaatattgcacaatgggcgcaagcctgatgcagcgacgccgcgtgcgggatgacggccttcgggttgtaaaccgcttttgactgggagcaagcccttcggggtgagtgtacctttcgaataagcaccggctaactacgtgccagcagccgcggtaatacgtagggtgcaagcgttatccggaattattgggcgtaaagggctcgtaggcggttcgtcgcgtccggtgtgaaagtccatcgcttaacggtggatccgcgccgggtacgggcgggcttgagtgcggtaggggagactggaattcccggtgtaacggtggaatgtgtagatatcgggaagaacaccaatggcgaaggcaggtctctgggccgtcactgacgctgaggagcgaaagcgtggggagcgaacaggattagataccctggtagtccacgccgtaaacggtggatgctggatgtggggaccattccacggtctccgtgtcggagccaacgcgttaagcatcccgcctggggagtacggccgcaaggctaaaactcaaagaaattgacgggggcccgcacaagcggcggagcatgcggattaattcgatgcaacgcgaagaaccttacctgggcttgacatgttcccgacagccccagagatggggcctcccttcggggcgggttcacaggtggtgcatggtcgtcgtcagctcgtgtcgtgagatgttgggttaagtcccgcaacgagcgcaaccctcgccctgtgttgccagcacgtcgtggtgggaactcacgggggaccgccggggtcaactcggaggaaggtggggatgacgtcagatcatcatgccccttacgtccagggcttcacgcatgctacaatggccggtacaacgggatgcgacaccgcgaggtggagcggatcccttaaaaccggtctcagttcggattggagtctgcaacccgactccatgaaggcggagtcgctagtaatcgcggatcagcaacgccgcggtgaatgcgttcccgggccttgtacacaccgcccgtcaagtcatgaaagtgggtagcacccgaagccgg

SEQ ID NO: 12 - DNA, 1079 bp, Bifidobacterium adolescentis
  misc_feature: HB-179_Bifidobacterium_adolescentis
ggcgcagcctgatgcagcgacgccgcgtgcgggatgacggccttcgggttgtaaaccgcttttgactgggagcaagcccttcggggtgagtgtacctttcgaataagcaccggctaactacgtgccagcagccgcggtaatacgtagggtgcaagcgttatccggaattattgggcgtaaagggctcgtaggcggttcgtcgcgtccggtgtgaaagtccatcgcttaacggtggatccgcgccgggtacgggcgggcttgagtgcggtaggggagactggaattcccggtgtaacggtggaatgtgtagatatcgggaagaacaccaatggcgaaggcaggtctctgggccgtcactgacgctgaggagcgaaagcgtggggagcgaacaggattagataccctggtagtccacgccgtaaacggtggatgctggatgtggggaccattccacggtctccgtgtcggagccaacgcgttaagcatcccgcctggggagtacggccgcaaggctaaaactcaaagaaattgacgggggcccgcacaagcggcggagcatgcggattaattcgatgcaacgcgaagaaccttacctgggcttgacatgttcccgacagccccagagatggggcctcccttcggggcgggttcacaggtggtgcatggtcgtcgtcagctcgtgtcgtgagatgttgggttaagtcccgcaacgagcgcaaccctcgccctgtgttgccagcacgtcatggtgggaactcacgggggaccgccggggtcaactcggaggaaggtggggatgacgtcagatcatcatgccccttacgtccagggcttcacgcatgctacaatggccggtacaacgggatgcgacctcgtgagggggagcggatcccttaaaaccggtctcagttcggattggagtctgcaacccgactccatgaaggcggagtcgctagtaatcgcggatcagcaacgccgcggtgaatgcgttcccgggccttgtacacaccgcccgtcaagtcatgaaagtgggtagcacccgaagccggtggcccaaccttttggggggagccgtctaa

SEQ ID NO: 13 - DNA, 1073 bp, Parabacteroides distasonis
  misc_feature: HB-20_Parabacteroides_distasonis
  misc_feature (16)..(16): n is a, c, g, or t
  misc_feature (20)..(20): n is a, c, g, or t
ggcgcgagcctgaccngccnagtcgcgtgagggatgaaggttctatggatcgtaaacctcttttataagggaataaagtgcgggacgtgtcccgttttgtatgtaccttatgaataaggatcggctaactccgtgccagcagccgcggtaatacggaggatccgagcgttatccggatttattgggtttaaagggtgcgtaggcggccttttaagtcagcggtgaaagtctgtggctcaaccatagaattgccgttgaaactggggggcttgagtatgtttgaggcaggcggaatgcgtggtgtagcggtgaaatgcttagatatcacgcagaaccccgattgcgaaggcagcctgccaagccatgactgacgctgatgcacgaaagcgtggggatcaaacaggattagataccctggtagtccacgcagtaaacgatgatcactagctgtttgcgatacagtgtaagcggcacagcgaaagcgttaagtgatccacctggggagtacgccggcaacggtgaaactcaaaggaattgacgggggcccgcacaagcggaggaacatgtggtttaattcgatgatacgcgaggaaccttacccgggtttgaacgcattcggaccgaggtggaaacaccttttctagcaatagccgtttgcgaggtgctgcatggttgtcgtcagctcgtgccgtgaggtgtcggcttaagtgccataacgagcgcaacccttgccactagttactaacaggtgatgctgaggactctggtgggactgccagcgtaagctgcgaggaaggcggggatgacgtcaaatcagcacggcccttacatccggggcgacacacgtgttacaatggcgtggacaaagggaagccacctggcgacagggagcgaatccccaaaccacgtctcagttcggatcggagtctgcaacccgactccgtgaagctggattcgctagtaatcgcgcatcagccatggcgcggtgaatacgttcccgggccttgtacacaccgcccgtcaagccatgggagccgggggtacctgaagtccgtaaccgaaaggatcggcct

SEQ ID NO: 14 - DNA, 1411 bp, Eubacterium eligens
  misc_feature: HB-252_Eubacterium_eligens
aagtcgaacgaagcatttagcagacagattatcttcggtgtatgaagttcgtcttatgactgagtggcggacgggtgagtaacgcgtgggtaacctgccttgtactgggggatagcagctggaaacggctggtaataccgcataagcgcacaatgttgcatgacatggtgtgaaaaactccggtggtataagatggacccgcgtctgattagctagttggtgagataacagcccaccaaggcgacgatcagtagccgacctgagagggtgaccggccacattgggactgagacacggcccagactcctacgggaggcagcagtggggaatattgcacaatggaggaaactctgatgcagcgacgccgcgtgagtgaagaagtaattcgttatgtaaagctctatcagcagggaagatagtgacggtacctgactaagaagctccggctaaatacgtgccagcagccgcggtaatacgtatggagcaagcgttatccggatttactgggtgtaaagggagtgtaggtggccatgcaagtcagaagtgaaaatccggggctcaaccccggaactgcttttgaaactgtaaggctrgagtgcaggaggggtgagtggaattcctagtgtagcggtgaaatgcgtagatattaggaggaacaccagtggcgaaggcggctcactggactgtaactgacactgaggctcgaaagcgtggggagcaaacaggattagataccctggtagtccacgccgtaaacgatgaatactaggtgtcggggcccataagggcttcggtgccgcagcaaacgcaataagtattccacctggggagtacgttcgcaagaatgaaactcaaaggaattgacggggacccgcacaagcggtggagcatgtggtttaattcgaagcaacgcgaagaaccttaccaagtcttgacatcccactgaccggacagtaatgtgtcctttccttcgggacagtggagacaggtggtgcatggttgtcgtcagctcgtgtcgtgagatgttgggttaagtcccgcaacgagcgcaacccctatccttagtagccagcagtaagatgggcactctagggagactgccagggataacctggaggaaggtggggatgacgtcaaatcatcatgccccttatgacttgggctacacacgtgctacaatggcgtaaacaaagtgaagcgaagtcgtgaggccaagcaaatcacaaaaataacgtctcagttcggattgtagtctgcaactcgactacatgaagctggaatcgctagtaatcgcagatcagaatgctgcggtgaatacgttcccgggtcttgtacacaccgcccgtcacaccatgggagtcgaaaatgcccgaagtcggtgacctaacgaaagaaggagccgccgaaggca

SEQ ID NO: 15 - DNA, 1391 bp, Bacteroides finegoldii
  misc_feature: HB-31_Bacteroides_finegoldii
gcagtcgaggggcagcattttagtttgcttgcaaactaaagatggcgaccggcgcacgggtgagtaacacgtatccaacctgccgataactcagggatagcctttcgaaagaaagattaatacctgatggcataggattatcgcatgataatcctattaaagaatttcggttatcgatggggatgcgttccattaggcagttggtgaggtaacggctcaccaaaccttcgatggataggggttctgagaggaaggtcccccacattggaactgagacacggtccaaactcctacgggaggcagcagtgaggaatattggtcaatggacgggagtctgaaccagccaagtagcgtgaaggatgactgccctatgggttgtaaacttcttttatacgggaataaagtgatccacgtgtgggtttttgtatgtaccgtatgaataaggatcggctaactccgtgccagcagccgcggtaatacggaggatccgagcgttatccggatttattgggtttaaagggagcgtaggtggattgttaagtcagttgtgaaagtttgcggctcaaccgtaaaattgcagttgatactggcagtcttgagtacagtagaggtgggcggaattcgtggtgtagcggtgaaatgcttagatatcacgaagaactccgattgcgaaggcagctcactggactgcaactgacactgatgctcgaaagtgtgggtatcaaacaggattagataccctggtagtccacacagtaaacgatgaatactcgctgtttgcgatatacggtaagcggccaagcgaaagcgttaagtattccacctggggagtacgccggcaacggtgaaactcaaaggaattgacgggggcccgcacaagcggaggaacatgtggtttaattcgatgatacgcgaggaaccttacccgggcttaaattacatttgaataatctggaaacaggttagccgtaaggcaaatgtgaaggtgctgcatggttgtcgtcagctcgtgccgtgaggtgtcggcttaagtgccataacgagcgcaacccttatcttcagttactaacaggtcatgctgaggactctggagagactgccgtcgtaagatgtgaggaaggtggggatgacgtcaaatcagcacggcccttacgtccggggctacacacgtgttacaatggggggtacagaaggcagctacctggtgacaggatgctaatcccaaaaacctctctcagttcggatcgaagtctgcaacccgacttcgtgaagctggattcgctagtaatcgcgcatcagccatggcgcggtgaatacgttcccgggccttgtacacaccgcccgtcaagccatgaaagccgggggtacctgaagtacgtaaccgcgaggag

SEQ ID NO: 16 - DNA, 1400 bp, Bacteroides salyersiae
  misc_feature: HB-32_Bacteroides_salyersiae
  misc_feature (1387)..(1387): n is a, c, g, or t
gcagtcgaggggcatcagggtgtagcaatacaccgctggcgaccggcgcacgggtgagtaacacgtatccaacctgccctttactcggggatagcctttcgaaagaaagattaatacccgatggcataacataacctcctggttttgttattaaagaatttcggtagaggatggggatgcgttccattaggcagttggcggggtaacggcccaccaaaccttcgatggataggggttctgagaggaaggtcccccacattggaactgagacacggtccaaactcctacgggaggcagcagtgaggaatattggtcaatgggcgagagcctgaaccagccaagtagcgtgaaggatgaccgccctatgggttgtaaacttcttttatatgggaataaagtttgccacgtgtggcattttgtatgtaccatatgaataaggatcggctaactccgtgccagcagccgcggtaatacggaggatccgagcgttatccggatttattgggtttaaagggagcgtaggtggacatgtaagtcagttgtgaaagtttgcggctcaaccgtaaaattgcagttgatactgcgtgtcttgagtacagtagaggtgggcggaattcgtggtgtagcggtgaaatgcttagatatcacgaagaactccgattgcgaaggcagctcactggactgcaactgacactgatgctcgaaagtgtgggtatcaaacaggattagataccctggtagtccacacagtaaacgatgaatactcgctgtttgcgatatacagtaagcggccaagcgaaagcattaagtattccacctggggagtacgccggcaacggtgaaactcaaaggaattgacgggggcccgcacaagcggaggaacatgtggtttaattcgatgatacgcgaggaaccttacccgggcttaaattgcaaatgaatatgccggaaacggcatagccgcaaggcatttgtgaaggtgctgcatggttgtcgtcagctcgtgccgtgaggtgtcggcttaagtgccataacgagcgcaacccttatcttcagttactaacaggttatgctgaggactctggagagactgccgtcgtaagatgtgaggaaggtggggatgacgtcaaatcagcacggcccttacgtccggggctacacacgtgttacaatggggggtacagaaggccgctacacagcgatgtgatgccaatccctaaagcccctctcagttcggatcgaagtctgcaacccgacttcgtgaagctggattcgctagtaatcgcgcatcagccacggcgcggtgaatacgttcccgggccttgtacacaccgcccgtcaagccatgggagccgggggtacctgaagtacgtaaccgcaaggagcntcctagggtaaaa

SEQ ID NO: 17 - DNA, 1364 bp, Gemmiger formicilis
  misc_feature: HB-325_Gemmiger_formicilis
  misc_feature (125)..(125): n is a, c, g, or t
aggagcttgcttttcttgacttagtggcgaacgggtgagtaacgcgtgagtaacctgccctggagtgggggacaacagttggaaacgactgctaataccgcataagcccacggtccggcatcggnttgcgggaaaaggatttattcgcttcaggatggactcgcgtccaattagctagttggtgaggtaacggcccaccaaggcgacgattggtagccggactgagaggttgaacggccacattgggactgagacacggcccagactcctacgggaggcagcagtgggggatattgcacaatgggggaaaccctgatgcagcgacgccgcgtggaggaagaaggttttcggattgtaaactcctgtcgttagggacgataatgacggtacctaacaagaaagcaccggctaactacgtgccagcagccgcggtaaaacgtagggtgcaagcgttgtccggaattactgggtgtaaagggagcgcaggcggaccggcaagttggaagtgaaaactatgggctcaacccataaattgctttcaaaactgctggccttgagtagtgcagaggtaggtggaattcccggtgtagcggtggaatgcgtagatatcgggaggaacaccagtggcgaaggcgacctactgggcaccaactgacgctgaggctcgaaagcatgggtagcaaacaggattagataccctggtagtccatgccgtaaacgatgattactaggtgttggaggattgaccccttcagtgccgcagttaacacaataagtaatccacctggggagtacgaccgcaaggttgaaactcaaaggaattgacgggggcccgcacaagcagtggagtatgtggtttaattcgaagcaacgcgaagaaccttaccaggtcttgacatccgatgcatagcgcagagatgcgtgaagtccttcgggacatcgagacaggtggtgcatggttgtcgtcagctcgtgtcgtgagatgttgggttaagtcccgcaacgagcgcaacccttattgccagttactacgcaagaggactctggcgagactgccgttgacaaaacggaggaaggtggggatgacgtcaaatcatcatgccctttatgacctgggctacacacgtactacaatggcgtttaacaaagagaagcaagaccgcgaggtggagcaaaactcaaaaacaacgtctcagttcagattgcaggctgcaactcgcctgcatgaagtcggaattgctagtaatcgcggatcagcatgccgcggtgaatacgttcccgggccttgtacacaccgcccgtcacaccatgagagccggggggacccgaagtcggtagtctaaccgcaaggaggacgccgccga

SEQ ID NO: 18 - DNA, 1413 bp, Parasutterella excrementihominis
  misc_feature: HB-330_Parasutterella_excrementihominis
tgcaagtcgaacggtaacgcggagagaagcttgcttctcatccggcgacgagtggcgaacgggtgagtaatacatcggaacgtgtccgctcgtgggggacaaccagccgaaaggttggctaataccgcatgagttctacggaagaaagagggggacccgcaagggcctctcgcgagcggagcggccgatgactgattagccggttggtgaggtaacggctcaccaaagcaacgatcagtagctggtctgagaggacgaccagccacactgggactgagacacggcccagactcctacgggaggcagcagtggggaattttggacaatgggcgcaagcctgatccagctattccgcgtgtgggatgaaggccctcgggttgtaaaccacttttgtagagaacgaaaagacaccttttaaaaaagggtgttgctgacggtactctaagaataagcaccggctaactacgtgccagcagccgcggtaatacgtagggtgcgagcgttaatcggaattactgggcgtaaagggtgcgcaggcggttgagtaagacagatgtgaaatccccgagcttaactcgggaatggcatatgtgactgctcgactagagtgtgtcagagggaggtggaattccacgtgtagcagtgaaatgcgtagatatgtggaagaacaccgatggcgaaggcagcctcctgggacataactgacgctcaggcacgaaagcgtggggagcaaacaggattagataccctggtagtccacgccctaaacgatgttaactagttgttgggaagtaaaattctcagtaacgcagccaacgcgagaagttaaccgcctgggaagtacggtcgcaagactaaaactcaaaggaattgacggggacccgcacaagcggtggatgatgtggattaattcgatgcaacgcgaaaaaccttacctacccttgacatgtcaggaagctcttgtaatgagagcgtgcccgcaagggagcctgaacacaggtgctgcatggctgtcgtcagctcgtgtcgtgagatgttgggttaagtcccgcaacgagcgcaacccttgtcactagttgctacgaaagggcactctagtgagactgccggtgacaaaccggaggaaggtggggatgacgtcaagtcctcatggcccttatgggtagggcttcacacgtcatacaatggtcggaacagagggcagcgaagccgtgaggcggagccaatcccagaaaaccgatcgtagtccggattgcagtctgcaactcgactgcatgaagtcggaatcgctagtaatcgcggatcagcatgccgcggtgaatacgttcccgggtcttgtacacaccgcccgtcaaacaatgggagtggtgtttaccagaagtcgttagcctaaccgcaaggagggcggcgacc

SEQ ID NO: 19 - DNA, 1392 bp, Bacteroides xylanisolvens
  misc_feature: HB-35_Bacteroides_xylanisolvens
tgcagtcgaggggcagcattttagtttgcttgcaaactaaagatggcgaccggcgcacgggtgagtaacacgtatccaacctgccgataactcggggatagcctttcgaaagaaagattaatatccgatagtatattaaaaccgcatggttttactattaaagaatttcggttatcgatggggatgcgttccattagtttgttggcggggtaacggcccaccaagactacgatggataggggttctgagaggaaggtcccccacattggaactgagacacggtccaaactcctacgggaggcagcagtgaggaatattggtcaatggacgagagtctgaaccagccaagtagcgtgaaggatgactgccctatgggttgtaaacttcttttatatgggaataaagtattccacgtgtgggattttgtatgtaccatatgaataaggatcggctaactccgtgccagcagccgcggtaatacggaggatccgagcgttatccggatttattgggtttaaagggagcgtaggtggattgttaagtcagttgtgaaagtttgcggctcaaccgtaaaattgcagttgaaactggcagtcttgagtacagtagaggtgggcggaattcgtggtgtagcggtgaaatgcttagatatcacgaagaactccgattgcgaaggcagctcactagactgcaactgacactgatgctcgaaagtgtgggtatcaaacaggattagataccctggtagtccacacagtaaacgatgaatactcgctgtttgcgatatacagtaagcggccaagcgaaagcattaagtattccacctggggagtacgccggcaacggtgaaactcaaaggaattgacgggggcccgcacaagcggaggaacatgtggtttaattcgatgatacgcgaggaaccttacccgggcttaaattgcaaatgaataatctggaaacaggttagccgcaaggcatttgtgaaggtgctgcatggttgtcgtcagctcgtgccgtgaggtgtcggcttaagtgccataacgagcgcaacccttatctttagttactaacaggttatgctgaggactctagagagactgccgtcgtaagatgtgaggaaggtggggatgacgtcaaatcagcacggcccttacgtccggggctacacacgtgttacaatggggggtacagaaggcagctacctggcgacaggatgctaatcccaaaaacctctctcagttcggatcgaagtctgcaacccgacttcgtgaagctggattcgctagtaatcgcgcatcagccatggcgcggtgaatacgttcccgggccttgtacacaccgcccgtcaagccatgaaagccgggggtacctgaagtacgtaaccgcaaggag

SEQ ID NO: 20 - DNA, 1358 bp, Mediterraneibacter faecis
  misc_feature: HB-364_Mediterraneibacter_faecis
ttggtgactgagtggcggacgggtgagtaacgcgtgggtaacctgcctcatacagggggataacagttagaaatgactgctaataccgcataagaccacagcaccgcatggtgcaggggtaaaaactccggtggtatgagatggacccgcgtctgattaggtagttggtggggtaacggcctaccaagccgacgatcagtagccgacctgagagggtgaccggccacattgggactgagacacggcccaaactcctacgggaggcagcagtggggaatattgcacaatgggggaaaccctgatgcagcgacgccgcgtgagcgatgaagtatttcggtatgtaaagctctatcagcagggaagaaaatgacggtacctgactaagaagcaccggctaaatacgtgccagcagccgcggtaatacgtatggtgcaagcgttatccggatttactgggtgtaaagggagcgtagacggagtggcaagtctgatgtgaaaacccggggctcaaccccgggactgcattggaaactgtcaatctagagtaccggagaggtaagcggaattcctagtgtagcggtgaaatgcgtagatattaggaggaacaccagtggcgaaggcggcttactggacggtaactgacgttgaggctcgaaagcgtggggagcaaacaggattagataccctggtagtccacgccgtaaacgatgactactaggtgtcgggcagcaaagctgttcggtgccgcagcaaacgcaataagtagtccacctggggagtacgttcgcaagaatgaaactcaaaggaattgacggggacccgcacaagcggtggagcatgtggtttaattcgaagcaacgcgaagaaccttacctgctcttgacatctccctgaccggcaagtaatgttgcctttccttcgggacagggatgacaggtggtgcatggttgtcgtcagctcgtgtcgtgagatgttgggttaagtcccgcaacgagcgcaacccctatctttagtagccagcggtttggccgggcactctagagagactgccagggataacctggaggaaggtggggatgacgtcaaatcatcatgccccttatgagcagggctacacacgtgctacaatggcgtaaacaaagggaggcagaaccgcgaggtcgagcaaatcccaaaaataacgtctcagttcggattgtagtctgcaactcgactacatgaagctggaatcgctagtaatcgcgaatcagaatgtcgcggtgaatacgttcccgggtcttgtacacaccgcccgtcacaccatgggagtcagtaacgcccgaagtcagtgacccaaccgtaaggagggagctgccgaag

SEQ ID NO: 21 - DNA, 1403 bp, Clostridium aldenese
  misc_feature: HB-440_Clostridium_aldenese
  misc_feature (264)..(264): n is a, c, g, or t
tgcagtcgaacgaagcatcccataggaagttttcggatggaatatgggatgactgagtggcggacgggtgagtaacgcgtggataacctgcctcacactgggggataacagttagaaatggctgctaataccgcataagcgcacagtaccgcatggtacggtgtgaaaaacccaggtggtgtgagatggatccgcgtctgattagccagttggcggggtaacggcccaccaaagcgacgatcagtagccgacctgagagggtgnccggccacattgggactgagacacggcccaaactcctacgggaggcagcagtggggaatattgcacaatgggcgaaagcctgatgcagcgacgccgcgtgagtgaagaagtatctcggtatgtaaagctctatcagcagggaagaaaatgacggtacctgactaagaagccccggctaactacgtgccagcagccgcggtaatacgtagggggcaagcgttatccggatttactgggtgtaaagggagcgtagacggcgatgcaagtctgaagtgaaagcccggggctcaaccccgggactgctttggaaactgtgtggctggagtgcaggagaggtaagtggaattcctagtgtagcggtgaaatgcgtagatattaggaggaacaccagtggcgaaggcggcttactggactgtaactgacgttgaggctcgaaagcgtggggagcaaacaggattagataccctggtagtccacgccgtaaacgatgaatgctaggtgtcggggggcaaagcccctcggtgccgccgctaacgcaataagcattccacctggggagtacgttcgcaagaatgaaactcaaaggaattgacggggacccgcacaagcggtggagcatgtggtttaattcgaagcaacgcgaagaaccttaccaagtcttgacatccccctgaccggacagtaacgtgtcccttccttcgggacaggggagacaggtggtgcatggttgtcgtcagctcgtgtcgtgagatgttgggttaagtcccgcaacgagcgcaacccttatccttagtagccagcacctcgggtgggcactctagggagactgccagggataacctggaggaaggtggggatgacgtcaaatcatcatgccccttatgatttgggctacacacgtgctacaatggcgtaaacaaagggaggcggccctgcgaaggtgagcaaatcccaaaaataacgtcccagttcggactgtagtctgcaacccgactacacgaagctggaatcgctagtaatcgcggatcagaatgccgcggtgaatacgttcccgggtcttgtacacaccgcccgtcacaccatgggagtcagcaacgcccgaagtcagtgacccaaccgataaggagggagctgccgaa

SEQ ID NO: 22 - DNA, 1406 bp, Clostridium bolteae
  misc_feature: HB-442_Clostridium_bolteae
tgcagtcgaacgaagcaattaaaatgaagttttcggatggatttttgattgactgagtggcggacgggtgagtaacgcgtggataacctgcctcacactgggggataacagttagaaatgactgctaataccgcataagcgcacagtaccgcatggtacggtgtgaaaaactccggtggtgtgagatggatccgcgtctgattagccagttggcggggtaacggcccaccaaagcgacgatcagtagccgacctgagagggtgaccggccacattgggactgagacacggcccaaactcctacgggaggcagcagtggggaatattgcacaatgggcgaaagcctgatgcagcgacgccgcgtgagtgaagaagtatttcggtatgtaaagctctatcagcagggaagaaaatgacggtacctgactaagaagccccggctaactacgtgccagcagccgcggtaatacgtagggggcaagcgttatccggatttactgggtgtaaagggagcgtagacggcgaagcaagtctgaagtgaaaacccagggctcaaccctgggactgctttggaaactgttttgctagagtgtcggagaggtaagtggaattcctagtgtagcggtgaaatgcgtagatattargaggaacaccagtggcgaaggcggcttactggacgataactgacgttgaggctcgaaagcgtggggagcaaacaggattakataccctggtagtccacgccgtaaacgatgaatgctaggtgttgggggggcaaagcccttcggtgccgtcgcaaacgcagtaagcattccacctggggagtacgttcgcaagaatgaaactcaaaggaattgacggggacccgcacaagcggtggagcatgtggtttaattcgaagcaacgcgaagaaccttaccaagtcttgacatcctcttgaccggcgtgtaacggcgccttcccttcggggcaagagagacaggtggtgcatggttgtcgtcagctcgtgtcgtgagatgttgggttaagtcccgcaacgagcgcaacccttatccttagtagccagcaggtagagctgggcactctagggagactgccagggataacctggaggaaggtggggatgacgtcaaatcatcatgccccttatgatttgggctacacacgtgctacaatggcgtaaacaaagggaagcaagacagtgatgtggagcaaatcccaaaaataacgtcccagttcggactgtagtctgcaacccgactacacgaagctggaatcgctagtaatcgcgaatcagaatgtcgcggtgaatacgttcccgggtcttgtacacaccgcccgtcacaccatgggagtcagcaacgcccgaagtcagtgacccaactcgcaagagagggagctgccgaa

SEQ ID NO: 23 - DNA, 1057 bp, Clostridium scindens
  misc_feature: HB-444_Clostridium_scindens
tgcagcgacgccgcgtgaaggatgaagtatttcggtatgtaaacttctatcagcagggaagaagatgacggtacctgactaagaagccccggctaactacgtgccagcagccgcggtaatacgtagggggcaagcgttatccggatttactgggtgtaaagggagcgtagacggcgatgcaagccagatgtgaaagcccggggctcaaccccgggactgcatttggaactgcgtggctggagtgtcggagaggcaggcggaattcctagtgtagcggtgaaatgcgtagatattaggaggaacaccagtggcgaaggcggcctgctggacgatgactgacgttgaggctcgaaagcgtggggagcaaacaggattagataccctggtagtccacgccgtaaacgatgactactaggtgtcgggtggcaaggccattcggtgccgcagcaaacgcaataagtagtccacctggggagtacgttcgcaagaatgaaactcaaaggaattgacggggacccgcacaagcggtggagcatgtggtttaattcgaagcaacgcgaagaaccttacctgatcttgacatcccgatgccaaagcgcgtaacgcgctctttcttcggaacatcggtgacaggtggtgcatggttgtcgtcagctcgtgtcgtgagatgttgggttaagtcccgcaacgagcgcaacccctatcttcagtagccagcatttyggatgggcactctggagagactgccagggacaacctggaggaaggtggggatgacgtcaaatcatcatgccccttatgaccagggctacacacgtgctacaatggcgtaaacaaagggaggcgaacccgcgagggtgggcaaatcccaaaaataacgtctcagttcggattgtagtctgcaactcgactacatgaagctggaatcgctagtaatcgcgaatcagaatgtcgcggtgaatacgttcccgggtcttgtacacaccgcccgtcacaccatgggagtcagtaacgcccgaagccggtgacccaacccgcaagggaggagccgtcgaag

SEQ ID NO: 24 - DNA, 1381 bp, Clostridium tyrobutyricum
  misc_feature: HB-469_Clostridium_tyrobutyricum
tgcagtcgagcgatgaaccccttcgggggtggattagcggcggacgggtgagtaacacgtgggtaacctgcctcaaagtgggggatagccttccgaaaggaagattaataccgcataaagccaagtttcacatggaatttggatgaaaggagtaattcgctttgagatggacccgcggcgcattagttagttggtggggtaatggcctaccaagacagcgatgcgtagccgacctgagagggtgatcggccacattggaactgagatacggtccagactcctacgggaggcagcagtggggaatattgcacaatgggcgaaagcctgatgcagcaacgccgcgtgagtgatgaaggtcttcggattgtaaagctctgtcttttgggacgataatgacggtaccaaaggaggaagccacggctaactacgtgccagcagccgcggtaatacgtaggtggcgagcgttgtccggatttactgggcgtaaagggtgcgtaggcggatgtttaagtgagatgtgaaatacccgggcttaacttgggtgctgcatttcaaactggatatctagagtgcaggagaggagaatggaattcctagtgtagcggtgaaatgcgtagagattaggaagaacaccagtggcgaaggcgattctctggactgtaactgacgctgaggcacgaaagcgtgggtagcaaacaggattagataccctggtagtccacgccgtaaacgatgagtactaggtgtaggaggtatcgaccccttctgtgccgcagtaaacacattaagtactccgcctgggaagtacgatcgcaagattaaaactcaaaggaattgacgggggcccgcacaagcagcggagcatgtggtttaattcgaagcaacgcgaagaaccttacctggacttgacatcccctgaataacctagagataggcgaagcccttcggggcagggagacaggtggtgcatggttgtcgtcagctcgtgtcgtgagatgttaggttaagtcctgcaacgagcgcaacccttattgttagttgctaacattcagttgagcactctaacgagactgccgcggttaacgcggaggaaggtggggatgacgtcaaatcatcatgccccttatgtccagggcaacacacgtgctacaatgggcagaacaaagagaagcaataccgcgaggtggagccaaactcaaaaactgctctcagttcggattgcaggctgaaactcgcctgcatgaagctggagttgctagtaatcgcgaatcagcatgtcgcggtgaatacgttcccgggccttgtacacaccgcccgtcacaccatgagagctggcaacacccgaagtccgtagtctaacgtaagaggacgcggccgaagg

SEQ ID NO: 25 - DNA, 1402 bp, Eisenbergiella tayi
  misc_feature: HB-612_Eisenbergiella_tayi
tgcagtcgaacggagttatgcagaggaagttttcggatggaatcggcgtaacttagtggcggacgggtgagtaacgcgtgggaaacctgccctgtaccgggggataacacttagaaataggtgctaataccgcataagcgcacagcttcacatgaagcagtgtgaaaaactccggtggtacaggatggtcccgcgtctgattagccagttggcagggtaacggcctaccaaagcgacgatcagtagccggcctgagagggtgaacggccacattgggactgagacacggcccaaactcctacgggaggcagcagtggggaatattgcacaatgggggaaaccctgatgcagcgacgccgcgtgagtgaagaagtatttcggtatgtaaagctctatcagcagggaagaaaatgacggtacctgactaagaagccccggctaactacgtgccagcagccgcggtaatacgtagggggcaagcgttatccggatttactgggtgtaaagggagcgtagacggcatgacaagccagatgtgaaaacccagggctcaaccctgggactgcatttggaactgccaggctggagtgcaggagaggtaagcggaattcctagtgtagcggtgaaatgcgtagatattaggaggaacaccagtggcgaaggcggcttactggactgtaactgacgttgaggctcgaaagcgtggggagcaaacaggattagataccctggtagtccacgcggtaaacgatgattgctaggtgtaggtgggtatggacccatcggtgccgcagctaacgcaataagcaatccacctggggagtacgttcgcaagaatgaaactcaaaggaattgacggggacccgcacaagcggtggagcatgtggtttaattcgaagcaacgcgaagaaccttaccaagtcttgacatcccaatgacgtgtccgtaacggggcattctcttcggagcattggagacaggtggtgcatggttgtcgtcagctcgtgtcgtgagatgttgggttaagtcccgcaacgagcgcaacccttatccttagtagccagcaggtagagctgggcactctagggagactgccggggataacccggaggaaggcggggatgacgtcaaatcatcatgccccttatgatttgggctacacacgtgctacaatggcgtaaacaaagggaagcgagacagtgatgttgagcaaatcccagaaataacgtctcagttcggattgtagtctgcaactcgactacatgaagctggaatcgctagtaatcgcgaatcagcatgtcgcggtgaatacgttcccgggtcttgtacacaccgcccgtcacaccatgggagttggaaatgcccgaagcctgtgacctaaccgcaagggaggagcagtcga

SEQ ID NO: 26 - DNA, 1400 bp, Parabacteroides merdae
  misc_feature: HB-63_Parabacteroides_merdae
tgcagtcgaggggcagcatgatttgtagcaatacagattgatggcgaccggcgcacgggtgagtaacgcgtatgcaacttacctatcagagggggatagcccggcgaaagtcggattaataccccataaaacaggggtcccgcatgggaatatttgttaaagattcatcgctgatagataggcatgcgttccattaggcagttggcggggtaacggcccaccaaaccgacgatggataggggttctgagaggaaggtcccccacattggtactgagacacggaccaaactcctacgggaggcagcagtgaggaatattggtcaatggccgagaggctgaaccagccaagtcgcgtgaaggaagaaggatctatggtttgtaaacttcttttataggggaataaagtggaggacgtgtccttttttgtatgtaccctatgaataagcatcggctaactccgtgccagcagccgcggtaatacggaggatgcgagcgttatccggatttattgggtttaaagggtgcgtaggtggtgatttaagtcagcggtgaaagtttgtggctcaaccataaaattgccgttgaaactgggttacttgagtgtgtttgaggtaggcggaatgcgtggtgtagcggtgaaatgcatagatatcacgcagaactccgattgcgaaggcagcttactaaaccataactgacactgaagcacgaaagcgtggggatcaaacaggattagataccctggtagtccacgcagtaaacgatgattactaggagtttgcgatacaatgtaagctctacagcgaaagcgttaagtaatccacctggggagtacgccggcaacggtgaaactcaaaggaattgacgggggcccgcacaagcggaggaacatgtggtttaattcgatgatacgcgaggaaccttacccgggtttgaacgtagtctgaccggagtggaaacactctttctagcaatagcagattacgaggtgctgcatggttgtcgtcagctcgtgccgtgaggtgtcggcttaagtgccataacgagcgcaacccttatcactagttactaacaggtgaagctgaggactctggtgagactgccagcgtaagctgtgaggaaggtggggatgacgtcaaatcagcacggcccttacatccggggcgacacacgtgttacaatggcatggacaaagggcagctacctggtgacaggatgctaatctccaaaccatgtctcagttcggatcggagtctgcaactcgactccgtgaagctggattcgctagtaatcgcgcatcagccatggcgcggtgaatacgttcccgggccttgtacacaccgcccgtcaagccatgggagccgggggtacctgaagtccgtaaccgcaaggatcggcct

SEQ ID NO: 27 - DNA, 1393 bp, Bacteroides ovatus
  misc_feature: HB-70_Bacteroides_ovatus
tgcagtcgaggggcagcattttagtttgcttgcaaactgaagatggcgaccggcgcacgggtgagtaacacgtatccaacctgccgataactccgggatagcctttcgaaagaaagattaataccggatagcatacgaatatcgcatgatatttttattaaagaatttcggttatcgatggggatgcgttccattagtttgttggcggggtaacggcccaccaagacaacgatggataggggttctgagaggaaggtcccccacattggaactgagacacggtccaaactcctacgggaggcagcagtgaggaatattggtcaatgggcgagagcctgaaccagccaagtagcgtgaaggatgaaggctctatgggtcgtaaacttcttttatatgggaataaagtattccacgtgtggaattttgtatgtaccatatgaataaggatcggctaactccgtgccagcagccgcggtaatacggaggatccgagcgttatccggatttattgggtttaaagggagcgtaggtggattgttaagtcagttgtgaaagtttgcggctcaaccgtaaaattgcagttgaaactggcagtcttgagtacagtagaggtgggcggaattcgtggtgtagcggtgaaatgcttagatatcacgaagaactccgattgcgaaggcagctcactagactgtcactgacactgatgctcgaaagtgtgggtatcaaacaggattagataccctggtagtccacacagtaaacgatgaatactcgctgtttgcgatatacagtaagcggccaagcgaaagcattaagtattccacctggggagtacgccggcaacggtgaaactcaaaggaattgacgggggcccgcacaagcggaggaacatgtggtttaattcgatgatacgcgaggaaccttacccgggcttaaattgcaacagaatatattggaaacagtatagccgtaaggctgttgtgaaggtgctgcatggttgtcgtcagctcgtgccgtgaggtgtcggcttaagtgccataacgagcgcaacccttatctttagttactaacaggtcatgctgaggactctagagagactgccgtcgtaagatgtgaggaaggtggggatgacgtcaaatcagcacggcccttacgtccggggctacacacgtgttacaatggggggtacagaaggcggctacctggtgacaggatgctaatcccaaaaacctctctcagttcggatcgaagtctgcaacccgacttcgtgaagctggattcgctagtaatcgcgcatcagccatggcgcggtgaatacgttcccgggccttgtacacaccgcccgtcaagccatgaaagccgggggtacctgaagtacgtaaccgcaaggagc

SEQ ID NO: 28 - DNA, 865 bp, Anaerotruncus colihominis
  misc_feature: HB-83_Anaerotruncus_colihominis
cggagcttacgttttgaagttttcggatggatgaatgtaagcttagtggcggacgggtgagtaacacgtgagcaacctgcctttcagagggggataacagccggaaacggctgctaataccgcatgatgttgcgggggcacatgcccctgcaaccaaaggagcaatccgctgaaagatgggctcgcgtccgattagccagttggcggggtaacggcccaccaaagcgacgatcggtagccggactgagaggttgaacggccacattgggactgagacacggcccagactcctacgggaggcagcagtgggggatattgcacaatgggcgaaagcctgatgcagcgacgccgcgtgagggaagacggtcttcggattgtaaacctctgtctttggggaagaaaatgacggtacccaaagaggaagctccggctaactacgtgccagcagccgcggtaatacgtagggagcaagcgttgtccggaattactgggtgtaaagggagcgtaggcgggatggcaagtagaatgttaaatccatcggctcaaccggtggctgcgttctaaactgccgttcttgagtgaagtagaggcaggcggaattcctagtgtagcggtgaaatgcgtagatattaggaggaacaccagtggcgaaggcggcctgctgggctttaactgacgctgaggctcgaaagcgtggggagcaaacaggattagataccctggtagtccacgccgtaaacgatgattactaggtgtggggggactgaccccttccgtgccgcagttaacacaataagtaatccacctggggagtacggccgcaaggttgaaactcaaaggaattgacgggggcccgcacaagcag

SEQ ID NO: 29 - DNA, 678 bp, Enterococcus faecium
  misc_feature: HB-85_Enterococcus_faecium
gtcgtacgcttctttttccaccggagcttgctccaccggaaaaagaagagtggcgaacgggtgagtaacacgtgggtaacctgcccatcagaaggggataacacttggaaacaggtgctaataccgtataacaatcgaaaccgcatggttttgatttgaaaggcgctttcgggtgtcgctgatggatggacccgcggtgcattagctagttggtgaggtaacggctcaccaaggccacgatgcatagccgacctgagagggtgatcggccacattgggactgagacacggcccaaactcctacgggaggcagcagtagggaatcttcggcaatggacgaaagtctgaccgagcaacgccgcgtgagtgaagaaggttttcggatcgtaaaactctgttgttagagaagaacaaggatgagagtaactgttcatcccttgacggtatctaaccagaaagccacggctaactacgtgccagcagccgcggtaatacgtaggtggcaagcgttgtccggatttattgggcgtaaagcgagcgcaggcggtttcttaagtctgatgtgaaagcccccggctcaaccggggagggtcattggaaactgggagacttgagtgcagaagaggagagtggaattccatgtgtagcggtgaaatgcgtagatatatggaggaacacc

SEQ ID NO: 30 - DNA, 771 bp, Lactobacillus brevis
  misc_feature: HB-87_Lactobacillus_brevis
cgagcttccgttgaatgacgtgcttgcactgatttcaacaatgaagcgagtggcgaactggtgagtaacacgtggggaatctgcccagaagcaggggataacacttggaaacaggtgctaataccgtataacaacaaaatccgcatggattttgtttgaaaggtggcttcggctatcacttctggatgatcccgcggcgtattagttagttggtgaggtaaaggcccaccaagacgatgatacgtagccgacctgagagggtaatcggccacattgggactgagacacggcccaaactcctacgggaggcagcagtagggaatcttccacaatggacgaaagtctgatggagcaatgccgcgtgagtgaagaagggtttcggctcgtaaaactctgttgttaaagaagaacacctttgagagtaactgttcaagggttgacggtatttaaccagaaagccacggctaactacgtgccagcagccgcggtaatacgtaggtggcaagcgttgtccggatttattgggcgtaaagcgagcgcaggcggttttttaagtctgatgtgaaagccttcggcttaaccggagaagtgcatcggaaactgggagacttgagtgcagaagaggacagtggaactccatgtgtagcggtggaatgcgtagatatatggaagaacaccagtggcgaaggcggctgtctagtctgtaactgacgctgaggctcgaaagcatgggtagcgaacaggattagataccctggtagtccatgccgt

SEQ ID NO: 31 - DNA, 671 bp, Hungatella hathewayi
  misc_feature: HB-01_Hungatella_hathewayi
ttgacttagcggcggacgggtgagtaacgcgtgggtaacctgccttacactgggggataacagttagaaatgactgctaataccgcataagcgcacagggccgcatggtctggtgtgaaaaactccggtggtgtaagatggacccgcgtctgattaggtagttggcggggtaacggcccaccaagccgacgatcagtagccgacctgagagggtgaccggccacattgggactgagacacggcccaaactcctacgggaggcagcagtggggaatattggacaatgggcgaaagcctgatccagcgacgccgcgtgagtgaagaagtatttcggtatgtaaagctctatcagcagggaagaaaatgacggtacctgactaagaagccccggctaactacgtgccagcagccgcggtaatacgtagggggcaagcgttatccggatttactgggtgtaaagggagcgtagacggtttagcaagtctgaagtgaaagcccggggctcaaccccggtactgctttggaaactgttagacttgagtgcaggagaggtaagtggaattcctagtgtagcggtgaaatgcgtagatattaggaggaacaccagtggcgaaggcggcttactggactgtaactgacgttgaggctcgaaagcgtggggagcaaacagg

SEQ ID NO: 32 - DNA, 788 bp, Parabacteroides johnsonii
  misc_feature: HB-03_Parabacteroides_johnsonii
  misc_feature (730)..(730): n is a, c, g, or t
tgcagtcgaggggcagcgggggcagcaatgcctgccggcgaccggcgcacgggtgagtaacgcgtatgcaacttgcctatcagaggggaatagcccggcgaaagtcggattaatgctccataaaacaggggtcccgcatgggaatatttgttaaagattcatcgctgatagataggcatgcgttccattaggcagttggcggggtaacggcccaccaaaccgacgatggataggggttctgagaggaaggtcccccacattggaactgagacacggtccaaactcctacgggaggcagcagtgaggaatattggtcaatggccgggaggctgaaccagccaagtcgcgtgaaggatgaaggatctatggtttgtaaacttcttttatacgggaataaagtgtgggacgtgtcccattttgtatgtaccgtatgaataagcatcggctaactccgtgccagcagccgcggtaatacggaggatgcgagcgttatccggatttattgggtttaaagggtgcgtaggtggttgtttaagtcagcggtgaaagtttgtggctcaaccataaaattgccgttgaaactggatgacttgagtatatttgaggtaggcggaatgcgtggtgtagcggtgaaatgcatagatatcacgcagaactccgattgcgaaggcagcttactaaactataactgacactgaagcacgaaagcgtggggatcaaacaggattanataccctggtagtccacgcagtaaacgatgattactaggagtttgcgatagacagtaa

SEQ ID NO: 33 - DNA, 1396 bp, Bacteroides vulgatus
  misc_feature: HB-10_Bacteroides_vulgatus
tgcagtcgaggggcagcatggtcttagcttgctaaggccgatggcgaccggcgcacgggtgagtaacacgtatccaacctgccgtctactcttggacagccttctgaaaggaagattaatacaagatggcatcatgagtccgcatgttcacatgattaaaggtattccggtagacgatggggatgcgttccattagatagtaggcggggtaacggcccacctagtcttcgatggataggggttctgagaggaaggtcccccacattggaactgagacacggtccaaactcctacgggaggcagcagtgaggaatattggtcaatgggcgagagcctgaaccagccaagtagcgtgaaggatgactgccctatgggttgtaaacttcttttataaaggaataaagtcgggtatgcatacccgtttgcatgtactttatgaataaggatcggctaactccgtgccagcagccgcggtaatacggaggatccgagcgttatccggatttattgggtttaaagggagcgtagatggatgtttaagtcagttgtgaaagtttgcggctcaaccgtaaaattgcagttgatactggatatcttgagtgcagttgaggcaggcggaattcgtggtgtagcggtgaaatgcttagatatcacgaagaactccgattgcgaaggcagcctgctaagctgcaactgacattgaggctcgaaagtgtgggtatcaaacaggattagataccctggtagtccacacggtaaacgatgaatactcgctgtttgcgatatactgcaagcggccaagcgaaagcgttaagtattccacctggggagtacgccggcaacggtgaaactcaaaggaattgacgggggcccgcacaagcggaggaacatgtggtttaattcgatgatacgcgaggaaccttacccgggcttaaattgcagatgaattacggtgaaagccgtaagccgcaaggcatctgtgaaggtgctgcatggttgtcgtcagctcgtgccgtgaggtgtcggcttaagtgccataacgagcgcaacccttgttgtcagttactaacaggttctgctgaggactctgacaagactgccatcgtaagatgtgaggaaggtggggatgacgtcaaatcagcacggcccttacgtccggggctacacacgtgttacaatggggggtacagagggccgctaccacgcgagtggatgccaatcccaaaaacctctctcagttcggactggagtctgcaacccgactccacgaagctggattcgctagtaatcgcgcatcagccacggcgcggtgaatacgttcccgggccttgtacacaccgcccgtcaagccatgggagccgggggtacctgaagtgcgtaaccgcgaggagcgccc

SEQ ID NO: 34 - DNA, 1398 bp, Bacteroides uniformis
  misc_feature: HB-13_Bacteroides_uniformis
catgcagtcgaggggcagcatgaacttagcttgctaagtttgatggcgaccggcgcacgggtgagtaacacgtatccaacctgccgatgactcggggatagcctttcgaaagaaagattaatacccgatggcatagttcttccgcatggtggaactattaaagaatttcggtcatcgatggggatgcgttccattaggttgttggcggggtaacggcccaccaagccttcgatggataggggttctgagaggaaggtcccccacattggaactgagacacggtccaaactcctacgggaggcagcagtgaggaatattggtcaatggacgagagtctgaaccagccaagtagcgtgaaggatgactgccctatgggttgtaaacttcttttatacgggaataaagtgaggcacgtgtgcctttttgtatgtaccgtatgaataaggatcggctaactccgtgccagcagccgcggtaatacggaggatccgagcgttatccggatttattgggtttaaagggagcgtaggcggacgcttaagtcagttgtgaaagtttgcggctcaaccgtaaaattgcagttgatactgggtgtcttgagtacagtagaggcaggcggaattcgtggtgtagcggtgaaatgcttagatatcacgaagaactccgattgcgaaggcagcttgctggactgtaactgacgctgatgctcgaaagtgtgggtatcaaacaggattagataccctggtagtccacacagtaaacgatgaatactcgctgtttgcgatatacagtaagcggccaagcgaaagcgttaagtattccacctggggagtacgccggcaacggtgaaactcaaaggaattgacgggggcccgcacaagcggaggaacatgtggtttaattcgatgatacgcgaggaaccttacccgggcttgaattgcaactgaatgatgtggagacatgtcagccgcaaggcagttgtgaaggtgctgcatggttgtcgtcagctcgtgccgtgaggtgtcggcttaagtgccataacgagcgcaacccttatcgatagttaccatcaggttatgctggggactctgtcgagactgccgtcgtaagatgtgaggaaggtggggatgacgtcaaatcagcacggcccttacgtccggggctacacacgtgttacaatggggggtacagaaggcagctacacggtgacgtgatgctaatcccgaaagcctctctcagttcggattggagtctgcaacccgactccatgaagctggattcgctagtaatcgcgcatcagccacggcgcggtgaatacgttcccgggccttgtacacaccgcccgtcaagccatgaaagccgggggtacctgaagtgcgtaaccgcaaggagcgccct

SEQ ID NO: 35 - DNA, 1388 bp, Turicibacter sanguinis
  misc_feature: HB-147_Turicibacter_sanguinis
tgcagtcgagcgaaccacttcggtggtgagcggcgaacgggtgagtaacacgtaggttatctgcccatcagacggggacaacgattggaaacgatcgctaataccggataggacgaaagtttaaaggtgcttcggcaccactgatggatgagcctgcggcgcattagctagttggtagggtaaaggcctaccaaggcgacgatgcgtagccgacctgagagggtgaacggccacactgggactgagacacggcccagactcctacgggaggcagcagtagggaatcttcggcaatgggcgaaagcctgaccgagcaacgccgcgtgaatgatgaaggccttcgggttgtaaaattctgttataagggaagaatggctctagtaggaaatggctagagtgtgacggtaccttatgagaaagccacggctaactacgtgccagcagccgcggtaatacgtaggtggcgagcgttatccggaattattgggcgtaaagagcgcgcaggtggttgattaagtctgatgtgaaagcccacggcttaaccgtggagggtcattggaaactggtcaacttgagtgcagaagagggaagtggaattccatgtgtagcggtgaaatgcgtagagatatggaggaacaccagtggcgaaggcggcttcctggtctgtaactgacactgaggcgcgaaagcgtggggagcaaacaggattagataccctggtagtccacgccgtaaacgatgagtgctaagtgttgggggtcgaacctcagtgctgaagttaacgcattaagcactccgcctggggagtacggtcgcaagactgaaactcaaaggaattgacggggacccgcacaagcggtggagcatgtggtttaattcgaagcaacgcgaagaaccttaccaggtcttgacataccagtgaccgtcctagagataggattttcccttcggggacaatggatacaggtggtgcatggttgtcgtcagctcgtgtcgtgagatgttgggttaagtcccgcaacgagcgcaacccctgtcgttagttgccagcattcagttggggactctaacgagactgccagtgacaaactggaggaaggtggggatgacgtcaaatcatcatgccccttatgacctgggctacacacgtgctacaatggttggtacaaagagaagcgaagcggtgacgtggagcaaacctcataaagccaatctcagttcggattgtaggctgcaactcgcctacatgaagttggaatcgctagtaatcgcgaatcagcatgtcgcggtgaatacgttcccgggtcttgtacacaccgcccgtcacaccacgagagtttacaacacccgaagtcagtggcctaaccgcaaggagggagctgcctaag

SEQ ID NO: 36 - DNA, 1389 bp, Coprococcus eutactus
  misc_feature: HB-155_Coprococcus_eutactus
tgcagtcgaacgattgaagctggagcttgctccggccgatttagtggcggacgggtgagtaacgcgtgggtaacctgcctcatacagggggatagcagttggaaacgactggtaaaaccgcataagcgcacggtgtcgcatgacacagtgtgaaaacactccggtggtatgagatggacccgcgtctgattagctagttggtgaggtaacggcccaccaaggcgacgatcagtagccggcctgagagggtgaacggccacattgggactgagacacggcccaaactcctacgggaggcagcagtggggaatattgcacaatgggggaaaccctgatgcagcgacgccgcgtgagtgatgaagtatttcggtatgtaaagctctatcagcagggaagataatgacggtacctgactaagaagcaccggctaaatacgtgccagcagccgcggtaatacgtatggtgcaagcgttatccggatttactgggtgtaaagggtgcgtaggtggcaaggcaagtctgaagtgaaaatccggggctcaaccccggaactgctttggaaactgtttagctggagtacaggagaggtaagtggaattcctagtgtagcggtgaaatgcgtagatattaggaggaacaccagtggcgaaggcgacttactggactgctactgacactgaggcacgaaagcgtggggagcaaacaggattagataccctggtagtccacgccgtaaacgatgaatactaggtgttgggttccaaaggaactcggtgccgtcgcaaacgcattaagtattccacctggggagtacgttcgcaagaatgaaactcaaaggaattgacggggacccgcacaagcggtggagcatgtggtttaattcgaagcaacgcgaaaaaccttaccaagtcttgacatcctgatgacggttccttaaccggaacttttcttcggaacatcagagacaggtggtgcatggttgtcgtcagctcgtgtcgtgagatgttgggttaagtcccgcaacgagcgcaacccctatttccagtagccagcagtaagatgggcactctggagagactgccggggataacccggaggaaggtggggatgacgtcaaatcatcatgccccttatgatttgggctacacacgtgctacaatggcagttacaaagagaagcaaacctgtgaaggcgagcaaacctcaaaaaggctgtctcagttcggattgtagtctgcaactcgactacatgaagctggaatcgctagtaatcgcagatcagcatgctgcggtgaatacgttcccgggtcttgtacacaccgcccgtcacaccatgggagtcggtaacgcccgaagtcagtgacccaaccgcaaggagggagctgccgaa

SEQ ID NO: 37 - DNA, 1380 bp, Bacteroides cellulosilyticus
  misc_feature: HB-227_Bacteroides_cellulosilyticus
atgcagtcgaggggcagcatgacctagcaataggttgatggcgaccggcgcacgggtgagtaacacgtatccaacctaccggttattccgggatagcctttcgaaagaaagattaataccggatagtataacgagaaggcatctttttgttattaaagaatttcgataaccgatggggatgcgttccattagtttgttggcggggtaacggcccaccaagacatcgatggataggggttctgagaggaaggtcccccacattggaactgagacacggtccaaactcctacgggaggcagcagtgaggaatattggtcaatggacgagagtctgaaccagccaagtagcgtgaaggatgactgccctatgggttgtaaacttcttttataatgggaataaagtgagccacgtgtggctttttgtatgtaccatacgaataaggatcggctaactccgtgcccagcagccgcggtaatacggaggatccgagcgttatccggatttattgggtttaaagggagcgtaggcggactattaagtcagctgtgaaagtttgcggctcaaccgtaaaattgcagttgatactggtcgtcttgagtgcagtagaggtaggcggaattcgtggtgtagcggtgaaatgcttagatatcacgaagaactccgattgcgaaggcagcttactggactgtaactgacgctgatgctcgaaagtgtgggtatcaaacaggattagataccctggtagtccacacagtaaacgatgaatactcgctgtttgcgatatacagcaagcggccaagcgaaagcattaagtattccacctggggagtacgccggcaacggtgaaactcaaaggaattgacgggggcccgcacaagcggaggaacatgtggtttaattcgatgatacgcgaggaaccttacccgggcttaaattgcatctgaataatttggaaacagattagccgtaaggcagatgtgaaggtgctgcatggttgtcgtcagctcgtgccgtgaggtgtcggcttaagtgccataacgagcgcaacccttatctttagttactaacaggtcatgctgaggactctagagagactgccgtcgtaagatgtgaggaaggtggggatgacgtcaaatcagcacggcccttacgtccggggctacacacgtgttacaatggggggtacagaaggcagctacacagcgatgtgatgctaatcccaaaagcctctctcagttcggattggagtctgcaacccgactccatgaagctggattcgctagtaatcgcgcatcagccacggcgcggtgaatacgttcccgggccttgtacacaccgcccgtcaagccatgaaagccgggggtacctgaagtccgtaac

SEQ ID NO: 38 - DNA, 1400 bp, Bacteroides plebeius
  misc_feature: HB-237_Bacteroides_plebeius
tgcagtcgaggggcagcagtggattgaagcttgcttcaattgctcggcgaccggcgcacgggtgagtaacgcgtatccaaccttccgtacactcagggatagcctttcgaaagaaagattaatacctgatggtatgatgagattgcatgatatcatcattaaagatttatcggtgtacgatggggatgcgttccattaggtagtaggcggggtaacggcccacctagcctacgatggataggggttctgagaggaaggtcccccacattggaactgagacacggtccaaactcctacgggaggcagcagtgaggaatattggtcaatggacgagagtctgaaccagccaagtagcgtgaaggatgaaggtcctacggattgtaaacttcttttataagggaataaaccctcccacgtgtgggagcttgtatgtaccttatgaataagcatcggctaactccgtgccagcagccgcggtaatacggaggatgcgagcgttatccggatttattgggtttaaagggagcgcagacgggtcgttaagtcagctgtgaaagtttggggctcaaccttaaaattgcagttgatactggcgtccttgagtgcggttgaggtgtgcggaattcgtggtgtagcggtgaaatgcttagatatcacgaagaactccgattgcgaaggcagcacactaagccgtaactgacgttcatgctcgaaagtgtgggtatcaaacaggattagataccctggtagtccacacggtaaacgatggatactcgctgttggcgatatacggtcagcggcttagcgaaagcgttaagtatcccacctggggagtacgccggcaacggtgaaactcaaaggaattgacgggggcccgcacaagcggaggaacatgtggtttaattcgatgatacgcgaggaaccttacccgggcttaaattgcaaaggaattgtgtggaaacatgcaagtcttcggacctttgtgaaggtgctgcatggttgtcgtcagctcgtgccgtgaggtgtcggcttaagtgccataacgagcgcaaccctcgccgccagttactaacagttaaagctgaggactctggcgggactgccatcgtaagatgtgaggaaggtggggatgacgtcaaatcagcacggcccttacgtccggggctacacacgtgttacaatgggcggtacagaaggcagctacctggcgacaggatgccaatcccgaaagccgctctcagttcggactggagtctgcaacccgactccacgaagctggattcgctagtaatcgcgcatcagccacggcgcggtgaatacgttcccgggccttgtacacaccgcccgtcaagccatgaaagccgggggtacctgaagtgcgtaaccgcaaggagcgccct

SEQ ID NO: 39 - DNA, 770 bp, Erysipelatoclostridium ramosum
  misc_feature: HB-24_Erysipelatoclostridium_ramosum
gtcgaacgcgagcacttgtgctcgagtggcgaacgggtgagtaatacataagtaacctgccctagacagggggataactattggaaacgatagctaagaccgcataggtacggacactgcatggtgaccgtattaaaagtgcctcaaagcactggtagaggatggacttatggcgcattagctggttggcggggtaacggcccaccaaggcgacgatgcgtagccgacctgagagggtgaccggccacactgggactgagacacggcccagactcctacgggaggcagcagtagggaattttcggcaatgggggaaaccctgaccgagcaacgccgcgtgaaggaagaaggttttcggattgtaaacttctgttataaaggaagaacggcggctacaggaaatggtagccgagtgacggtactttattagaaagccacggctaactacgtgccagcagccgcggtaatacgtaggtggcaagcgttatccggaattattgggcgtaaagagggagcaggcggcagcaagggtctgtggtgaaagcctgaagcttaacttcagtaagccatagaaaccaggcagctagagtgcaggagaggatcgtggaattccatgtgtagcggtgaaatgcgtagatatatggaggaacaccagtggcgaaggcgacgatctggcctgcaactgacgctcagtcccgaaagcgtggggagcaaataggattagataccctagtagtccacgccgtaaacgatgagtactaagtgttgga

SEQ ID NO: 40 - DNA, 784 bp, Bacillus cereus
  misc_feature: HB-25_Bacillus_cereus
tgcagtcgagcgaatggattaagagcttgctcttatgaagttagcggcggacgggtgagtaacacgtgggtaacctgcccataagactgggataactccgggaaaccggggctaataccggataacattttgaaccgcatggttcgaaattgaaaggcggcttcggctgtcacttatggatggacccgcgtcgcattagctagttggtgaggtaacggctcaccaaggcaacgatgcgtagccgacctgagaggggatcggccacactgggactgagacacggcccagactcctacgggaggcagcagtagggaatcttccgcaatggacgaaagtctgacggagcaacgccgcgtgagtgatgaaggctttcgggtcgtaaaactctgttgttagggaagaacaagtgctagttgaataagctggcaccttgacggtacctaaccagaaagccacggctaactacgtgccagcagccgcggtaatacgtaggtggcaagcgttatccggaattattgggcgtaaagcgcgcgcaggtggtttcttaagtctgatgtgaaagcccacggctcaaccgtggagggtcattggaaactgggagacttgagtgcagaagaggaaagtggaattccatgtgtagcggtgaaatgcgtagagatatggaggaacaccagtggcgaaggcgactttctggtctgtaactgacactgaggcgcgaaagcgtggggagcaaacaggattagataccctggtagtccacgccgtaaacgatgagtgctaagtg

SEQ ID NO: 41 - DNA, 1397 bp, Agathobacter rectalis
  misc_feature: HB-257_Agathobacter_rectalis
tgcagtcgaacgaagcactttatttgatttccttcgggactgattattttgtgactgagtggcggacgggtgagtaacgcgtgggtaacctgccttgtacagggggataacagttggaaacggctgctaataccgcataagcgcacggcatcgcatgatgcagtgtgaaaaactccggtggtataagatggacccgcgttggattagctagttggtgaggtaacggcccaccaaggcgacgatccatagccgacctgagagggtgaccggccacattgggactgagacacggcccaaactcctacgggaggcagcagtggggaatattgcacaatgggcgaaagcctgatgcagcgacgccgcgtgagcgaagaagtatttcggtatgtaaagctctatcagcagggaagataatgacggtacctgactaagaagcaccggctaaatacgtgccagcagccgcggtaatacgtatggtgcaagcgttatccggatttactgggtgtaaagggagcgcaggcggtgcggcaagtctgatgtgaaagcccggggctcaaccccggtactgcattggaaactgtcgtactagagtgtcggaggggtaagcggaattcctagtgtagcggtgaaatgcgtagatattaggaggaacaccagtggcgaaggcggcttactggacgataactgacgctgaggctcgaaagcgtggggagcaaacaggattagataccctggtagtccacgccgtaaacgatgaatactaggtgttgggaagcattgcttctcggtgccgtcgcaaacgcagtaagtattccacctggggagtacgttcgcaagaatgaaactcaaaggaattgacggggacccgcacaagcggtggagcatgtggtttaattcgaagcaacgcgaagaaccttaccaagtcttgacatccttctgaccggtacttaatcgtaccttctcttcggagcaggagtgacaggtggtgcatggttgtcgtcagctcgtgtcgtgagatgttgggttaagtcccgcaacgagcgcaacccttatctttagtagccagcggtcaagccgggcactctagagagactgccagggataacctggaggaaggcggggatgacgtcaaatcatcatgccccttatgacttgggctacacacgtgctacaatggcgtaaacaaagggaagcaaagctgtgaagccgagcaaatctcaaaaataacgtctcagttcggactgtagtctgcaacccgactacacgaagctggaatcgctagtaatcgcagatcagaatgctgcggtgaatacgttcccgggtcttgtacacaccgcccgtcacaccatgggagttgggaatgcccgaagccagtgacctaaccgaaaggaaggagc

SEQ ID NO: 42 - DNA, 1384 bp, Ruminococcus bicirculans
  misc_feature: HB-268_Ruminococcus_bicirculans
gcagtcgaacgagagaagagaagcttgcttttctgatctagtggcggacgggtgagtaacacgtgagcaatctgcctttcagagggggataccgattggaaacgatcgttaataccgcataacataattgaaccgcatgatttgattatcaaagatttatcgctgaaagatgagctcgcgtctgattagctagttggtaaggtaacggcttaccaaggcgacgatcagtagccggactgagaggttgatcggccacattgggactgagacacggcccagactcctacgggaggcagcagtggggaatattgcacaatggaggaaactctgatgcagcgatgccgcgtgagggaagaaggttttaggattgtaaacctctgtcttcagggacgaaaaaagacggtacctgaggaggaagctccggctaactacgtgccagcagccgcggtaatacgtagggagcgagcgttgtccggaattactgggtgtaaagggagcgtaggcgggatcgcaagtcagatgtgaaaactatgggcttaacccataaactgcatttgaaactgtggttcttgagtgaagtagaggtaagcggaattcctagtgtagcggtgaaatgcgtagatattaggaggaacatcagtggcgaaggcggcttactgggctttaactgacgctgaggctcgaaagcgtggggagcaaacaggattagataccctggtagtccacgccgtaaacgatgattactaggtgtggggggactgaccccttccgtgccgcagcaaacgcaataagtaatccacctggggagtacgaccgcaaggttgaaactcaaaggaattgacgggggcccgcacaagcagtggagtatgtggattaattcgaagcaacgcgaagaaccttaccaggtcttgacatcgtatgcatagctcagagatgagtgaaatctcttcggagacatatagacaggtggtgcatggttgtcgtcagctcgtgtcgtgagatgttgggttaagtcccgcaacgagcgcaacccttactgttagttgctacgcaagagcactctagcaggactgccgttgacaaaacggaggaaggtggggatgacgtcaaatcatcatgccccttatgacctgggcctcacacgtactacaatggctgtcaacagagggatgcaaagccgcgaggtggagcgaacccctaaaagcagtcttagttcggattgtaggctgcaacccgcctacatgaagtcggaattgctagtaatcgcagatcagcatgctgcggtgaatacgttcccgggccttgtacacaccgcccgtcacgccatgggagtcggtaacacccgaagcctgtagtctaaccgcaaggaggacgcagtcgaagg

SEQ ID NO: 43 - DNA, 909 bp, Clostridium paraputrificum
  misc_feature: HB-27_Clostridium_paraputrificum
tgcagtcgagcgatgaagttccttcgggaacggattagcggcggacgggtgagtaacacgtgggcaacctgccttatagaggggaatagccttccgaaaggaagattaataccgcataagattgtagcttcgcatgaagtagcaattaaaggagcaatccgctataagatgggcccgcggcgcattagctagttggtgaggtaacggctcaccaaggcgacgatgcgtagccgacctgagagggtgatcggccacattgggactgagacacggcccagactcctacgggaggcagcagtggggaatattgcacaatgggggaaaccctgatgcagcaacgccgcgtgagtgatgacggccttcgggttgtaaagctctgtctttggggacgataatgacggtacccaaggaggaagccacggctaactacgtgccagcagccgcggtaatacgtaggtggcaagcgttgtccggatttactgggcgtaaagggagcgtaggcggatttttaagtgggatgtgaaatacccgggctcaacctgggtgctgcattccaaactggaaatctagagtgcaggaggggaaagtggaattcctagtgtagcggtgaaatgcgtagagattaggaagaacaccagtggcgaaggcgactttctggactgtaactgacgctgaggctcgaaagcgtggggagcaaacaggattagataccctggtagtccacgccgtaaacgatgaatactaggtgtaggggttgtcatgacctctgtgccgccgctaacgcattaagtattccgcctggggagtacggtcgcaagattaaaactcaaaggaatttgacgggggcccgcacaagtagcggagcatgtggtttaattcgaagcaacgcgaagaaccttacctagactt

SEQ ID NO: 44 - DNA, 1360 bp, Collinsella aerofaciens
  misc_feature: HB-274_Collinsella_aerofaciens
  misc_feature (1249)..(1249): n is a, c, g, or t
  misc_feature (1349)..(1349): n is a, c, g, or t
  misc_feature (1360)..(1360): n is a, c, g, or t
tgcagtcgaacggcacccctctccggagggaagcgagtggcgaacggctgagtaacacgtggagaacctgccccctcccccgggatagccgcccgaaaggacgggtaataccggatacccccgggcgccgcatggcgcccgggctaaagccccgacgggaggggatggctccgcggcccatcaggtagacggcggggtgacggcccaccgtgccgacaacgggtagccgggttgagagaccgaccggccagattgggactgagacacggcccagactcctacgggaggcagcagtggggaatcttgcgcaatggggggaaccctgacgcagcgacgccgcgtgcgggacggaggccttcgggtcgtaaaccgctttcagcagggaagagtcaagactgtacctgcagaagaagccccggctaactacgtgccagcagccgcggtaatacgtagggggcgagcgttatccggattcattgggcgtaaagcgcgcgtaggcggcccggcaggccgggggtcgaagcggggggctcaaccccccgaagcccccggaacctccgcggcttgggtccggtaggggagggtggaacacccggtgtagcggtggaatgcgcagatatcgggtggaacaccggtggcgaaggcggccctctgggccgagaccgacgctgaggcgcgaaagctgggggagcgaacaggattagataccctggtagtcccagccgtaaacgatggacgctaggtgtgggggggacgatccccccgtgccgcagccaacgcattaagcgtcccgcctggggagtacggccgcaaggctaaaactcaaaggaattgacgggggcccgcacaagcagcggagcatgtggcttaattcgaagcaacgcgaagaaccttaccagggcttgacatatgggtgaagcgggggagaccccgtggccgagaggagcccatacaggtggtgcatggctgtcgtcagctcgtgtcgtgagatgttgggttaagtcccgcaacgagcgcaacccccgccgcgtgttgccatcgggtgatgccgggaacccacgcgggaccgccgccgtcaaggcggaggagggcggggacgacgtcaagtcatcatgccccttatgccctgggctgcacacgtgctacaatggccggtacagagggatgccaccccgcgagggggagcggatcccggaaagccggcccagttcgggattgggggctgcaacccgcccccatgaagtcgganttgctagtaatcgcggatcagcatgccgcggtgaatgcgttcccgggccttgtacacaccgcccgtcacaccacccgagtcgtctgcacccgaagtcgcnggcccaaccgn

SEQ ID NO: 45 - DNA, 967 bp, Oscillibacter sp.
  misc_feature: HB-28_Oscillibacter_sp.
  misc_feature (8)..(8): n is a, c, g, or t
ctgacccngcaacgccgcgtgaaggaagaaggctttcgggttgtaaacttcttttgtcagggaagagtagaagacggtacctgacgaataagccacggctaactacgtgccagcagccgcggtaatacgtaggtggcaagcgttgtccggatttactgggtgtaaagggcgtgcagccgggccggcaagtcagatgtgaaatctggaggcttaacctccaaactgcatttgaaactgtaggtcttgagtaccggagaggttatcggaattccttgtgtagcggtgaaatgcgtagatataaggaagaacaccagtggcgaaggcggataactggacggcaactgacggtgaggcgcgaaagcgtggggagcaaacaggattagataccctggtagtccacgctgtaaacgatggatactaggtgtgcggggactgaccccctgcgtgccgcagttaacacaataagtatcccacctggggagtacgatcgcaaggttgaaactcaaaggaattgacgggggcccgcacaagcggtggattatgtggtttaattcgaagcaacgcgaagaaccttaccagggcttgacatcctactaacgaagtagagatacatcaggtgcccttcggggaaagtagagacaggtggtgcatggttgtcgtcagctcgtgtcgtgagatgttgggttaagtcccgcaacgagcgcaacccctattgttagttgctacgcaagagcactctagcgagactgccgttgacaaaacggaggaaggtggggacgacgtcaaatcatcatgccccttatgtcctggggctacacacgtaatacaatggcggtcaacagagggaggcaaagccgcgaggcagagcaaacccccaaaagccgtcccagttcggatcgcaggctgcaacccgcctgcgtgaagtcggaatcgctagtaatcgcggatcagcatgccgcggtgaata

SEQ ID NO: 46 - DNA, 1394 bp, Alistipes onderdonkii
  misc_feature: HB-311_Alistipes_onderdonkii
tgcagtcgaggggcatcgggattgaagcttgcttcaattgccggcgaccggcgcacgggtgcgtaacgcgtatgtaacctacctataacaggggcataacactgagaaattggtactaattccccataatattcggagaggcatctctccgggttgaaaactccggtggttatagatggacatgcgttgtattagctagttggtgaggtaacggctcaccaaggcaacgatacatagggggactgagaggttaaccccccacactggtactgagacacggaccagactcctacgggaggcagcagtgaggaatattggtcaatggacgcaagtctgaaccagccatgccgcgtgcaggaagacggctctatgagttgtaaactgcttttgtacgagggtaaactcacctacgtgtaggtgactgaaagtatcgtacgaataaggatcggctaactccgtgccagcagccgcggtaatacggaggattcaagcgttatccggatttattgggtttaaagggtgcgtaggcggtttgataagttagaggtgaaatcccggggcttaactccggaactgcctctaatactgttagactagagagtagttgcggtaggcggaatgtatggtgtagcggtgaaatgcttagagatcatacagaacaccgattgcgaaggcagcttaccaaactatatctgacgttgaggcacgaaagcgtggggagcaaacaggattagataccctggtagtccacgcagtaaacgatgataactcgttgtcggcgatacacagtcggtgactaagcgaaagcgataagttatccacctggggagtacgttcgcaagaatgaaactcaaaggaattgacgggggcccgcacaagcggaggaacatgtggtttaattcgatgatacgcgaggaaccttacccgggcttgaaagttactgacgattctggaaacaggatttccctttggggcaggaaactaggtgctgcatggttgtcgtcagctcgtgccgtgaggtgtcgggttaagtcccataacgagcgcaacccctaccgttagttgccatcaggtcaagctgggcactctggcgggactgccggtgtaagccgagaggaaggtggggatgacgtcaaatcagcacggcccttacgtccggggctacacacgtgttacaatggtaggtacagagggtcgctacyccgtgaggggatgccaatctcgaaagcctatctcagttcggattggaggctgaaacccgcctccatgaagttggattcgctagtaatcgcgcatcagccatggcgcggtgaatacgttcccgggccttgtacacaccgcccgtcaagccatggaagctgggggtgcctgaagttcgtgaccgcaaggagcga

SEQ ID NO: 47 - DNA, 1395 bp, Alistipes putredinis
  misc_feature: HB-324_Alistipes_putredinis
tgcagtcgaggggcagcataatggatagcaatatctatggtggcgaccggcgcacgggtgcgtaacgcgtatgcaacctacctttaacagggggataacactgagaaattggtactaataccccataatatcatagaaggcatcttttatggttgaaaattccgatggttagagatgggcatgcgttgtattagctagttggtggggtaacggctcaccaaggcgacgatacatagggggactgagaggttaaccccccacactggtactgagacacggaccagactcctacgggaggcagcagtgaggaatattggtcaatggacgcaagtctgaaccagccatgccgcgtgcaggatgacggctctatgagttgtaaactgcttttgtacgagggtaaacgcagatacgtgtatctgtctgaaagtatcgtacgaataaggatcggctaactccgtgccagcagccgcggtaatacggaggattcaagcgttatccggatttattgggtttaaagggtgcgtaggcggtttgataagttagaggtgaaatttcggggctcaaccctgaacgtgcctctaatactgttgagctagagagtagttgcggtaggcggaatgtatggtgtagcggtgaaatgcttagagatcatacagaacaccgattgcgaaggcagcttaccaaactatatctgacgttgaggcacgaaagcgtggggagcaaacaggattagataccctggtagtccacgcagtaaacgatgataactcgttgtcggcgatacacagtcggtgactaagcgaaagcgataagttatccacctggggagtacgttcgcaagaatgaaactcaaaggaattgacgggggcccgcacaagcggaggaacatgtggtttaattcgatgatacgcgaggaaccttacccgggcttgaaagttagcgacgattcttgaaagaggatttcccttcggggcgcgaaactaggtgctgcatggttgtcgtcagctcgtgccgtgaggtgtcgggttaagtcccataacgagcgcaacccctaccgttagttgccatcaggtgaagctgggcactctggcgggactgccggtgtaagccgagaggaaggtggggatgacgtcaaatcagcacggcccttacgtccggggctacacacgtgttacaatggtaggtacagagggcagctacccagcgatgggatgcgaatctcgaaagcctatctcagttcggattggaggctgaaacccgcctccatgaagttggattcgctagtaatcgcgcatcagccatggcgcggtgaatacgttcccgggccttgtacacaccgcccgtcaagccatgggagccgggggtgcctgaagttcgtgaccgcaaggagcgacc

SEQ ID NO: 48 - DNA, 1371 bp, Bacteroides stercoris
  misc_feature: HB-33_Bacteroides_stercoris
  misc_feature (1192)..(1194): n is a, c, g, or t
  misc_feature (1310)..(1310): n is a, c, g, or t
  misc_feature (1334)..(1335): n is a, c, g, or t
  misc_feature (1365)..(1365): n is a, c, g, or t
tgcagtcgaggggcagcatcatcaaagcttgctttgatggatggcgaccggcgcacgggtgagtaacacgtatccaacctgccgacaacactgggatagcctttcgaaagaaagattaataccggatggtatagttttcccgcatggggtgactattaaagaatttcggttgtcgatggggatgcgttccattaggcagttggcggggtaacggcccaccaaaccaacgatggataggggttctgagaggaaggtcccccacattggaactgagacacggtccaaactcctacgggaggcagcagtgaggaatattggtcaatggacgagagtctgaaccagccaagtagcgtgaaggatgactgccctatgggttgtaaacttcttttatacgggaataaagtgagccacgtgtggctttttgtatgtaccgtatgaataaggatcggctaactccgtgccagcagccgcggtaatacggaggatccgagcgttatccggatttattgggtttaaagggagcgtaggcgggttgttaagtcagttgtgaaagtttgcggctcaaccgtaaaattgcagttgatactggcgaccttgagtgcaacagaggtaggcggaattcgtggtgtagcggtgaaatgcttagatatcacgaagaactccgattgcgaaggcagcttactggattgtaactgacgctgatgctcgaaagtgtgggtatcaaacaggattagataccctggtagtccacacagtaaacgatgaatactcgctgttggcgatatacagtcagcggccaagcgaaagcattaagtattccacctggggagtacgccggcaacggtgaaactcaaaggaattgacgggggcccgcacaagcggaggaacatgtggtttaattcgatgatacgcgaggaaccttacccgggcttaaattgcaactgacggaatcggaaacggttctttcttcggacagttgtgaaggtgctgcatggttgtcgtcagctcgtgccgtgaggtgtcggcttaagtgccataacgagcgcaacccttatcgatagttactagcaggttatgctgaggactctatcgagactgccgtcgtaagatgtgaggaaggtggggatgacgtcaaatcagcacggcccttacgtccggggctacacacgtgttacaatggggggtacagaannnagctacacggcgacgtggtgctaatcccgaaagcctctctcagttcggattggagtctgcaacccgactccatgaagctggattcgctagtaatcgcgcatcagccacggcgcggngaatacgttcccgggccttgtacnnaccgcccgtcaagccatgaaagccgggggnacctga

SEQ ID NO: 49 - DNA, 1372 bp, Flintibacter butyricus
  misc_feature: HB-344_Flintibacter_butyricus
gaggtttcggccaagtagaataggatgtttagtggcggacgggtgagtaacgcgtgagtaacctgccttggagtggggaataacacagtgaaaattgtgctaataccgcataatgcatttgggtcgcatggttctgaatgccaaagatttatcgctctgagatggactcgcgtctgattagctggttggcggggcaacggcccaccaaggcgacgatcagtagccggactgagaggttggccggccacattgggactgagacacggcccagactcctacgggaggcagcagtggggaatattgggcaatgggcgcaagcctgacccagcaacgccgcgtgaaggaagaaggctttcgggttgtaaacttcttttgtcagggacgaagcaagtgacggtacctgacgaataagccacggctaactacgtgccagcagccgcggtaatacgtaggtggcaagcgttatccggatttactgggtgtaaagggcgtgtaggcgggaaagcaagtcagatgtgaaaactgtgggctcaacccacagcctgcatttgaaactgtttttcttgagtactggagaggcagatggaattcctagtgtagcggtgaaatgcgtagatattaggaggaacaccagtggcgaaggcgatctgctggacagcaactgacgctgaggcgcgaaagcgtggggagcaaacaggattagataccctggtagtccacgctgtaaacgatggatactaggtgtggggggtctgaccccctccgtgccgcagttaacacaataagtatcccacctggggagtacgatcgcaaggttgaaactcaaaggaattgacgggggcccgcacaagcggtggagtatgtggtttaattcgaagcaacgcgaagaaccttaccagggcttgacatcccggtgaccggcttagagataagcctttctcttcggagacgccggtgacaggtggtgcatggttgtcgtcagctcgtgtcgtgagatgttgggttaagtcccgcaacgagcgcaacccttattgttagttgctacgcaagagcactctagcgagactgccgttgacaaaacggaggaaggtggggatgacgtcaaatcatcatgccccttatgtcctgggccacacacgtactacaatggtggtcaacagagggaagcaagactgtgaagtggagcaaacccctaaaagccatcccagttcggatcgcaggctgcaacccgcctgcgtgaagttggaatcgctagtaatcgcggatcagcatgccgcggtgaatacgttcccgggccttgtacacaccgcccgtcacaccatgagagtcgggaacacccgaagtccgtagcctaaccgcaaggagggcgcgg

SEQ ID NO: 50 - DNA, 1357 bp, Coprococcus comes
  misc_feature: HB-376_Coprococcus_comes
ttgtgactgagtggcggacgggtgagtaacgcgtgggtaacctgcctcatacagggggataacagttagaaatgactgctaataccgcataagaccacagagccgcatggctcggtgggaaaaactccggtggtatgagatggacccgcgtctgattaggtagttggtggggtaacggcctaccaagccaacgatcagtagccgacctgagagggtgaccggccacattgggactgagacacggcccaaactcctacgggaggcagcagtggggaatattgcacaatgggggaaaccctgatgcagcgacgccgcgtgagcgaagaagtatttcggtatgtaaagctctatcagcagggaagaaaatgacggtacctgactaagaagcaccggctaaatacgtgccagcagccgcggtaatacgtatggtgcaagcgttatccggatttactgggtgtaaagggagcgtagacggctgtgtaagtctgaagtgaaagcccggggctcaaccccgggactgctttggaaactatgcagctagagtgtcggagaggtaagtggaattcccagtgtagcggtgaaatgcgtagatattgggaggaacaccagtggcgaaggcggcttactggacgatgactgacgttgaggctcgaaagcgtggggagcaaacaggattagataccctggtagtccacgccgtaaacgatgactactaggtgtcggggagcaaagctcttcggtgccgcagcaaacgcaataagtagtccacctggggagtacgttcgcaagaatgaaactcaaaggaattgacggggacccgcacaagcggtggagcatgtggtttaattcgaagcaacgcgaagaaccttacctgctcttgacatcccggtgaccggcgtgtaatgacgccttttcttcggaacaccggtgacaggtggtgcatggttgtcgtcagctcgtgtcgtgagatgttgggttaagtcccgcaacgagcgcaacccttatcttcagtagccagcaattcggatgggcactctggagagactgccagggataacctggaggaaggtggggatgacgtcaaatcatcatgccccttatgagcagggctacacacgtgctacaatggcgtaaacaaagggaagcgagcctgcgagggtaagcaaatctcaaaaataacgtctcagttcggattgtagtctgcaactcgactacatgaagctggaatcgctagtaatcgcgaatcagcatgtcgcggtgaatacgttcccgggtcttgtacacaccgcccgtcacaccatgggagttggtaacgcccgaagtcagtgacccaaccgtaaggagggagctgccgaag

SEQ ID NO: 51 - DNA, 1396 bp, Bacteroides koreensis
  misc_feature: HB-385_Bacteroides_koreensis
catgcaagtcgaggggcagcattttagtttgcttgcaaactgaagatggcgaccggcgcacgggtgagtaacacgtatccaacctgccgataactccggaatagcctttcgaaagaaagattaataccggatagcatacgaatatcgcatgatatttttattaaagaatttcggttatcgatggggatgcgttccattagtttgttggcggggtaacggcccaccaagactacgatggataggggttctgagaggaaggtcccccacattggaactgagacacggtccaaactcctacgggaggcagcagtgaggaatattggtcaatgggcgagagcctgaaccagccaagtagcgtgaaggatgaaggctctatgggtcgtaaacttcttttatatgggaataaagttttccacgtgtggaattttgtatgtaccatatgaataaggatcggctaactccgtgccagcagccgcggtaatacggaggatccgagcgttatccggatttattgggtttaaagggagcgtaggtggattgttaagtcagttgtgaaagtttgcggctcaaccgtaaaattgcagttgaaactggcagtcttgagtacagtagaggtgggcggaattcgtggtgtagcggtgaaatgcttagatatcacgaagaactccgattgcgaaggcagctcactagactgtcactgacactgatgctcgaaagtgtgggtatcaaacaggattagataccctggtagtccacacagtaaacgatgaatactcgctgtttgcgatatacagtaagcggccaagcgaaagcattaagtattccacctggggagtacgccggcaacggtgaaactcaaaggaattgacgggggcccgcacaagcggaggaacatgtggtttaattcgatgatacgcgaggaaccttacccgggcttaaattgcaacagaatatattggaaacagtatagccgtaaggctgttgtgaaggtgctgcatggttgtcgtcagctcgtgccgtgaggtgtcggcttaagtgccataacgagcgcaacccttatctttagttactaacaggtcatgctgaggactctagagagactgccgtcgtaagatgtgaggaaggtggggatgacgtcaaatcagcacggcccttacgtccggggctacacacgtgttacaatggggggtacagaaggcggctacctggtgacaggatgctaatcccaaaaacctctctcagttcggatcgaagtctgcaacccgacttcgtgaagctggattcgctagtaatcgcgcatcagccatggcgcggtgaatacgttcccgggccttgtacacaccgcccgtcaagccatgaaagccgggggtacctgaagtacgtaaccgcaaggagc

SEQ ID NO: 52 - DNA, 1383 bp, Eisenbergiella tayi
  misc_feature: HB-437_Eisenbergiella_tayi
cgaggaagttttcggatggaatcaggtataacttagtggcggacgggtgagtaacgcgtgggaaacctgccctgtaccgggggataacacttagaaataggtgctaataccgcataagcgcacggaactgcatggttctgtgtgaaaaactccggtggtacaggatggtcccgcgtctgattagccagttggcagggtaacggcctaccaaagcgacgatcagtagccggcctgagagggtgaacggccacattgggactgagacacggcccaaactcctacgggaggcagcagtggggaatattgcacaatgggggaaaccctgatgcagcgacgccgcgtgagtgaagaagtatttcggtatgtaaagctctatcagcagggaagaaaatgacggtacctgactaagaagccccggctaactacgtgccagcagccgcggtaatacgtagggggcaagcgttatccggatttactgggtgtaaagggagcgtagacggcatggcaagccagatgtgaaaacccagggctcaaccttgggattgcatttggaactgccaggctggagtgcaggagaggtaagcggaattcctagtgtagcggtgaaatgcgtagatattaggaggaacaccagtggcgaaggcggcttactggactgtaactgacgttgaggctcgaaagcgtggggagcaaacaggattagataccctggtagtccacgcggtaaacgatgattgctaggtgtaggtgggtatggacccatcggtgccgcagctaacgcaataagcaatccacctggggagtacgttcgcaagaatgaaactcaaaggaattgacggggacccgcacaagcggtggagcatgtggtttaattcgaagcaacgcgaagaaccttaccaagtcttgacatcccaatgacgtgcctgtaaagaggcattcccttcggggcattggagacaggtggtgcatggttgtcgtcagctcgtgtcgtgagatgttgggttaagtcccgcaacgagcgcaacccttattcttagtagccagcaggtagagctgggcactctaaggagactgccggggataacccggaggaaggcggggatgacgtcaaatcatcatgccccttatgatttgggctacacacgtgctacaatggcgtaaacaaagggaagcgagacagtgatgtggagcaaatcccagaaataacgtctcagttcggattgtagtctgcaactcgactacatgaagctggaatcgctagtaatcgcgaatcagcatgtcgcggtgaatacgttcccgggtcttgtacacaccgcccgtcacaccatgggagttggaaatgcccgaagtctgtgacctaaccgaaagggaggagcagccgaa

SEQ ID NO: 53 - DNA, 1403 bp, Dysosmobacter welbionis
  misc_feature: HB-45_Dysosmobacter_welbionis
tgcagtcgaacggagcacccttgactgaggtttcggccaaatgataggaatgcttagtggcggactggtgagtaacgcgtgaggaacctaccttccagagggggacaacagttggaaacgactgctaataccgcatgacgcatgaccggggcatcccgggcatgtcaaagattttatcgctggaagatggcctcgcgtctgattagctagatggtggggtaacggcccaccatggcgacgatcagtagccggactgagaggttgaccggccacattgggactgagatacggcccagactcctacgggaggcagcagtggggaatattgggcaatggacgcaagtctgacccagcaacgccgcgtgaaggaagaaggctttcgggttgtaaacttcttttgtcagggaagagtagaagacggtacctgacgaataagccacggctaactacgtgccagcagccgcggtaatacgtaggtggcaagcgttgtccggatttactgggtgtaaagggcgtgcagccgggccggcaagtcagatgtgaaatctggaggcttaacctccaaactgcatttgaaactgtaggtcttgagtaccggagaggttatcggaattccttgtgtagcggtgaaatgcgtagatataaggaagaacaccagtggcgaaggcggataactggacggcaactgacggtgaggcgcgaaagcgtggggagcaaacaggattagataccctggtagtccacgctgtaaacgatggatactaggtgtgcggggactgaccccctgcgtgccgcagttaacacaataagtatcccaccctggggagtacgatcgcaaggttgaaactcaaaggaattgacgggggcccgcacaagcggtggattatgtggtttaattcgaagcaacgcgaagaaccttaccagggcttgacatcctactaacgaagtagagatacatcaggtgcccttcggggaaagtagagacaggtggtgcatggttgtcgtcagctcgtgtcgtgagatgttgggttaagtcccgcaacgagcgcaacccctattgttagttgctacgcaagagcactctagcgagactgccgttgacaaaacggaggaaggtggggacgacgtcaaatcatcatgccccttatgtcctgggctacacacgtaatacaatggcggtcaacagagggaggcaaagccgcgaggcagagcaaacccccaaaagccgtcccagttcggatcgcaggctgcaacccgcctgcgtgaagtcggaatcgctagtaatcgcggatcagcatgccgcggtgaatacgttcccgggccttgtacacaccgcccgtcacaccatgagagtcgggaacacccgaagtccgtagcctaaccgcaaggagggcgcggccgaa

SEQ ID NO: 54 - DNA, 1400 bp, Butyricimonas paravirosa
  misc_feature: HB-453_Butyricimonas_paravirosa
  misc_feature (18)..(18): n is a, c, g, or t
  misc_feature (45)..(45): n is a, c, g, or t
  misc_feature (1078)..(1078): n is a, c, g, or t
tgcagtcgaggggcagcngcgggtagtagcaatacatcttcggcncggcgaccggcgcacgggtgagtaacacgtgtgcaaccaaccccgtaccgggagataacccgcggaaacgtggactaacatcccataacactgtatagtcgcatggtgctgcatttaaaattccggtggtacgggacgggcacgcgcgacattaggtagttggcggggtaacggcccaccaagccgacgatgtctaggggttctgagaggaaggtcccccacactggaactgagacacggtccagactcctacgggaggcagcagtgaggaatattggtcaatgggcgagagcctgaaccagccaagtcgcgtgagggaagaatggtctatggcctgtaaacctcttttgccagggaagaataaaaggtacgtgtaccttcttgccagtacctgacgaataagcatcggctaactccgtgccagcagccgcggtaatacgggggatgcgagcgttatccggatttattgggtttaaagggcgcgtaggcgggacgccaagtcagcggtaaaagactgcagctaaactgtagcacgccgttgaaactggcgacctggagacgagacgagggaggcggaacaagtgaagtagcggtgaaatgcttagatatcacttggaaccccgatagcgaaggcagcttcccaggctcgatctgacgctgatgcgcgagagcgtgggtagcgaacaggattagataccctggtagtccacgccgtaaacgatgctcactggatcttggcgatacaccgccagggttcaagcgaaagtattaagtgagccacctggggagtacgtcggcaacgatgaaactcaaaggaattgacgggggcccgcacaagcggaggaacatgtggtttaattcgatgatacgcgaggaaccttacccgggtttaaatgtggagtgcatgaggtggaaacagttctttccctcgggactattcacaaggtgctgcatggttgtcgtcagctcgtgccgtgaggtgtcgggttaagtcccataacgagcgcaacccctatcgccagttgccagcggntgaagccgggcactctgtcgagactgccaccgtaaggtgcgaggaaggcggggatgacgtcaaatcagcacggcccttacacccggggcgacacacgtgttacaatggccggtacagagggcagccacggggtgacccggagcgaatctctaaagccggtcgtagttcggactggagtctgcaacccgactccacgaagttggattcgctagtaatcgcgcatcagccatggcgcggtgaatacgttcccgggccttgtacacaccgcccgtcaagccatggaagccgggagtacctgaagatcgtgaccgcgagaacggct

SEQ ID NO: 55 - DNA, 1402 bp, Flavonifractor plautii
  misc_feature: HB-472_Flavonifractor_plautii
gcagtcgaacggggtgctcatgacggaggattcgtccaacggattgagttacctagtggcggacgggtgagtaacgcgtgaggaacctgccttggagaggggaataacactccgaaaggagtgctaataccgcatgatgcagttgggtcgcatggctctgactgccaaagatttatcgctctgagatggcctcgcgtctgattagctagtaggcggggtaacggcccacctaggcgacgatcagtagccggactgagaggttgaccggccacattgggactgagacacggcccagactcctacgggaggcagcagtggggaatattgggcaatgggcgcaagcctgacccagcaacgccgcgtgaaggaagaaggctttcgggttgtaaacttcttttgtcggggacgaaacaaatgacggtacccgacgaataagccacggctaactacgtgccagcagccgcggtaatacgtaggtggcaagcgttatccggatttactgggtgtaaagggcgtgtaggcgggattgcaagtcagatgtgaaaactgggggctcaacctccagcctgcatttgaaactgtagttcttgagtgctggagaggcaatcggaattccgtgtgtagcggtgaaatgcgtagatatacggaggaacaccagtggcgaaggcggattgctggacagtaactgacgctgaggcgcgaaagcgtggggagcaaacaggattagataccctggtagtccacgccgtaaacgatggatactaggtgtggggggtctgaccccctccgtgccgcagttaacacaataagtatcccacctggggagtacgatcgcaaggttgaaactcaaaggaattgacgggggcccgcacaagcggtggagtatgtggtttaattcgaagcaacgcgaagaaccttaccagggcttgacatcccactaacgaagcagagatgcattaggtgcccttcggggaaagtggagacaggtggtgcatggttgtcgtcagctcgtgtcgtgagatgttgggttaagtcccgcaacgagcgcaacccttattgttagttgctacgcaagagcactctagcgagactgccgttgacaaaacggaggaaggtggggacgacgtcaaatcatcatgccccttatgtcctgggccacacacgtactacaatggtggttaacagagggaggcaataccgcgaggtggagcaaatccctaaaagccatcccagttcggattgcaggctgaaacccgcctgtatgaagttggaatcgctagtaatcgcggatcagcatgccgcggtgaatacgttcccgggccttgtacacaccgcccgtcacaccatgagagtcgggaacacccgaagtccgtagcctaaccgcaaggagggcgcgccgaag

SEQ ID NO: 56 - DNA, 1382 bp, Anaerotruncus colihominis
  misc_feature: HB-474_Anaerotruncus_colihominis
tgcagtcgaacggagcttacgttttgaagttttcggatggatgaatgtaagcttagtggcggacgggtgagtaacacgtgagcaacctgcctttcagagggggataacagccggaaacggctgctaataccgcatgatgttgcgggggcacatgcccctgcaaccaaaggagcaatccgctgaaagatgggctcgcgtccgattagccagttggcggggtaacggcccaccaaagcgacgatcggtagccggactgagaggttgaacggccacattgggactgagacacggcccagactcctacgggaggcagcagtgggggatattgcacaatgggcgaaagcctgatgcagcgacgccgcgtgagggaagacggtcttcggattgtaaacctctgtctttggggaagaaaatgacggtacccaaagaggaagctccggctaactacgtgccagcagccgcggtaatacgtagggagcaagcgttgtccggaattactgggtgtaaagggagcgtaggcgggatggcaagtagaatgttaaatccatcggctcaaccggtggctgcgttctaaactgccgttcttgagtgaagtagaggcaggcggaattcctagtgtagcggtgaaatgcgtagatattaggaggaacaccagtggcgaaggcggcctgctgggctttaactgacgctgaggctcgaaagcgtggggagcaaacaggattagataccctggtagtccacgccgtaaacgatgattactaggtgtggggggactgaccccttccgtgccgcagttaacacaataagtaatccacctggggagtacggccgcaaggttgaaactcaaaggaattgacgggggcccgcacaagcagtggagtatgtggtttaattcgaagcaacgcgaagaaccttaccaggtcttgacatcggatgcatagcctagagataggtgaagcccttcggggcatccagacaggtggtgcatggttgtcgtcagctcgtgtcgtgagatgttgggttaagtcccgcaacgagcgcaacccttattattagttgctacgcaagagcactctaatgagactgccgttgacaaaacggaggaaggtggggatgacgtcaaatcatcatgccccttatgacctgggctacacacgtactacaatggcactaaaacagagggcggcgacaccgcgaggtgaagcgaatcccgaaaaagtgtctcagttcagattgcaggctgcaacccgcctgcatgaagtcggaattgctagtaatcgcggatcagcatgccgcggtgaatacgttcccgggccttgtacacaccgcccgtcacaccatgggagtcggtaacacccgaagccagtagcctaaccgcaagg

SEQ ID NO: 57 - DNA, 1403 bp, Intestinimonas butyriciproducens
  misc_feature: HB-478_Intestinimonas_butyriciproducens
tgcagtcgaacggagcacccctgacggagttttcggacaacgaaagggaatgcttagtggcggacgggtgagtaacgcgtgagtaacctgccttggagtggggaataacagccggaaacggctgctaataccgcatgatgtatctggatcgcatggttctggataccaaagatttatcgctctgagatggactcgcgtctgattagctagttggtgaggtaatggctcaccaaggcgacgatcagtagccggactgagaggttggccggccacattgggactgagacacggcccagactcctacgggaggcagcagtggggaatattgggcaatgggcgaaagcctgacccagcaacgccgcgtgaaggaagaaggccctcgggttgtaaacttcttttgtcagggacgaagcaagtgacggtacctgacgaataagccacggctaactacgtgccagcagccgcggtaatacgtaggtggcaagcgttatccggatttactgggtgtaaagggcgtgtaggcgggagtgcaagtcagatgtgaaaactatgggctcaacccatagcctgcatttgaaactgtacttcttgagtgatggagaggcaggcggaattccctgtgtagcggtgaaatgcgtagatatagggaggaacaccagtggcgaaggcggcctgctggacattaactgacgctgaggcgcgaaagcgtggggagcaaacaggattagataccctggtagtccacgccgtaaacgatggatactaggtgtggggggtctgaccccctccgtgccgcagttaacacaataagtatcccacctggggagtacgatcgcaaggttgaaactcaaaggaattgacgggggcccgcacaagcggtggagtatgtggtttaattcgaagcaacgcgaagaaccttaccaggacttgacatcctactaacgaagcagagatgcataaggtgcccttcggggaaagtagagacaggtggtgcatggttgtcgtcagctcgtgtcgtgagatgttgggttaagtcccgcaacgagcgcaacccttattgttagttgctacgcaagagcactctagcgagactgccgttgacaaaacggaggaaggtggggacgacgtcaaatcatcatgccccttatgtcctgggccacacacgtactacaatggcggtcaacagagggaagcaaagccgcgaggtggagcaaatccctaaaagccgtcccagttcggattgcaggctgaaactcgcctgtatgaagtcggaatcgctagtaatcgcggatcagcatgccgcggtgaatacgttcccgggccttgtacacaccgcccgtcacaccatgagagtcgggaacacccgaagtccgtagcctaacagcaatgggggcgcggccgaa

SEQ ID NO: 58 - DNA, 906 bp, Bacteroides fragilis
  misc_feature: HB-58_Bacteroides_fragilis
gcttgctttctttgctggcgaccggcgcacgggtgagtaacacgtatccaacctgccctttactcggggatagcctttcgaaagaaagattaatacccgatagcataatgattccgcatggtttcattattaaaggattccggtaaaggatggggatgcgttccattaggttgttggtgaggtaacggctcaccaagccttcgatggataggggttctgagaggaaggtcccccacattggaactgagacacggtccaaactcctacgggaggcagcagtgaggaatattggtcaatgggcgctagcctgaaccagccaagtagcgtgaaggatgaaggctctatgggtcgtaaacttcttttatataagaataaagtgcagtatgtatactgttttgtatgtattatatgaataaggatcggctaactccgtgccagcagccgcggtaatacggaggatccgagcgttatccggatttattgggtttaaagggagcgtaggtggactggtaagtcagttgtgaaagtttgcggctcaaccgtaaaattgcagttgatactgtcagtcttgagtacagtagaggtgggcggaattcgtggtgtagcggtgaaatgcttagatatcacgaagaactccgattgcgaaggcagctcactggactgcaactgacactgatgctcgaaagtgtgggtatcaaacaggattagataccctggtagtccacacagtaaacgatgaatactcgctgtttgcgatatacagtaagcggccaagcgaaagcattaagtattccacctggggagtacgccggcaacggtgaaactcaaaggaattgacgggggcccgcacaagcggaggaacatgtggtttaattcgatgatacgcgaggaaccttacccgggctt

SEQ ID NO: 59 - DNA, 775 bp, Streptococcus gordonii
  misc_feature: HB-62_Streptococcus_gordonii
tagaacgcacagtttataccgtagcttgctacaccatagactgtgagttgcgaacgggtgagtaacgcgtaggtaacctgcctggtagcgggggataactattggaaacgatagctaataccgcataatattaattattgcatgataattgattgaaaggtgcaattgcaccactaccagatggacctgcgttgtattagctagtaggtgaggtaacggctcacctaggcgacgatacatagccgacctgagagggtgatcggccacactgggactgagacacggcccagactcctacgggaggcagcagtagggaatcttcggcaatggacgaaagtctgaccgagcaacgccgcgtgagtgaagaaggttttcggatcgtaaagctctgttgtaagagaagaacgggtgtgagagtggaaagttcacactgtgacggtatcttaccagaaagggacggctaactacgtgccagcagccgcggtaatacgtaggtcccgagcgttgtccggatttattgggcgtaaagcgagcgcaggcggttagataagtctgaagttaaaggctgtggcttaaccatagtacgctttggaaactgtttaacttgagtgcagaaggggagagtggaattccatgtgtagcggtgaaatgcgtagatatatggaggaacaccggtggcgaaagcggctctctggtctgtaactgacgctgaggctcgaaagcgtggggagcgaacaggattagataccctggtagtccacgccgtaaacgatga

SEQ ID NO: 60 - DNA, 1409 bp, Enterococcus faecium
  misc_feature: HB-640_Enterococcus_faecium
tgcaagtcgtacgcttctttttccaccggagcttgctccaccggaaaaagaagagtggcgaacgggtgagtaacacgtgggtaacctgcccatcagaaggggataacacttggaaacaggtgctaataccgtataacaatcgaaaccgcatggttttgatttgaaaggcgctttcgggtgtcgctgatggatggacccgcggtgcattagctagttggtgaggtaacggctcaccaaggccacgatgcatagccgacctgagagggtgatcggccacattgggactgagacacggcccaaactcctacgggaggcagcagtagggaatcttcggcaatggacgaaagtctgaccgagcaacgccgcgtgagtgaagaaggttttcggatcgtaaaactctgttgttagagaagaacaaggatgagagtaactgttcatcccttgacggtatctaaccagaaagccacggctaactacgtgccagcagccgcggtaatacgtaggtggcaagcgttgtccggatttattgggcgtaaagcgagcgcaggcggtttcttaagtctgatgtgaaagcccccggctcaaccggggagggtcattggaaactgggagacttgagtgcagaagaggagagtggaattccatgtgtagcggtgaaatgcgtagatatatggaggaacaccagtggcgaaggcggctctctggtctgtaactgacgctgaggctcgaaagcgtggggagcaaacaggattagataccctggtagtccacgccgtaaacgatgagtgctaagtgttggagggtttccgcccttcagtgctgcagctaacgcattaagcactccgcctggggagtacgaccgcaaggttgaaactcaaaggaattgacgggggcccgcacaagcggtggagcatgtggtttaattcgaagcaacgcgaagaaccttaccaggtcttgacatcctttgaccactctagagatagagcttccccttcgggggcaaagtgacaggtggtgcatggttgtcgtcagctcgtgtcgtgagatgttgggttaagtcccgcaacgagcgcaacccttattgttagttgccatcattcagttgggcactctagcaagactgccggtgacaaaccggaggaaggtggggatgacgtcaaatcatcatgccccttatgacctgggctacacacgtgctacaatgggaagtacaacgagttgcgaagtcgcgaggctaagctaatctcttaaagcttctctcagttcggattgcaggctgcaactcgcctgcatgaagccggaatcgctagtaatcgcggatcagcacgccgcggtgaatacgttcccgggccttgtacacaccgcccgtcacaccacgagagtttgtaacacccgaagtcggtgaggta

SEQ ID NO: 61 - DNA, 1397 bp, Clostridium clostridioforme
  misc_feature: HB-642_Clostridium_clostridioforme
  misc_feature (1327)..(1327): n is a, c, g, or t
caggatgaacgctggcggcgtgcctaacacatgcaagtcgaacgaagcaattaagatgaagttttcggatggaatcttgattgactgagtggcggacgggtgagtaacgcgtggataacctgcctcacactgggggataacagttagaaatgactgctaataccgcataagcgcacagtgccgcatggcagtgtgtgaaaaactccggtggtgtgagatggatccgcgtctgattagccagttggcggggtaacggcccaccaaagcgacgatcagtagccgacctgagagggtgaccggccacattgggactgagacacggcccaaactcctacgggaggcagcagtggggaatattgcacaatgggcgaaagcctgatgcagcgacgccgcgtgagtgaagaagtatttcggtatgtaaagctctatcagcagggaagaaaatgacggtacctgactaagaagccccggctaactacgtgccagcagccgcggtaatacgtagggggcaagcgttatccggatttactgggtgtaaagggagcgtagacggcgaagcaagtctgaagtgaaaacccagggctcaaccctgggactgctttggaaactgttttgctagagtgtcggagaggtaagtggaattcctagtgtagcggtgaaatgcgtagatattaggaggaacaccagtggcgaaggcggcttactggacgataactgacgttgaggctcgaaagcgtggggagcaaacaggattagataccctggtagtccacgccgtaaacgatgaatgctaggtgttggggggcaaagcccttcggtgccgccgcaaacgcagtaagcattccacctggggagtacgttcgcaagaatgaaactcaaaggaattgacggggacccgcacaagcggtggagcatgtggtttaattcgaagcaacgcgaagaaccttaccaagtcttgacatccccctgacgggccggtaacgcggcctttccttcgggacaggggagacaggtggtgcatggttgtcgtcagctcgtgtcgtgagatgttgggttaagtcccgcaacgagcgcaacccttatccttagtagccagcacgtgaaggtgggcactctagggagactgccagggataacctggaggaaggtggggatgacgtcaaatcatcatgccccttatgatttgggctacacacgtgctacaatggcgtaaacaaagggaagcgagacagtgatgtggagcaaatcccaaaaataacgtcccagttcggactgtagtctgcaacccgactacacgaagctggaatcgctagtaatcgcgaatcanaatgtcgcggtgaatacgttcccgggtcttgtacacaccgcccgtcacaccatgggagtcagcaacgccc

SEQ ID NO: 62 - DNA, 1384 bp, Faecalitalea cylindroides
  misc_feature: HB-664_Faecalitalea_cylindroides
  misc_feature (1227)..(1227): n is a, c, g, or t
  misc_feature (1338)..(1338): n is a, c, g, or t
gctcaggatgaacgctggcggcatgcctaatacatgcaagtcgaacggaccgaagggaaagcttgcttttcctgaagtcagtggcgaacgggtgagtaacacgtaggtaacctgcccatgtgcccgggacaacgcctggaaacgggtgctaaaaccggataggtagagagatcgcatgaactttctattaaaggggctacggccctgaacatggatggacctgcggtgcattagctagttggtgaggtaacggcccaccaaggcgacgatgcatagccgacctgagagggcgaacggccacattgggactgagacacggcccaaactcctacgggaggcagcagtagggaattttcgtcaatggggggaaccctgaacgagcaatgccgcgtgagtgaagacggtcttcggattgtaaagctctgttgcgagagaaaaacgacacggatagggaatgatccgtgggtgatggtatctcgccagaaagtcacggctaactacgtgccagcagccgcggtaatacgtaggtggcgagcgttatccggaatgattgggcgtaaagggtgcgtaggcggcccgttaagtctggagtcaaagccggcagctcaactgtcgtacgctttggaaactggcgggctggagtgcagaagagggcgatggaactccatgtgtagcggtaaaatgcgtagatatatggaagaacaccagtggcgaaggcggtcgcctggtctgcaactgacgctgaagcacgaaagcgtggggagcaaataggattagataccctagtagtccacgccgtaaacgatgagaactaagtgttgggggaataactcagtgctgcagttaacgcaataagttctccgcctggggagtatgcacgcaagtgtgaaactcaaaggaattgacgggggcccgcacaagcggtggagtatgtggtttaattcgaagcaacgcgaagaaccttaccaggccttgacatcccgggcaagggcgtagagatacgccggaggctatcccggtgacaggtggtgcatggttgtcgtcagctcgtgtcgtgagatgttgggttaagtcccgcaacgagcgcaacccttgtgatatgttactaccagtaagatgaggactcatatcagactgccggtgacaaaccggaggaaggtggggatgacgtcaaatcatcatgccccttatggcctgggctacacacgtactacaatggcgcatacnaagagcagcgaacccgtgagggcaggcgaatctcgcaaagtgcgtctcagttcggattgaagtctgcaacccgacttcatgaagttggaatcgctagtaatcgcgaatcancatgtcgcggtgaatacgttctcgggccttgtacacaccgcccgtc

SEQ ID NO: 63 - DNA, 1290 bp, Bilophila wadsworthia
  misc_feature: HB-693_Bilophila_wadsworthia
  misc_feature (944)..(944): n is a, c, g, or t
  misc_feature (1117)..(1117): n is a, c, g, or t
  misc_feature (1122)..(1122): n is a, c, g, or t
  misc_feature (1165)..(1165): n is a, c, g, or t
  misc_feature (1246)..(1246): n is a, c, g, or t
  misc_feature (1260)..(1260): n is a, c, g, or t
  misc_feature (1264)..(1264): n is a, c, g, or t
  misc_feature (1276)..(1276): n is a, c, g, or t
tcagattgaacgctggcggcgtgcttaacacatgcaagtcgaacgtgaaagtccttcgggacgagtaaagtggcgcacgggtgagtaacgcgtggataatctacccttaagatggggataacggctggaaacggtcgctaataccgaatacgctcccgattttatcattggggggaaagatggcctctgcttgcaagctatcgcttaaggatgagtccgcgtcccattagctagttggcggggtaacggcccaccaaggcgacgatgggtagccggtctgagaggatgaccggccacactggaactggaacacggtccagactcctacgggaggcagcagtggggaatattgcgcaatgggcgaaagcctgacgcagcgacgccgcgtgagggatgaaggttctcggatcgtaaacctctgtcaggggggaagaaaccccctcgtgtgaataatgcgagggcttgacggtacccccaaaggaagcaccggctaactccgtgccagcagccgcggtaatacggagggtgcaagcgttaatcggaatcactgggcgtaaagcgcacgtaggcggcttggtaagtcaggggtgaaatcccacagcccaactgtggaactgcctttgatactgccaggcttgagtaccggagagggtggcggaattccaggtgtaggagtgaaatccgtagatatctggaggaacaccggtggcgaaggcggccacctggacggtaactgacgctgaggtgcgaaagcgtgggtagcaaacaggattagataccctggtagtccacgctgtaaacgatgggtgctgggtgctgggatgtatgtctcggtgccgtagctaacgcgataagcaccccgcctggggagtacggtcgcaaggctgaaactcaaagaaattgacgggggcccgcacaagcggtggagtatgtggtttaattcnatgcaacgcgaagaaccttacccaggcttgacatctagggaacccttcggaaatgaaggggtgcccttcggggagccctaagacaggtgctgcatggctgtcgtcagctcgtgccgtgaggtgttgggttaagtcccgcaacgagcgcaacccctatcttcagttgccagcangtaangctgggcactctggagagaccgccccggtcaacggggaggaangtggggacgacgtcaagtcatcatggcccttacgcctggggctacacacgtactacaatggcgcgcacaaagggtagcganaccgcgaggtgganccantcccaaaaacgngtcccagtccggat

SEQ ID NO: 64 - DNA, 731 bp, Bifidobacterium longum
  misc_feature: HB-71_Bifidobacterium_longum
tttgcttggtggtgagagtggcgaacgggtgagtaatgcgtgaccgacctgccccatacaccggaatagctcctggaaacgggtggtaatgccggatgctccagttgatcgcatggtcttctgggaaagctttcgcggtatgggatggggtcgcgtcctatcagcttgacggcggggtaacggcccaccgtggcttcgacgggtagccggcctgagagggcgaccggccacattgggactgagatacggcccagactcctacgggaggcagcagtggggaatattgcacaatgggcgcaagcctgatgcagcgacgccgcgtgagggatggaggccttcgggttgtaaacctcttttatcggggagcaagcgagagtgagtttacccgttgaataagcaccggctaactacgtgccagcagccgcggtaatacgtagggtgcaagcgttatccggaattattgggcgtaaagggctcgtaggcggttcgtcgcgtccggtgtgaaagtccatcgcttaacggtggatccgcgccgggtacgggcgggcttgagtgcggtaggggagactggaattcccggtgtaacggtggaatgtgtagatatcgggaagaacaccaatggcgaaggcaggtctctgggccgttactgacgctgaggagcgaaagcgtggggagcgaacaggattagataccctggtagtccacgccgtaaacggtggatgctggatgtg

SEQ ID NO: 65 - DNA, 853 bp, Clostridium hylemonae
  misc_feature: HB-73_Clostridium_hylemonae
  misc_feature (787)..(789): n is a, c, g, or t
ctgtgtgaagagattagcttgctaagatcagaactttgtattgactgagtggcggacgggtgagtaacgcgtgggcaacctgccttacacagggggataacagctagaaatggctgctaataccgcataagacctcagtaccgcatggtagaggggtaaaaactccggtggtgtaagatgggcccgcgtctgattaggtagttggtagggtaacggcctaccaagccgacgatcagtagccgacctgagagggtgaccggccacattgggactgagacacggcccaaactcctacgggaggcagcagtggggaatattgcacaatgggggaaaccctgatgcagcgacgccgcgtgaaggatgaagtatttcggtatgtaaacttctatcagcagggaagaagatgacggtacctgactaagaagccccggctaactacgtgccagcagccgcggtaatacgtagggggcaagcgttatccggatttactgggtgtaaagggagcgtagacggcatggcaagtctgaagtgaaagcccggggctcaaccccgggactgctttggaaactgtcaggctagagtgtcggagaggcaagtggaattcctagtgtagcggtgaaatgcgtagatattaggaggaacaccagtggcgaaggcggcttgctggacgatgactgacgttgaggctcgaaagcgtggggagcaaacaggattagataccctggtagtccacgccgtaaacgatgattactaggtgtcgggaagcaaagcttttcggtgccgcagnnnacgcaataagtaatccacctggggagtacgttcgcaagaatgaaactcaaaggaattgacgggg

SEQ ID NO: 66 - DNA, 752 bp, Clostridium innoculum
  misc_feature: HB-82_Clostridium_innoculum
ggaagcttgcttccaaagagacttagtggcgaacgggtgagtaacacgtaggtaacctgcccatgtgcccgggataactgctggaaacggtagctaaaaccggataggtatacagagcgcatgctctgtatattaaagcgcccatcaaggcgtgaacatggatggacctgcggcgcattagctagttggtgaggtaacggcccaccaaggcgatgatgcgtagccggcctgagagggtaaacggccacattgggactgagacacggcccaaactcctacgggaggcagcagtagggaattttcgtcaatgggggaaaccctgaacgagcaatgccgcgtgagtgaagaaggtcttcggatcgtaaagctctgttgtaagtgaagaacggctcatagaggaaatgctatgggagtgacggtagcttaccagaaagccacggctaactacgtgccagcagccgcggtaatacgtaggtggcaagcgttatccggaatcattgggcgtaaagggtgcgtaggtggcgtactaagtctgtagtaaaaggcaatggctcaaccattgtaagctatggaaactggtatgctggagtgcagaagagggcgatggaattccatgtgtagcggtaaaatgcgtagatatatggaggaacaccagtggcgaaggcggtcgcctggtctgtaactgacactgaggcacgaaagcgtggggagcaaataggattagataccctagtagtccacgccgtaaacga

SEQ ID NO: 67 - DNA, 761 bp, Bifidobacterium breve
  misc_feature: HB-90_Bifidobacterium_breve
tgcagtcgaacgggatccatcaggctttgcttggtggtgagagtggcgaacgggtgagtaatgcgtgaccgacctgccccatgcaccggaatagctcctggaaacgggtggtaatgccggatgctccatcacatcgcatggtgtgttgggaaagcctttgcggcatgggatggggtcgcgtcctatcagcttgatggcggggtaacggcccaccatggcttcgacgggtagccggcctgagagggcgaccggccacattgggactgagatacggcccagactcctacgggaggcagcagtggggaatattgcacaatgggcgcaagcctgatgcagcgacgccgcgtgagggatggaggccttcgggttgtaaacctcttttgttagggagcaaggcactttgtgttgagtgtacctttcgaataagcaccggctaactacgtgccagcagccgcggtaatacgtagggtgcaagcgttatccggaattattgggcgtaaagggctcgtaggcggttcgtcgcgtccggtgtgaaagtccatcgcttaacggtggatccgcgccgggtacgggcgggcttgagtgcggtaggggagactggaattcccggtgtaacggtggaatgtgtagatatcgggaagaacaccaatggcgaaggcaggtctctgggccgttactgacgctgaggagcgaaagcgtggggagcgaacaggattagataccctggtagtccacgccgtaaacggtggatgctggatgtg

SEQ ID NO: 68 - DNA, 862 bp, Acidaminococcus intestini
  misc_feature: HB-95_Acidaminococcus_intestini
gttcttagtggcgaacgggtgagtaacgcgtgggcaacctgccctccagttggggacaacattccgaaagggatgctaataccgaatgtgctccctcctccgcatggaggagggaggaaagatggcctctgcttgcaagctatcgctggaagatgggcccgcgtctgattagctagttggtggggtaacggctcaccaaggcgatgatcagtagccggtctgagaggatgaacggccacattgggactgagacacggcccaaactcctacgggaggcagcagtggggaatcttccgcaatggacgaaagtctgacggagcaacgccgcgtgagtgatgaaggtcttcggattgtaaagctctgttgttagggacgaaagcaccgtgttcgaacaggtcatggtgttgacggtacctaacgaggaagccacggctaactacgtgccagcagccgcggtaatacgtaggtggcaagcgttgtccggaattattgggcgtaaagagcatgtaggcgggcttttaagtctgacgtgaaaatgcggggcttaaccccgtatggcgttggatactggaagtcttgagtgcaggagaggaaaggggaattcccagtgtagcggtgaaatgcgtagatattgggaggaacaccagtggcgaaggcgcctttctggactgtgtctgacgctgagatgcgaaagccagggtagcaaacgggattagataccccggtagtcctggccgtaaacgatggatactaggtgtaggaggtatcgaccccttctgtgccggagttaacgcaataagtatcccgcctggggactacgatcgcaagattgaaactcaaaggaattgacgggggcccgcac

SEQ ID NO: 69 - DNA, 673 bp, Streptococcus gordonii
  misc_feature: HB-98_Streptococcus_gordonii
cagttaagaaccttcgggggaataagtgattggaaagtggcgaacgggtgagtaacgcgtgggtaacctgccctatggaaaggaatagcctcgggaaactgggagtaaagccttatattatggagagatcgcatggtcatttcatgaaaactccggtgccataggatggacccgcgtcccattagctagttggtgagataacagcccaccaaggcgacgatgggtaaccggtctgagagggcgaacggtcacactggaactgagacacggtccagactcctacgggaggcagcagtggggaatattgcgcaatgggggcaaccctgacgcagcaataccgcgtgagtgaagaaggttttcggatcgtaaagctctgttattggggaagaagaatgacggtacccaatgaggaagtcccggctaactacgtgccagcagccgcggtaatacgtaggggacaagcgttgtccggaatgactgggcgtaaagggcgcgtaggcggtctgataagtcagatgtgaaaggtaccggctcaaccggtgacgtgcatttgaaactgtcagacttgagtattggagaggcaagtggaattcctagtgtagcggtgaaatgcgtagatattaggaggaacaccagtggcgaaggcggcttgctggacaaatactgacgctga

SEQ ID NO: 70 - DNA, 1374 bp, Romboutsia lituseburensis
  misc_feature: HB-102_Romboutsia_lituseburensis
  misc_feature (1352)..(1352): n is a, c, g, or t
tgcagtcgagcgaacttcttcggaagtgagcggcggacgggtgagtaacgcgtgggtaacctgccctatacacacggataacgtaccgaaaggtacgctaatacgagatgatatatttttatcgcatggtagaaatatcaaagctccggcggtataggatggacccgcgtctgattagctagttggtaaggtaacggcttaccaaggcgacgatcagtagccgacctgagagggtgatcggccacattggaactgagacacggtccaaactcctacgggaggcagcagtggggaatattgcacaatgggcgaaagcctgatgcagcaacgccgcgtgagcgatgaaggccttcgggtcgtaaagctctgtcctcaaggaagataatgacggtacttgaggaggaagccccggctaactacgtgccagcagccgcggtaatacgtagggggctagcgttatccggaattactgggcgtaaagggtgcgtaggtggtttcttaagtcagaggtgaaaggctacggctcaaccgtagtaagcctttgaaactgggaaacttgagtgcaggagaggagagtagaattcctagtgtagcggtgaaatgcgtagatattaggaggaataccagttgcgaaggcggctctctggactgtaactgacactgaggcacgaaagcgtggggagcaaacaggattagataccctggtagtccacgccgtaaacgatgagtactagctgtcggaggttacccccttcggtggcgcagctaacgcattaagtactccgcctgggaagtacgctcgcaagagtgaaactcaaaggaattgacggggacccgcacaagtagcggagcatgtggtttaattcgaagcaacgcgaagaaccttacctaagcttgacatccttttgacctctccctaatcggagatttcccttcggggacagaagtgacaggtggtgcatggttgtcgtcagctcgtgtcgtgagatgttgggttaagtcccgcaacgagcgcaacccttgcctttagttgccagcattaagttgggcactctagagggactgccagggataacctggaggaaggtggggatgacgtcaaatcatcatgccccttatgcttagggctacacacgtgctacaatgggtggtacagagggcagccaagtcgtgaggcggagctaatcccttaaagccattctcagttcggattgtaggctgaaactcgcctacatgaagctggagttactagtaatcgcagatcagaatgctgcggtgaatgcgttcccgggtcttgtacacaccgcccgtcacaccatgggagttgggggcgcccgaagccggttagctanccttttaggaagcggccgtcga

SEQ ID NO: 71 - DNA, 1341 bp, Blautia wexlerae
  misc_feature: HB-16_Blautia_wexlerae
  misc_feature (879)..(879): n is a, c, g, or t
  misc_feature (931)..(931): n is a, c, g, or t
  misc_feature (933)..(933): n is a, c, g, or t
  misc_feature (1287)..(1287): n is a, c, g, or t
gattctaatttctagtggcggacgggtgagtaacgcgtgggtaacctgccttatacagggggataacagtcagaaatggctgctaataccgcataagcgcacagagctgcatggctcagtgtgaaaaactccggtggtataagatggacccgcgttggattagcttgttggtggggtaacggcccaccaaggcgacgatccatagccggcctgagagggtgaacggccacattgggactgagacacggcccagactcctacgggaggcagcagtggggaatattgcacaatgggggaaaccctgatgcagcgacgccgcgtgaaggaagaagtatctcggtatgtaaacttctatcagcagggaagatagtgacggtacctgactaagaagccccggctaactacgtgccagcagccgcggtaatacgtagggggcaagcgttatccggatttactgggtgtaaagggagcgtagacggtgtggcaagtctgatgtgaaaggcatgggctcaacctgtggactgcattggaaactgtcatacttgagtgccggaggggtaagcggaattcctagtgtagcggtgaaatgcgtagatattaggaggaacaccagtggcgaaggcggcttactggacggtaactgacgttgaggctcgaaagcgtggggagcaaacaggattagataccctggtagtccacgccgtaaacgatgaatactaggtgtcggggggcatggcccttcggtgccgtcgcaaacgcagtaagtattccacctggggagtacgttcgcaagaatgaaactcaaaggaattgacggggacccgcacaagcggtggagcatgtggtttaattcgaagcaacgcgaagaaccttaccaantcttgacatccctctgaccggtctttaatcggaccttctcttcggagcagangngacaggtggtgcatggttgtcgtcagctcgtgtcgtgagatgttgggttaagtcccgcaacgagcgcaacccctatcctcagtagccagcatttaaggtgggcactctggggagactgccagggataacctggaggaaggcggggatgacgtcaaatcatcatgccccttatgatttgggctacacacgtgctacaatggcgtaaacaaagggaagcgagatcgtgagatggagcaaatcccaaaaataacgtcccagttcggactgtagtctgcaacccgactacacgaagctggaatcgctagtaatcgcggatcagaatgccgcggtgaatacgttcccgggtcttgtacncaccgcccgtcacaccatggggagtcagtaacgcccgaagtcagtgacctaact

SEQ ID NO: 72 - DNA, 1327 bp, Sutterella wadsworthensis
  misc_feature: HB-259_Sutterella_wadsworthensis
tgcagtcgaacggcagcgcggggagcttgctccctggcggcgagtggcgcacgggtgagtaatacatcggaacgtgtcttctagtgggggataactgcccgaaagggcagctaataccgcatgagacctgagggtgaaagcgggggatcgcaagacctcgcgctggaagagcggccgatgtccgattagctagttggtgaggtaaaggctcaccaaggcgacgatcggtagctggtctgagaggacgaccagccacactgggactgagacacggcccagactcctacgggaggcagcagtggggaattttggacaatgggggcaaccctgatccagccatgccgcgtgcaggatgaaggtcttcggattgtaaactgcttttgtcagggacgaaaagggatgcgataacaccgcattccgctgacggtacctgaagaataagcaccggctaactacgtgccagcagccgcggtaatacgtagggtgcaagcgttaatcggaattactgggcgtaaagcgtgcgcaggcggttctgtaagatagatgtgaaatccccgggctcaacctgggaattgcatatatgactgcaggacttgagtttgtcagaggagggtggaattccacgtgtagcagtgaaatgcgtagatatgtggaagaacaccgatggcgaaggcagccctctgggacatgactgacgctcatgcacgaaagcgtggggagcaaacaggattagataccctggtagtccacgccctaaacgatgtctactagttgttggggacgatagtccttggtaacgcagctaacgcgtgaagtagaccgcctggggagtacggtcgcaagattaaaactcaaaggaattgacggggacccgcacaagcggtggatgatgtggattaattcgatgcaacgcgaaaaaccttacctagccttgacatgccaggaaggcctgagagatcaggccgtgcccgcaagggaatctggacacaggtgctgcatggctgtcgtcagctcgtgtcgtgagatgttgggttaagtcccgcaacgagcgcaacccttgtcattagttgctacgaaagggcactctaatgagactgccggtgacaaaccggaggaaggtggggatgacgtcaagtcctcatggcccttatggctagggcctcacacgtcatacaatggtcggaacagagggaagcgaagccgcgaggtgaagccaatcccagaaaaccgatcgtagtccggattgcagtctgcaactcgactgcatgaagtcggaatcgctagtaatcgcggatcagcatgccgcggtgatacgttcccgggtctg

SEQ ID NO: 73 - DNA, 1391 bp, Bacteroides clarus
  misc_feature: HB-30_Bacteroides_clarus
tgcagtcgaggggcagcggggttgaagcttgcttcaaccgccggcgaccggcgcacgggtgagtaacacgtatccaacctgccgataactccgggatagcctttcgaaagaaagattaataccggatggcatagttttcccgcatggaataactattaaagaatttcggttatcgatggggatgcgttccattaggcagttggcggggtaacggcccaccaaaccgacgatggataggggttctgagaggaaggtcccccacattggaactgagacacggtccaaactcctacgggaggcagcagtgaggaatattggtcaatggacgagagtctgaaccagccaagtagcgtgaaggatgactgccctatgggttgtaaacttcttttatacgggaataaagttggccacgtgtggttttttgcatgtaccgtatgaataaggatcggctaactccgtgccagcagccgcggtaatacggaggatccgagcgttatccggatttattgggtttaaagggagcgtaggcggggtattaagtcagttgtgaaagtttgcggctcaaccgtaaaattgcagttgatactggtatccttgagtgcagcagaggtgggcggaattcgtggtgtagcggtgaaatgcttagatatcacgaagaactccgattgcgaaggcagctcactggagtgtaactgacgctgatgctcgaaagtgtgggtatcaaacaggattagataccctggtagtccacacagtaaacgatgaatactcgctgttggcgatacaatgtcagcggccaagcgaaagcattaagtattccacctggggagtacgccggcaacggtgaaactcaaaggaattgacgggggcccgcacaagcggaggaacatgtggtttaattcgatgatacgcgaggaaccttacccgggcttgaattgcaactgactgagctggaaacagttctttcttcggacagttgtgaaggtgctgcatggttgtcgtcagctcgtgccgtgaggtgtcggcttaagtgccataacgagcgcaacccttatctatagttaccatcaggtcatgctggggactctatggagactgccgtcgtaagatgtgaggaaggtggggatgacgtcaaatcagcacggcccttacgtccggggctacacacgtgttacaatggggggtacagaaggcagctacacggcgacgtgatgctaatcccaaaaacctctctcagttcggattggagtctgcaacccgactccatgaagctggattcgctagtaatcgcgcatcagccacggcgcggtgaatacgttcccgggccttgtacacaccgcccgtcaagccatgaaagccgggggtacctgaagtacgtaaccgcaaggagc

SEQ ID NO: 74 - DNA, 1407 bp, Prevotella sp.
  misc_feature: HB-333_Prevotella_sp.
catgcagtcgtggggcagcatgggagatgcttgcatttcccgatggcgaccggcgcacgggtgagtaacgcgtatccaacctgcccttcactccggcacagcccgtcgaaagacggattaatgcgggatgcagtcagaaagatgcatattaatttgacgaaaggctttatgccggtgagggatggggatgcgtccgattagcttgacggcggggtaacggcccaccgtggcgacgatcggtaggggttctgaaaggaaggtcccccacattggaactgaaacacggtccaaactcctacgggaggcagcagtgaggaatattggtcaatgggcgaaagcctgaaccagccaagtagcgtgcaggaagacggccctatgggttgtaaactgcttttgtgcgggaataaagggggtcacgtgtggccctttgcatgtaccgtacgaataaggaccggctaattccgtgccagcagccgcggtaatacggaaggtccgggcgttatccggatttattgggtttaaagggagcgtaggccgtctcaaaagcgtgttgtgaaatgtccgggctcaaccggggccgtgcagcgcgaactgtgagacttgagtgtgcggaaggtatgcggaattcgtggtgtagcggtgaaatgcatagatatcacgaagaactccgattgcgaaggcagcatgccgcagcattactgacgctgatgctcgaaggtgcgggtatcgaacaggattagataccctggtagtccgcacagtaaacgatggatgcccgctgtgagcctgtttaatggtttgcggccaagcgaaagcgttaagcatcccacctggggagtacgccggcaacggtgaaactcaaaggaattgacgggggcccgcacaagcggaggaacatgtggtttaattcgatgatacgcgaggaaccttacccgggcttgaattgcaggtgcatggggtggagacatccctttccttcgggactcctgtgaaggtgctgcatggttgtcgtcagctcgtgccgtgaggtgtcggcttaagtgccataacgagcgcaacccctctccgtagttgccatcgggttaagccgggcactctgcggacactgcctccgcaaggagtgaggaaggtggggatgacgtcaaatcagcacggcccttacgtccggggctacacacgtgttacaatggggcatacagagagcagggtgtacgcaagtgcgcccgaatcaagaaagtgcctctcagttcggactggggtctgcaacccgaccccacgaagctggattcgctagtaatcgcgcatcagccatggcgcggtgaatacgttcccgggccttgtacacaccgcccgtcaagccatgaaagccgggggcgcctgaagtccgtgaccgcgagggtcggccta

SEQ ID NO: 75 - DNA, 1406 bp, Bacteroides thetaiotaomicron
  misc_feature: HB-34_Bacteroides_thetaiotaomicron
  misc_feature (954)..(954): n is a, c, g, or t
catgcagtcgaggggcagcatttcagtttgcttgcaaactggagatggcgaccggcgcacgggtgagtaacacgtatccaacctgccgataactcggggatagcctttcgaaagaaagattaatacccgatggtataatcagaccgcatggtcttgttattaaagaatttcggttatcgatggggatgcgttccattaggcagttggtgaggtaacggctcaccaaaccttcgatggataggggttctgagaggaaggtcccccacattggaactgagacacggtccaaactcctacgggaggcagcagtgaggaatattggtcaatgggcgcaggcctgaaccagccaagtagcgtgaaggatgactgccctatgggttgtaaacttcttttatatgggaataaagttttccacgtgtggaattttgtatgtaccatatgaataaggatcggctaactccgtgccagcagccgcggtaatacggaggatccgagcgttatccggatttattgggtttaaagggagcgtaggtggacagttaagtcagttgtgaaagtttgcggctcaaccgtaaaattgcagttgatactggctgtcttgagtacagtagaggtgggcggaattcgtggtgtagcggtgaaatgcttagatatcacgaagaactccgattgcgaaggcagctcactggactgcaactgacactgatgctcgaaagtgtgggtatcaaacaggattagataccctggtagtccacacagtaaacgatgaatactcgctgtttgcgatatacagtaagcggccaagcgaaagcattaagtattccacctggggagtacgccggcaacggtgaaactcaaaggaattgacgggggcccgcacaagcggaggaacatgtggtttaattcgatgatacgcgaggaaccttacccgggcttaaattgcatttgaatatnttggaaacagkatagccgtaaggcaaatgtgaaggtgctgcatggttgtcgtcagctcgtgccgtgaggtgtcggcttaagtgccataacgagcgcaacccttatctttagttactaacaggtcatgctgaggactctagagagactgccgtcgtaagatgtgaggaaggtggggatgacgtcaaatcagcacggcccttacgtccggggctacacacgtgttacaatggggggtacagaaggcagctacctggtgacaggatgctaatcccaaaagcctctctcagttcggatcgaagtctgcaacccgacttcgtgaagctggattcgctagtaatcgcgcatcagccatggcgcggtgaatacgttcccgggccttgtacacaccgcccgtcaagccatgaaagccgggggtacctgaagtacgtaaccgcaaggagcgtcctagggta

SEQ ID NO: 76 - DNA, 1373 bp, Prevotella copri
  misc_feature: HB-373_Prevotella_copri
cgtgcttccgataggcgtcgaccggcgcacgggtgagtaacgcgtatccaacctgcccaccacttggggataaccttgcgaaagtaagactaatacccaatgatatctctagaagacatctgaaagagattaaagatttatcggtgatggatggggatgcgtctgattagcttgttggcggggtaacggcccaccaaggcaacgatcagtaggggttctgagaggaaggtcccccacattggaactgagacacggtccaaactcctacgggaggcagcagtgaggaatattggtcaatggacgagagtctgaaccagccaagtagcgtgcaggaagacggccctatgggttgtaaactgcttttataagggaataaagtgagtctcgtgagactttttgcatgtaccttatgaataaggaccggctaattccgtgccagcagccgcggtaatacggaaggtccgggcgttatccggatttattgggtttaaagggagcgtaggccggagattaagcgtgttgtgaaatgtagatgctcaacatctgmactgcagcgcgaactggtttccttgagtacgcacaaagtgggcggaattcgtggtgtagcggtgaaatgcttagatatcacgaagaactccgattgcgaaggcagctcactggagcgcaactgacgctgaagctcgaaagtgcgggtatcgaacaggattagataccctggtagtccgcacggtaaacgatggatgcccgctgttggtctgaataggtcagcggccaagcgaaagcattaagcatcccacctggggagtacgccggcaacggtgaaactcaaaggaattgacgggggcccgcacaagcggaggaacatgtggtttaattcgatgatacgcgaggaaccttacccgggcttgaattgcagaggaaggatttggagacaatgacgcccttcggggcctctgtgaaggtgctgcatggttgtcgtcagctcgtgccgtgaggtgtcggcttaagtgccataacgagcgcaacccctctccttagttgccatcaggtgaagctgggcactctggggacactgccaccgtaaggtgtgaggaaggtggggatgacgtcaaatcagcacggcccttacgtccggggctacacacgtgttacaatggcaggtacagagagacggtcccttgcaaaatggatcaaatccttaaagcctgtctcagttcggactggggtctgcaacccgaccccacgaagctggattcgctagtaatcgcgcatcagccatggcgcggtgaatacgttcccgggccttgtacacaccgcccgtcaagccatgaaagccgggggcgcctaaagtccgtgaccgtaaggagcggcct

SEQ ID NO: 77 - DNA, 1439 bp, Dialister invisus
  misc_feature: HB-387_Dialister_invisus
  misc_feature (928)..(928): n is a, c, g, or t
tgcagtcgaacgaaagacggaaagagcttgctcttttcagaattgagtggcaaacgggtgagtaacacgtaaacaacctgccttcaggatggggacaacagacggaaacgactgctaataccgaataagttccaagagccgcatggcccatggaagaaaaggtggcctctacctgtaagctatcgcctgaagaggggtttgcgtctgattagctggttggaggggtaacggcccaccaaggcgacgatcagtagccggtctgagaggatgaacggccacactggaactgagacacggtccagactcctacgggaggcagcagtggggaatcttccgcaatgggcgaaagcctgacggagcaacgccgcgtgagtgatgacggccttcgggttgtaaaactctgtgatccgggacgaaaaggcagagtgcgaagaacaaactgcattgacggtaccggaaaagcaagccacggctaactacgtgccagcagccgcggtaatacgtaggtggcaagcgttgtccggaattattgggcgtaaagcgcgcgcaggcggcttcccaagtccctcttaaaagtgcggggcttaaccccgtgatgggaaggaaactgggaagctggagtatcggagaggaaagtggaattcctagtgtagcggtgaaatgcgtagagattaggaagaacaccggtggcgaaggcgactttctggacgaaaactgacgctgaggcgcgaaagcgtggggagcaaacaggattagataccctggtagtccacgccgtaaacgatggatactaggtgtaggaggtatcgacccctcctgtgccggagttaacgcaataagtatcccgcctgggaagtacgatcgcaagattaaaactcaaaggaattgacgggggcccgcacaagcggtggagtatgtggtttaattngacgcaacgcgaagaaccttaccaggtcttgacattgatcgcgatctgcagaaatgcggagttcttcttcggaagacgagaaaacaggtggtgcacggctgtcgtcagctcgtgtcgtgagatgttgggttaagtcccgcaacgagcgcaacccctatcatttgttgccagcacgtaaaggtggggactcaaatgagaccgccgcggacaacgcggaggaaggcggggacgacgtcaagtcatcatgccccttatgacctgggctacacacgtactacaatgggtgtcaacaaagagaagcgaaaccgcgaggaagagcaaacctcaaaaacacacccccagttcagatcgcaggctgcaacccgcctgcgtgaagcaggaatcgctagtaatcgcgggtcagcataccgcggtgaatacgttcccgggccttgtacacaccgcccgtcacactatgagagtcagaaacacccgaagccggtgaggtaaccgcaaggagccagccgtcgaa

SEQ ID NO: 78 - DNA, 1396 bp, Sellimonas intestinalis
  misc_feature: HB-443_Sellimonas_intestinalis
tgcagtcgagcgaagcgctgttttcagaatcttcggaggaagaggacagcgactgagcggcggacgggtgagtaacgcgtgggcaacctgcctcatacagggggataacagttagaaatgactgctaataccgcataagcgcacaggaccgcatggtgtagtgtgaaaaactccggtggtatgagatggacccgcgtctgattaggtagttggtggggtaaaggcctaccaagccgacgatcagtagccgacctgagagggtgaccggccacattgggactgagacacggcccaaactcctacgggaggcagcagtggggaatattgcacaatgggggaaaccctgatgcagcgacgccgcgtgaaggaagaagtatttcggtatgtaaacttctatcagcagggaagaagatgacggtacctgagtaagaagcaccggctaaatacgtgccagcagccgcggtaatacgtatggtgcaagcgttatccggatttactgggtgtaaagggagcgtagacggataggcaagtctggagtgaaaacccagggctcaaccctgggactgctttggaaactgcagatctggagtgccggagaggtaagcggaattcctagtgtagcggtgaaatgcgtagatattaggaggaacaccagtggcgaaggcggcttactggacggtgactgacgttgaggctcgaaagcgtggggagcaaacaggattagataccctggtagtccacgccgtaaacgatgactactaggtgtcggtgtgcaaagcacatcggtgccgcagcaaacgcaataagtagtccacctggggagtacgttcgcaagaatgaaactcaaaggaattgacggggacccgcacaagcggtggagcatgtggtttaattcgaagcaacgcgaagaaccttacctggtcttgacatccggatgacgggcgagtaatgtcgccgtcccttcggggcatccgagacaggtggtgcatggttgtcgtcagctcgtgtcgtgagatgttgggttaagtcccgcaacgagcgcaacccttatcttcagtagccagcatataaggtgggcactctggagagactgccagggagaacctggaggaaggtggggatgacgtcaaatcatcatgccccttatggccagggctacacacgtgctacaatggcgtaaacaaagggaagcgagagggtgacctggagcgaatcccaaaaataacgtctcagttcggattgtagtctgcaactcgactacatgaagctggaatcgctagtaatcgcggatcagcatgccgcggtgaatacgttcccgggtcttgtacacaccgcccgtcacaccatgggagtcagtaacgcccgaagccagtgacccaaccttagaggagggagc

SEQ ID NO: 79 - DNA, 1399 bp, Bittarella massiliensis
  misc_feature: HB-477_Bittarella_massiliensis
cttcggcagcgtcccccttgcggttagactactgacttcgggtgttaccggctctcatggtgtgacgggcggtgtgtacaaggcccgggaacgtattcaccgcggcatgctgatccgcgattactagcaattccgacttcatgcaggcgagttgcagcctgcaatccgaactgagaccgcttttatagattcgctccacctcgcggttttgctgctcattgttagcggccattgtagtacgtgtgtagcccaggtcataaagggcatgatgatttgacgtcatccccaccttcctccgttttgtcaacggcagtcccgttagagtgctcttgcgtagcaactaacagtaagggttgcgctcgttgcgggacttaacccaacatctcacgacacgagctgacgacaaccatgcaccacctgtcttaccgtccccgaagggaactcccaatctcttgggatagcgatagatgtcaagacctggtaaggtttttcgcgttgcttcgaattaaaccacatactccactgcttgtgcgggcccccgtcaattcctttgagtttcaaccttgcggtcgtactccccaggtggattacttattgtgttaactccggcacggagggggttatccccccacacctagtaatcatcgtttacggcatggactaccagggtatctaatcctgtttgctccccatgctttcgagcctcagcgtcagtaaaagcccagcaggccgccttcgccactggtgttcctcccgatatctacgcattccaccgctacaccgggaattccgcctgcctctacttcactcaagaaccacagttttgcgcgcagttcaggggttgagcccctgcatttcacacgcaacttatgatcccgcctacactccctttacacccagtaattccggacaacgctcgccatctacgtattaccgcggctgctggcacgtagttagccatggcttcctcctgtggtaccgtcattatcgtcccacaagacagaggtttacaatccgaaaaccttcttccctcacgcggcgtcgctgcatcagggttccccccattgtgcaatattccccactgctgcctcccgtaggagtctgggccgtgtctcagtcccaatgtggccgttcaacctctcagtccggctactgatcgttgccttggtgggccgttaccccgccaactagctaatcagacgcgagcccatctcagagcggatataatcctttcttggccaggggatgtcccccgaccaccgtatgcggtattagcagtcgtttccaactgttgtcccccactccgaggcaggttgctcacgtgttactcacccgtccgccactaacaacatccatcttcctagcaagctattccgtcggatgtgtccgttcgactgca

SEQ ID NO: 80 - DNA, 1346 bp, Ruminococcus sp.
  misc_feature: HB-626_Ruminococcus_sp.
gctcaggatgaacgctggcggcgtgcctaacacatgcaagtcgagcgaagcgcattgctttgaatctttcgggaggaagagcgaagtgacttagcggcggacgggtgagtaacgcgtgggtaacctgcctcatacagggggataacagttagaaatgactgctaataccgcataagcgcacaggaccgcatggtcaagtgtgaaaaactgaggtggtatgagatggacccgcgtctgattagctagttggtgaggtaagggctcaccaaggcaacgatcagtagccgacctgagagggtgaccggccacattgggactgagacacggcccaaactcctacgggaggcagcagtggggaatattgcacaatgggggaaaccctgatgcagcgacgccgcgtgagcgaagaagtatttcggtatgtaaagctctatcagcagggaagaaaatgacggtacctgactaagaagcaccggctaaatacgtgccagcagccgcggtaatacgtatggtgcaagcgttatccggatttactgggtgtaaagggagcgtagacggataagcaagtctggagtgaaagcccggggctcaaccccgggactgctttggaaactgttaatctagagtgctggagaggtaagtggaattcctagtgtagcggtgaaatgcgtagatattaggaggaacaccagtggcgaaggcggcttactggacagtgactgacgttgaggctcgaaagcgtggggagcaaacaggattagataccctggtagtccacgccgtaaacgatgactactaggtgtcgggtggcaaagccattcggtgccgcagccaacgcaataagtagtccacctggggagtacgttcgcaagaatgaaactcaaaggaattgacggggacccgcacaagcggtggagcatgtggtttaattcgaagcaacgcgaagaaccttacctgcccttgacatccgaatgacgagcgagtaatgtcgtttttccttcgggacattcgagacaggtggtgcatggttgtcgtcagctcgtgtcgtgagatgttgggttaagtcccgcaacgagcgcaacccttatctttagtagccagcaggtaaagctggggactctagagagactgccagggataacctggaggaaggtggggatgacgtcaaatcatcatgccccttatgggcagggctacacacgtgctacaatggcgtaaacaaagggaagcgagactgtgaagtggagcaaatcccaaaaataacgtctcagttcggattgtagtctgcaactcgactacatgaagctggaatcgctagtaatcgcgaatcagaatgtcgcgggtga

SEQ ID NO: 81 - DNA, 1439 bp, Prevotella sp.
  misc_feature: HB-649_Prevotella_sp.
ctcaggatgaacgctagctacaggcttaacacatgcaagtcgtggggcagcatgggagatgcttgcatttcccgatggcgaccggcgcacgggtgagtaacgcgtatccaacctgcccttcactccggcacagcccgtcgaaagacggattaatgcgggatgcagtcagaaagatgcatattaatttgacgaaaggctttatgccggtgagggatggggatgcgtccgattagcttgacggcggggtaacggcccaccgtggcgacgatcggtaggggttctgaraggaaggtcccccacattggaactgaracacggtccaaactcctacgggaggcagcagtgaggaatattggtcaatgggcgagagcctgaaccagccaagtagcgtgcaggaagacggccctatgggttgtaaactgcttttgtgcgggaataaagsgggtcacgtgtggccctttgcatgtaccgtacgaataaggaccggctaattccgtgccagcagccgcggtaatacggaaggtccgggcgttatccggatttattgggtttaaagggagcgtaggccgtctcaaaagcgtgttgtgaaatgtccgggctcaaccggggccgtgcagcgcgaactgtgagacttgagtgtgcggaaggtatgcggaattcgtggtgtagcggtgaaatgcatagatatcacgaagaactccgattgcgaaggcagcatgccgcagcattactgacgctgatgctcgaaggtgcgggtatcgaacaggattagataccctggtagtccgcacagtaaacgatggatgcccgctgtgagcctgtttaatggtttgcggccaagcgaaagcgttaagcatcccacctggggagtacgccggcaacggtgaaactcaaaggaattgacgggggcccgcacaagcggaggaacatgtggtttaattcgatgatacgcgaggaaccttacccgggcttgaattgcaggtgcatggggtggagacatccctttccttcgggactcctgtgaaggtgctgcatggttgtcgtcagctcgtgccgtgaggtgtcggcttaagtgccataacgagcgcaacccctctccgtagttgccatcgggttaagccgggcactctgcggacactgcctccgcaaggagtgaggaaggtggggatgacgtcaaatcagcacggcccttacgtccggggctacacacgtgttacaatggggcatacagagagcagggtgtacgcaagtgcgcccgaatcaagaaagtgcctctcagttcggactggggtctgcaacccgaccccacgaagctggattcgctagtaatcgcgcatcagccatggcgcggtgaatacgttcccgggccttgtacacaccgcccgtcaagccatgaaagccgggggcgcctgaagtccgtgaccgcgagggtcggccta

SEQ ID NO: 82 - DNA, 1396 bp, Longibaculum muris
  misc_feature: HB-79_Longibaculum_muris
tgcaagtcgaacgcaccgcttcggtggtgagtggcgaacgggtgagtaatacataagtaacctggcctttcgagggggataactattggaaacgatagctaagaccgcataggcataattctcgcatgagagttatgttaaatatcccaagggatagcgagaggatggacttatggcgcattagctagttggtgagggtaacggcccaccaaggcgacgatgcgtagccgacctgagagggtggacggccacactgggactgagacacggcccagactcctacgggaggcagcagtagggaattttcggcaatgggcgaaagcctgaccgagcaacgccgcgtgagggaggaagtatttcggtatgtaaacctctgttataaaggaagaacggcgcatgtagggaatgacatgcgagtgacggtactttatgagaaagccacggctaactacgtgccagcagccgcggtaatacgtaggtggcgagcgttatccggaatcattgggcgtaaagagggagcaggcggcaactagggtctgcggtaaaagaccgaagctaaacttcggtaagccgtggaaaccgaggagctagagtgcagtagaggatcgtggaattccatgtgtagcggtgaaatgcgtagatatatggaggaacaccagtggcgaaggcgacgatctgggctgcaactgacgctcagtcccgaaagcgtggggagcaaataggattagataccctagtagtccacgccgtaaacgatgagtactaagtgttgggggtcagacctcagtgctgcagttaacgcaataagtactccgcctgagtagtacgttcgcaagaatgaaactcaaaggaattgacgggggcccgcacaagcggtggagcatgtggtttaattcgaagcaacgcgaagaaccttaccaggtcttgacatacccttaaaggctctagagatagagagatagctataggggatacaggtggtgcatggttgtcgtcagctcgtgtcgtgagatgttgggttaagtcccgcaacgagcgcaacccctgttgccagttgccagcattaagttggggactctggcgagactgcctctgcaaggaggaggaaggcggggatgacgtcaaatcatcatgccccttatgacctgggctacacacgtgctacaatggacggatcagagggaagcgaagccgcgaggtggagcgaaacccagaaacccgttctcagttcggactgcagtctgcaactcgactgcacgaagttggaatcgctagtaatcgcgaatcagcatgtcgcggtgaatacgttctcgggccttgtacacaccgcccgtcacaccatgagagttggtaacacccgaagccggtggcctaaccgcaaggaaggagctgtcta

SEQ ID NO: 83 - DNA, 1381 bp, Ruminococcus bicirculans
  misc_feature: HB-105_Ruminococcus_bicirculans
cagtcgaacgagagaagagaagcttgcttttctgatctagtggcggacgggtgagtaacacgtgagcaatctgcctttcagagggggataccgattggaaacgatcgttaataccgcataacataattgaaccgcatgatttgattatcaaagatttatcgctgaaagatgagctcgcgtctgattagctagttggtaaggtaacggcttaccaaggcgacgatcagtagccggactgagaggttgatcggccacattgggactgagacacggcccagactcctacgggaggcagcagtggggaatattgcacaatggaggaaactctgatgcagcgatgccgcgtgagggaagaaggttttaggattgtaaacctctgtcttcagggacgaaaaaagacggtacctgaggaggaagctccggctaactacgtgccagcagccgcggtaatacgtagggagcgagcgttgtccggaattactgggtgtaaagggagcgtaggcgggatcgcaagtcagatgtgaaaactatgggcttaacccataaactgcatttgaaactgtggttcttgagtgaagtagaggtaagcggaattcctagtgtagcggtgaaatgcgtagatattaggaggaacatcagtggcgaaggcggcttactgggctttaactgacgctgaggctcgaaagcgtggggagcaaacaggattagataccctggtagtccacgccgtaaacgatgattactaggtgtggggggactgaccccttccgtgccgcagcaaacgcaataagtaatccacctggggagtacgaccgcaaggttgaaactcaaaggaattgacgggggcccgcacaagcagtggagtatgtggattaattcgaagcaacgcgaagaaccttaccaggtcttgacatcgtatgcatagctcagagatgagtgaaatctcttcggagacatatagacaggtggtgcatggttgtcgtcagctcgtgtcgtgagatgttgggttaagtcccgcaacgagcgcaacccttactgttagttgctacgcaagagcactctagcaggactgccgttgacaaaacggaggaaggtggggatgacgtcaaatcatcatgccccttatgacctgggcctcacacgtactacaatggctgtcaacagagggatgcaaagccgcgaggtggagcgaacccctaaaagcagtcttagttcggattgtaggctgcaacccgcctacatgaagtcggaattgctagtaatcgcagatcagcatgctgcggtgaatacgttcccgggccttgtacacaccgcccgtcacgccatgggagtcggtaacacccgaagcctgtagtctaaccgcaaggaggacgcagtcgaa

SEQ ID NO: 84 - DNA, 1357 bp, Parabacteroides distasonis
  misc_feature: HB-214_Parabacteroides_distasonis
gcgaccggcgcacgggtgagtaacgcgtatgcaacttgcctatcagagggggataacccggcgaaagtcggactaataccgcatgaagcaggggccccgcatggggatatttgctaaagattcatcgctgatagataggcatgcgttccattaggcagttggcggggtaacggcccaccaaaccgacgatggataggggttctgagaggaaggtcccccacattggtactgagacacggaccaaactcctacgggaggcagcagtgaggaatattggtcaatggccgagaggctgaaccagccaagtcgcgtgagggatgaaggttctatggatcgtaaacctcttttataagggaataaagtgcgggacgtgtcccgttttgtatgtaccttatgaataaggatcggctaactccgtgccagcagccgcggtaatacggaggatccgagcgttatccggatttattgggtttaaagggtgcgtaggcggccttttaagtcagcggtgaaagtctgtggctcaaccatagaattgccgttgaaactggggggcttgagtatgtttgaggcaggcggaatgcgtggtgtagcggtgaaatgcatagatatcacgcagaaccccgattgcgaaggcagcctgccaagccattactgacgctgatgcacgaaagcgtggggatcaaacaggattagataccctggtagtccacgcagtaaacgatgatcactagctgtttgcgatacactgtaagcggcacagcgaaagcgttaagtgatccacctggggagtacgccggcaacggtgaaactcaaaggaattgacgggggcccgcacaagcggaggaacatgtggtttaattcgatgatacgcgaggaaccttacccgggtttgaacgcatttcgacagctctggaaacagagtctctagcgatagcgatttgcgaggtgctgcatggttgtcgtcagctcgtgccgtgaggtgtcggcttaagtgccataacgagcgcaacccttgccactagttactaacaggtgatgctgaggactctggtgggactgccagcgtaagctgcgaggaaggcggggatgacgtcaaatcagcacggcccttacatccggggcgacacacgtgttacaatggcgtggacaaagggaggccacctggcgacagggagcgaatccccaaaccacgtctcagttcggatcggagtctgcaacccgactccgtgaagctggattcgctagtaatcgcgcatcagccatggcgcggtgaatacgttcccgggccttgtacacaccgcccgtcaagccatgggagccgggggtacctgaagtccgtaaccgaaaggatcggcct

SEQ ID NO: 85 - DNA, 1425 bp, Veillonella atypica
  misc_feature: HB-251_Veillonella_atypica
tgcagtcgaacgaagagcgatggaagcttgcttctatcaatcttagtggcgaacgggtgagtaacgcgtaatcaacctgcccttcagagggggacaacagttggaaacgactgctaataccgcatacgatccaatctcggcatcgagactggatgaaaggtggcctctatttataagctatcactgaaggaggggattgcgtctgattagctagttggaggggtaacggcccaccaaggcgatgatcagtagccggtctgagaggatgaacggccacattgggactgagacacggcccagactcctacgggaggcagcagtggggaatcttccgcaatggacgaaagtctgacggagcaacgccgcgtgagtgatgacggccttcgggttgtaaagctctgttaatcgggacgaatggttcttgtgcgaatagtgcgaggatttgacggtaccggaatagaaagccacggctaactacgtgccagcagccgcggtaatacgtaggtggcaagcgttgtccggaattattgggcgtaaagcgcgcgcaggcggattggtcagtctgtcttaaaagttcggggcttaaccccgtgatgggatggaaactgccaatctagagtatcggagaggaaagtggaattcctagtgtagcggtgaaatgcgtagatattaggaagaacaccagtggcgaaggcgactttctggacgaaaactgacgctgaggcgcgaaagccaggggagcgaacgggattagataccccggtagtcctggccgtaaacgatgggtactaggtgtaggaggtatcgaccccttctgtgccggagttaacgcaataagtaccccgcctggggagtacgaccgcaaggttgaaactcaaaggaattgacgggggcccgcacaagcggtggagtatgtggtttaattcgacgcaacgcgaagaaccttaccaggtcttgacattgatggacagaactagagatagttcctcttcttcggaagccagaaaacaggtggtgcacggttgtcgtcagctcgtgtcgtgagatgttgggttaagtcccgcaacgagcgcaacccctatcttatgttgccagcacttcgggtgggaactcatgagagactgccgcagacaatgcggaggaaggcggggatgacgtcaaatcatcatgccccttatgacctgggctacacacgtactacaatgggagttaatagacggaagcgaaaccgcgaggtggagcaaacccgagaaacactctctcagttcggatcgtaggctgcaactcgcctacgtgaagtcggaatcgctagtaatcgcaggtcagcatactgcggtgaatacgttcccgggccttgtacacaccgcccgtcacaccacgaaagtcggaagtgcccaaagccggtggggtaaccttcgggagcca

SEQ ID NO: 86 - DNA, 1388 bp, Clostridium sp.
  misc_feature: HB-358_Clostridium_sp.
tgcagtcgaacggacgagaaggtgcttgcaccttcaagttagtggcggacgggtgagtaacgcgtgagcaacctgcctcaaagagggggataacgtctggaaacggacgctaataccgcatgacgtattcgataggcatctattggataccaaaggagcaatccgctttgagatgggctcgcgtctgattagctagttggtggggtaaaggcctaccaaggcgacgatcagtagccggactgagaggttgaacggccacattgggactgagacacggcccagactcctacgggaggcagcagtgggggatattgcacaatgggggaaaccctgatgcagcaacgccgcgtgaaggaagacggttttcggattgtaaacttctgttcttagtgacgataatgacggtagctaaggagaaagctccggctaactacgtgccagcagccgcggtaatacgtagggagcgagcgttgtccggaattactgggtgtaaagggagcgtaggcgggagatcaagtcagatgtgaaaactatgggctcaacccataacctgcatttgaaactgttcttcttgagtgaagtagaggcaggcggaattccgagtgtagcggtgaaatgcgtagatattcggaggaacaccagtggcgaaggcggcctgctgggcttttactgacgctgaggctcgaaagcatggggagcaaacaggattagataccctggtagtccatgccgtaaacgatgattactaggtgtggggtggctgacccattccgtgccggagttaacacaataagtaatccacctggggagtacggccgcaaggttgaaactcaaaggaattgacgggggcccgcacaagcagtggagtatgtggtttaattcgaagcaacgcgaagaaccttaccaggtcttgacatccgactaacgaagtagagatacattaggtgcccttcggggaaagtcgagacaggtggtgcatggttgtcgtcagctcgtgtcgtgagatgttgggttaagtcccgcaacgagcgcaacccttgtcattagttgctacgcaagagcactctaatgagactgccgttgacaaaacggaggaaggtggggacgacgtcaaatcatcatgccccttatgacctgggctacacacgtactacaatggccgttaacagagggaagcaatactgtgaagtggagcaaacccctaaaaacggtcccagttcagattgcaggctgcaacccgcctgcatgaagtcggaattgctagtaatcgcggatcagcatgccgcggtgaatacgttcccgggccttgtacacaccgcccgtcacaccatgggagccggtaatacccgaagtcggtagtctaaccgcaaggaggacgccgccgaagg

SEQ ID NO: 87 - DNA, 800 bp, Parabacteroides goldsteinii
  misc_feature: HB-44_Parabacteroides_goldsteinii
gcagtcgaggggcagcacggtgtagcaatacattggtggcgaccggcgcacgggtgagtaacgcgtatgcaacctgcctatcagaggggaataacccggcgaaagtcggactaataccgcataaaacaggggttccacatggaaatatttgttaaagaattatcgctgatagatgggcatgcgttccattagatagttggtgaggtaacggctcaccaagtccacgatggataggggttctgagaggaaggtcccccacactggtactgagacacggaccagactcctacgggaggcagcagtgaggaatattggtcaatgggcgagagcctgaaccagccaagtcgcgtgaaggatgaaggatctatggtttgtaaacttcttttatatgggaataaagtgaggaacgtgttcctttttgtatgtaccatatgaataagcatcggctaactccgtgccagcagccgcggtaatacggaggatgcgagcgttatccggatttattgggtttaaagggtgcgtaggtggttaattaagtcagcggtgaaagtttgtggctcaaccataaaattgccgttgaaactggttgacttgagtatatttgaggtaggcggaatgcgtggtgtagcggtgaaatgcatagatatcacgcagaactccgattgcgaaggcagcttactaaactataactgacactgaagcacgaaagcgtggggatcaaacaggattagataccctggtagtccacgcagtaaacgatgattactagctgtttgcgatacacagtaagcggcacagcg

SEQ ID NO: 88 - DNA, 1251 bp, Clostridium sphenoides
  misc_feature: HB-470_Clostridium_sphenoides
tcaggatgaacgctggcggcgtgcttaacacatgcaagtcgaacgaagcaatgaagcggaagttttcggacagaagcttgattgactgagtggcggacgggtgagtaacgcgtgggaaacctgccttatacagggggataacagttagaaatgactgctaataccgcataagcctgcagcatcgcatgatgcaggaggaaaaggagcaatccggtataagatggtcccgcgtctgattagctagttggtaaggtaacggcctaccaaggcgacgatcagtagccgacctgagagggtgaccggccacattgggactgagacacggcccaaactcctacgggaggcagcagtggggaatattgcacaatgggggaaaccctgatgcagcgacgccgcgtgagtgaagaagtcattcgtgatgtaaagctctatcagcagggaagaaagtgacagtacctgagtaagaagccccggctaactacgtgccagcagccgcggtaatacgtagggggcaagcgttatccggatttactgggtgtaaagggagcgtaggtggtactgcaagtcagaagtgaaagccccgtgcttaacgtggggactgcttttgaaactgtgggactagagtgcaggagaggtaagtggaattcctagtgtagcggtgaaatgcgtagatattaggaggaacaccggtggcgaaggcggcttactggactgtaactgacactgaggctcgaaagcgtggggagcaaacaggattagataccctggtagtccacgctgtaaacgatgaatactaggtgttggggagcgaagctcctcggtgccgtcgcaaacgcaataagtattccacctggggagtacgttcgcaagaatgaaactcaaaggaattgacggggacccgcacaagcggtggagcatgtggtttaattcgaagcaacgcgaagaaccttaccaagtcttgacatctggatgaccgggctagagatagcccttctcttcggagcatccaagacaggtggtgcatggttgtcgtcagctcgtgtcgtgagatgttgggttaagtcccgcaacgagcgcaacccttatctttagtagccagcaggtaaagctgggaactctagagagactgccgtggataacgcggaggaaggtggggatgacgtcaaatcatcatgccccttatgacttgggctacacacgtgctacaatggcgtaaacagagggaagcgaacccgcgaggggagcgaatcc

SEQ ID NO: 89 - DNA, 927 bp, Escherichia coli
  misc_feature: HB-490_Escherichia_coli
gcagcctgatgcagccatgccgcgtgtatgaagaaggccttcgggttgtaaagtactttcagcggggaggaagggagtaaagttaatacctttgctcattgacgttacccgcagaagaagcaccggctaactccgtgccagcagccgcggtaatacggagggtgcaagcgttaatcggaattactgggcgtaaagcgcacgcaggcggtttgttaagtcagatgtgaaatccccgggctcaacctgggaactgcatctgatactggcaagcttgagtctcgtagaggggggtagaattccaggtgtagcggtgaaatgcgtagagatctggaggaataccggtggcgaaggcggccccctggacgaagactgacgctcaggtgcgaaagcgtggggagcaaacaggattagataccctggtagtccacgccgtaaacgatgtcgacttggaggttgtgcccttgaggcgtggcttccggagctaacgcgttaagtcgaccgcctggggagtacggccgcaaggttaaaactcaaatgaattgacgggggcccgcacaagcggtggagcatgtggtttaattcgatgcaacgcgaagaaccttacctggtcttgacatccacggaagttttcagagatgagaatgtgccttcgggaaccgtgagacaggtgctgcatggctgtcgtcagctcgtgttgtgaaatgttgggttaagtcccgcaacgagcgcaacccttatcctttgttgccagcggtccggccgggaactcaaaggagactgccagtgataaactggaggaaggtggggatgacgtcaagtcatcatggcccttacgaccagggctacacacgtgctacaatgggcgcatacaaagagaagcgacctcgcgagagcaagcggacctcataaagtgcgtcgtagtccgga

SEQ ID NO: 90 - DNA, 1237 bp, Lawsonibacter asaccharolyticus
  misc_feature: HB-521_Lawsonibacter_asaccharolyticus
ctcaggatgaacgctggcggcgtgcttaacacatgcaagtcgaacggagtgccttagaaagaggattcgtccaattgataaggttacttagtggcggacgggtgagtaacgcgtgaggaacctgccttggagtggggaataacagaccgaaaggcctgctaataccgcatgatgcagttggaccgcatggtcctgactgccaaagatttatcgctctgagatggcctcgcgtctgattagcttgttggcggggtaatggcccaccaaggcgacgatcagtagccggactgagaggttggccggccacattgggactgagacacggcccagactcctacgggaggcagcagtggggaatattgggcaatgggcgcaagcctgacccagcaacgccgcgtgaaggaagaaggctttcgggttgtaaacttcttttctcagggacgaacaaatgacggtacctgaggaataagccacggctaactacgtgccagcagccgcggtaatacgtaggtggcaagcgttatccggatttactgggtgtaaagggcgtgtaggcgggaaggcaagtcagatgtgaaaactatgggctcaacccatagcctgcatttgaaactgtttttcttgagtgctggagaggcaatcggaattccgtgtgtagcggtgaaatgcgtagatatacggaggaacaccagtggcgaaggcggattgctggacagtaactgacgctgaggcgcgaaagcgtggggagcaaacaggattagataccctggtagtccacgctgtaaacgatggatactaggtgtggggggtctgaccccctccgtgccgcagttaacacaataagtatcccacctggggagtacgatcgcaaggttgaaactcaaaggaattgacgggggcccgcacaagcggtggagtatgtggtttaattcgaagcaacgcgaagaaccttaccagggcttgacatcctactaacgaagcagagatgcattaggtgcccttcggggaaagtagagacaggtggtgcatggttgtcgtcagctcgtgtcgtgagatgttgggttaagtcccgcaacgagcgcaacccttattgttagttgctacgcaagagcactctagcgagactgccgttgacaaaacggaggaaggcggggacgacgtcaaatcatcatgccccttatgtcctgggctacacacgtactacaatggtggtaaacagagggaagcaagaccgcga

SEQ ID NO: 91 - DNA, 1390 bp, Gemmiger sp.
  misc_feature: HB-567_Gemmiger_sp.
tgcagtcgaacggagcttagagagcttgctttttaagcttagtggcgaacgggtgagtaacgcgtgagtaacctgccctagagtgggggacaacagttggaaacgactgctaataccgcataagcccacggtaccgcatggcactgagggaaaaggatttattcgctttaggatggactcgcgtccaattagctagttggtgaggtaacggcccaccaaggcgacgattggtagccggactgagaggttgaacggccacattgggactgagacacggcccagactcctacgggaggcagcagtgggggatattgcacaatgggggaaaccctgatgcagcgacgccgcgtggaggaagaaggttttcggattgtaaactcctgtcgttagggacgataatgacggtacctaacaagaaagcaccggctaactacgtgccagcagccgcggtaaaacgtagggtgcaagcgttgtccggaattactgggtgtaaagggagcgcaggcgggaagacaagttggaagtgaaaaccatgggctcaacccatgaattgctttcaaaactgtttttcttgagtagtgcagaggtagatggaattcccggtgtagcggtggaatgcgtagatatcgggaggaacaccagtggcgaaggcggtctactgggcaccaactgacgctgaggctcgaaagcatgggtagcaaacaggattagataccctggtagtccatgccgtaaacgatgattactaggtgttgggggattgaccccctcagtgccgcagttaacacaataagtaatccacctggggagtacgaccgcaaggttgaaactcaaaggaattgacgggggcccgcacaagcagtggagtatgtggtttaattcgaagcaacgcgaagaaccttaccaggtcttgacatccgatgcataacacagagatgtgtgaaatccttcgggacatcgagacaggtggtgcatggttgtcgtcagctcgtgtcgtgagatgttgggttaagtcccgcaacgagcgcaacccttattgccagttactacgttaagaggactctggcgagactgccgttgacaaaacggaggaaggtggggatgacgtcaaatcatcatgccctttatgacctgggctacacacgtactacaatggcgttaaacaaagagaagcaagaccgcgaggtggagcaaaactcaaaaacaacgtctcagttcagattgcaggctgcaactcgcctgcatgaagtcggaattgctagtaatcgcggatcagcatgccgcggtgaatacgttcccgggccttgtacacaccgcccgtcacaccatgagagccggggggacccgaagtcgatagtctaaccgcaaggaggacgtcgccgaaggtaaa

SEQ ID NO: 92 - DNA, 688 bp, Eubacterium callanderi
  misc_feature: HB-59_Eubacterium_callanderi
gtcgaacgagaaagttttgaatgatccttcgggtgaaattagaactggaaagtggcgaacgggtgagtaacgcgtgggtaacctgccctatggaaaggaatagcctcgggaaactgggagtaaagccttatattatggttttgtcgcatggcaagatcatgaaaactccggtgccataggatggacccgcgtcccattagctagttggtgagataacagcccaccaaggcgacgatgggtaaccggtctgagagggcgaacggtcacactggaactgagacacggtccagactcctacgggaggcagcagtggggaatattgcgcaatgggggcaaccctgacgcagcaataccgcgtgagtgaagaaggttttcggatcgtaaagctctgttattggggaagaatacatgacggtacccaatgaggaagtcccggctaactacgtgccagcagccgcggtaatacgtaggggacaagcgttgtccggaatgactgggcgtaaagggcgcgtaggcggtctattaagtctgatgtgaaaggtaccggctcaaccggtgaagtgcattggaaactggtagacttgagtattggagaggcaagtggaattcctagtgtagcggtgaaatgcgtagatattaggaggaacaccagtggcgaaggcggcttgctggacaaatactgacgctg

SEQ ID NO: 93 - DNA, 1403 bp, Intestinimonas massiliensis
  misc_feature: HB-651_Intestinimonas_massiliensis
tcaggatgaacgctggcggcgtgcttaacacatgcaagtcgaacggaacgccaaggaaagagttttcggacaatggaattggctgtttagtggcggacgggtgagtaacgcgtgagtaacctgccttggagtggggaataacacagtgaaaactgtgctaataccgcatgacatattggtgtcgcatggcactgatatcaaagatttatcgctctgagatggactcgcgtctgattagatagttggcggggtaacggcccaccaagtcgacgatcagtagccggactgagaggttggccggccacattgggactgagacacggcccagactcctacgggaggcagcagtggggaatattgggcaatgggcgcaagcctgacccagcaacgccgcgtgaaggaagaaggctttcgggttgtaaacttcttttaacagggacgaagcaagtgacggtacctgttgaataagccacggctaactacgtgccagcagccgcggtaatacgtaggtggcaagcgttatccggatttactgggtgtaaagggcgtgtaggcgggactgcaagtcagatgtgaaaactatgggctcaacccatagcctgcatttgaaactgtagttcttgagtgtcggagaggcaatcggaattccgtgtgtagcggtgaaatgcgtagatatacggaggaacaccagtggcgaaggcggattgctggacgataactgacgctgaggcgcgaaagcgtggggagcaaacaggattagataccctggtagtccacgccgtaaacgatggatactaggtgtggggggtctgaccccctccgtgccgcagctaacgcaataagtatcccacctggggagtacgatcgcaaggttgaaactcaaaggaattgacgggggcccgcacaagcggtggagtatgtggtttaattcgaagcaacgcgaagaaccttaccagggcttgacatcctactaacgaaccagagatggattaggtgcccttcggggaaagtagagacaggtggtgcatggttgtcgtcagctcgtgtcgtgagatgttgggttaagtcccgcaacgagcgcaacccttattgttagttgctacgcaagagcactctagcgagactgccgttgacaaaacggaggaaggtggggacgacgtcaaatcatcatgccccttatgtcctgggccacacacgtactacaatggcggttaacagagggaggcaaagccgcgaggcagagcaaacccctaaaagccgtcccagttcggattgcaggctgaaacccgcctgtatgaagtcggaatcgctagtaatcgcggatcagcatgccgcggtgaatacgttcccgggccttgtacacaccgcccgtcacaccatgagagtcgggaacacccgaagtc

SEQ ID NO: 94 - DNA, 809 bp, Clostridium symbiosum
  misc_feature: HB-67_Clostridium_symbiosum
  misc_feature (763)..(763): n is a, c, g, or t
tgcagtcgaacgaagcgatttaacggaagttttcggatggaagttgagattgactgagtggcggacgggtgagtaacgcgtgggtaacctgccttgtactgggggacaacagttagaaatgactgctaataccgcataagcgcacagtatcgcatgatacagtgtgaaaaactccggtggtacaagatggacccgcgtctgattagctagttggtaaggtaacggcttaccaaggcgacgatcagtagccgacctgagagggtgaccggccacattgggactgagacacggcccaaactcctacgggaggcagcagtggggaatattgcacaatgggcgaaagcctgatgcagcgacgccgcgtgagtgaagaagtatttcggtatgtaaagctctatcagcagggaagaaaatgacggtacctgactaagaagccccggctaactacgtgccagcagccgcggtaatacgtagggggcaagcgttatccggatttactgggtgtaaagggagcgtagacggtaaagcaagtctgaagtgaaagcccgcggctcaactgcgggactgctttggaaactgtttaactggagtgtcggagaggtaagtggaattcctagtgtagcggtgaaatgcgtagatattaggaggaacaccagtggcgaaggcgacttactggacgataactgacgttgaggctcgaaagcgtggggagcaaacaggattagataccctggtagtccacgccgtaaacgatgaatactanggtgttgggggagcaaagctcttcggtgccgtcgcaaacgcagtaa

SEQ ID NO: 95 - DNA, 1287 bp, Longibaculum sp.
  misc_feature: HB-681_Longibaculum_sp.
  misc_feature (182)..(182): n is a, c, g, or t
  misc_feature (976)..(976): n is a, c, g, or t
  misc_feature (1091)..(1091): n is a, c, g, or t
  misc_feature (1101)..(1101): n is a, c, g, or t
  misc_feature (1140)..(1140): n is a, c, g, or t
  misc_feature (1212)..(1212): n is a, c, g, or t
gctcaggatgaacgctggcggcgtgcctaatacatgcaagtcggacgcaatgcttcggcattgagtggcgaacgggtgagtaatacataagcaacctgcccctgtgagggggataactgctggaaacggcagctaagaccgcataggcatacatgacgcatgtcgagtatgttaaatatccnacgggatagcacagggatgggcttatgacgcattagctagctggtgaggtagaggctcaccagggcgacgatgcgtagccggcctgagagggtggacggccacactgggactgagacacggcccagactcctacgggaggcagcagtagggaattttcggcaatgggcgaaagcctgaccgagcaacgccgcgtgaaggaagaagtcattcgtgatgtaaacttctgttataaaggaagaacggcgcctgtagggaatgacaggcgagtgacggtactttatgaggaagccacggctaactacgtgccagcagccgcggtaatacgtaggtggcgagcgttatccggaatcattgggcgtaaagagggagcaggcggcagtgcaggtctgcggtgaaagcccgaagctaaacttcggtaagccgtggaaaccgcacagctagagagcatcagaggatcgcggaattccatgtgtagcggtgaaatgcgtagatatatggaggaacaccagtggcgaaggcggcggtctggggtgcagctgacgctcagtcccgaaagcgtggggagcaaataggattagataccctagtagtccacgccgtaaacgatgagtgctaagtgttgggggtcagacctcagtgctgcagttaacgcaataagcactccgcctgagtagtacgttcgcaagaatgaaactcaaaggaattgacgggggcccgcacaagcggtggagcatgtggtttaattcgaagcaacgcgaagaaccttaccaggtcttgacatggagataaaggntctggagacagagagatagttatatctcacacaggtggtgcatggttgtcgtcagctcgtgtcgtgagatgttgggttaagtcccgcaacgagcgcaacccctgttgccagttgncagcattcgntggggactctggcgagactgcctctgcaagaggaggaangcggggatgacgtcaaatcatcatgccccttatgacctgggctacacacgtgctacaatggacggatcaganggaggcgaagccgcgaggtggagcgaaacccagaaacccgttcacagttcggactgcagtctgcaactcgactgc

SEQ ID NO: 96 - DNA, 1392 bp, Blautia obeum
  misc_feature: HB-14_Blautia_obeum
  misc_feature (944)..(944): n is a, c, g, or t
  misc_feature (1295)..(1295): n is a, c, g, or t
  misc_feature (1355)..(1355): n is a, c, g, or t
catgcagtcgaacgggaaaccttttattgaagcttcggcagatttagctggtttctagtggcggacgggtgagtaacgcgtgggtaacctgccttatacagggggataacaaccagaaatggttgctaataccgcataagcgcacaggaccgcatggtccggtgtgaaaaactccggtggtataagatggacccgcgttggattagctagttggcagggtaacggcctaccaaggcgacgatccatagccggcctgagagggtgaacggccacattgggactgagacacggcccagactcctacgggaggcagcagtggggaatattgcacaatgggggaaaccctgatgcagcgacgccgcgtgaaggaagaagtatctcggtatgtaaacttctatcagcagggaagatagtgacggtacctgactaagaagccccggctaactacgtgccagcagccgcggtaatacgtagggggcaagcgttatccggatttactgggtgtaaagggagcgtagacggactggcaagtctgatgtgaaaggcgggggctcaacccctggactgcattggaaactgttagtcttgagtgccggagaggtaagcggaattcctagtgtagcggtgaaatgcgtagatattaggaggaacaccagtggcgaaggcggcttactggacggtaactgacgttgaggctcgaaagcgtggggagcaaacaggattagataccctggtagtccacgccgtaaacgatgaatactaggtgttggggagcaaagctcttcggtgccgccgcaaacgcattaagtattccacctggggagtacgttcgcaagaatgaaactcaaaggaattgacggggacccgcacaagcggtggagcatgtggtttaattcgaagcaacgcgaagaaccttaccaagtcttgacatccctctgaccgantcttaaccgaatctttccttcgggacaggggagacaggtggtgcatggttgtcgtcagctcgtgtcgtgagatgttgggttaagtcccgcaacgagcgcaacccctatccccagtagccagcagtccggctgggcactctgaggagactgccagggataacctggaggaaggcggggatgacgtcaaatcatcatgccccttatgatttgggctacacacgtgctacaatggcgtaaacaaagggaagcaagcctgcgaaggtaagcaaatcccaaaaataacgtcccagttcggactgcagtctgcaactcgactgcacgaagctggaatcgctagtaatcgcggatcanaatgccgcggtgaatacgttccccgggtcttgtacacaccgcccgtcacaccatggggantcagtaacgcccgaagtcagtgacctaactgcaaaga

SEQ ID NO: 97 - DNA, 1361 bp, Dorea longicatena
  misc_feature: HB-17_Dorea_longicatena
acctttgtgactgagcggcggacgggtgagtaacgcgtgggtaacctgcctcatacagggggataacagttagaaatgactgctaataccgcataagaccacggtaccgcatggtacagtggtaaaaactccggtggtatgagatggacccgcgtctgattaggtagttggtggggtaacggcctaccaagccgacgatcagtagccgacctgagagggtgaccggccacattgggactgagacacggcccagactcctacgggaggcagcagtggggaatattgcacaatggaggaaactctgatgcagcgacgccgcgtgaaggatgaagtatttcggtatgtaaacttctatcagcagggaagaaaatgacggtacctgactaagaagccccggctaactacgtgccagcagccgcggtaatacgtagggggcaagcgttatccggatttactgggtgtaaagggagcgtagacggcacggcaagccagatgtgaaagcccggggctcaaccccgggactgcatttggaactgctgagctagagtgtcggagaggcaagtggaattcctagtgtagcggtgaaatgcgtagatattaggaggaacaccagtggcgaaggcggcttgctggacgatgactgacgttgaggctcgaaagcgtggggagcaaacaggattagataccctggtagtccacgccgtaaacgatgactgctaggtgtcgggtggcaaagccattcggtgccgcagctaacgcaataagcagtccacctggggagtacgttcgcaagaatgaaactcaaaggaattgacggggacccgcacaagcggtggagcatgtggtttaattcgaagcaacgcgaagaaccttacctgatcttgacatcccgatgaccgcttcgtaatggaagcttttcttcggaacatcggtgacaggtggtgcatggttgtcgtcagctcgtgtcgtgagatgttgggttaagtcccgcaacgagcgcaacccctatcttcagtagccagcaggttaagctgggcactctggagagactgccagggataacctggaggaaggtggggatgacgtcaaatcatcatgccccttatgaccagggctacacacgtgctacaatggcgtaaacaaagagaagcgaactcgcgagggtaagcaaatctcaaaaataacgtctcagttcggattgtagtctgcaactcgactacatgaagctggaatcgctagtaatcgcagatcagaatgctgcggtgaatacgttcccgggtcttgtacacaccgcccgtcacaccatgggagtcagtaacgcccgaagtcagtgacccaaccgtaaggagggagctgccgaa

SEQ ID NO: 98 - DNA, 1396 bp, Eubacterium rectale
  misc_feature: HB-22_Eubacterium_rectale
gcaagtcgaacgaagcactttatttgatttccttcgggactgattattttgtgactgagtggcggacgggtgagtaacgcgtgggtaacctgccttgtacagggggataacagttggaaacggctgctaataccgcataagcgcacggcatcgcatgatgcagtgtgaaaaactccggtggtataagatggacccgcgttggattagctagttggtgaggtaacggcccaccaaggcgacgatccatagccgacctgagagggtgaccggccacattgggactgagacacggcccaaactcctacgggaggcagcagtggggaatattgcacaatgggcgaaagcctgatgcagcgacgccgcgtgagcgaagaagtatttcggtatgtaaagctctatcagcagggaagataatgacggtacctgactaagaagcaccggctaaatacgtgccagcagccgcggtaatacgtatggtgcaagcgttatccggatttactgggtgtaaagggagcgcaggcggtgcggcaagtctgatgtgaaagcccggggctcaaccccggtactgcattggaaactgtcgtactagagtgtcggaggggtaagcggaattcctagtgtagcggtgaaatgcgtagatattaggaggaacaccagtggcgaaggcggcttactggacgataactgacgctgaggctcgaaagcgtggggagcaaacaggattagataccctggtagtccacgccgtaaacgatgaatactaggtgttgggaagcattgcttctcggtgccgtcgcaaacgcagtaagtattccacctggggagtacgttcgcaagaatgaaactcaaaggaattgacggggacccgcacaagcggtggagcatgtggtttaattcgaagcaacgcgaagaaccttaccaagtcttgacatccttctgaccggtacttaaccgtaccttctcttcggagcaggagtgacaggtggtgcatggttgtcgtcagctcgtgtcgtgagatgttgggttaagtcccgcaacgagcgcaacccttatctttagtagccagcggtccggccgggcactctagagagactgccagggataacctggaggaaggcggggatgacgtcaaatcatcatgccccttatgacttgggctacacacgtgctacaatggcgtaaacaaagggaagcaaagctgtgaagccgagcaaatctcaaaaataacgtctcagttcggactgtagtctgcaacccgactacacgaagctggaatcgctagtaatcgcagatcagaatgctgcggtgaatacgttcccgggtcttgtacacaccgcccgtcacaccatgggagttgggaatgcccgaagccagtgacctaaccgaaaggaaggag

SEQ ID NO: 99 - DNA, 758 bp, Blautia coccoides
  misc_feature: HB-23_Blautia_coccoides
tgcagtcgagcgaagcagctaagacaggatttcttcggattgaagtctttgtgactgagcggcggacgggtgagtaacgcgtgggtaacctgcctcatacagggggataacagttagaaatgactgctaataccgcataagcgcacaggaccgcatggtctggtgtgaaaaactccggtggtatgagatggacccgcgtctgattagctagttggaggggtaacggcccaccaaggcgacgatcagtagccggcctgagagggtgaacggccacattgggactgagacacggcccagactcctacgggaggcagcagtggggaatattgcacaatgggggaaaccctgatgcagcgacgccgcgtgaaggaagaagtatctcggtatgtaaacttctatcagcagggaagaaaatgacggtacctgactaagaagccccggctaactacgtgccagcagccgcggtaatacgtagggggcaagcgttatccggatttactgggtgtaaagggagcgtagacggaagagcaagtctgatgtgaaaggctggggcttaaccccaggactgcattggaaactgttgttctagagtgccggagaggtaagcggaattcctagtgtagcggtgaaatgcgtagatattaggaggaacaccagtggcgaaggcggcttactggacggtaactgacgttgaggctcgaaagcgtggggagcaaacaggattagataccctggtagtccacgccgtaaacgatgaa

SEQ ID NO: 100 - DNA, 1064 bp, Bifidobacterium longum
  misc_feature: HB-234_Bifidobacterium_longum
gcgcagcctgatgcagcgacgccgcgtgagggatggaggccttcgggttgtaaacctcttttatcggggagcaagcgagagtgagtttacccgttgaataagcaccggctaactacgtgccagcagccgcggtaatacgtagggtgcaagcgttatccggaattattgggcgtaaagggctcgtaggcggttcgtcgcgtccggtgtgaaagtccatcgcttaacggtggatccgcgccgggtacgggcgggcttgagtgcggtaggggagactggaattcccggtgtaacggtggaatgtgtagatatcgggaagaacaccaatggcgaaggcaggtctctgggccgttactgacgctgaggagcgaaagcgtggggagcgaacaggattagataccctggtagtccacgccgtaaacggtggatgctggatgtggggcccgttccacgggttccgtgtcggagctaacgcgttaagcatcccgcctggggagtacggccgcaaggctaaaactcaaagaaattgacgggggcccgcacaagcggcggagcatgcggattaattcgatgcaacgcgaagaaccttacctgggcttgacatgttcccgacggtcgtagagatacggcttcccttcggggcgggttcacaggtggtgcatggtcgtcgtcagctcgtgtcgtgagatgttgggttaagtcccgcaacgagcgcaaccctcgccccgtgttgccagcggattatgccgggaactcacgggggaccgccggggttaactcggaggaaggtggggatgacgtcagatcatcatgccccttacgtccagggcttcacgcatgctacaatggccggtacaacgggatgcgacgcggcgacgcggagcggatccctgaaaaccggtctcagttcggatcgcagtctgcaactcgactgcgtgaaggcggagtcgctagtaatcgcgaatcagcaacgtcgcggtgaatgcgttcccgggccttgtacacaccgcccgtcaagtcatgaaagtgggcagcacccgaagccggtggcctaaccccttgtggga

SEQ ID NO: 101 - DNA, 1382 bp, Slackia isoflavoniconvertens
  misc_feature: HB-326_Slackia_isoflavoniconvertens
gcagtcgaacgagtaagacgccttcgggcgtggatagagtggcgaacgggtgagtaacacgtgaccaacctgccccctcctccgggacaacctcgggaaaccgaggctaataccggatactccgggccccccgcatggggggcccgggaaagccctggcgggaggggatggggtcgcggcccatcagggagacgggggggtaacggcccaccgtgcctgcaacgggtagccgggctgaaaggccgatcggccacattgggactgaaacacggcccagactcctacgggaggcagcagtggggaattttgcgcaatgggggcaaccctgacgcagcgacgccgcgtgcgggacgaagtcattcgtgacgtaaaccgctttcagcgaggaagaaccatgacggtactcgcagaagaagccccggctaactacgtgccagcagccgcggtaatacgtagggggcgagcgttatccggaatcattgggcgtaaagcgcgcgcaggcgggctttcaagcggcggcgtcgaagccgggggctcaacccccggaagcgccccgaactggaagcctcggatgcggcagggggaggcggaattcccggtgtagcggtgaaatgcgcagatatcgggaagaacaccgacggcgaaggcagcctcctgggccggcatcgacgctgaggcgcgaaagctgggggagcgaacaggattagataccctggtagtcccagccgtaaacgatggacgctaggtgtggggggaacgatccctccgtgccgaagccaacgcattaagcgtcccgcctggggagtacggccgcaaggctaaaactcaaaggaattgacgggggcccgcacaagcagcggagcatgtggcttaattcgaagcaacgcgaagaaccttaccagggcttgacatacaggtgaagccggagagatccggtggccgagaggagcctatacaggtggtgcatggctgtcgtcagctcgtgccgtgaggtgttgggttaagtcccgcaacgagcgcaacccccgccgcgtgttgccagcattcagttgggcactcacgcgggactgccggcgtcaagccggaggaaggcggggacgacgtcaagtcatcatgcccctcatgccctgggccgcacacgtgctacaatggccggcacaacgggttgccaccccgcgagggggagcggatccctaaagccggccccagttcggatcgcaggctgcaacccgcctgcgtgaagccggagttgctagtaatcgcggatcagcacgccgcggtgaatacgttcccgggccttgtacacaccgcccgtcacaccacccgagtcgtctgcacccgaagccgccggccgaaccccgcaagggggcggaggcgtcgaa

SEQ ID NO: 102 - DNA, 1403 bp, Lachnoclostridium sp.
  misc_feature: HB-698_Lachnoclostridium_sp.
  misc_feature (23)..(23): n is a, c, g, or t
tgcagtcgaacgaagcaatctancggaagttttcggatggaagctggattgactgagtggcggacgggtgagtaacgcgtgggtaacctgcctcacactgggggacaacagttagaaatgactgctaataccgcataagcgcacaggaccgcatggtccggtgtgaaaaactccggtggtgtgagatggacccgcgtttgattagctggttggtggggtaacggcctaccaaggcgacgatcaatagccgacctgagagggtgaccggccacattgggactgagacacggcccaaactcctacgggaggcagcagtggggaatattgcacaatgggggaaaccctgatgcagcgacgccgcgtgagtgaagaagtatttcggtatgtaaagctctatcagcagggaagaaaatgacggtacctgactaagaagccccggctaactacgtgccagcagccgcggtaatacgtagggggcaagcgttatccggatttactgggtgtaaagggagcgtagacggtaaagcaagtctgaagtgaaagcccggggctcaaccccgggactgctttggaaactgtttaactagagtgctggagaggtaagcggaattcctagtgtagcggtgaaatgcgtagatattaggaggaacaccagtggcgaaggcggcttactggacagtaactgacgttgaggctcgaaagcgtggggagcaaacaggattagataccctggtagtccacgccgtaaacgatgaatactaggtgttgggggacaaagtccttcggtgccgtcgcaaacgcaataagtattccacctggggagtacgttcgcaagaatgaaactcaaaggaattgacggggacccgcacaagcggtggagcatgtggtttaattcgaagcaacgcgaagaaccttaccaaatcttgacatccctctgaccggcgagtaatgtcgcctttccttcgggacagaggagacaggtggtgcatggttgtcgtcagctcgtgtcgtgagatgttgggttaagtcccgcaacgagcgcaacccttatcctaagtagccagcagttcggctgggcactcttgggagactgccagggataacctggaggaaggtggggatgacgtcaaatcatcatgccccttatgatttgggctacacacgtgctacaatggcgtaaacaaagagaagcgagaccgcgaggtggagcaaatctcaaaaataacgtctcagttcggactgcaggctgcaactcgcctgcacgaagctggaatcgctagtaatcgcgaatcagaatgtcgcggtgaatacgttcccgggtcttgtacacaccgcccgtcacaccatgggagtcagtaatgcccgaagtcagtgacccaaccgaaaggagggagctgccgaag

SEQ ID NO: 103 - DNA, 1255 bp, Coprococcus comes
  misc_feature: HB-80_Coprococcus_comes
  misc_feature (1150)..(1150): n is a, c, g, or t
tttgtgactgagtggcggacgggtgagtaacgcgtgggtaacctgcctcatacagggggataacagttagaaatgactgctaataccgcataagaccacggagccgcatggctcagtgggaaaaactccggtggtatgagatggacccgcgtctgattaggtagttggtggggtaacggcctaccaagccaacgatcagtagccgacctgagagggtgaccggccacattgggactgagacacggcccaaactcctacgggaggcagcagtggggaatattgcacaatgggggaaaccctgatgcagcgacgccgcgtgagcgaagaagtatttcggtatgtaaagctctatcagcagggaagaaaatgacggtacctgactaagaagcaccggctaaatacgtgccagcagccgcggtaatacgtatggtgcaagcgttatccggatttactgggtgtaaagggagcgtagacggctgtgtaagtctgaagtgaaagcccggggctcaaccccgggactgctttggaaactatgcagctagagtgtcggagaggtaagtggaattcccagtgtagcggtgaaatgcgtagatattgggaggaacaccagtggcgaaggcggcttactggacgatgactgacgttgaggctcgaaagcgtggggagcaaacaggattagataccctggtagtccacgccgtaaacgatgactactaggtgtcggggagcaaagctcttcggtgccgcagcaaacgcaataagtagtccacctggggagtacgttcgcaagaatgaaactcaaaggaattgacggggacccgcacaagcggtggagcatgtggtttaattcgaagcaacgcgaagaaccttacctgctcttgacatcccggtgaccggcgtgtaatgacgccttttcttcggaacaccggtgacaggtggtgcatggttgtcgtcagctcgtgtcgtgagatgttgggttaagtcccgcaacgagcgcaacccttatcttcagtagccagcattttggatgggcactctggagagactgccagggataacctggaggaaggtggggatgacgtcaaatcatcatgccccttatgagcagggctacacacgtgctacaatggcgtaaacaaagggaagcaancctgcgagggtaagcaaatctcaaaaataacgtctcagttcggattgtagtctgcaactcgactacatgaagctggaatcgctagtaatcgcgaatcagcatgtc

SEQ ID NO: 104 - DNA, 740 bp, Clostridium butyricum
  misc_feature: 104_HB-88_Clostridium_butyricum
tgcagtcgagcgatgaagctccttcgggagtggattagcggcggacgggtgagtaacacgtgggtaacctgcctcatagaggggaatagcctttcgaaaggaagattaataccgcataagattgtagtaccgcatggtacagcaattaaaggagtaatccgctatgagatggacccgcgtcgcattagctagttggtgaggtaacggctcaccaaggcgacgatgcgtagccgacctgagagggtgatcggccacattgggactgagacacggcccagactcctacgggaggcagcagtggggaatattgcacaatgggggaaaccctgatgcagcaacgccgcgtgagtgatgacggtcttcggattgtaaagctctgtctttagggacgataatgacggtacctaaggaggaagccacggctaactacgtgccagcagccgcggtaatacgtaggtggcaagcgttgtccggatttactgggcgtaaagggagcgtaggtggatatttaagtgggatgtgaaatacccgggcttaacctgggtgctgcattccaaactggatatctagagtgcaggagaggaaaggagaattcctagtgtagcggtgaaatgcgtagagattaggaagaataccagtggcgaaggcgcctttctggactgtaactgacactgaggctcgaaagcgtggggagcaaacaggattagataccctggtagtccacgccgtaaacgatgaat

SEQ ID NO: 105 - DNA, 1473 bp, Clostridium sporogenes
  misc_feature: LC037213.1 Clostridium sporogenes gene for 16S ribosomal RNA, partial sequence, strain JCM 7836
agagtttgatcctggctcaggacgaacgctggcggcgtgcttaacacatgcaagtcgagcgatgaagcttccttcgggaagtggattagcggcggacgggtgagtaacacgtgggtaacctgcctcaaagtgggggatagccttccgaaaggaagattaataccgcataacataagagaatcgcatgattttcttatcaaagatttattgctttgagatggacccgcggcgcattagctagttggtaaggtaacggcttaccaaggcaacgatgcgtagccgacctgagagggtgatcggccacattggaactgagacacggtccagactcctacgggaggcagcagtggggaatattgcgcaatgggggaaaccctgacgcagcaacgccgcgtgggtgatgaaggtcttcggattgtaaagccctgttttctgggacgataatgacggtaccagaggaggaagccacggctaactacgtgccagcagccgcggtaatacgtaggtggcgagcgttgtccggatttactgggcgtaaagggtgcgtaggcggatgtttaagtgggatgtgaaatccccgggcttaacctgggggctgcattccaaactggatatctagagtgcaggagaggaaagcggaattcctagtgtagcggtgaaatgcgtagagattaggaagaacaccagtggcgaaggcggctttctggactgtaactgacgctgaggcacgaaagcgtgggtagcaaacaggattagataccctggtagtccacgccgtaaacgatggatactaggtgtagggggtatcaactccccctgtgccgcagttaacacaataagtatcccgcctggggagtacggtcgcaagattaaaactcaaaggaattgacgggggcccgcacaagcagcggagcatgtggtttaattcgaagcaacgcgaagaaccttacctggacttgacatcccttgcatagcctagagataggtgaagcccttcggggcaaggagacaggtggtgcatggttgtcgtcagctcgtgtcgtgagatgttaggttaagtcctgcaacgagcgcaacccttgttattagttgctaccattaagttgagcactctaatgagactgcctgggtaaccaggaggaaggtggggatgacgtcaaatcatcatgccccttatgtccagggctacacacgtgctacaatggtaggtacaataagacgcaagaccgtgaggtggagcaaaacttataaaacctatctcagttcggattgtaggctgcaactcgcctacatgaagctggagttgctagtaatcgcgaatcagaatgtcgcggtgaatacgttcccgggccttgtacacaccgcccgtcacaccatgagagctggtaacacccgaagtccgtgaggtaaccgtaaggagccagcggccgaaggtgggattagtgattggggtgaagtcgtaacaaggtaacc

SEQ ID NO: 106 - DNA, 1433 bp, Akkermansia muciniphila
  misc_feature: NR_042817.1 Akkermansia muciniphila ATCC BAA-835 strain Muc 16S ribosomal RNA, partial sequence
aacgaacgctggcggcgtggataagacatgcaagtcgaacgagagaattgctagcttgctaataattctctagtggcgcacgggtgagtaacacgtgagtaacctgcccccgagagcgggatagccctgggaaactgggattaataccgcatagtatcgaaagattaaagcagcaatgcgcttggggatgggctcgcggcctattagttagttggtgaggtaacggctcaccaaggcgatgacgggtagccggtctgagaggatgtccggccacactggaactgagacacggtccagacacctacgggtggcagcagtcgagaatcattcacaatgggggaaaccctgatggtgcgacgccgcgtgggggaatgaaggtcttcggattgtaaacccctgtcatgtgggagcaaattaaaaagatagtaccacaagaggaagagacggctaactctgtgccagcagccgcggtaatacagaggtctcaagcgttgttcggaatcactgggcgtaaagcgtgcgtaggctgtttcgtaagtcgtgtgtgaaaggcgcgggctcaacccgcggacggcacatgatactgcgagactagagtaatggagggggaaccggaattctcggtgtagcagtgaaatgcgtagatatcgagaggaacactcgtggcgaaggcgggttcctggacattaactgacgctgaggcacgaaggccaggggagcgaaagggattagatacccctgtagtcctggcagtaaacggtgcacgcttggtgtgcggggaatcgaccccctgcgtgccggagtaacgcgttaagcgtgccgcctggggagtacggtcgcaagattaaaactcaaagaaattgacggggacccgcacaagcggtggagtatgtggcttaattcgatgcaacgcgaagaaccttacctgggcttgacatgtaatgaacaacatgtgaaagcatgcgactcttcggaggcgttacacaggtgctgcatggccgtcgtcagctcgtgtcgtgagatgtttggttaagtccagcaacgagcgcaacccctgttgccagttaccagcacgtgaaggtggggactctggcgagactgcccagatcaactgggaggaaggtggggacgacgtcaggtcagtatggcccttatgcccagggctgcacacgtactacaatgcccagtacagagggggccgaagccgcgaggcggaggaaatcctaaaaactgggcccagttcggactgtaggctgcaacccgcctacacgaagccggaatcgctagtaatggcgcatcagctacggcgccgtgaatacgttcccgggtcttgtacacaccgcccgtcacatcatggaagctggtcgcacccgaagtatctgaagccaaccgcaaggaggcagggtcctaaggtgagactggtaactgggatg

SEQ ID NO: 107 - DNA, 1503 bp, Clostridium sporogenes
  misc_feature: NR_029231.1 Clostridium sporogenes strain McClung 2004 16S ribosomal RNA, partial sequence
gagagtttgatcctggctcaggacgaacgctggcggcgtgcttaacacatgcaagtcgagcgatgaagcttccttcgggaagtggattagcggcggacgggtgagtaacacgtgggtaacctgcctcaaagtgggggatagccttccgaaaggaagattaataccgcataacataagagaatcgcatgattttcttatcaaagatttattgctttgagatggacccgcggcgcattagctagttggtaaggtaacggcttaccaaggcaacgatgcgtagccgacctgagagggtgatcggccacattggaactgagacacggtccagactcctacgggaggcagcagtggggaatattgcgcaatgggggaaaccctgacgcagcaacgccgcgtgggtgatgaaggtcttcggattgtaaagccctgttttctgggacgataatgacggtaccagaggaggaagccacggctaactacgtgccagcagccgcggtaatacgtaggtggcgagcgttgtccggatttactgggcgtaaagggtgcgtaggcggatgtttaagtgggatgtgaaatccccgggcttaacctgggggctgcattccaaactggatatctagagtgcaggagaggaaagcggaattcctagtgtagcggtgaaatgcgtagagattaggaagaacaccagtggcgaaggcggctttctggactgtaactgacgctgaggcacgaaagcgtgggtagcaaacaggattagataccctggtagtccacgccgtaaacgatggatactaggtgtagggggtatcaactccccctgtgccgcagttaacacaataagtatcccgcctggggagtacggtcgcaagattaaaactcaaaggaattgacgggggcccgcacaagcagcggagcatgtggtttaattcgaagcaacgcgaagaaccttacctggacttgacatcccttgcatagcctagagataggtgaagcccttcggggcaaggagacaggtggtgcatggttgtcgtcagctcgtgtcgtgagatgttaggttaagtcctgcaacgagcgcaacccttgttattagttgctaccattaagttgagcactctaatgagactgcctgggtaaccaggaggaaggtggggatgacgtcaaatcatcatgccccttatgtccagggctacacacgtgctacaatggtaggtacaataagacgcaagaccgtgaggtggagcaaaacttataaaacctatctcagttcggattgtaggctgcaactcgcctacatgaagctggagttgctagtaatcgcgaatcagaatgtcgcggtgaatacgttcccgggccttgtacacaccgcccgtcacaccatgagagctggtaacacccgaagtccgtgaggtaaccgtaaggagccagcggccgaaggtgggattagtgattggggtgaagtcgtaacaaggtagccgtaggagaacctgcggctggatcacctcc

SEQ ID NO: 108 - DNA, 1509 bp, Peptostreptococcus russellii
  misc_feature: NR_115155.1 Peptostreptococcus russellii strain RT-10B 16S ribosomal RNA, partial sequence
gagagtttgattctggctcaggatgaacgctggcggcgtgcttaacacatgcaagtcgagcgcggtccctttggtgcttgcaccaaagaagactgagcggcggacgggtgagtaacgcgtgggtaacctgccctatacacatggataacatgctgaaaagcatactaatacatgataacgtacactgatggcatcttcggtgtatcaaggcgttagcggtataggatggacccgcgtctgattagctagttggtggggtaaaagcctaccaaggccacgatcagtagccgacctgagagggtgatcggccacattggaactgagacacggtccaaactcctacgggaggcagcagtggggaatattgcacaatgggcgaaagcctgatgcagcaacgccgcgtgaacgatgaaggtcttcggatcgtaaagttctgtcgcaggggaagataatgacggtaccctgtgaggaagcaccggctaactacgtgccagcagccgcggtaatacgtagggtgctagcgttatccggatttactgggcgtaaagggtgcgtaggtggtctttcaagtcggtggttaaaggctacggctcaaccgtattaagccgccgaaactggaagacttgagtgcaggagaggaaagtggaattctcagtgtagcggtgaaatgcgtagatattgagaagaacaccagtagcgaaggcggctttctggactgtaactgacactgaggcacgaaagcgtgggtagcaaacaggattagataccctggtagtccacgccgtaaacgatgagtattaggtgtcgggggttacccccctcggtgccgcagttaacgcattaaatactccgcctggggagtacgcacgcaagtgtgaaactcaaaggaattgacggggacccgcacaagtagcggagcatgtggtttaattcgaagcaacgcgaagaaccttacctaagcttgacatctctcagaccggtagttaatcctaccttcccttcggggctgagatgacaggtggtgcatggttgtcgtcagctcgtgtcgtgagatgttgggttaagtcccgcaacgagcgcaacccttgtctttagttgccagcattaagttgggcactctagagagactgccagggataacctggaggaaggtggggatgacgtcaaatcatcatgccccttatgctcagggctacacacgtgctacaatgggtggtacagagggttgccaagccgcagggtggagctaatcccttaaagccattctcagttcggattgtaggctgaaactcgcctacatgaagctggagttactagtaatcgcagatcagaatgctgcggtgaatgcgttcccgggtcttgtacacaccgcccgtcacaccatggaagttggaaacacccgaagccgattatctaaccgcaaggaagaagtcgtcgaaggtggaatcaataactagggtgaagtcgtaacaaggtagccgtatcggaaggtgcggctggatcacctcc

SEQ ID NO: 109 - DNA, 1487 bp, Mycolicibacterium smegmatis
  misc_feature: NR_025311.1 Mycolicibacterium smegmatis strain ATCC 19420 16S ribosomal RNA, partial sequence
  misc_feature (300)..(300): n is a, c, g, or t
  misc_feature (749)..(749): n is a, c, g, or t
  misc_feature (818)..(818): n is a, c, g, or t
  misc_feature (837)..(837): n is a, c, g, or t
  misc_feature (871)..(871): n is a, c, g, or t
ggcggcgtgcttaacacatgcaagtcgaacggaaaggccctttcgggggtactcgagtggcgaacgggtgagtaacacgtgggtgatctgccctgcactttgggataagcctgggaaactgggtctaataccgaatacaccctgctggtcgcatggcctggtaggggaaagcttttgcggtgtgggatgggcccgcggcctatcagcttgttggtggggtgatggcctaccaaggcgacgacgggtagccggcctgagagggtgaccggccacactgggactgagatacggcccagactnctacgggaggcagcagtggggaatattgcacaatgggcgcaagcctgatgcagcgacgccgcgtgagggatgacggccttcgggttgtaaacctctttcagcacagacgaagcgcaagtgacggtatgtgcagaagaaggaccggccaactacgtgccagcagccgcggtaatacgtagggtccgagcgttgtccggaattactgggcgtaaagagctcgtaggtggtttgtcgcgttgttcgtgaaaactcacagcttaactgtgggcgtgcgggcgatacgggcagactagagtactgcaggggagactggaattcctggtgtagcggtggaatgcgcagatatcaggaggaacaccggtggcgaaggcgggtctctgggcagtaactgacgctgaggagcgaaagcgtggggagcgaacaggattagataccctggtagtccacgncgtaaacggtgggtactaggtgtgggtttccttccttgggatccgtgccgtagctaacgcattaagtancccgcctggggagtacggncgcaaggctaaaactcaaaggaattgacgggggnccgcacaagcggcggagcatgtggattaattcgatgcaacgcgaagaaccttacctgggtttgacatgcacaggacgccggcagagatgtcggttcccttgtggcctgtgtgcaggtggtgcatggctgtcgtcagctcgtgtcgtgagatgttgggttaagtcccgcaacgagcgcaacccttgtctcatgttgccagcacgttatggtggggactcgtgagagactgccggggtcaactcggaggaaggtggggatgacgtcaagtcatcatgccccttatgtccagggcttcacacatgctacaatggccggtacaaagggctgcgatgccgtgaggtggagcgaatcctttcaaagccggtctcagttcggatcggggtctgcaactcgaccccgtgaagtcggagtcgctagtaatcgcagatcagcaacgctgcggtgaatacgttcccgggccttgtacacaccgcccgtcacgtcatgaaagtcggtaacacccgaagccggtggcctaacccttgtggagggagccgtcgaaggtgggatcggcgattgggacgaagtcgtaacaaggtagccgtaccggaaggtgcggctggatcacctcctttct

SEQ ID NO: 110 - DNA, 1399 bp, Enterorhabdus muris
  misc_feature: NR_144609.1 Enterorhabdus muris strain WCA-131- CoC-2 16S ribosomal RNA, partial sequence
tggctcaggatgaacgctggcggcgtgcttaacacatgcaagtcgaacgattaaaccgccctcgggcggacatagagtggcgaacgggtgagtaacacgtgaccaacctgccccgcgctccgggacaaccgctggaaacggcggctaataccggatactccgggagggccccatggccctgccgggaaagccgagacggcgcgggatggggtcgcggcccattaggtagacggcggggtaacggcccaccgtgcccgcgatgggtagccggactgagaggtcgaccggccacattgggactgagatacggcccagactcctacgggaggcagcagtggggaattttgcgcaatggggggaaccctgacgcagcaacgccgcgtgcgggacgaaggccctcgggttgtaaaccgctttcagcagggaagactttacgacggtacctgcagaagaagctccggctaactacgtgccagcagccgcggtaatacgtagggggcgagcgttatccggattcattgggcgtaaagcgcgcgtaggcggccgcctaagcgggacctctaaccccggggctcaaccccgggccgggtcccggactgggcggctcgagtgcggtagaggagagcggaattcccggtgtagcggtggaatgcgcagatatcgggaagaacaccgatggcgaaggcagctctctgggccgtcactgacgctgaggcgcgaaagctgggggagcgaacaggattagataccctggtagtcccagctgtaaacgatgggcgctaggtgtggggggacgatccctccgtgccgcagccaacgcattaagcgccccgcctggggagtacggccgcaaggctaaaactcaaaggaattgacgggggcccgcacaagcagcggagcatgtggcttaattcgaagcaacgcgaagaaccttaccagggcttgacatgccgatgaagccggggagacccggtggccgagaggagtcggcgcaggtggtgcatggctgtcgtcagctcgtgtcgtgagatgttgggttaagtcccgcaacgagcgcaacccccgccccgtgttgccagcattcagttggggactcgcgggggactgccggcgtcaagccggaggaaggtggggacgacgtcaagtcatcatgccccttatgccctgggctgcacacgtgctacaatggccggtacagagggttgccaccccgcgagggggagcggatcccggaaagccggtcccagttcggatcgcaggctgcaacccgcctgcgtgaagccggagttgctagtaatcgcggatcagcacgccgcggtgaatacgttcccgggccttgtacacaccgcccgtcacaccacccgagtcgtctgcacccgaagccgccggccgaaccccc

SEQ ID NO: 111 - DNA, 1469 bp, Adlercreutzia equolifaciens
  misc_feature: NR_041609.1 Adlercreutzia equolifaciens strain FJC-B9 16S ribosomal RNA, partial sequence
agagtttgatcctggctcaggatgaacgctggcggcgtgcttaacacatgcaagtcgaacgattaagacggcttcggccgtgtatagagtggcgaacgggtgagtaacacgtgaccaacctgccccgcgctccgggacaaccgctggaaacggcggctaataccggatgctccggggaggccccatggcctccccgggaaagccccgacggcgcgggatggggtcgcggcccattaggtagacggcggggtaacggcccaccgtgcccgcgatgggtagccggactgagaggtcgaccggccacattgggactgagatacggcccagactcctacgggaggcagcagtggggaattttgcgcaatgggggcaaccctgacgcagcaacgccgcgtgcgggacgaaggccttcgggttgtaaaccgctttcagcagggaagacatagacggtacctgcagaagaagctccggctaactacgtgccagcagccgcggtaatacgtagggggcgagcgttatccggattcattgggcgtaaagcgcgcgtaggcggccgcctaagcggaacctctaatcccggggctcaacctcgggccgggttccggactgggcggctcgagtgcggtagaggcaggcggaattcccggtgtagcggtggaatgcgcagatatcgggaagaacaccgatggcgaaggcagcctgctgggccgccactgacgctgaggcgcgaaagctgggggagcgaacaggattagataccctggtagtcccagccgtaaacgatggacgctaggtgtggggggaccatccccccgtgccgcagccaacgcattaagcgtcccgcctggggagtacggccgcaaggctaaaactcaaaggaattgacgggggcccgcacaagcagcggagcatgtggcttaattcgaagcaacgcgaagaaccttaccagggcttgacatgcgagtgaagccgcggagacgcggtggccgagaggagctcgcgcaggtggtgcatggctgtcgtcagctcgtgtcgtgagatgttgggttaagtcccgcaacgagcgcaacccccgtcccgtgttgccagcattcagttggggactcgcgggagactgccggcgtcaagccggaggaaggtggggacgacgtcaagtcatcatgccccttatgccctgggctgcacacgtgctacaatggccggtacagagggttgccaccccgcgagggggagcggatcccggaaagccggtcccagttcggatcgcaggctgcaacccgcctgcgtgaagtcggagttgctagtaatcgcggatcagcatgccgcggtgaatacgttcccgggccttgtacacaccgcccgtcacaccacccgagtcgtctgcacccgaagccgccggccgaacccttctggggcggaggcgtcgaaggtgtggagggtaaggggggtgaagtcgtaacaaggtaacc

SEQ ID NO: 112 - DNA, 1474 bp, Enterorhabdus caecimuris
  misc_feature: NR_115868.1 Enterorhabdus caecimuris B7 16S ribosomal RNA, partial sequence
agagtttgatcatggctcaggatgaacgctggcggcgtgcctaacacatgcaagtcgaacggttaaaccgccctcgggcggacatacagtggcgaacgggtgagtaacacgtgaccaacctgccccgcgctccgggacaaccgctggaaacggcggctaataccggatactccgcggcccccgcatgggggcggcgggaaagccccgacggcgcgggatggggtcgcggcccattaggtagtaggcggggtagaggcccacctagccgacgatgggtagccggactgagaggtcgatcggccacattgggactgagatacggcccagactcctacgggaggcagcagtggggaattttgcgcaatggggggaaccctgacgcagcaacgccgcgtgcgggacgaaggccctcgggtcgtaaaccgctttcagcagggaagaccttacgacggtacctgcagaagaagctccggctaactacgtgccagcagccgcggtaatacgtagggggcgagcgttatccggattcattgggcgtaaagcgcgcgtaggcggccgcctaagcggaacctctaaccccggggctcaacctcgggccgggttccggactgggcggctcgagtgcggtagaggagagcggaattcccggtgtagcggtggaatgcgcagatatcgggaagaacaccgatggcgaaggcagctctctgggccgtcactgacgctgaggcgcgaaagctgggggagcgaacaggattagataccctggtagtcccagccgtaaacgatgggcgctaggtgtggggggatcccccctccgtgccgcagccaacgcattaagcgccccgcctggggagtacggccgcaaggctaaaactcaaaggaattgacgggggcccgcacaagcagcggagcatgtggcttaattcgaagcaacgcgaagaaccttaccagggcttgacatgcgcctgaagccgcggagacgcggtggccgagaggagggcgcgcaggtggtgcatggctgtcgtcagctcgtgtcgtgagatgttgggttaagtcccgcaacgagcgcaacccccgtcccgtgttgccagcattaggttggggactcgcgggagactgccggcgtcaagccggaggaaggtggggacgacgtcaagtcatcatgccccttatgccctgggctgcacacgtgctacaatggccggtacagagggttgccaccccgcgagggggagcggatcccggaaagccggccccagttcggatcgcaggctgcaacccgcctgcgtgaagccggagttgctagtaatcgcggatcagcacgccgcggtgaatacgttcccgggccttgtacacaccgcccgtcacaccacccgagtcgtctgcacccgaagccgccggccgaacccccccctgggggcggaggcgtcgaaggtgtggagggtaaggggggtgaagtcgtaacaaggtaacc

SEQ ID NO: 113 - DNA, 1440 bp, Coprococcus eutactus
  misc_feature: NR_044049.1 Coprococcus eutactus strain ATCC 27759 16S ribosomal RNA, partial sequence
gatgaacgctggcggcgtgcttaacacatgcaagtcgaacgattgaagctggagcttgctccggccgatttagtggcggacgggtgagtaacgcgtgggtaacctgcctcatacagggggatagcagttggaaacgactggtaaaaccgcataagcgcacrgtatcgcatgatacagtgtgaaaacactccggtggtatgagatggacccgcgtctgattagctagttggtgaggtaacggcccaccaaggcgacgatcagtagccggcctgagagggtgaacggccacattgggactgagacacggcccaaactcctacgggaggcagcagtggggaatattgcacaatgggggaaaccctgatgcagcgacgccgcgtgagtgaagaagtatttcggtatgtaaagctctatcagcagggaagataatgacggtacctgactaagaagcaccggctaaatacgtgccagcagccgcggtaatacgtatggtgcaagcgttatccggatttactgggtgtaaagggtgcgtaggtggcaaggcaagtctgaagtgaaaatccggggctcaaccccggaactgctttggaaactgtttagctagagtacaggagaggtaagtggaattcctagtgtagcggtgaaatgcgtagatattaggaggaacaccagtggcgaaggcgacttactggactgctactgacactgaggcacgaaagcgtggggagcaaacaggattagataccctggtagtccacgccgtaaacgatgaatactaggtgttgggttccaaagggactcggtgccgtcgcaaacgcattaagtattccacctggggagtacgttcgcaagaatgaaactcaaaggaattgacggggacccgcacaagcggtggagcatgtggtttaattcgaagcaacgcgaaaaaccttaccaagtcttgacatcctgatgacggttccttaaccggaacttctcttcggagcatcagagacaggtggtgcatggttgtcgtcagctcgtgtcgtgagatgttgggttaagtcccgcaacgagcgcaacccctatttccagtagccagcagtaagatgggcactctggagagactgccggggataacccggaggaaggtggggatgacgtcaaatcatcatgccccttatgatttgggctacacacgtgctacaatggcagttacaaagagaagcraacctgtgaaggcgagcaaacctcaaaaaggctgtctcagttcggattgtagtctgcaactcgactacatgaagctggaatcgctagtaatcgcagatcagcatgctgcggtgaatacgttcccgggtcttgtacacaccgcccgtcacaccatgggagtcggtaacgcccgaagtcagtgacccaaccgcaaggagggagctgccgaaggcaggatcgataactggggtg

SEQ ID NO: 114 - DNA, 1453 bp, Coprococcus comes
  misc_feature: NR_044048.1 Coprococcus comes ATCC 27758 16S ribosomal RNA, partial sequence
gatgaacgctggcggcgtgcttaacacatgcaagtcgaacgaagcacttatmcttgattcttcggatgaagrggtttgtgactgagtggcggacgggtgagtaacgcgtgggtaacctgcctcatacagggggataacagttagaaatgactgctaataccgcataagaccacrgagccgcatggctcggtgggaaaaactccggtggtatgagatggacccgcgtctgattaggtagttggtggggtaacggcctaccaagccaacgatcagtagccgacctgagagggtgaccggccacattgggactgagacacggcccaaactcctacgggaggcagcagtggggaatattgcacaatgggggaaaccctgatgcagcgacgccgcgtgagcgaagaagtatttcggtatgtaaagctctatcagcagggaagaaaatgacggtacctgactaagaagcaccggctaaatacgtgccagcagccgcggtaatacgtatggtgcaagcgttatccggatttactgggtgtaaagggagcgtagacggctgtgtaagtctgaagtgaaagcccggggctcaaccccgggactgctttggaaactatgcagctagagtgtcggagaggtaagtggaattcccagtgtagcggtgaaatgcgtagatattgggaggaacaccagtggcgaaggcggcttactggacgatgactgacgttgaggctcgaaagcgtggggagcaaacaggattagataccctggtagtccacgccgtaaacgatgactactaggtgtcggggagcaaagctcttcggtgccgcagcaaacgcaataagtagtccacctggggagtacgttcgcaagaatgaaactcaaaggaattgacggggacccgcacaagcggtggagcatgtggtttaattcgaagcaacgcgaagaaccttacctgctcttgacatcccggtgaccggcgtgtaatgacgccttttcttcggaacaccggtgacaggtggtgcatggttgtcgtcagctcgtgtcgtgagatgttgggttaagtcccgcaacgagcgcaacccttatcttcagtagccagcaattcggatgggcactctggagagactgccagggataacctggaggaaggtggggatgacgtcaaatcatcatgccccttatgagcagggctacacacgtgctacaatggcgtaaacaaagggaagcgagcctgcgagggtaagcaaatctcaaaaataacgtctcagttcggattgtagtctgcaactcgactacatgaagctggaatcgctagtaatcgcgaatcagcatgtcgcggtgaatacgttcccgggtcttgtacacaccgcccgtcacaccatgggagttggtaacgcccgaagtcagtgacccaaccgtaaggagggagctgccgaaggtgggaccgataactggggtg

SEQ ID NO: 115 - protein, 477 aa, Ruminococcus gnavus
  misc_feature: Tryptophan decarboxylase (EC 4.1.1.105) Ruminococcus gnavus HB-40
MIGTEYILNSTQLEEAIKSFVHDFCAEKHEIHDQPVVVEAKEHQEDKIKQIKIPEKGRPVNEVVSEMMNEVYRYRGDANHPRFFSFVPGPASSVSWLGDIMTSAYNIHAGGSKLAPMVNCIEQEVLKWLAKQVGFTGNPGGVFVSGGSMANITALTAARDNKLTDINLHLGTAYISDQTHSSVAKGLRIIGITDSRIRRIPTNSHFQMDTAKLEEVIEADKKSGYIPFVVIGTAGTTNTGSIDPLTEISALCKKHDMWFHIDGAYGASVLLSPKYKSLLNGTELADSISWDAHKWLFQTYGCAMVLVKDIRNLFHSFHVNPEYLKDLENDIDNVNTWDIGMELTRPARGLKLWLTLQVLGSDLIGSAIEHGFQLAVWAEEALNSKKDWEIVSPAQMAMINFRYAPKDLTKEEQDILNEKISHRILESGYAAIFTTVLNGKTVLRICAIHPEATQEDMQHTIDLLDQYGREIYTEMKG

SEQ ID NO: 116 - protein, 477 aa, Ruminococcus gnavus
  misc_feature: Tryptophan decarboxylase (EC 4.1.1.105) Ruminococcus gnavus HB-516
MIGTEYILNSTQLEEAIKSFVHDFCAEKHKIHDQPVVVEAKEHQEDKIKQIKIPEKGRPVNEVVSEMMNEVYRYRGDANHPRFFSFVPGPASSVSWLGDIMTSAYNIHAGGSKLAPMVNCIEQEVLKWLAKQVGFTGNPGGVFVSGGSMANITALTAARDNKLTDINLHLGTAYISDQTHSSVAKGLRIIGITDSRIRRIPTNSHFQMDTAKLEEVIEADKKSGYIPFVVIGTAGTTNTGSIDPLTEISALCKKHDMWFHIDGAYGASVLLSPKYKSLLNGTELADSISWDAHKWLFQTYGCAMVLVKDIRNLFHSFHVNPEYLKDLENDIDNVNTWDIGMELTRPARGLKLWLTLQVLGSDLIGSAIEHGFQLAVWAEEALNSKKDWEIVSPAQMAMINFRYAPKDLTKEEQDILNEKISHRILESGYAAIFTTVLNGKTVLRICAIHPEATQEDMQHTIDLLDQYGREIYTEMKG

SEQ ID NO: 117 - protein, 490 aa, Ruminococcus gnavus
  misc_feature: Tryptophan decarboxylase (EC 4.1.1.105) Ruminococcus gnavus ATCC 29149
MSQVIKKKRNTFMIGTEYILNSTQLEEAIKSFVHDFCAEKHEIHDQPVVVEAKEHQEDKIKQIKIPEKGRPVNEVVSEMMNEVYRYRGDANHPRFFSFVPGPASSVSWLGDIMTSAYNIHAGGSKLAPMVNCIEQEVLKWLAKQVGFTENPGGVFVSGGSMANITALTAARDNKLTDINLHLGTAYISDQTHSSVAKGLRIIGITDSRIRRIPTNSHFQMDTTKLEEAIETDKKSGYIPFVVIGTAGTTNTGSIDPLTEISALCKKHDMWFHIDGAYGASVLLSPKYKSLLTGTGLADSISWDAHKWLFQTYGCAMVLVKDIRNLFHSFHVNPEYLKDLENDIDNVNTWDIGMELTRPARGLKLWLTLQVLGSDLIGSAIEHGFQLAVWAEEALNPKKDWEIVSPAQMAMINFRYAPKDLTKEEQDILNEKISHRILESGYAAIFTTVLNGKTVLRICAIHPEATQEDMQHTIDLLDQYGREIYTEMKKA

SEQ ID NO: 118 - protein, 477 aa, Enterocloster lavalensis
  misc_feature: Tryptophan decarboxylase (EC 4.1.1.105) Enterocloster lavalensis (Clostridium lavalense) HB452c
MDSFMEAQTQRAGELMERFIGQTLDFYENIKREDVFRKADGAAISRLLKKGIPQKGRPVDEVYREMMDDVYANTSLVQHPRCFACIPSPVSLFSWMGDVMTNAFDPHAGCVMNASAAGCIERELIRWMCGLAGYPKGCGGLFVSGGSMANLTALTAARDDRLTEAERALAVAYVSDQTHSSIAKGLHIIGFRADQVRKIPSDTNFCMDMETLRAAVRADLAAGRKPFAVIATAGTTNTGSVDPLPEIAEICRTYGMWMHVDGAFGASILLSSNERKRLDGIEYSDSLSWDAHKWLRQTYGCSMVLVRDQSHLVRSFAVHPEYLTDAGAFNEAPDFWDLGPELTRPARSLKLWITLQVMGSEAMGQMIDHGCAMARLTEELIRRYPGWEIVSPARLGIVNFRCAPVNIPPSRIDRLNQDIAREVTDSGYAQILTTELNGKRVLRMCTLHPETTEEDIRNTVRLLCESRAASMGQCRTA

SEQ ID NO: 119 - protein, 610 aa, Enterococcus durans
  misc_feature: Tryptophan decarboxylase (EC 4.1.1.105) Enterococcus durans HB-48
MKDMDIKAVFIGDKAENGAVYKMLLNKMVDEHLGWRENYLPGDLPAISEEDKLAPRYIATRERMMTVLDEVSERMRAGSIPWHSAGRYWGQMNAETLMPSLLAYNYAMLWNPNNVALESSMATSQMEAEVGQDFADLFNMTDGWGHITADGSIANLEGLWYARCIKSIPLAVKEVFPEKVENKTEWELLNLSVEEILEMIEKFTDEELDAVKAASSRSGKHIQELGKWLVPQTKHYSWMKALDICGVGLDQMIAIPVQEDYRMDVAELEKTIRELAEQKIPILGVVAVVGTTEEGQVDSVDKIVELREKLRGEGIYFYLHVDAAYGGYARSLFLNEAGEFVPYESLTEFFEEHQVFHHQVKIDKSVYDGFKAITEADSVTIDPHKMGYVPYAAGGIVIKHKNMRNIISYFAPYVFEKSVKAPDMLGAYILEGSKAGATAAAVWTAHRVLPLNVTGYGQLIGASIEAAQRFREFLGQLSFTVKGKTIEVHPLNHPDFNMFDWVFKVQDCTDLKKINDLNEKMFDVSSYMDGDVYDERFITSHTTFTKADYGDSPVKFIESLGLSKEEWQKEQQVTLLRAAIMTPYLNDDKIFKFYTDAITKAIEKKLNELL

SEQ ID NO: 120 - protein, 285 aa, Ruminococcus gnavus
  misc_feature: Putative Tryptophan hydroxylase (EC 1.14.16.4) Ruminococcus gnavus HB-40
MGLGLRYKNEEQIFQEQYTEPPYLEYPMFRETGIVKHGFSTRIGGVSKGCFSSLNLSFTRGDEEEAVRENFRRIGSAMGIRCEDMVFTHQTHTINVRVVTEADRGCGIVKPRTYSDVDGLVTDIPGICLVTFYADCVPLYFVDPVKKVIGLSHSGWRGTVGKIGKVTVETMCREFKSRPEDILAAVGPSICQDCYEVSEDVIRQFRKNFPEDCWDALFYQKENQKYQLNLWKANELIFREAGILPEHIAVTNLCTHCNSEILYSHRQAGDARGNLCAFLALDQEV

SEQ ID NO: 121 - protein, 324 aa, Enterococcus durans
  misc_feature: Putative Tryptophan hydroxylase (EC 1.14.16.4) Enterococcus durans HB-48
MDTQIRGIHHVTAMTSSATKIYRFFTDILGMRLVKKTVNQDDIETYHLYFTDEHGAPGTDMTFFDFPNQAKGKKRTDTISRASFRVPNDEALTFWLERFEKYGITHSEIKERFSKKYLEFEDFDSQAYQLISDENNHGIASGIPWKNSNVPSKYGITGLGPVFVRVKNLDHIRVILENILGFKETAHADAFYQFEVGEGGNGASIIIEHRTDLPDAIEGFGNIHHLALRVADKEALEHWIRLINKVDLPNSGFVDRFYFQSEYFLAASHVLFELATDGPGFFGDEPTETAGEILSLPPFLEEKRAEIEEYVRPFDTSDANKKRE

SEQ ID NO: 122 - protein, 95 aa, Enterococcus durans
  misc_feature: Putative Tryptophan hydroxylase (EC 1.14.16.4) Enterococcus durans HB-48
MIVINAKFSIKPEKRNEFLAEVKNLIEATKKEDGCLSYKLFESIDTENEFVMIENWRDQQAVEGHNQSPLLQKLFHSMPEYSSKKSEIVVSKTID

SEQ ID NO: 123 - protein, 288 aa, Enterocloster lavalensis
  misc_feature: Putative Tryptophan hydroxylase (EC 1.14.16.4) Enterocloster lavalensis (Clostridium lavalense) HB452c
MKIQWKYKNDLEIFETKGQESTPYLSFRALDDLGMVKNGFSTRMGGASRGKFATMNFSYSRGDDPDHVLENFTRMAAALGVERDRMVASYQTHTTNVRRVTEEDLGKGVVRTRDYRDVDGLITNLPGVTLVTFYADCVPLYLVDPVKKAIGLSHSGWRGTVARMGRETVEAMKREFGTDPGDLVACIGPSICEDCFEVGGEVVEAFCQAFAPEQHPALFRQGVGPGKYQLDLWAANRIILKEAGVPAKQIHTTNICTKCNSDYLFSHRTVGEERGNLAAFLCLKGGAL

SEQ ID NO: 124 - protein, 493 aa, Bacillus velezensis
  misc_feature: Putative Tryptophan hydroxylase (EC 1.14.16.4) Bacillus velezensis SB-9
MKTQVAIIGGGPVGLMLASELALAGVKTTVIERLKKTVPYSKALIMHPRTLELFAMRGILERFVDQGTKVSSGHFSMLDTRLDFSRLDTKQNYSLMLPQAETERMLEEYASSLGANIIRGAEAVAVTQTDDGVETVFRDESGIGTLHSLYAAGTDGAGSIVRKQAGIAFSGTDADLTGTLCDAALKHPPESSFFSTCRLEGSVVIIPLTDGLFRVLIVSPHVPQKRKEERVTEDELKEDLRRICGTDFGLSDPVWMSRFGNAERQAEQYRSGRIFVAGDAAHIHFPAGGQGLNTGLQDAVNLGWKLAAQMKGKAAPHLLDSYHQERHSTGKEVLHNIKTQIKLMDFTESGMALRETMTALLDFQESNRLLAGKVSALDLHYEPDAELPPHTLNGKRLPDIELTLHDGRPARLYDFLRKGGFVLLSLRGSIPAGIDTDSVAAVRAELAETRADWNDVHTVLIRPDGHAAWAAGGSGREAEEAVLAGLERWGLFI

SEQ ID NO: 125 - protein, 482 aa, Bacillus velezensis
  misc_feature: Putative Tryptophan hydroxylase (EC 1.14.16.4) Bacillus velezensis SB-9
MMGIINGKMFIDRLNQLGNEIWLDGEKIEGQISEHPAFKGLLETKRSLYDLQTKPDMKDQMTYSLPGKKERAGLSYLQPKTKDDVKRRRNMFEHWARHTHGMMGRSPDYMNTVLMSFASSAALLKDREHCFPEHVQALYERAAAQDLSFTHTFITPQVNRSQMGVNVSPTPISAKVVDKNEKGLIIHGARILATQGGLTDEVLVVSLPKMYFESDEAFAFSIPSNTKGVKFICRESFVLGKSAFDYPLSSRYEEMDSIVVFDHVLVPWERVFFYNNYEAAFDFNMKSSFNAFAFHQVITRQIVKTEFLLGLAQTLVEAIDVGEYQHIQAKMAEIITGLETMKALLEKSENDAETDEWGYMRPSVTPLRVVSAIFPDIYPRFTEIIQLIGASGMVTLPTEKAFRSDIRGDLDQYLQAAELNAEDRVKVFRLAWDLTMSPFGTRQTQYERYFFGDPVRLTSHLYRSYPKEEQTAVIKEFLHIHK

SEQ ID NO: 126 - protein, 373 aa, Chromobacterium sp.
  misc_feature: Putative Tryptophan hydroxylase (EC 1.14.16.4) Chromobacterium sp. IIBBL 274-1
MKILVIGAGPAGLVFASQMKQARPQWTIDIVEKNDEQEVLGWGVVLPGKPGQHPANPLSYLDAPERLSPQFLEEFKLVHHNEPSLMSTGVLLCGVERRGLVHALRDKCRSQGISIRFESPLLERGELPLADYDLVVLANGVNHKTSHFTEALAPQVDYGRNKYIWYGTSQLFDQMNLVFRTHGKDVFIAHAYKYSDTMSTFVVECSEETYARTRLGEMSDEASAEYVAKVFQAELGGHGLASQPGIGWRNFMTLSHDRCHDGKLVLLGDALQSGHFSIGHGTTMAVVVAQLLVKALCAEDGVPAALRSFEERALPLVQLFRGHADNSRVWFETVDERMHLSSAEFVQSFDARRKSLPPMPEALAQNLRYALQR

SEQ ID NO: 127 - protein, 481 aa, Acetobacter pasteurianus
  misc_feature: tr|A0A401WSJ6|A0A401WSJ6_ACEPA Aromatic-L- amino-acid decarboxylase OS=Acetobacter pasteurianus NBRC 3188 OX=1226663 GN=NBRC3188_0995 PE=3 SV=1
MAGLDPENWEELRTLGHRMLDDMLESIKNVAKGPVWQPMPDTLRQNFKQAPLPHGAQPLEGLYEQFANTVQPYAVGNRHPRFMGWVHGGGTAQGMLAEMLAAGLNANLGGRNHAPVEVERMVIRWAAEMFGFPDTASGVLVTGSSMANFIAILTASRAVPDGTALREQGVAGRKLVGYAAQTAHGCVARAFDMAGLGMKALRRVPVDAAFRMDMQALRRMVAQDRADGYEPFLVVGTAGTVDTGSVDPLDELADYAEAEKLWFHVDGAFGALAQLAPDYAPLLKGLERADSLAFDFHKWAQVPYDAGCVLVREQGRQAATFAQSLAYLSREDRGLAADAPWFCDFGPDLSRGFRALKVWFTLAGFGTEGLGNVVAQSCTVAQYLASRVVATPGLTLLAPVTLNIVCFRVDMPDVGDLDTLNAELVKDLQESGIAAPSTTRIHGKLAIRAAIVNHRTTTEDADIMLAGLADMVQARLGRPLA

SEQ ID NO: 128 - protein, 472 aa, Actinobacteria bacterium
  misc_feature: tr|A0A4P5SJ26|A0A4P5SJ26_9ACTN Aromatic-L- amino-acid decarboxylase OS=Actinobacteria bacterium OX=1883427 GN=LBMAG12_16630 PE=3 SV=1
MDAQEFRANGQAVIELIAQYLETIEERPVTADVKPGDVRHQLPAHPPTAPESFESVLADVSNIIVPGLTHWQHPNFFAFFPGNSSYPSILADLLTAGLGVQGMSWLSSPACTEVETLMLDWMQELLGLPESFRSTTASGGGVIQGSASEATLSSILAARWRITEGHVNRDGDTTKLIAYCTSQSHSSIEKGLRIAGIGTQNIRIIEHDASFAMVADALEAQILADQAAGLIPFWVCSTHGTTSSGAFDPTSAIGAITQRHHLWLHVDAAMHGIAALAEEYRWVNDGLELADSYCTNPHKWMGVNFDCDLFWTQDRASLLGALSILPEYLRSAAAETGAAIDYRDWQIPLGRRFRSLKLWFAIRCDGTAVFQEIIRRHIQITQELADLVAQDHRFEIMAPHPLNLLCIRLRGESPQDGDARTDALIEAANATRSVFFTRTILEGRSVLRFSIGGRTTHGQHALSAWGLLRSFA

SEQ ID NO: 129 - protein, 480 aa, Bacillus atrophaeus
  misc_feature: tr|I0DFJ0|I0DFJ0_BACAT Aromatic-L-amino-acid decarboxylase OS=Bacillus atrophaeus OX=1452 PE=3 SV=1
MSENLQLSAEEMRQLGYQAVDLIIDHMNHLKSKPVSETIDSDILRNKLTESIPENGSDPKELLHFLNRNVFNQITHVDHPHFLAFVPGPNNYVGVVADFLASGFNVFPTAWIAGAGAEQIELTTINWLKSMLGFPDSAEGLFVSGGSMANLTALTVARQAKLNNDIENAVVYFSDQTHFSVDRALKVLGFKHHQICRIETDEHLRISVSALKKQIKEDRTKGKKPFCVIANAGTTNCGAVDSLNELADLCNDEDVWLHADGSYGAPAILSEKGSAMLQGIHRADSLTLDPHKWLFQPYDVGCVLIRNSQYLSKTFRMMPEYIKDSETNVEGEINFGECGIELSRRFRALKVWLSFKVFGVAAFRQAIDHGIMLAEQVEAFLGKAKDWEVVTPAQLGIVTFRYIPSELASTDTINEINKKLVKEITHRGFAMLSTTELKEKVVIRLCSINPRTTTEEMLQIMMKIKALAEEVSISYPCVAE

SEQ ID NO: 130 - protein, 454 aa, Bacteroidetes bacterium
  misc_feature: tr|A0A136NI40|A0A136NI40_9BACT Aromatic-L- amino-acid decarboxylase OS=Bacteroidetes bacterium OLB10 OX=1617421 GN=UZ10_BCD003001760 PE=3 SV=1
MQEAERNTITDAVTSYTNHFIAGLKHVKGFQQNELNSLAIDNKKKTIEELLSIYQKEVAENGINAASGTHLGYIPGGGVFTGAVADFIAAVTNPFASVYYASPGAAMVENEVVNWLKRVFSFPESATGCLSSGGSISTLIAFTAARDKHQVKNERVRKSVVYLSEQVHHSTQKALRIIGLEDVLIRYVPLDSHHRIQVDALEQLIAKDTAQGLIPFLIVATAGTTDTGAIDPLNDIADLAKKYHTWFHVDAAYGGFFILTSRKSLFKGIERADSMIVDPHKGMFLPYGVGAVLIKDISATLHSNYYEANYMQDAAEDELAKSPANLSPELTKHFRGLRVWLPLQYHGIEPFIACLEEKLLLVKYFRQQLQERGFWIGPEPDLSVSYFRYPFEKDADRKNQQLMNEIHADGDVFLSSSVIEGKFVIRIAILAFRTKKETVDRAVAMIERCLKKVK

SEQ ID NO: 131 - protein, 461 aa, Gemmobacter megaterium
  misc_feature: tr|A0A1N7QKD0|A0A1N7QKD0_9RHOB Aromatic-L- amino-acid decarboxylase OS=Gemmobacter megaterium OX=1086013 GN=SAMN05421774_1143 PE=3 SV=1
MNHDDLRDWSKRAADWAHHYHAGLRDRPVRATIAPGSLRDALPAAAPEAGEPMEQIWKDFQALVPDAMTHWQHPRFFAYFPANAAPVSMLAEQLANAMAAQAMLWQTAPAATEIEQAMMVWLRKAIGLPARFTGTIHDSATSATLSAVLTMRERALGWRGIAEGLSGAPRLRLYASAETHSSVDKAARLAGIGQDNLVKVPTDASLSMDPAALADAISADRAAGALPIGVVICAGGTSVGAFDRVGDVIAVARTEGLPTHVDAAWAGSAMICPEFRALWAGVEGADSLVFNPHKWLGAQFDCAVQFIADPGAQIRTMGLRPTYLETPDDEEIVNFNEWTVPLGRRFRALKLWFLLRAHGLEDLRQRIRNHVAWAHQARDALAALPGVEIVTEPSLSLFSFALRDGDAATEALLRRINDDGRIYLTQTRHGGRYVIRVQVGAFDCTRADVEVIAQVVQDLLP

SEQ ID NO: 132 - protein, 477 aa, Micrococcus lylae
  misc_feature: tr|A0A1R4J257|A0A1R4J257_9MICC Aromatic-L- amino-acid decarboxylase OS=Micrococcus lylae OX=1273 GN=FM125_05970 PE=3 SV=1
MTPEEFRTHGHALIDWIADYRAALHERPVMSPLRPGEVAAQLPAAAPETGEDPATVLADLDRVILPGLSLWQHPEFFHFFPANAELSSVLGDIASTGLGVLGLSWESSPALSEVEIRTMDWMRRLLGLSGAWRGVVQDSASGCALTAMIEARERASGFAMHAGGTDQGRDRMVVYCAAQAHSSVPKAMLLAGFGRQNVREVPVDADYAMDAQALRAIVAEDRAAGLRPAAVVATCGTTATTALDPLAEIGRIAQDEDLWFHVDAAMAGSAMLLPECRWMWEGVELADSLVVNAHKWLGVAFDCSLFFVKDPVFLEKVMATHPSYLQSSHDEDVVNLRDWGVPLGRRFRALKLWFMLRMEGAEALRARLRRDLDNAAWLAAQVDAAEGWERVAPTPLQTVCVRHVPAGLDPADPADTEAIDAHTRAWAQAVNASGKAAVSLATLEGRWMVRLSVGALGGTEREHVERLWKVLGEAVAR

SEQ ID NO: 133 - protein, 470 aa, Pseudomonas putida
  misc_feature: tr|A0A059UV10|A0A059UV10_PSEPU Aromatic-L- amino-acid decarboxylase OS=Pseudomonas putida OX=303 GN=DW66_2355 PE=3 SV=1
MTPEQFRQYGHQLIDLIADYRQTVGERPVMAQVEPGYLKAALPAQAPRQGEPFAAILDDVNQLVMPGLSHWQHPDFYGYFPSNGTLSSVLGDFLSTGLGVLGLSWQSSPALSELEETTLDWLRQLLGLSGQWSGVIQDTASTSTLVALICARERASDYALVRGGLQAQAKPLIVYVSAHAHSSVDKAALLAGFGRDNIRLIPTDERYALRPEALQVAIEQDLAAGNQPCAVVATTGTTATTALDPLRPIGEIAQAHGLWLHVDSAMAGSAMILPECRWMWDGIELADSLVVNAHKWLGVAFDCSIYYVRDPQHLIRVMSTNPSYLQSAVDGEVKNLRDWGIPLGRRFRALKLWFMLRSEGVEALQARLRRDLDNAQWLAGQIGAAAEWEVLAPVQLQTLCIRHRPAGLEGEALDAHTKGWAERLNASGDAYVTPATLDGRWMVRVSIGALPTEREHVEQLWARLQEVVKG

SEQ ID NO: 134 - protein, 962 aa, Sterolibacterium denitrificans
  misc_feature: tr|H9NN89|H9NN89_9PROT Steroid C25 dehydrogenase alpha subunit OS=Sterolibacterium denitrificans OX=157592 GN=s25dA PE=3 SV=1
MQISRRQFIVGSAVAAAGLGLYSLRPKHYVPAAPRPADKPGIPAKKVKYNDYSDIWREKWKWDRVVKGTHTRANCIGACSWDVYVKDGIAWREEQAAIYEPHRPDIPDFNPRGCQKGACYTHLQVNESRIKYPLKRVGERGEGKWKRITWDEALTEIADKLIDAAVAEGTESIIFDDGTTNAGYGPETAGDVRFATSLQTTKIDSWAGVSDMPMGLVQTWGMYNCEGTSDDWFRSDYIVIWVGNPIYTRIPEAHFLHEARYRGAKLVVIAPDLNPSTVHADTWLKINPETDAALGLAAAQVMITENLIKKDYVLEQTDMPFLVRKDDKRFLRGADMVKGGADNAVYIWDEAKQAAVVAPGCEGDGEGGRSLKLNGIKPALSGTFTVKLANGESVEVHTVFDMLKEKLDTEYTPELAEKVTGIHPNVIRTFAREMAAAPNAMIYCSWGACKHHHSDLYQRAMALLMTFTGNQGKPGSGVRVAAWWGVDGMEAMTAGLFSTMDKIKLIPKAIKGLTPRDYEDLFIDISDREANTPMIVFLYVHGGYKEMWDKPHLQDPALPRNLAEYMRESIDKGWTKVYPPENRSPKAYIFTGCNPLRRWPSPQIAKKVLWPKFDVVVSVNFRMSTSTMFADYVLPVAGYYEKYGIKYGQSYVPYLITSDKATEPLGESKSDWEVFGLLSKTIAERAKARNIPPVRGFKDRPHDLTKVYDIHTENGLYDPTHPEDPVKLLDAIFEGSPSLGANSGREALEMGAVPIISTGRPSLIYQNYTDYDPKDTHWPHGDFVTKKTAWPTLTGRQQFYIDHPWFLEGGESLPTHKPPPHAGSKYPLRIYGGHNRWSIHAIWRDVKLLLRLQRGQPACWLNPLEAEKRGIKDGDMARLYNHHGEFECMVKVAPITAPGEVIVYHAWEPYQFKNWKGNQEPAEAPWKALHLAGGYHQIHYRMYYGGPSHAPRGVPIEIEKVK

SEQ ID NO: 135 - protein, 256 aa, Ruminococcus bromii
  misc_feature: tr|A0A2N0V092|A0A2N0V092_9FIRM Tryptophan synthase alpha chain OS=Ruminococcus bromii OX=40518 GN=trpA PE=3 SV=1
MTDIKSAFQNGKAFIPFVTAGDPDLETTEKLLIEMSKNGADIIEIGIPFSDPIAEGVVIQEADLRSLSAGTTTDKIFDMVKRIRPQIDAVLAVMTYMNPIFVYGTERFMAKCQECGISAVIVPDTPYEEKHELTDYSGKYGIDVISLIAPTSHERIKMIAKEAEGFVYCVSSMGVTGVRSEITTDVGKMVRLVKSVKDIPCAIGFGISTPEQAKKMSESADGVIVGSAIVKIIAKYGKDSVKPVCDYVKSMKEAIS

SEQ ID NO: 136 - protein, 256 aa, Acetoanaerobium sticklandii
  misc_feature: 136>tr|E3PTA4|E3PTA4_ACESD Tryptophan synthase alpha chain OS=Acetoanaerobium sticklandii (strain ATCC 12662 / DSM 519 / JCM 1433 / NCIMB 10654) OX=499177 GN=trpA PE=3 SV=1
MNNIERVFKNKKAFIPFITCADPNLETTREIIFELVKNGADIIELGIPFSDPTAEGEVIQSANTRALLNGVTTDEILDFVQEVKKNLSTVLVFMTYANVIFSYGAEKFIKRCSEIGIEGIIVPDLPYEEKNEFYPFCKKYEVNLISMIAPTSEDRISMIAKEAEGFLYIVSSMGVTGERTNITTDLEKIINIAKLNKDIPCAVGFGISTPSQVSKILEIADGVIVGSAIVKIIEKHSINSAKYVGEFVKQMKSEIK

SEQ ID NO: 137 - protein, 256 aa, Bacteroides xylanisolvens
  misc_feature: tr|D6D4J0|D6D4J0_9BACE Tryptophan synthase alpha chain OS=Bacteroides xylanisolvens XB1A OX=657309 GN=trpA PE=3 SV=1
MNRINQLFNSNKKDILSIYFCAGTPTLDGTANVIRTLEKHGVSMIEIGIPFSDPMADGIVIQNAATQALRNGMSLKLLFEQLRDIRKDVKIPLVFMGYLNPIMQFGFENFCRKCVECGIDGVIIPDLPFRDYQEHYRIIAERYNIKVIMLITPETSEERVREIDTHTDGFIYMVSSAATTGAQQNFNEQKQAYFKKIKDMHLNNPLMIGFGISNKATFQAACEHASGAIIGSKFVTLLEEEKDPEKAILKLKEAVK

SEQ ID NO: 138 - protein, 250 aa, Enterococcus durans
  misc_feature: tr|A0A367CA00|A0A367CA00_9ENTE Tryptophan synthase alpha chain OS=Enterococcus durans OX=53345 GN=trpA PE=3 SV=1
MKPLTLYLENQTTKPLFIPYIMAGAQGLEHLPEEIEMLAKSGATAIELGIPFSDPVADGPIIQEAGLNAFKHQVTLKKIIAVLQDYHSPVPLILMGYSNSFFHYGVKQLVDDLKDCHVQGVIIPDLPYEHRDLVLPDLGDIALIQLVSLTSPSERIDTLVKEAEGFIYAVTINGTTGVNKTYQQNLTEHLKEIKEKSAIPVLAGFGISKAEHVRRFAQCCDGVVVGSKIVHSLQVDGLTKTQELIGELLR

SEQ ID NO: 139 - protein, 291 aa, Bifidobacterium adolescentis
  misc_feature: tr|A1A1I9|A1A1I9_BIFAA Tryptophan synthase alpha chain OS=Bifidobacterium adolescentis (strain ATCC 15703 / DSM 20083 / NCTC 11814 / E194a) OX=367928 GN=trpA PE=3 SV=1
MTNATSSTPSGQPLGISHKPSPTETMFDEFKAADKPAFIGYLPYGFPDPDYSLKAFRTMVEHGVNAVEIGLPYSDPVMDGPVIQAASQIAIDNGEKIANVFKAVETVANAGGVPLVMSYWNLIFHYGVERFARDFENAGGAGLITPDLIPDEAGEWIEASDRHGLDRIFLVSPDSTEDRLKVVADNARGFVYAAARMGVTGERSTIDASPKELVARTRKAGAKNVCVGIGVSTAEQGARVGSYADGVIVGSALVHTMIDESGKHAVDEATGLKALAAKTEELAEGIHNARN

SEQ ID NO: 140 - protein, 394 aa, Ruminococcus bromii
  misc_feature: tr|A0A413K7N7|A0A413K7N7_9FIRM Tryptophan synthase beta chain OS=Ruminococcus bromii OX=40518 GN=trpB PE=3 SV=1
MTKGKFGIHGGQFVPETLMNAINEFEEAYNRYKDDPEFVAELDTLMREYAGRPSLLYYAEKMTKDLGGAKIYLKREDLNHTGSHKLNNCLGQCLLAKRMGKTRVIAETGAGQHGVATATVAALLGLECEIFMGKEDTIRQALNVYRMKLLGAKVNAVETGTMTLKDAVNEAMREWTNRVSDTHYVLGSVMGPHPFPTVVRDFQSVIGKEIKSQMLEKEGRLPDVVMACVGGGSNAMGAFYEFIKDENVKLIGCEAAGLGTDTPKTAATIATGTLGIFHGMKSYFCQNEYGQIAPVYSISAGLDYPGIGPEHANLADTGRASYVPVTDEEAVNAFEYLSRTEGIIPAIESAHAVAHCIKIAPTMDKDKIIVVNLSGRGDKDVAAIARYRGEDLHD

SEQ ID NO: 141 - protein, 395 aa, Acetoanaerobium sticklandii
  misc_feature: tr|E3PT95|E3PT95_ACESD Tryptophan synthase beta chain OS=Acetoanaerobium sticklandii (strain ATCC 12662 / DSM 519 / JCM 1433 / NCIMB 10654) OX=499177 GN=trpB PE=3 SV=1
MERNNGYFGQFGGAFVPEELKNVLKDVEEAFYKYIEDEEFLSELAYYQRQYIGRENPLYLAKRLSEQLGGAKIYLKREDLNHTGAHKINNAMGQVLLAKRMGKKRIIAETGAGQHGVATATVCALMGLECIIYMGAIDIKRQELNVFRMEMLGAKVVPVTTGTATLKDAVDEALADFVANAHDTFYLLGSAVGPHPYPTMVREFQSVIGKEARKQILEVEGKLPDYIVACVGGGSNAIGLFAPFVDDLSVKIVGVEPGGKGLDTNEHAASISKGSVGVIHGFKCYTLQDENGEPLPVHSIAAGLDYPGVGPEHSYYKESNRAEYIAITDDEAMQAFFLLSRTEGIIPAIESAHAVAYAVKLAKTLSKDQTIIVNLSGRGDKDVNQVKEMNLLDKN

SEQ ID NO: 142 - protein, 395 aa, Bacteroides xylanisolvens
  misc_feature: tr|D6D4J6|D6D4J6_9BACE Tryptophan synthase beta chain OS=Bacteroides xylanisolvens XB1A OX=657309 GN=trpB PE=3 SV=1
MKSFLVDQDGYYGEFGGAYVPEILHKCVEELTNKYLEVIESEDFKKEFDQLLRDYVGRPSPLYLAKRLSEKYGCKLYLKREDLNHTGAHKINNTIGQILLARRMGKKRIIAETGAGQHGVATATVCALMDMECIVYMGKTDVERQHINVEKMKMLGATVIPVTSGNMTLKDATNEAIRDWCCHPADTYYIIGSTVGPHPYPDMVARLQSVISEEIKKQLQEKEGRDYPDYLIACVGGGSNAAGTIYHYINDERVGIILAEAGGKGIETGMTAATIQLGKMGIIHGARTYVIQNEDGQIEEPYSISAGLDYPGIGPIHANLAAQSRANVLAINDDEAIEAAYELTKLEGIIPALESAHALGALKKLKFKPEDIVVLTVSGRGDKDIETYLSFNEQL

SEQ ID NO: 143 - protein, 395 aa, Enterococcus durans
  misc_feature: tr|A0A367CAD3|A0A367CAD3_9ENTE Tryptophan synthase beta chain OS=Enterococcus durans OX=53345 GN=trpB PE=3 SV=1
MYQQPNEQGFYGEFGGQFVPETLMYAVKELTETYEETKTDEEFQKEFAYYLKQYVGRENPLYFAQRLTEHLGGAKIYLKREDLNHTGAHKINNALGQVLLAKRMGKKKVVAETGAGQHGVATATAAALFGMECTIFMGEIDVKRQELNVFRMELLGAKVVSVTSGSKTLKDAVNEALRYWVEHVEDTHYVMGSVLGPHPFPEIVRDYQSVIGTEARRQILEAEGRLPDVAIACIGGGSNSMGLFYPFLNDPVRLIGVEASGHGLDTNEHAASINKGSVGVLHGAKMHLLQDDDGQVIEAFSISAGLDYPGIGPEHSYLYTSGRAEYAAITDREAVEAFHLLSEKEGIIPALESSHALAHVMKLAPTMAKDELIVVCLSGRGDKDVQQIKEMEEQA

SEQ ID NO: 144 - protein, 697 aa, Bifidobacterium adolescentis
  misc_feature: tr|A1A1I8|A1A1I8_BIFAA Multifunctional fusion protein OS=Bifidobacterium adolescentis (strain ATCC 15703 / DSM 20083 / NCTC 11814 / E194a) OX=367928 GN=trpBC PE=3 SV=1
MSVLDELVAGAVEDQRSRENNVPLAEVKRMAFEASAPIDARQWLKKADGIPVIAEIKRASPSKGHLSDIPDPAALAREYEQGGASAISVLTEGRRFLGSLEDFDKVRAAVRIPVLRKDFIVTEYQIWEARAHGADLVLLIVAALDDVKLKSLLDLAHSLNMTVLVETHTREEIQRAINAGAKVIGINARNLKDLKVDVNKYNELAADLPDDVIRVAESGVFGSVELEDYARAGADAVLVGEGVATAANHEQAVERLVKAGARVKASEQTPLASHEGPYFGQFGGRYVPEALITALDELERVYTEAKADPEFHKELARLNQQYVGRPSPLTEAPRFAQRLKEKTGLDARVFLKREDLNHTGAHKINNALGQALLVKRMGKTRVIAETGAGQHGVATATVCAMLGLKCRIYMGQIDARRQALNVARMRMLGAEVVEVTLGDRILKDAINEALRDWVTNVKDTHYLLGTVAGPHPFPSMVRDFQKIIGEEAKQQLQDWYGIDHPDAICACVGGGSNAIGIMNAFLDDERVNLYGYEAGGNGPESGRHAIRFAPGTGELGMFQGAKSYLLENPEGQTLDTYSISAGLDYASVGPEHAWLKDIGRVNYSWATDQEAMSAFKDLCETEGIIPAIESSHAVAGAYKAAADLKAKGYEHPVMIINISGRGDKDMNTAGKWFGYLTDEQAKALESNGAQGNNADGE

SEQ ID NO: 145 - protein, 260 aa, Ruminococcus bromii
  misc_feature: tr|A0A2N0UX30|A0A2N0UX30_9FIRM Indole-3- glycerol phosphate synthase OS=Ruminococcus bromii OX=40518 GN=trpC PE=3 SV=1
MILETIADANRVRYEEIEKQVPLEVIKQKALSMETDNEFPFEKALAGKDISFICEVKKASPSKGIIAEDFPYVQIAKDYENAGASAISVLTEPKWFKGENAFLEEISKNVSIPLLRKDFTVCEYQIYEAKTIGASAVLLICSLLDTDTIRRWIKLCDSLGLSALVEAHTADEVYSAIAAGARVIGVNNRNLRDFTVDINNCTKLRKLVPDNIIFVAESGIKTRDDIKVLENAGVNAVLIGETLMRNPDKKAALDELSGRK

SEQ ID NO: 146 - protein, 262 aa, Acetoanaerobium sticklandii
  misc_feature: tr|E3PTA8|E3PTA8_ACESD Indole-3-glycerol phosphate synthase OS=Acetoanaerobium sticklandii (strain ATCC 12662 / DSM 519 / JCM 1433 / NCIMB 10654) OX=499177 GN=trpC PE=3 SV=1
MNNILKEIVGYTKFRIDEEKMKISLEKTIELAFSIQTESNFRFENALKTKEISLICECKKASPSMGIIDYNYNYINIAREYEASGASCISVLTEPKWFMGSDDHLVDVVKNVSIPCIRKDFIVDEYMIYQSKIIGASAVLLICSVLDKDSLRRYLRICQDLGLSALVEVHTENEIDIALEVGARIIGVNNRNLKDFSVDIRTSERLRKVVPSDVLFVSESGIKTESDVRRLIEIGVDAMLIGEAFMKSDNKSKLIDSWRGKK

SEQ ID NO: 147 - protein, 260 aa, Bacteroides xylanisolvens
  misc_feature: tr|I9UUT1|I9UUT1_9BACE Indole-3-glycerol phosphate synthase OS=Bacteroides xylanisolvens CL03T12C04 OX=997892 GN=trpC PE=3 SV=1
MKDILSEIIANKRFEVDLQKQAISIEQLQEGISESPTLRSMKQALASSASGIIAEFKRRSPSKGWIQEEACPEEIVPSYAAAGASALSILTDEKFFGGSLKDIRAARPLVEIPILRKDFIIDEYQLYQAKIVGADAVLLIAAALEPEKCNELAEKAHELGMEVLLEIHSSEELAYINKGIDMVGINNRNLGTFFTDVENSFRLAGQLPQDSVLVSESGISDPEIVKRLRAAGFRGFLIGETFMKTQRPGETLQNFLQAIQ

SEQ ID NO: 148 - protein, 252 aa, Enterococcus durans
  misc_feature: tr|A0A367CBM8|A0A367CBM8_9ENTE Indole-3- glycerol phosphate synthase OS=Enterococcus durans OX=53345 GN=trpC PE=3 SV=1
MDFLEKILKEKANEVNNLTPVEKRVEKQRPSFYQTVKGNPKKVHIIGEIKRASPSKGLINGTVDILQQAKTYEKSGVSAISVLTDPVFFNGTIHDLTAVAQVVDVPVLCKDFIIDEQQIVRAKQAGASLILLIVAALPKVRLKELHEYATELGLEVLVETHDAQELAIAKEIGATIIGVNNRNLKTFQVSLQTSIDLSQPEGQCIYISESGFKTAQDVEKISQNYQGILVGETLMRAGNVEEKVKELQVSRR

SEQ ID NO: 149 - protein, 336 aa, Ruminococcus bromii
  misc_feature: tr|A0A2N0V079|A0A2N0V079_9FIRM Anthranilate phosphoribosyltransferase OS=Ruminococcus bromii OX=40518 GN=trpD PE=3 SV=1
MIKEAIKKLVAGNDLTFDEAAQVMDEMFSGTATQSQMAAYLTALRIKGETIDEITASAQVMREKALHIKPNRDVLDIVGTGGDGTGTFNISTTAAFIIAAAGIPVAKHGNRSMSSKSGSADCLEQLGININITPEKSEEVLNKAGICFMFAQGYHSSMKYVGPVRKEIGIRNIFNVLGPLTNPAGADLQVTGVYSEALVEPIAQVFSNLGVKKGYVFYGMDGMDEVTLTTTTKVCEIDNGKFNTFILNPEDYGLKLCAPEDLAGGDGKENAEITKEILSGEIKDAKRDIVVLNAALGLCTGGKADSIQDGIKLANEIIDSGKAYAKIEEFAKASNE

SEQ ID NO: 150 - protein, 338 aa, Acetoanaerobium sticklandii
  misc_feature: tr|E3PTA9|E3PTA9_ACESD Anthranilate phosphoribosyltransferase OS=Acetoanaerobium sticklandii (strain ATCC 12662 / DSM 519 / JCM 1433 / NCIMB 10654) OX=499177 GN=trpD PE=3 SV=1
MIKDVIIKLVNKQDISYQETYEVISQIISGKSTMVQNAAFLTALSTKNTKSETIEEILGAALAVRDNALEIKHDMEVLDIVGTGGDNSGSFNISTTSAIVLAAGGIKVAKHGNRAASSKCGTADCLEALGININLSPEKCKTLLEKIGICFIFAQNYHESMKNVAQVRKELGFRTIFNVIGPLANPSKPSIQFLGVYDESLVEPMAKVLASLGVERGMVVYGHDKLDEISISSETTICEFAGKQINKYLIKPEDFGLRRYKKEDIFGGNPKENADILKSILEGEQGARRDIVSINAGACFYSAGKVGSISNGISLANQIIDSGLALKKLEEFRKMSNE

SEQ ID NO: 151 - protein, 331 aa, Bacteroides xylanisolvens
  misc_feature: tr|D4VH46|D4VH46_9BACE Anthranilate phosphoribosyltransferase OS=Bacteroides xylanisolvens SD CC 1b OX=702447 GN=trpD PE=3 SV=1
MKQILYKLFEHQYLGRDEARTILQNIAQGKYNDVQVASLITVFLMRNISVEELCGFRDALLEMRIPVDLSDFAPIDIVGTGGDGKNTFNISTASCFTVAGAGFPVVKHGNYGATSVSGASNVMEQHGVKFTSDVDQLRRSMEKCNLAYLHAPLFNPALKAVAPVRKGLAVRTFFNMLGPLVNPVLPAYQLLGVYNLPLLRLYTYTYQESKTKFAVVHSLDGYDEISLTNEFKVATSDHEKIYTPESLGFSRYKDIDLDGGQTPEDAAKIFDHIMNNTATEAQKNVVIVNSAFAIHVIRPEKTIEECIALAKESLESGRALATLKKFIELNS

SEQ ID NO: 152 - protein, 333 aa, Enterococcus durans
  misc_feature: tr|A0A377L8Z1|A0A377L8Z1_9ENTE Anthranilate phosphoribosyltransferase OS=Enterococcus durans OX=53345 GN=trpD2 PE=3 SV=1
MQQLFERIYQQKDLSQAEMEQLATAMFDQEVTANQISGVLIGLKVKGASTEELTGLAHVMQKRAIPMEKAPTGVMDNCGTGGDHSQSFNISTTAAFVLAGGGIPMAKHGNRSISSRSGSADVLEALGVTITNSKEQIDHLLKNVGIAFLFAPAMHPAMRAVMSVRKELATPTIFNLIGPLINPYPLEYQLMGTYAGDSLASTAETLGKLGRKQALVLHGYQGMDEANLGGKTHCAFYQSGKVSEFSFSPEEVGLESLPLTAITGGDVQKNKEILLSVLKGEKTPYYETVLLNAGLGFLAGAKVQSIREGIKEAEQVIQSGAAFDTLQNLLKEQ

SEQ ID NO: 153 - protein, 377 aa, Bifidobacterium adolescentis
  misc_feature: tr|A1A267|A1A267_BIFAA Anthranilate phosphoribosyltransferase OS=Bifidobacterium adolescentis (strain ATCC 15703 / DSM 20083 / NCTC 11814 / E194a) OX=367928 GN=trpD PE=3 SV=1
MGERPIVTIGGIRTTIKACDSAKEGIMAEITWKSILTKLVGGDHLSAEESEWFVDDLMNGNADPAAVGAVLATQQQLGLTPDEVRGAAKAMVAHAIPLHIDGETTDIVGTGGDGASTVNLSSMGAVVAAAAGVKVVKHGNRAASSKCGTADCFEALGLPMDLTPEQVSEVGNECGIAFAFARTFHPAMRFVGPIRAALGVPCVFNVLGPLTNPASPKHMAVGCANRKMSPIMAAVYAANGQIGMVYTSHEGLDEMAPTGPVSIWEFKDGKVTENKFDPTVELGLAKVTIADLKGGEPTLNAQLARDFFAGKDVSFRTTALLNAASAIVADGHLVPGDASLADRFKAAYAIAEQTVDSGKATALLDKWIATAQAAKKA

SEQ ID NO: 154 - protein, 486 aa, Ruminococcus bromii
  misc_feature: tr|A0A2N0ULM0|A0A2N0ULM0_9FIRM Anthranilate synthase component 1 OS=Ruminococcus bromii OX=40518 GN=trpE PE=3 SV=1
MIKPSYDEVLNLKDNYSVIPIYKEIYADAFTPINLLRKIAGKSDKFFLLESIEGGEKWARYSFIGFNPKARLSYKNGTLTVTGEGARVVKTAKPYDALREYLADYKTPHFKDIPPFTGGLVGYFGYAMISVAEPVLRLKRGDTNDFDMMLFDKIIAYDHLKEKLIIIVNMKTNDTKAQYELAKKDIAEIISTITSPEPLPKLESDGNVEFTSTYSKEEFCKMVEKTKEYIFDGDIFQCVVSRRFEADYKNSLINAYRVLRSTNPSPYMVYMSIDGDEIISTSPETLVKLQNGVLNTFPIAGSRPRGKTDTEDNALADELIHDEKELAEHNMLVDLGRNDIGKISEFNSVKVTKYQEVLRYSKIMHICSEVEGKLKDGLDAFDAIESLLPAGTLSGAPKIRACEIIEELERTPRGVYGGALGYVDFNGNLDTCIAIRMAVKKNDKVYVQAGAGIVADSVPESEYEETYNKALAVMNAVKNAGEVNAL

SEQ ID NO: 155 - protein, 484 aa, Acetoanaerobium sticklandii
  misc_feature: tr|E3PTB1|E3PTB1_ACESD Anthranilate synthase component I OS=Acetoanaerobium sticklandii (strain ATCC 12662 / DSM 519 / JCM 1433 / NCIMB 10654) OX=499177 GN=trpE PE=4 SV=1
MIKPDFKLLKELSKQGKFDITPVSISFKSNSKPIDVFNLLKEKFECTFILDVDDEYGNYDRYTFIGFNPKMKVSCKDSNMCLGEKRIFTKKPSEVLRGILKENRSPKFDFLPPFTGGFAGYFSYDYFKYIEPSVKFTSDNTCNFNDLELFLFDKIVVLDNLNNLITIIVNTSLLEIEKNYTKTIEDLNCIKQIISQENENLMKKGKLKSEFSPAVSKNDYMQMLEKSKSHIFEGDIFQIVLSNKVEAEFEGSLLNVYENLRLINPSPYIFYLSTEDIEIAGASPETLVKLKDKTLNTYPLAGTRPRGKTAEEDFNLEKELLLDEKEVAEHNMLVDLGRNDLGKISKFNSVEVEKYKMVERYSHVMHIASSVKGEICDGKDALDAIEAVLPAGTLSGAPKIKAVELIDKLEKDRRGIYGGALGYISFTGDMDMCIGIRLAYKKGDKVFVRSGAGIVSDSIPESEYNECINKSGAMIATINLLKEG

SEQ ID NO: 156 - protein, 468 aa, Bacteroides fragilis
  misc_feature: tr|Q64SX8|Q64SX8_BACFR Anthranilate synthase component I OS=Bacteroides fragilis (strain YCH46) OX=295405 GN=BF2651 PE=4 SV=1
MNAFNYTTHSKQVLGDLHTPVSIYLKVRDMYPQSALMESSDYHAGENSLSFIALCPLASIGINSGIVTTTYPDNTRREEPLSQSFRVENALNRFINRFHVEGDDKKFCGLYGYTTFNAVKYFEHIPVKESHDEQNDAPDLLYILYKYIIVFNHFKNELTLVEMLAEGEESNLSQLESAIENRNYASYNFSVTGPVTSTITDEEHKANVRKGIAHCLRGDVFQIVLSRRFIQPYAGDDFKVYRALRSINPSPYLFYFDFGGYRIFGSSPETHCKVESGQAYIDPIAGTTRRTGDTIKDKELTEALLADPKENAEHVMLVDLARNDLSRNCHDVRVVFYKEPQYYSHVIHLVSRVSGALNNGANPLKTFIDTFPAGTLSGAPKVRAMQLISEIEPHNRGAYGGCIGFIGLNGELNQAITIRTFVSRNNELWFQAGGGIVARSQDEYELQEVNNKLGALKKAIDLAVKLKN

SEQ ID NO: 157 - protein, 454 aa, Enterococcus durans
  misc_feature: tr|A0A367CCZ8|A0A367CCZ8_9ENTE Anthranilate synthase component 1 OS=Enterococcus durans OX=53345 GN=trpE PE=3 SV=1
MRKTKKINGDCLTPINLYYRLRDEKSCLLESIPREKENGRYSVIAFEPAHHVKYAEGEFSFDETTYRCKDPLKELEKYVLVEEELHEELPFQGGAIGYVGYDIAACYEEIGTIPEDELGLPDMQFYLYESFVIYDHQQQEITVVVSNSYSHETEEVLAQRLETIEQKIRTMNEAEGSELPSLSLQFKSNFSQEKFEQIVAEAKQRIREGDLFQVVPSQRLSVDFTIDPFDYYRQLRVTSPSSYLYFLPFPQATIIGSSPESLVRVKGEIVTTNPIAGTRKRGASKELDDQLAKELISDPKEIAEHQMLVDLGRNDLGKVAQHGSVTVPLFMTVERYRFVMHIVSLVQARLKTGFSAMDALKATLPAGTVSGAPKIRAMTRIYEWEPVKRSIYAGAVGYFSQDNQADFAIAIRTMVVKGKKAYVQAGAGIVYDSDPTSEYFETLQKAKGLLEVRK

SEQ ID NO: 158 - protein, 518 aa, Bifidobacterium adolescentis
  misc_feature: tr|A1A1I7|A1A1I7_BIFAA Anthranilate synthase component 1 OS=Bifidobacterium adolescentis (strain ATCC 15703 / DSM 20083 / NCTC 11814 / E194a) OX=367928 GN=trpE PE=3 SV=1
MSECSVQTLKWGATWPSREQFHELADQGYRVIPIVRRLLADSLTPVGFYERLAGGRSGTFILESAEFGGAWSRYSFIGVHSIAQLRSNNGKADWLGQVPVGVPTDGDVVEVAHAALKTLKAPHVEGLPNLTSGLVGTVGWDSIRHWEPTLRAEAPNETGQPETVLALATDIAVVDHVSGSVWLIANAVNVDDRPTRADAAYDEALARLDDMQRKAAAPVENEARVNVLDESVGQPQLRFRTEKSDYERSVEEAKRHIIDGDVFQVVISQRLDIDSPADPFDVYRVLRTLNPSPYMYFMALTDAQGRDFNVIGSSPETLIKVDNGHAMSFPIAGSRPRGATPEEDERLAKELLADPKERSEHIMLVDLARNDLSRVCRPESVEVVSLMDIKRFSHIMHICSTVTGQVDPHMTAFDVFTSAFPAGTLSGAPKPRAIEIIDELEPADRGIYGGTVGYFDFSGNLDMAIAIRTAFIRDHEASVQAGAGIVLDSVPASEWQETRNKAEASVEAVQIAAQLRQL

SEQ ID NO: 159 - protein, 202 aa, Ruminococcus bromii
  misc_feature: tr|A0A2N0UU08|A0A2N0UU08_9FIRM N-(5′- phosphoribosyl)anthranilate isomerase OS=Ruminococcus bromii OX=40518 GN=trpF PE=3 SV=1
MKVKICGITSAEDIKIVNACEPDFAGFVMFFPKSKRNLSPETAKSLIEMLDKNVLSVAVTVSPTLEQVKTAYDCGFDYIQIHGEVGGDVLSNPYLKVIRAFNVSDLEKFEEYRMNPNIVGYVFDAHEPGSGKTFDWTMLENLPRDDKLFMLAGGLNPETVAKAVKAVKPDGVDVSSGVENSNGNGKDFEKVKKFIISARSEN

SEQ ID NO: 160 - protein, 201 aa, Acetoanaerobium sticklandii
  misc_feature: tr|E3PTA7|E3PTA7_ACESD N-(5′- phosphoribosyl)anthranilate isomerase OS=Acetoanaerobium sticklandii (strain ATCC 12662 / DSM 519 / JCM 1433 / NCIMB 10654) OX=499177 GN=trpF PE=3 SV=1
MSNLKIKICGLKHNEDIEYVNSVDIDYVGFVFVEKSSRFITAIKAKELKNKLSPTIKAVGVFVDKPIEYIVELANNNIIDMIQLHGNENEKYILDLKKITDKSIIKAFQVNSSEDLIKAEKSSADYILLDGGSGGSGICIDWSIIYELDRPFFLAGGLNADNVARAVSIVNPYAVDVSSGVEVDSKKDLDEIKKFVIAARR

SEQ ID NO: 161 - protein, 209 aa, Bacteroides xylanisolvens
  misc_feature: tr|D6D4J1|D6D4J1_9BACE N-(5′- phosphoribosyl)anthranilate isomerase OS=Bacteroides xylanisolvens XB1A OX=657309 GN=trpF PE=3 SV=1
MINGKIIKVCGMREAENIQDVEAIEGIDMLGFIFYPKSPRCVYELPAYLPTHARRVGVFVNEDKQVVSMYADRFGLNDVQLHGNESPEYCRSLHSTGLKIIKAFSVDRPKDLKKVYDYEKVCDLFLFDTKCEQYGGSGNQFDWSILHTYNRDVPFLLSGGINSYSANALKEFKHPRLAGYDLNSRFETKPGEKDPERIRTFLNELKSSL

SEQ ID NO: 162 - protein, 200 aa, Enterococcus durans
  misc_feature: tr|A0A367C9T6|A0A367C9T6_9ENTE N-(5′- phosphoribosyl)anthranilate isomerase OS=Enterococcus durans OX=53345 GN=trpF PE=3 SV=1
MTEIKICGLTTKTAIDAAIQSGASYLGFVFAESPRKITPENVRALTQDLPEKVKKVGVFVSPTKEFVEEIVKIAQLDFVQIHGEKKFQQLDVPIIRAKSVTKNVSLTVATGEDFLLLDAPPTKYMGGNGQRFDWSAVAVQQLEKEKLWLAGGLTAENVGEAIVYFQPKVVDVSSGVETDGKKDLAKISAFCEAVRIADNS

SEQ ID NO: 163 - protein, 241 aa, Bifidobacterium adolescentis
  misc_feature: sp|A1A2H2|HIS4_BIFAA 1-(5-phosphoribosyl)-5-[(5-phosphoribosylamino)methylideneamino] imidazole-4-carboxamide isomerase OS=Bifidobacterium adolescentis (strain ATCC 15703 / DSM 20083 / NCTC 11814 / E194a) OX=367928
MSLTLLPAVDVRDGKAVRLRQGESGSETDYGSPLEAARTWVESGAEWIHLVDLDAAFGTGNNRDQLRAIVKELGDKVNIEMSGGVRDDASLDAALEAGAARVNIGTAALENPDWTASVIKKYGDRVAVGLDVRGHTLAARGWVKEGGDLFETMKFLDSVGCSRYVVTDVARDGMMSGPNIELLREVASRTDAKVTASGGISKLDDLRNIKELAELGVDAAILGKSLYARAFTLEEALEVAR

SEQ ID NO: 164 - protein, 377 aa, Acidaminococcus intestini
  misc_feature: tr|G4Q917|G4Q917_ACIIR Acyl-CoA dehydrogenase OS=Acidaminococcus intestini (strain RyC-MR95) OX=568816 GN=bcd PE=3 SV=1
MDFALTEEQLELQAMVREFVEKEIVPYTDEMDKNNACRPEIIKKAGDMGLLNLVVPEEYGGPGLDSVTIAMIYEELGKGCVGVATSIAANALASYPILIAGNDEQKKYQCDLLNNGGLAAFALTEPDAGSDAGGVSTKAVKEGDHYVLNGSKVFITNGGIADSFLVFANTRKTGGIRGLTCFIVPKGTPGFSVGRKEDKMGIRPSNTCELILEDVVVPESMRVGREGQGFRIAMQTLDSARPFVASVAVGVAQSALDIAAHYARERRQFGQPISSFQLIQGMVADMAMKVHGARLMVQQACWMRDQGLEFGMEAAMSKCFASDVAMEVTTDAVQIMGGYGYMKDYPMEKKMRDAKILQIYEGTNQIQRLVIANKILY

SEQ ID NO: 165 - protein, 568 aa, Bacteroides uniformis
  misc_feature: tr|A0A139KHX6|A0A139KHX6_BACUN Acyl-CoA dehydrogenase OS=Bacteroides uniformis OX=820 GN=Bun01g_18050 PE=3 SV=1
MANFYTEVPELKYHLNNSMMERICELKERGYQDKDKYDYAPQDYADAMDSFDKVLEITGEITGEIIAPNAEGVDEEGPHCANGRVEYASGTKQNLDAMVKAGLNGMTMPRRFGGLNFPITPYTMCAEIVAAADAGFGNIWSLQDCIETLYEFGNEDQHSRFIPRVCAGETMSMDLTEPDAGSDLQSVMLKATYDEANNCWRLNGVKRFITNGDANLHLVLARSEEGTKDGRGLSMFIYDKNEGGVDVRRIENKLGIHGSPTCELVYKNAKAELCGDRKLGLIKYVMALMNGARLGIAAQSVGLSQAAYNEGLAYAKDRKQFGKAIIEFPAVYDMLAIMKAKLDAGRSLLYQTSRYVDIYKALDDIARERKLTPEERQEQKKYAKLADAFTPLAKGMNSEYANQNAYDSIQIHGGSGFMLEYACQRIYRDARITSIYEGTTQLQTVAAIRYVTNGSYSATLRDYEQVPCSEEMQPLMDRIKEMTNKFEACTNAVKEAQNQELLDFVARRLYEMAAVCIMSHLIIQDATKAPELFGKSALVYVNYAEAEVEKHFNFIRKFKAEELESYRK

SEQ ID NO: 166 - protein, 499 aa, Bifidobacterium longum
  misc_feature: tr|A0A564RYK6|A0A564RYK6_BIFLI Acyl-CoA reductase OS=Bifidobacterium longum subsp. infantis OX=1682 GN=luxC PE=3 SV=1
MPTADESVSQAIDVFHLPSGADVSDYEIYDVATSDGVKRLRYPRLDGPKVTSLAKQLADVRNRTLAAMSVNDILDIVADAAQLWADPDFELRRQAELLIPAITGYEPDMVRIELKRYMRQFRRRELLRFLDSEIGQPSMLDEFRPNKAGGYSKYVGPALTYQVFSSNVPGIPVWSMAMTLLVKGAILGKSSFSEPVMPAFFARSIAMVNSDLADAIAVVPWKGGSQQLEDSAIDVADAVIVYGSSQTTKLIAGKVAGSKPCLGYGAKVGLAFIGREALRPDTYADTVHRVAVDIATYDQQSCLAPQTVFVETDGALTAREVAQLLGGELENQQRKYPRSVLSDAENVAIQRARTDAEMRALMGGKAAVFASGHSTAWSVLYRELDGSGADEDVASLMSPLNRTVNVVAVPDLLDAARRLTSCRGWLQSCGVAVDSTRLFGLADILAEAGVNRICPLGEMDRAKSGWHHDGGFNLIDLLRAVDVERGSDAYGDSFDMDME

SEQ ID NO: 167 - protein, 451 aa, Clostridium acetobutylicum
  misc_feature: tr|Q97GS8|Q97GS8_CLOAB Acyl-CoA reductase OS=Clostridium acetobutylicum (strain ATCC 824 / DSM 792 / JCM 1419 / LMG 5710 / VKM B-1787) OX=272562 GN=CA_C2287 PE=3 SV=1
MKLEFTCGEEITIKEVNKLKLELMTSGKGDVEKELERLKKNKNKVHDIKTEETIDLLDRCAKLWLDDNYSKKHIETLAQITNQSFELVNYEFKSMMQMLLRENIQNTIKRELGEVDILDSWHKTNYGYEHRQPRGIIFHNISGNAFVVIPMSISMGLLSKNCNLVKVSKDEPYFAYAFYKSLCELDETVKDRLSVLYFDSSKTEIYDTIVKNSDGVIHWGGEYSSRIIGEMCAKYQKHMIMHGAKISFEVVDKCDDLNKVSEEIAKDIICWEQKACLSPRVVFLNDKIDVDEFSHCLSASLRKITEVIPKAYLSEWNSVKTIQDRQYCTLKYGMKNERRFKVYSSFNADYTVILSDGMPEKEDINKCFYRFIFVCPYSSKSDVVNYVSENLKDYLQTMGYSGNDEEFIEKMTLLGVSIVTKPGKMSLHEPGTSHDGIHNLKEFTFLVSRQI

SEQ ID NO: 168 - protein, 389 aa, Eubacterium rectale
  misc_feature: tr|A0A0M6WEG7|A0A0M6WEG7_9FIRM Acyl-CoA dehydrogenase OS=[Eubacterium] rectale OX=39491 GN=DW001_08475 PE=3 SV=1
MDFTLDQKHEMARDLFKQFAETEVKPLAQETDETEVFPAETVAKMGKYGFMGIPVPKEYGGQGCDPLTYVMCVEELSKVCGTTGVIVSAHTSLCIDPIMTFGTEEQKKKYVPDLASGKKIGAFGLTEPGAGTDAQGCQTKAVLDGDEWVLNGSKCFITNGKVADVYIIIAITSITEDKRGRKKKNFSAFIVDKGTPGFSFGTKEKKMGIRGSSTYELIFEDARIPKDALLGKEGRGFPIAMHTLDGGRIGIAAQALGIAEGALERTIEYTKERKQFGRSIAQQQNTQFKLADMATRIDAAKYLVYAAAMKKAEFAKNPKVSYSVDAAKAKLFAAETAMAVTTECVQLFGGYGYIREYDVERMMRDAKITEIYEGTSEVQRMVISGSLLR

SEQ ID NO: 169 - protein, 381 aa, Gordonibacter pamelaeae
  misc_feature: tr|D6EA99|D6EA99_9ACTN Acyl-CoA dehydrogenases OS=Gordonibacter pamelaeae 7-10-1-b OX=657308 GN=GPA_25140 PE=3 SV=1
MDFKKTEEQELLLDSLKTVMERGNFEDYFKECDRNHEYPQKAVDALVEAGFTTLGIPEEFGGTPTDTLTQVMVAEEAHALGYPSLCWINFATEVDDILTFGNKEQQEKILGYALEGKKPFTLGFTEPQAGSDSAAMATTATKRDGKVYINGNKTFNTSADRAPYMLCVCRSGVNESPYKDFSMYLFPMDRPGVAIEKLDKIGNNMCGTYEVHLDDVECEESDLVGEECKGFYQLMKNFEVERLTICAANVGMARCAYDEALRYAAQRMQFGKIIGSFQLVQEKLVDMRIKIENMQNLLYKTAWKKDNGESIMIDSSLVKRYTGQAAFEVIDDAMQIMGGIGYTHDCRISRLWRDQRVYRIMAGTEEIMVHTAGRALIKEAQ

SEQ ID NO: 170 - protein, 408 aa, Lactobacillus plantarum
  misc_feature: tr|A0A1E3KW51|A0A1E3KW51_LACPN Acyl-CoA dehydrogenase OS=Lactobacillus plantarum OX=1590 GN=GPJ80_14625 PE=3 SV=1
MEATETAPYEPTKKLVHTKPGTKMTDEEFALYLKQIKHLAENEFDYLQKEVEVTNRFPKKFFELAIDNDLYRFALPLKYGGFGLSCKQIFQVQEMFSRGPGGMRMHLHYAADLNWRIMDEFGRPELKAEVMPKLQDKSIFTCFALTEESGGTGADLHTTAVKDGDDYILNGEKTLISHTDCSDAAYVIAVTDPDAEGSKRLSAFYVPVDTPGYEIVPMPHMMGCRGAGHAGLKFTNMRVNKKYLLGEEGDGLHVAIASLSISRAHIAASNLGMSQRMLEMAIARAKDRVTFGKPLAKRQAIQQEIADMGTEIFALRTMLHNLADQYDRGEDIEEKAAMCKLFSINTVKLVSDYCLEIFGGIGYFEDNPYGPVERMYRDCRAMWLEEGPRSVQRLTAARKLIANDGIIE

SEQ ID NO: 171 - protein, 376 aa, Ruminococcus gnavus
  misc_feature: tr|A0A415S9V5|A0A415S9V5_RUMGN Acyl-CoA dehydrogenase OS=Ruminococcus gnavus OX=33038 GN=DWZ50_08895 PE=3 SV=1
MNMNVMDDKLFYNEIKEFARNYLDKQNSNEKHLWERLGEFGILGLSIDEKYGGLGESYRMCAEILEILGYTCWNNGLIFSINNHIWMAQNLINLYGSTWLKNKYVGNMVAGEKIGAFALTEPDAGSDPYSMKTCAIRAGDYYIINGRKTFVSNGPIADVFIVFAITSQTEMKKITAFVVDKTMPGVTIGKNIEKMGLECSPTSDICFENCEVPADNILGKVDLGNNILDAALEWERCFEFIPHIGSMKRIMEYCIKYSNERKQFGKRINENQSISHKIANMSMKIELAKNLMYRIVDLKEQGKSSYYESSIFKVFVSESYVETCRDAIQILGAYGYTKEYPVEKELRDAIASTIYSGTNEMQRNNIFNIESIRNMV

SEQ ID NO: 172 - protein, 544 aa, Enterocloster lavalensis
  misc_feature: Tryptophanase (indole production) (EC 4.1.99.1) Enterocloster lavalensis HB-452c
MKNYPLNVEAPKSYSYVKRNIPNVTVEQRERALKSTHYNEFAFPAGMLTVDMLSDSGTTAMTDVQWSAMFLGDESYGRNKGYYVLMDAFRDVFERGDDQKRVINLVRTDCQDIDKMMDEMYLCEYEGGLFNGGAAQMERPNTFIMPQGRAAESVLFAMVSKILGERHPGKNFTIPSNGHFDTTEGNIKQMGSTPRNCFDKQLLWEVPEGGVYEKNPFKGNMDTAKLEALIQEAGPENVPLVYTTITNNTVCGQPVSMANIRESSRIAHKYGIPFMLDAARWAENCYFIKVNEEGYGDKSIFAIAKELFSYCDGFTASLKKDGHANMGGILAFRDKGLFWKNFSDFDAEGNVVTDVGILLKVKQISSYGNDSYGGMSGRDIMALAAGLYECGRVEYLKERVEQCEYLAQGFYKNGVKGVVLPAGGHGVYINMDEFFDGKRGHQSFAGAGFSLELIRRYGIRVSELGDFSMEYDLKTPEQQKEVCNVVRFAINRSQLSREHLDYVIAAVTELYKDRKSIPNMKITLGHKLPMRHFHAFLEPYEAEE

SEQ ID NO: 173 - protein, 412 aa, Clostridium sporogenes
  misc_feature: Cinnamoyl-CoAphenyllactate CoA-transferase fldA (EC 2.8.3.17) Clostridium sporogenes
MENNTNMFSGVKVIELANFIAAPAAGRFFADGGAEVIKIESPAGDPLRYTAPSEGRPLSQEENTTYDLENANKKAIVLNLKSEKGKKILHEMLAEADILLTNWRTKALVKQGLDYETLKEKYPKLVFAQITGYGEKGPDKDLPGFDYTAFFARGGVSGTLYEKGTVPPNVVPGLGDHQAGMFLAAGMAGALYKAKTTGQGDKVTVSLMHSAMYGLGIMIQAAQYKDHGLVYPINRNETPNPFIVSYKSKDDYFVQVCMPPYDVFYDRFMTALGREDLVGDERYNKIENLKDGRAKEVYSIIEQQMVTKTKDEWDNIFRDADIPFAIAQTWEDLLEDEQAWANDYLYKMKYPTGNERALVRLPVFFKEAGLPEYNQSPQIAENTVEVLKEMGYTEQEIEELEKDKDIMVRKEK

SEQ ID NO: 174 - protein, 407 aa, Clostridium sporogenes
  misc_feature: (R)-3-(aryl)lactoyl-CoA dehydratase, alpha subunit fldB (EC 4.2.1.175) Clostridium sporogenes
MSDRNKEVKEKKAKHYLREITAKHYKEALEAKERGEKVGWCASNFPQEIATTLGVKVVYPENHAAAVAARGNGQNMCEHAEAMGFSNDVCGYARVNLAVMDIGHSEDQPIPMPDFVLCCNNICNQMIKWYEHIAKTLDIPMILIDIPYNTENTVSQDRIKYIRAQFDDAIKQLEEITGKKWDENKFEEVMKISQESAKQWLRAASYAKYKPSPFSGFDLFNHMAVAVCARGTQEAADAFKMLADEYEENVKTGKSTYRGEEKQRILFEGIACWPYLRHKLTKLSEYGMNVTATVYAEAFGVIYENMDELMAAYNKVPNSISFENALKMRLNAVTSTNTEGAVIHINRSCKLWSGFLYELARRLEKETGIPVVSFDGDQADPRNFSEAQYDTRIQGLNEVMVAKKEAE

SEQ ID NO: 175 - protein, 373 aa, Clostridium sporogenes
  misc_feature: (R)-3-(aryl)lactoyl-CoA dehydratase, beta subunit fldC (EC 4.2.1.175) Clostridium sporogenes
SNSDKFFNDFKDIVENPKKYIMKHMEQTGQKAIGCMPLYTPEELVLAAGMFPVGVWGSNTELSKAKTYFPAFICSILQTTLENALNGEYDMLSGMMITNYCDSLKCMGQNFKLTVENIEFIPVTVPQNRKMEAGKEFLKSQYKMNIEQLEKISGNKITDESLEKAIEIYDEHRKVMNDFSMLASKYPGIITPTKRNYVMKSAYYMDKKEHTEKVRQLMDEIKAIEPKPFEGKRVITTGIIADSEDLLKILEENNIAIVGDDIAHESRQYRTLTPEANTPMDRLAEQFANRECSTLYDPEKKRGQYIVEMAKERKADGIIFFMTKFCDPEEYDYPQMKKDFEEAGIPHVLIETDMQMKNYEQARTAIQAFSETL

SEQ ID NO: 176 - protein, 264 aa, Clostridium sporogenes
  misc_feature: (R)-phenyllactate dehydratase activating ATPase fldI, archerase (EC 5.6.1.9) Clostridium sporogenes
MADIYTMGVDIGSTASKTVVLKNGKEIVSQAVISVGAGTSGPKRAIDSVLKDAKLSIEDLDYIVSTGYGRNSFDFANKQISELSCHAKGVYFDNNKARTVIDIGGQDIKVLKLADSGRLLNFIMNDKCAAGTGRFLDVMSRVIEVPVDELGKKALESKNPCTISSTCTVFAESEVISQLARGVKTEDLIAGICKSVASRVASLAKRSGIEELVVMSGGVAKNIGVVKAMEAELGRDIYISKNSQLNGALGASLYAYESFQKERS

SEQ ID NO: 177 - protein, 412 aa, Peptostreptococcus russellii
  misc_feature: putative Cinnamoyl-CoAphenyllactate CoA-transferase fldA (EC 2.8.3.17) Peptostreptococcus russellii
MSTRKQPLKGIKVIELANFIAAATTGRFLADLGADVIKIESAKGDPLRYTAPTEGRPLDMYENTTWELENANKRCISLNMKDPAGKEAFFKLLDDADVLITNWRVQALERAGLDYETLKVKYPSLVYALCTGYGEFGPDKDLPGFDFTAFFARGGYLENLRQKSDVPMNVVPGLGDHNVGINLAAGVLAALYHAKLTGEGEKVETSLFESAIFNMGMMIQAAQYPDHGVKYPINIREASNPFNACWRTSEGRYIQTCMPDYNTYFKKFLTALGLTDMVEDERFFPVQNLHENGLGTTVYDRVMSTFEQKSHEEWAQILTEADIPFALAKTWEELLEDDQAWANDCFHKMQYPNGERVLVKHPIKFAEMGPTPYNKGPMIGEHGVEVMKELGYNEDQINEMIENKTLYIWEDK

SEQ ID NO: 178 - protein, 412 aa, Peptostreptococcus russellii
  misc_feature: putative (R)-3-(aryl)lactoyl-CoA dehydratase, alpha subunit fldB (EC 4.2.1.175) Peptostreptococcus russellii
MSNTGMVEEKPAKKMLGEIVAKHYQEAWDAKERGEKVGWCASNFPQEIFETLDIKVVYPENQAAAISAKGGGQRLCEVAENNGYSNDICAYARISLAYMDVKDAPELNMPQPDFVACCNNICNCMIKWYENIAKELNIPLILIDVPYNNEYEAGDDRVEYLKGQFDYAIKQLEDLTGKKWDEDKFNEVMEVSQRTGRAWLKATGYAKYTPSPFSGFDVFNHMAVAVCARGKLESALAFEKLAEEFDQNVKEGKSTFKADEKYRIVFEGIACWPHLRHTFKQLKDNGVNVCGTVYADAFGYIYNNTYELMQAYCGTPNAISYERATDMRLKLLEENNIDGMLFHINRSCKQWSAIMYEMEREIREKTGIPTATFDGDQADPRNFSEAQYDTRVQGLIEVMEASKAAKEAQRHE

SEQ ID NO: 179 - protein, 372 aa, Peptostreptococcus russellii
  misc_feature: putative (R)-3-(aryl)lactoyl-CoA dehydratase, beta subunit fldC (EC 4.2.1.175) Peptostreptococcus russellii
MSNINLLLEKLDVCPLEQLDKYTNDGKKVIGCAPVYAPEELVYAAGMIPMGIWGAEGEVTSAKEYFPAFYAAPILRLMDLGLEGKLDKMSGMIIGGLSDGLKGLSQNWKRAVKNVPCLYIGYGQNRKIKAGIDYNEKQYIKLKKELEEIAGCKIEDSKVEEAIVLYNNHRKAMSEFNQLASTHLNTITPTLRARVMSSAYFFDKAEHLAILEEINNILKSTPEEKFEGKKIVTTGILANSPDIMKILEEFKLGIVDDNVNHESGQFDYLVDEATDNPVRALAKWISDIEGSALLYDPSKLRGQIVIDKVKKHNADGVLYLMTKFSESDEFDYPIIRDELNEAGIMNILVEVDQQMTNFEQAKTALQTFADMI

SEQ ID NO: 180 - protein, 262 aa, Peptostreptococcus russellii
  misc_feature: putative (R)-phenyllactate dehydratase activating ATPase fldI, archerase (EC 5.6.1.9) Peptostreptococcus russellii
MSEIYTMGIDIGSTSSKCVILKNGKDIVSKGVVNLGAGTKGVDRVIDEVIKKANLTLKDIEVIVSTGYGRNSYDDAKKTMSELSCHAKGGSYIFGDVRTIIDIGGQDIKVLKLNNKGQLMNFLMNDKCAAGTGRFLEVMCGVLDVELGDLGKLDEQATEKTPISSTCTVFAESEVISCMAKKIPIPNIVKGIHYSVASRVAGLAKRGGLTQPVAMTGGVTKNSGIVRALSGELETEIMISEDSQMAGAIGAALYAYEEYLKS

SEQ ID NO: 181 - protein, 321 aa, Mycolicibacterium smegmatis
  misc_feature: Putative tryptophan N-methyltransferase Mycolicibacterium smegmatis
MTLSLANYLAADSAAEALRRDVRAGLTAAPKSLPPKWFYDAVGSDLFDQITRLPEYYPTRTEAQILRTRSAEIIAAAGADTLVELGSGTSEKTRMLLDAMRDAELLRRFIPFDVDAGVLRSAGAAIGAEYPGIEIDAVCGDFEEHLGKIPHVGRRLVVFLGSTIGNLTPAPRAEFLSTLADTLQPGDSLLLGTDLVKDTGRLVRAYDDAAGVTAAFNRNVLAVVNRELSADFDLDAFEHVAKWNSDEERIEMWLRARTAQHVRVAALDLEVDFAAGEEMLTEVSCKFRPENVVAELAEAGLRQTHWWTDPAGDFGLSLAVR

SEQ ID NO: 182 - protein, 247 aa, Akkermansia muciniphila
  misc_feature: Putative amino acid N-methyltransferase Akkermansia muciniphila
MDWNADLYENKHDFVAEYGRDLLSHVPENPGQAILDLGCGTGTLTHALLEKSTSVVGLDASPEMIGKARQLYPGMDFRVLDACLMPWNNWFDVIFSNAAFHWIPDHGALLKAISRALKPQGKLICEFGAHRNILRIREAFRPSLERMNLPYKTRFYFPTVEEYRSMLEQAGLRPEVVMDFDRPTPLKDGPDGLRNWARQFFWADLKCLHEKRRVRIFEEMEKALRDELWDGTQWVADYRRIRVIASK

SEQ ID NO: 183 - protein, 247 aa, Adlercreutzia equolifaciens
  misc_feature: Putative amino acid N-methyltransferase Adlercreutzia equolifaciens
MTIDTATGLEAPAELSSERVRDIFSHIARKYERFNALSSFGAYKLWLAGMMRQAPIDESHDVLDIAGGTGDVTFTVARTKHPSHIQCTDLVPEMLDVARMHIDEGDGAGVPIDFEVVDAQDIPYADASYDVITMAYGIRNMPDRPRALSEMFRVLKPGGSLVCLEFSTPPNGAWRALYHFYLRHLIPFWGGLVTGDKDGFVYLAKSIRAFPDQEGLAEMMREAGFTDVTWKNYTGGIAAVHVAKKPE

SEQ ID NO: 184 - protein, 254 aa, Enterorhabdus sp.
  misc_feature: Putative amino acid N-methyltransferase Enterorhabdus sp. NM05_H27
MSIDTATGLEAPPELSSERVRDIFSAIARKYERFNAISSFGAYKLWLAGMMRQAPIAEHHDVLDIAGGTGDVTFTVARTKHPAHIQCTDLVPEMLDVARMHVDEGDGAGVPIDFQVVDAQDIPYADAAYDVITMAYGIRNMPDRPRALAEMFRVLKPGGALVCLEFSTPPNRIWRALYHFYLRHLIPFWGGLITGDKEGFVYLAKSIRAFPDQEGLAALMGEAGFTDVTWKNYTGGIAAVHVARKPASGASGRS

SEQ ID NO: 185 - protein, 417 aa, Clostridium sporogenes
  misc_feature: Tryptophan_decarboxylase| C._sporogenes_ATCC_15579_(POSITIVE_CONTROL)
MKFWRKYTQQEMDEKITESLEKTLNYDNTKTIGIPGTKLDDTVFYDDHSFVKHSPYLRTFIQNPNHIGCHTYDKADILFGGTFDIERELIQLLAIDVLNGNDEEFDGYVTQGGTEANIQAMWVYRNYFKKERKAKHEEIAIITSADTHYSAYKGSDLLNIDIIKVPVDFYSRKIQENTLDSIVKEAKEIGKKYFIVISNMGTTMFGSVDDPDLYANIFDKYNLEYKIHVDGAFGGFIYPIDNKECKTDFSNKNVSSITLDGHKMLQAPYGTGIFVSRKNLIHNTLTKEATYIENLDVTLSGSRSGSNAVAIWMVLASYGPYGWMEKINKLRNRTKWLCKQLNDMRIKYYKEDSMNIVTIEEQYVNKEIAEKYFLVPEVHNPTNNWYKIVVMEHVELDILNSLVYDLRKFNKEHLKAM

SEQ ID NO: 186 - protein, 490 aa, Ruminococcus gnavus; identical to SEQ ID NO: 117
  misc_feature: Tryptophan_decarboxylase| Ruminococcus_gnavus_(strain_ATCC_29149_/_VPI_C79)_(POSITIVE_ CONTROL)

SEQ ID NO: 187 - protein, 491 aa, Enterococcus durans
  misc_feature: UbiD_family_decarboxylase| Enterococcus_durans_HB48
MTEQPYDLRKVIEELKTLPGQYHETDLEIDPEADLSGVYRYIGAGGTVKRPTQEGPAMTFNNIKGFPGKRVNIGTMASRKRVGQILHHDYRALGRFLKDAVEHPLKPVKVDKADAPAQEVVHLATDADFDIRNILPAPKNTPEDAGPYITMGVVLGSDPEKTMSDVTIHRMVLEDKDTIGMYIMPGGRHIGHFQKQFEALNQPMPITINIGLDPAIAIGTTFEPPTTPLGYNELWCAGALRNEPIQLVDGVAVNEVGIARAEFIIEAEIMPNQTMQEDINTNSGKAMPEFPGYTGDANPAVNVVKVKAITHRKDNPIMQTTIGPSEEHVSMAGIPTEASILALVDKAIPGKVVNVYNPPAGGGKLMTIMQIRKENEADEGIQRQAAILALSAFKELKTVILVDEDVDIFDMNDVMWTFNTRFQGDKDIIVIPGMRNHPLDPSELPQYNPGVIRFRGMSAKTIFDGTVPFDMKEHFIRAQFKEVDNWKDYLN

SEQ ID NO: 188 - protein, 191 aa, Enterococcus duran
  misc_feature: Lysine_decarboxylase_family| Enterococcus_durans_HB48
MKLTVFCGSRFGNKESYKFIAQMLGKYMAQENIELVYGGSDSGIMGIFSQTVLENNGKVTGIYPTGLFELETPKEEVTTFIPTDSIDERKVLLFEKGDAVLIFPGGLGTLEEFSQLLSWIAIGLTPDKPIGILDIGGYYSGLQTLLETFAKEGFMDAKWLNRIFFSNNPLKLVDLLREETTGLSTLLKEAK

SEQ ID NO: 189 - protein, 477 aa, Clostridium asparagiforme; identical to SEQ ID NO: 118
  misc_feature: Pyridoxaldependent_decarboxylase| Clostridium_asparagiforme_HB452c

SEQ ID NO: 190 - protein, 421 aa, Clostridium asparagiforme
  misc_feature: Diaminopimelate_decarboxylase_(EC_4.1.1.20)| Clostridium_asparagiforme_HB452c
MDKKPFVTLEKLEEIVKDYPTPFHLYDEKGIRENAMKLKQAFAWNKGFKEYFAVKATPNPFILQLLKECGCGTDCSSMTELMLSEACGFSGEEIMFSSNDTPPEEFAYAARLGAIINLDDFTHIQFLEDTLGYIPETISCRFNPGGVFKISNDIMDNPGDSKYGMTTEQMFEAFKILKAKGAKRFGIHAFLASNTVTNEYYPLLARQLFELAVKLKEETGAHIAFINLSGGIGIPYRPDQEPNDILVIGEGVRNAYQDVLVPAGMGDVALCTELGRFMLGPFGCLVTRAIHEKHTYKEYVGVDACAVNLMRPAMYGAYHHVTVMGKENLPCDHMYDVVGSLCENNDKFAIDRMLPKVDKGDLLVIHDTGAHGFAMGYNYNGKLKSAELLLKEDGSVQMIRRAETPKDYFVTFDFCDILKND

SEQ ID NO: 191 - protein, 389 aa, Clostridium asparagiforme
  misc_feature: NADP-dependent_malic_enzyme_(EC_1.1.1.40)| Clostridium_asparagiforme_HB452c
MDVNKASLDMHYELAGKIEVVSRKKIETREDLSLAYTPGVAEPCRVIAKDYEQSFKLTRRSNLVAVITDGTAVLGLGDIGPAAGMPVMEGKCVLFKEFADVDAFPLCIDSKDTDTIVQTIHLISKSFGGINLEDIAAPRCFEIERRLKEICDIPVFHDDQHGTAIVVAAALINAIKVTGKEMGKIKIVINGAGAAGIAIGKLLISMGFGNVVMCDINGIICEGDEGLNAGQEEISHISNLNKEHGKLADALKGADAFVGVSRPNLVTKEMVASMNNGIVFAMANPTPEIMPDEARAGGAAVIGTGRSDFPNQINNVLVFPGIFKGALSVRAKEITETMKQRAAYAIASMIPDEELNAENIIPSPLNKGVAAVVAKAVADTAVEEGIARI

SEQ ID NO: 192 - protein, 386 aa, Clostridium asparagiforme
  misc_feature: Oxaloacetate_decarboxylase_beta_chain_ (EC_4.1.1.3)|Clostridium_asparagiforme_HB452c
MEFLLDGIMAVLGPGGWKMIAMYIVGIALIVLAVKKEYEPSLLLPLGFGAILVNLPYSGVIDQLVQNKVEAAGIIQWLFEVGIEASEAMPILLFIGIGAMIDFGPLLSQPVLFLFGAAAQFGIFAAILIACLMGFDLKDAASIGIIGAADGPTSILVSQVLGSNYMGPIAVAAYSYMALVPIIQPFAIKLVTTKKERCIHMQYNPGDVSRGMRIAFPIAVTIIVGFISPQSVALVGFLMFGNLLRECGVLHSLSQAAQNELANIITLLLGITISFSMRAEQFVNPATLMIMALGLVAFVFDTIGGVLFAKVVNVFLKMAGKPPVNPMIGGCGISAFPMSSRVVQKMAAEAEPGNIILMQAAGTNVSGQVASVIAGGLVISIVTQYL

SEQ ID NO: 193 - protein, 363 aa, Clostridium asparagiforme
  misc_feature: L-threonine_3Ophosphate_decarboxylase_ (EC_4.1.1.81)|Clostridium_asparagiforme_HB452c
MEYQHGGDIYSQEVDLDFSANLNPFGLPEAVRRAAADSLGNCTVYPDSSSRKLTAALAAYHGVPQEQVICGNGAADLIFGLALALKPERALVTAPAFSEYEQALASVDCRVSWLDLREKDGFRLTPRELLEKLSGEPQLLFLCNPNNPTGVSIPREDMEWVAGVCRKQGVRLVVDECFCDFLDAPGACSLIPLLEKYPNVFVLKAFTKLYAMAGLRLGYGLCADGELMERLKQVRQPWSVSSVAQAAGLAALGETGYVRASTEAIARERRWLTGELTRLGLTVYGSQANYIFFRDPAGADGSARGRLYEALLERRILIRSCANYRGLDHTYYRVCVRLHEDNEILIRELKRILTGRDHLERQE

SEQ ID NO: 194 - protein, 389 aa, Clostridium asparagiforme
  misc_feature: NADH-dependent_butanol_dehydrogenase_A_ (EC_1.1.1.)|Clostridium_asparagiforme_HB452c
MLGNFSYCNPTKLYFGEDSLSNLREELPKYGKNVMLVYGGGSIKRSGIYDQVVKLLDEGGKNIFEDPGVMPNPTVEKLYEGCRIARENQVDLILAVGGGSVCDYAKAVSVSAHCEEDPWEKYYLRMEDVDNRIIPVGCVLTMVGTGSEMNGGAVITNHKTKLKIGHVFGENVFPKFSILNPAFTFTLPRYQMIAGFYDIMSHILEQYFSGEDDNTSDYIMEGLLKSLIHSARIAAEHPTDYEARSNIMWIATWALNTLVAKGKTTDWMVHMIGQSIGAYTDATHGMTLSAVSMAYYRFIMPYGLAKFRRFAVNVWDVNPEGQSDEAVAEEGLVRMERYMRELGLVMDIRELGVTEEMLDGIAAGTFAMDGGYRAPDHDEIVEILKASMA

SEQ ID NO: 195 - protein, 379 aa, Clostridium asparagiforme
  misc_feature: 4-carboxymuconolactone_decarboxylase_ (EC_4.1.1.44)|Clostridium_asparagiforme_HB452c
MEKREMSVFPTGKPNDAYARYFVGQSYLNMLSTDQVTMGNVTFEPGCRNNWHIHHARKGGGQMLLVTAGNGYYQEWGKEPVALYPGAVVHIPPEVKHWHGAAPDSWFQHLAIEVPGEGCTNEWCEAVSDEEYAACESNVPAFARHCGLSDTDPEFTERFNHFALREVADSGDLDGRTRMLAILAALAGSQGTDEFGALLPAALETGVRPVEVKELLYQAVAYLGIGRVLPFLRVVNEVFARRGIKLPLEGQLGTTLENRLEKGSQAQVDIFGERMRDFWKSGPEESRHINRWLAENCFGDYYTRNGLDYRQREMITFCFLAAQGGCEPQLASHIAANIRLGNDKAFLIQIISQWVPFIGYPRCLNALRCVDEAVDRTEV

SEQ ID NO: 196 - protein, 390 aa, Clostridium asparagiforme
  misc_feature: NADP-dependent_malic_enzyme_(EC_1.1.1.40)| Clostridium_asparagiforme_HB452c
MDYYEEALKIHEKAGGKLEIVSKVPVKTRDDMSLAYTPGVARPCMEISNDGRAAYRYTAKRNLVAVVSDGTAVLGLGDIGGQASMPVMEGKAILFKNFGGVDAFPICLDTKDTEEIIQTVKNIAPTFGGINLEDICSPKCFEIEKRLEEELDIPVFHDDQHGTAIVVTAALYNALKVVDKKIDQVRVVLSGPGAAGTAIVKMLFAAGVRHMIVCDENGILYRGKEGLKGHKVMLSETTNEEMRKGWLADAVVGADVFVGVSKPGVLTKEMVQTMGRDPIVFAMANPVPEIMYDEAVEAGVKVMGTGRSDFPNQINNVLAFPGIFRGALDAGARDINYEMKVAAASAIAELVEPEKLCPEYVIPSVFDERVAPHVAKRVAEAAVRSGAVRE

SEQ ID NO: 197 - protein, 483 aa, Clostridium asparagiforme
  misc_feature: Acetylcoenzyme_A_carboxyl_transferase_alpha_ chain_(EC_6.4.1.2)|Clostridium_asparagiforme_HB452c
MSNSAQASASSRIAALLDENSFVEVGAYITARTTDFNMTEQETPADGVVTGYGTIEGCLVYVYSQDASVLGGSMGEMHAKKISNIYSMAMKMGAPVIGLIDCAGLRLQEATDALAGFGQVYLNQTMASGVIPQISAIFGTCGGGMAVSAAITDFTFMEDKSAKLFVNAPNALKGNYTSKCDTSSAAFQSAEAGLVDFTGDEASILGQIRRLVSILPSNNEDDMSYGECQDDLNRISGELAGCAGDTALALSIISDNNFFMEVKKNYDPSMVTGFIRLNGTTVGCVANRSEVYENGEKTAEYEPVLSAKGCDKAADFINFCDSFNIPVLSLVNVKGYKASKCTEKLIAKAAGRLTYAYANASVPKVTVIVKDALGSAYLTMGSKSIGADVVYAWENAAIGMMDPAEAVKIMYAKEIEAAEDSVALISEKTAQYAALQSSALAAARRGYIDDIIKAGETRQRVIAAFEMLFTKREDRPSKKHGTV

SEQ ID NO: 198 - protein, 381 aa, Clostridium asparagiforme
  misc_feature: Oxaloacetate_decarboxylase_beta_chain_ (EC_4.1.1.3)_MethylmalonylCoA_decarboxylase_beta_chain_ (EC_4.1.1.41)|Clostridium_asparagiforme_HB452c
MDFGTIVNNLLDQMAFFHLQPGNYAMIGVALVFLFLAIKKGFEPLLLIPISFGMLLVNIYPEIMYSPEQTSNGTGGLLWYFFQLDEWSILPSLIFLGVGAMTDFGPLIANPISFLMGAAAQVGIYSAYFFAILLGFNGKEAAAISIIGGADGPTSLFLCSKLGQSQIMGPIAVAAYSYMALVPIIQPPVMKLLTTEKERKIKMEQLRPVSKLEKILFPIIVTIVVCLILPSTAPLVGMLMLGNLFRECGVVKQLAETASNALMYIVVILLGTSVGATTSAEAFLNWTTLKIVGLGLVAFIFGTAGGVLLGKLLCKITGGKINPLIGSAGVSAVPMAARVSQKVGAEADPTNFLLMHAMGPNVAGVIGTAVAAGTFMAIFGV

SEQ ID NO: 199 - protein, 470 aa, Clostridium asparagiforme
MAEIEKKPVKIVETVLRDAHQSLLATRMSTDQMLPIVDKMDQVGYYAVECWGGATFDSCLRFLKEDPWERLRKLRAGFKNTKLQMLFRGQNILGYNHYADDVVEYFVQKSIANGIDIIRIFDCLNDIRNLRTAVKATNKEKGHAQIALCYTLGDAYTLDYWKNIAREIEDMGADSLCIKDMAGLLTPYAAAELVTALKEGTSLPIDLHTHYTSGVASMTYLKAVEAGCDIIDCAMSPLALGTSQPATEVMVETFRGTPYDTGYDQTLLAEIADHFQPIRDQALKSGLLNPKVLGVNIKTLQYQVPGGMLSNLVNQLKEAGQEDKYRQVLEEIPRVRKDFGEPPLVTPSSQIVGTQAVLNVIMGERYKMVPKESKKIMLGEFGQTVKPFNPEVQKKIIGDETPITCRPADLIEPQLPQFEKDCAQWKQQDEDVLSYALFPQVAKDFFIYREAQQTKVDQTVADKNSKAYPV

SEQ ID NO: 200 - protein, 116 aa, Clostridium asparagiforme
  misc_feature: 4-carboxymuconolactone_decarboxylase_ (EC_4.1.1.44)|Clostridium_asparagiforme_HB452c
MKDVRTMLNDFVGGLGKLGETNEAHVQAFMNLLGTNYAEGALSVKTKELISVAIGAYNRCEYCIVYHVYKAYEAGATREEIIEAAMIAVAFGGGPSMAYSVTTLMDAVNEFEHDFD

SEQ ID NO: 201 - protein, 295 aa, Clostridium asparagiforme
  misc_feature: Phosphatidylserine_decarboxylase_(EC_4.1.1.65)| Clostridium_asparagiforme_HB452c
MKHNASTNAGPRDGFQDKLLQLIYNHALGRALILPFTHPVISKIGGYCMDSSLSRILIPSFIRTAHIDLSQCELPAGGRYRSFNDLFTRRLLPQARPFDPDPLTLASPCDARLTAYSISPRRVFHIKQTAYRLEDLLRDPKLARRFAGGTALVFRLCVDDYHRYAWPDGGLRAHYRKIPGRLHTVNPVANDHFPIYKENCREYTVVRSQHFGTYLMMEVGALMVGKIVNHHTGYTRLDVMRGQEKGYFAFGGSTIVLLFMPGAVELDEPILQASARGEETRVRMGQRIGRAARRQ

SEQ ID NO: 202 - protein, 395 aa, Clostridium asparagiforme
  misc_feature: NADH-dependent_butanol_dehydrogenase_A_ (EC_1.1.1.)|Clostridium_asparagiforme_HB452c_Assembly
MNNFEFYTPTRMIFGRGTHLQVGKIVKEYGFKKVLVHFGGASAKKTGLLDAVCGALEAEGIGYVQLGGVQANPTLSMAKKGIELCLAEKADFVLAVGGGSVIDSAKCIADGAGNPGVDVWKFFTKEAAPAGALPVGTILTLSASGSEMSASCVITNEENGLKRGFNSTTHRPLFSICNPELTYTVNRFQTGCGTVDIMMHTLERYMGGTTKETPLTDRIAEGLLKTVAEAGAVADRDPENYEARATLMWAGSLSHNDLTSAGRAFMMQVHQMEHELSGMYPQIAHGAGLSALWPSWARYVCAAWPERFAQYAVRVWNLEMNFENPMETALAGIKVTEDYFKSLNMPTNIRDLGVEPEKIDEMAEKCTNFGTRTLPGIRELGKAEMMEIYRMAMEA

SEQ ID NO: 203 - protein, 504 aa, Clostridium asparagiforme
  misc_feature: UbiD_family_decarboxylase_Lactobacillus_brevis_ type|Clostridium_asparagiforme_HB452c_Assembly
MNQINDLRSALELLRNLPGQLAETDVPVDPMAELAGVYRYVGAGGTVMRPTKEGPAMIFNNIKGHPGARIAIGLLASRDRVAALFGCKPQQLSRTLLEAVEHPIPPVLTAKVPPCQQVVHLASDPDFDLRTLIPAPTNTPVDAGPYLTLGMCYATHPDTGESDVTIHRMCILGRDELSIFFTPGIRHIGAMALRAEELGRPLPISISIGVDPVIEISSCFEPPTTPLGFDELSIAGALRGEPVQMCRCLTVEEKAIANAEYVLEGEIIPGVRVTEDCNTHTGYAMPEFPGYTGPSSSECCLIKVKAVTHRENPIMQTCIGPSEEHVSMTGICTEASILAMAEKAMPGKLTNICCPSSGGGKYMAVLQFKKSVPSDEGRQRQAALLAFAAFSELKHVFLVDEDVDCFDMKDVFWAMSTRFQADQDVIAIPGVRCHPLDPSNDPGFSPSIRDHGIACKAIFDCTVPYDQKARFKRAEFLEVDPSRWLEKTAGSPQWQEVKACAQND

SEQ ID NO: 204 - protein, 526 aa, Clostridium asparagiforme
  misc_feature: Arginine_decarboxylase_(EC_4.1.1.19)_Lysine_ decarboxylase_(EC_4.1.1.18)_Ornithine_decarboxylase_(EC_4.1.1.17)| Clostridium_asparagiforme_HB452c
MRREERLISRLKAYNETDMYPFHMPGHKRLAGAGDGEMGELEFPNPFTVDITEIEGFDNLHHPEGILRESMEWAAGVYGADRTYYLVNGSSGGILAAISAAAGFESRILTARNCHKSVYHAVYLNRLKPVYLYPQTVPGLGIQGGILPEDVEKSLRQYPDTRAVMVVSPTYDGIVSDIHKIAEIVHRAGLPLIVDEAHGAHFRYGREFPQSALELGADLVIQSIHKTLPSLTQTALLHVNLNRDKGGPYVDIGRLERFLQIYQSSSPSYVLMASIENAVWLMERLRLDRGAPGNAIDRYMERMGRLRDNLSKMRCLRLAGEWLKGSCGVWDTDMSKVVISTAGTGMTGTELDGILRELYHLEMEMCGPEYATAITSVMDSGEGLDRLEAAVMKIDRELAQKLEKTGNPNQGGWAGTGGADAAANSPALPLASQTCAPFPRLRSLMPIASAMDKDLERVALRESGGRISGEYIYIYPPGIPIVAPGEEISGEALDLVLRFMEQGLPVQGPQDRALEWLLVVRQNIDR

SEQ ID NO: 205 - protein, 131 aa, Clostridium asparagiforme
  misc_feature: Biotin_carboxyl_carrier_protein_ methylmalonylCoA_decarboxylase|Clostridium_asparagiforme_HB452c
MKSYTITVNGKAYAVTVEEGAASGVAAAPAAAPVAPAAPAAPAPAAAPAPAPAAAPAGAAGAVKVTAPMPGKVVGVKASAGQAVKKGDTILVLEAMKMENDIVAPQDGTVASINVSTGDSVESGAVLATLN

SEQ ID NO: 206 - protein, 305 aa, Clostridium asparagiforme
  misc_feature: Orotidine_5-phosphate_decarboxylase_ (EC_4.1.1.23)|Clostridium_asparagiforme_HB452c
MIQQLIEKIKKTKAPICVGLDPMLNYIPEHILKKSYSEFGETLEGAADAIWQFNKEIVDHTYDLIPSVKPQIAMYEQFGIEGLKSYQKTVDYCQEKGLLVIGDAKRGDIGSTSAAYATGHLGKVQVGGKTYSGFHTDFLTVNPYLGTDGVKPFVDVCNSDDKGLFVLVKTSNPSSGEFQDRLIDGRPLYELVAEKVVEWGDTCMDGDYSNVGAVVGATYPEMSKVLRKLMPRTYFLVPGYGAQGGTAADLAHCFNEDGLGAVVNSSRGIIAAYRQEKYKQFGPEHFAEASRQAVIDMVADINSVL

SEQ ID NO: 207 - protein, 395 aa, Clostridium asparagiforme
  misc_feature: Phosphopantothenoylcysteine_decarboxylase_ (EC_4.1.1.36)_/_Phosphopantothenoylcysteine_synthetase_ (EC_6.3.2.5)|Clostridium_asparagiforme_HB452c
MLKDKTVLLGVTGSIAAYKIASLASMLVKQGAKVQVLMTENATNFINPITFETLTGRKCLVDTFDRNFEFSVEHVSIAKAADVVMIAPASANVIAKLAHGLADDMLTTTVLACRCKKIIAPAMNTNMYENPIVQDNLKVCERYGMEVIAPATGYLACGDTGAGKMPEPEVLFEYIVREIAFGKDLAGKRVLVTAGPTRESIDPVRFITNRSTGKMGYAVARAAAYRGARVTLVTGPVNLKPPMFVDVVPVESAREMFEAVTSRSGEMDMIVKSAAVADYRPGTVGAEKIKKSDGDMSIALERTDDILGWLGSHRREGQILCGFSMETQNMLENSAAKLEKKHVDMIVANNLKVSGAGFGTDTNVVTFITKDGAEELPILTKDEVAHRLLDRLMGL

SEQ ID NO: 208 - protein, 482 aa, Ruminococcus gnavus
  misc_feature: Arginine_decarboxylase_(EC_4.1.1.19)| Ruminococcus_gnavus_HB40
MKNLSQENAPIYEALERFRRMRIVPFDVPGHKHGRGNKELVELLGERCVSIDVNSMKPLDNLCHPVSVIKDAEQLAAEAFHASHAFLMVGGTTSSVQAMVLSCCKKGDKIILPRNVHRSVINALVLCGAIPIYVNPDVDHDLGISLGMKVSQVERAIQKHPDAVAVFVNNPTYYGICSDLRSIVKKAHAHGMKVLVDEAHGTHFYFGENLPVSAMEAGADMASVSMHKSGGSLTQSSFLLVGPSMHAGYVRQIINLTQTTSGSYLLLSSLDISRRNLVLRGKESFARVAALADYAREEINRIGGYYAFSKELINGDSIYDFDTTKLSIHTLEIGLAGIEVYDILRDEYDIQIEFGDIGNILAYLSIGDRIQEIERLVSALAEVKRRYQKDKTGMLSQEYIEPEVIMSPQDSFYAEKEAVPIRESEGRICSEFVMCYPPGIPILAPGEKITKDVIEYILYAKEKGCSMTGPEDAAIEYLNVLV

SEQ ID NO: 209 - protein, 385 aa, Ruminococcus gnavus
  misc_feature: 209>NADHdependent_butanol_dehydrogenase_A_ (EC_1.1.1.)|Ruminococcus_gnavus_HB40_Assembly_20200511.fasta_| 33038.681
  misc_feature: NADHdependent_butanol_dehydrogenase_A_ (EC_1.1.1.)|Ruminococcus_gnavus_HB40_Assembly_20200511.fasta_| 33038.681
MENFNFCVGTRILFGKGQEEKLPELLRPYGNKVLLTYGGGSIKRTGLYDRIKNLLHEFEIFELGGIEPNPRIESVYAGAEICREEDIDVILAVGGGSTIDCSKAIAAAAYYEGDAWDLILHPDRIEKALPICTVLTLSATGSEMDNGGVITNLKTKEKLGFGNPLLLPKASVLNPENTFTVSASQTAAGSADIMSHILEVYFDTRRAAVPDRISEGLLKTVIQYAPIAVREPENYEARAELMWTSSLAINGICSTGKDCAWSCHPMEHELSAYYDITHGVGLAILTPRWMRYILNEHTVEKFAEYAKNVWGIEEKADLFETANAGIDATEEFLKSLGIPMTLSEVGITEEYFTEMAEHAVIFGGLSEAYVPLKAEDVVNIYRMCM

SEQ ID NO: 210 - protein, 382 aa, Ruminococcus gnavus
  misc_feature: L-threonine_phosphate_decarboxylase| Ruminococcus_gnavus_HB40
MTNSEKALQMHEQWNGKLETAAKAHVNSREDLAIAYTPGVAEPCKVIAKDPEAAYKYTIKSNTVAVVSDGSAVLGLGNIGALAAMPVMEGKAVLFKEFGGVNAVPICLDTQDTEEIIKTVVNIAPAFGGINLEDISAPRCFEIEERLKELLDIPVFHDDQHGTAIVVLAGIINALKVTGKKKEDCRVVVNGAGSAGVAITKLLLTYGFEHITMCDINGIISSASPNLNWMQKKMTEVTNLENKTGSLTDAMRGADIFVGVSAPGIVTKEMVASMNSDAILFAMANPVPEIMPDLAKEAGARVVGTGRSDFPNQVNNVVAFPGIFKGALEGRATQITEEMKLATANAIASLVSDEELNENNILPEAFDSRVADVVSKAVKELI

SEQ ID NO: 211 - protein, 371 aa, Ruminococcus gnavus
  misc_feature: L-threonine_phosphate_decarboxylase| Ruminococcus_gnavus_HB40
MNTKLEFHGSDIEKVCEHYGLKKDDIVNFGANVNPLGLSEQVKKALAAHLDLLSSYPDRSYTSLRNILSTYCQIPADFILPGNGSSELISLLIEERRPKHTMILGPTYSEYSRELGFSDSTIDCFYLKPEQDFQLSVEELCLALAKKQIDFLILCNPNNPTSSAIFHPELIKLLEFCQDHNIFVMIDETYVEFAPDVDAVTAVPLTQNFQNLMVLRGVSKFFAAPGMRFGYGITGNAEFLKKLKSKQIPWSLNSLGAFAGELLFQDKEYIKKTRNLILSEREYMYTKLRELPFFYVYPAYANFLLVQIQKDGLTSSDVFEACIREGLMIRDCSSFTGLEGEFVRFCIMDPEDNRRLLTVFQKISQSARKQV

SEQ ID NO: 212 - protein, 289 aa, Ruminococcus gnavus
  misc_feature: Phosphatidylserine_decarboxylase_(EC_4.1.1.65)| Ruminococcus_gnavus_HB40
MKLADRRGNIEECSSGQDRLLEWIYTHETGRFLMKGLINPHISEIGAAFLDSRASRVLIAWFIRKNKIPMYQYEQKKYRSFNEFFKRKALPGARRIIREPERLISPCDGRLSVYKIEENSRFQIKHTSYSTESLLKNEGLAKRYAGGYAWVFRLCVEDYHRYIYVDDGVKSENVKIPGVLHTVNPVANDSFPIYKENAREFSLLCSENFGTVLMMEVGAMMVGKIENRHQAARVRRGQEKGNFAFGGSTIILLTQKGKAMPDPDIWENSLNGIETKVRLGESVGRGKKR

SEQ ID NO: 213 - protein, 155 aa, Ruminococcus gnavus
  misc_feature: Uroporphyrinogen_decarboxylase_(UROD)| Ruminococcus_gnavus_HB40
MLDKMKGCKNFILAPGCDMPYDVPVENAIGVAQAVYETDSVREMLKNYVAEDEDIEVELPDYEHLEKPLVEVFTLDSATCAACTYMMGAANEAKKTFGDAIDMVEYKFTLKENIARCKKMGVPNLPSMYINGELKFRSLVPSKEELEGAIRAAMK

SEQ ID NO: 214 - protein, 471 aa, Ruminococcus gnavus
  misc_feature: Oxaloacetate_decarboxylase_alpha_chain_ (EC_4.1.1.3)_MethylmalonylCoA_decarboxylase_alpha_chain_ (EC_4.1.1.41)|Ruminococcus_gnavus_HB40
MAEIAKKPIKITETILRDAHQSLIATRMTTEQMMPIVDKMDKVGYHAVECWGGATFDASLRFLHEDPWERLRKFRDGFKNTKLQMLFRGQNILGYRPYADDVVEYFVQKSAANGIDIIRIFDCMNDMRNLQTAVTAANKEKAHAQVAMSYTLGDAYTLEYWVELAKRIEDMGANSICVKDMAGLLCPYQATELVTALKEAVEIPIEMHTHYTSGVASMTYLKSVEAGADIIDTAMSPFALGTSQPATEVMVETFKGTPYDTGLDQNLLAEIADYFRPIRDEALESGLLNPKNLGVNIKTLLYQVPGGMLSNLTSQLKEQGAEDKFYEVLEEVPRVRKDLGEPPLVTPSSQIVGTQAVFNVLMGERYKMATKETKDVLSGKYGATAKPFNPEVQKKVIGEDAEVITCRPADLIPNELDTLRKECAQYSQQDEDVLTYALFPQVATDFFKYRDAQQTKVDAKVADTEDGAYPV

SEQ ID NO: 215 - protein, 383 aa, Ruminococcus gnavus
  misc_feature: Oxaloacetate_decarboxylase_beta_chain_ (EC_4.1.1.3)_MethylmalonylCoA_decarboxylase_beta_chain_ (EC_4.1.1.41)|Ruminococcus_gnavus_HB40
MEYISNTLGNLVEQTAFMNLTFGNLIMIAVACFFLYLAIRHGFEPLLLVPIAFGMLLVNIYPDIMLHAEDSANGTGGLLYYFYVLDEWSILPSLIFLGVGAMTDFGPLIANPKSFLLGAAAQFGIFAAYLGAMAMGFSDKAAAAISIIGGADGPTSIFLAGKLQQTAILGPIAVAAYSYMSLVPIIQPPIMKLLTTEEERKIKMEQLRPVSKLERILFPIIVTIVVCTILPTTAPLVGMLMLGNLFKESGVVRQLTETASNALMYIVVILLGTSVGATTSAEAFLNMDTLKIVALGLIAFAFGTAAGVLFGKLMCWATKGKVNPLIGSAGVSAVPMAARVSQKVGAEADPTNFLLMHAMGPNVAGVIGTAVAAGTFMAIFGVK

SEQ ID NO: 216 - protein, 258 aa, Ruminococcus gnavus
  misc_feature: MethylmalonylCoA_decarboxylase_deltasubunit_ (EC_4.1.1.41)|Ruminococcus_gnavus_HB40
MKKKLSLIVLACALTLGVSGCGSEADTTTSNEKESMVSYSEAIVSSFSQTPDEVFDQYEEMSELQLDLMLLNTGLPVDSENFLSMIEAWKAGEAECGAFKSYGEFETEMTSSGIVVSTEAEYENKTADIEFTFDEEQQMDSLTINAHYSTAEILKKAGLNTILGMGTVFVVLIFISFIISLFRFIPELEKKFKNKKTAEPAKAPAPAPVPVAEPAAEEDSDDAELVAVISAAIAAAEGTSADGFVVRSIKRRKSNKWN

SEQ ID NO: 217 - protein, 477 aa, Ruminococcus gnavus; identical to SEQ ID NO: 115
  misc_feature: Pyridoxaldependent_decarboxylase| Ruminococcus_gnavus_HB40

SEQ ID NO: 218 - protein, 155 aa, Ruminococcus gnavus; identical to SEQ ID NO: 213
  misc_feature: Uroporphyrinogen_decarboxylase_(UROD)| Ruminococcus_gnavus_HB40

SEQ ID NO: 219 - protein, 372 aa, Ruminococcus gnavus
  misc_feature: Carboxynorspermidine_decarboxylase_ (EC_4.1.1.96)|Ruminococcus_gnavus_HB40
MKREELRTPCYVIMESEMKKNLQILQEIRQQSGCKILLAQKAFSMYSCYPLIGEYLDGTTASGLYEAKLGKECMGKENHIFSPAYREDEFEEVLESCEHLVFNSFSQLERFGERAAKAGKSVGIRVNPECSTQGEHAIYDPCAPGSRLGILVSEFPETLPSYVEGLHFHTLCEQNADALEITLKAVEEKFGKWMKQLKWVNFGGGHHITRADYDRELLISCVRRIQDTYNVQVYLEPGEAVALNAGHLITTVEDIVHNDIAVLDVSAACHMPDVIEMPYRPPLFDSGEAKEKEYTYLLGGPTCLAGDVIGSYSFDQPLKVGDRLTFGDMAIYTTVKNNTFNGMRLPDIVWERESGECEIVKSFGYEDFKCRL

SEQ ID NO: 220 - protein, 397 aa, Ruminococcus gnavus
  misc_feature: Phosphopantothenoylcysteine_decarboxylase_ (EC_4.1.1.36)_/_Phosphopantothenoylcysteine_synthetase_ (EC_6.3.2.5)|Ruminococcus_gnavus
MLKGKTVVLAVSGSIAAYKIASLASALKKLHANVQVLMTKNAVNFINPITFESLTGNKCLVDTFDRNFQYSVEHVALAKQADVVLVAPASANVIGKIAHGIADDMLTTTVMACKCKKIIAPAMNTNMFDNPILQDNLKILEHYGYEVISPAVGYLACGDTGAGKMPEPELLLQYILREIAYEKDMQGKRVLVTAGPTQESIDPVRFITNHSTGKMGYAIAKMCMLRGAEVTLVSGPTSIAKPEFVHVVDVVTAKEMYEEVTKRAKDQDIIIKAAAVADYRPKSVSSEKMKKKDDDLAIPMERTDDILKFLGEHKKEHQFLCGFSMETENMLENSRKKLEKKHLDMIVANNLKVEGAGFAGDTNVVTIITGQEEVSLGKMTKEETALRILDEILKATN

SEQ ID NO: 221 - protein, 424 aa, Ruminococcus gnavus
  misc_feature: Diaminopimelate_decarboxylase_(EC_4.1.1.20)| Ruminococcus_gnavus_HB40
MNKTPFVTKEQLDEIVKTYPTPFHLYDEMGIRENVKALREAFSWNEGYKEYFAVKACPNPFLIDILREYDCGCDCSSYTELMLSDAIGVKGQDIMFSSNDTPAEDYVLAEKLGAIINLDDFTHIDFLEKTIGYIPETISCRYNPGGLFKISNDIMDNPGDAKYGMTTEQLFEAFKVLKSKGAKEFGIHAFLASNTVTNDYYPMLAKVLFEQAVKLQKETGVHIKFINLSGGIGIPYTPDQEPNDIRVIGEGVRRVYEEVLVPAGMGDVAIYTELGRFMMGPYGCLVTTAIHEKHTHKEYIGVDACAVDLMRPAMYGAYHHITVMGKEDAPHDHKYDVTGSLCENNDKFAIDRMLPEIEKGDYLVIHDTGAHGFAMGYSYNGKLKHAELLLKEDGSVQMIRRAERPADYFATFDCFDIGKKLIKN

SEQ ID NO: 222 - protein, 321 aa, Ruminococcus gnavus
  misc_feature: Orotidine_5phosphate_decarboxylase_ (EC_4.1.1.23)|Ruminococcus_gnavus_HB40
MFCPFLENREEEKMINKLVAKIKETKAPIVVGLDPMLNYIPEHVQKKAFAEFGETLEGAAEAIWQFNKEIVDKTYDLIPAVKPQIAMYEQFGVPGIEAFKKTVDYCKSKDLVVIGDIKRGDIGSTSAAYAVGHLGSVKVGSKEYVPFDEDFATVNPYLGSDGVNPFIDVCKEHKKGLFILVKTSNPSSGEFQDQLIDGKPLYELVGEKVAQWGADCMGDEYSYIGAVVGATYPEMGKVLRKVMPKSYILVPGYGAQGGQGKDLVHFFNEDGLGAIVNSSRGIIAAYKQEAYAKFGAENFGDASRAAVEAMVADIQGALEAR

SEQ ID NO: 223 - protein, 122 aa, Ruminococcus gnavus
  misc_feature: Biotin_carboxyl_carrier_protein_ methylmalonylCoA_decarboxylase_Biotin_carboxyl_carrier_protein| Ruminococcus_gnavus_HB40
MKNYTITVNGNVYDVTVEEKGAGAVPVAPTVPKAAPKAAPAPAPKAAPAGAGSIQVKAGAAGKVFSIDASVGQAVKAGDAIVTVEAMKMEIPVVAPEDGTVASIDVAVGDAVESGAVLATLN

SEQ ID NO: 224 - protein, 478 aa, Ruminococcus gnavus
  misc_feature: Acetylcoenzyme_A_carboxyl_transferase_alpha_ chain_(EC_6.4.1.2)_/_Acetylcoenzyme_A_carboxyl_transferase_beta_ chain_(EC_6.4.1.2)|Ruminococcus_gnavus_HB40
MSNNATENSASRRIATLLDGGSFVEIGSAVTARNTDFNLQEKETPADGVITGYGVIDGNLVYVYSQDASVLKGAIGEMHAKKIANIYDMAMKMGAPVIGLIDCAGLRLQEATDALEAFGSLYFKQAMASGVIPQITAVFGMCGGGLAVVPGLTDFTFMENKEGKLFVNSPNALDGNIDSKCNTASAEYQSKTAGLVDAAGTEEEILGQIRSLICMLPANFEDDASYDECTDDLNRVCADLANAAEDTGIALATISDNNIFFETKREYAKEMVTGFIRLNGMTVGAVANRSKVYDAEGNAESYGQVLTVDGCKKAADFINFCDAFSIPVLSLTNVTGFEATLEAEKDMARAVAKLTYAFANASVPKVNVIVGKAYGSAYIAMNSKSIGADLVYAWPTAEIGMMDASMAAKIMYADADAETLKEKAAEYKELQSSPVSAARRGYVDAIIDAADTRKYVIGAFEMLFTKREDRPAKKHGTV

SEQ ID NO: 225 - protein, 610 aa, Enterococcus durans; identical to SEQ ID NO: 119
  misc_feature: decarboxylase_putative|Enterococcus_durans_HB48

SEQ ID NO: 226 - protein, 188 aa, Enterococcus durans
  misc_feature: Lysine_decarboxylase_family|Enterococcus_ durans_HB48
MKITIFCGASNGNNPIYSQRTVELGEWMIKNNHDLVYGGGKVGLMGVIADTIINNGGQAIGVMPTFLKDREIAHINLSELIVVENMPQRKGKMMALGEAFIALPGGPGTLEEISEVISWSRIGQNDSPCVLYNIDGYFNHLRNMFDHMVSEGFLSQDDRNNILFSDDIIEIESFIKDYKSPTIRKYSN

SEQ ID NO: 227 - protein, 300 aa, Cupriavidus taiwanensis
  misc_feature: RALTA_RS15325 phenylalanine 4-monooxygenase [ Cupriavidus taiwanensis LMG 19424 ] Gene ID 29760330
MSIAMATEAPGAFQGTLTDKLKEQFDAGLLSGQELRPDFTIAQPVHRYTSIDHAIWRKLYERQAEMLRGRVSDEFLQGLATLGMEKDRVPDFDQLNETLMRATGWQVVAVPGLVPDEVFFEHLANRRFPASWWMRKPEQLDYLQEPDCFHDVFGHVPLLINPVFADYMEAYGKGGLKAAGLGALDMLSRLYWYTVEFGLIRTEQGLRIYGAGIVSSQGESIYSLDSASPNRIGFDVRRIMRTRYRIDTFQKTYFVIDSFEQLFDATRPDFAPLYEELRAQPTLGAGDVAPGDQVLNVGTR

SEQ ID NO: 228 - protein, 321 aa, Mycolicibacterium smegmatis; identical to SEQ ID NO: 181
  misc_feature: Mycolicibacterium smegmatis (strain ATCC 700084 / mc(2)155); Histidine N-alpha-methyltransferase, uniprot A0R5M8

SEQ ID NO: 229 - protein, 206 aa, Akkermansia sp.
  misc_feature: Putative aromatic amino acid N-methyltransferase (EC 2.1.1.-) A0A139TQU2 Akkermansia sp. KLE1798 >tr|A0A139TQU2|A0A139TQU2_9BACT Uncharacterized protein OS=Akkermansia sp. KLE1798 OX=1574265 GN=HMPREF3039_01924 PE=4
MNTHAKSLNERLRTLISLIFGLFIGCALLFGETRWETSPVVEESLMLIACFMAGIGAFGRIWCSLYIAGYKNNVLVIEGPYSMCRNPLYFFSFIGGVGVACATETFTVPLLTALAFGIYYPAIIRREQERLISLFGNAYRDYCRTVPSFIPSLSLLKPPPATYSVNPATFTHNIFDALWFIWFIGIFEFISGLHDAGILPVWFFIP

(19) United States
(12) Patent Application Publication
(10) Pub. No.: US 2022/0370518 A1
(43) Pub. Date: Nov. 24, 2022

(54) THERAPEUTIC COMPOSITIONS AND METHODS OF USING SEROTONIN MODULATING MICROBIOME-BASED INTERVENTIONS TO TREAT SEROTONIN-RELATED DISEASES OR DISORDERS

(71) Applicant: HOLOBIOME, INC., Cambridge, MA (US)

(72) Inventors: Philip STRANDWITZ, Cambridge, MA (US); Mariaelena CABONI, Cambridge, MA (US); Stephen SKOLNICK, Cambridge, MA (US)

(73) Assignee: HOLOBIOME, INC., Cambridge, MA (US)

(21) Appl. No.: 17/621,767

(22) PCT Filed: Jun. 26, 2020
(86) PCT No.: PCT/US2020/039947

§ 371 Date: Dec. 22, 2021

Related U.S. Application Data
(60) Provisional application No. 62/867,592, filed on Jun. 27, 2019.

(51) Int. Cl. A61K 35/745; A61K 35/744; A61K 35/742; A61K 35/741; A61K 38/51; A61K 38/44

(52) U.S. Cl. CPC A61K 35/745; A61K 35/744; A61K 35/742; A61K 35/741; A61K 38/51; A61K 38/44

(57) ABSTRACT

The technology described herein is directed to compositions and methods for modulating serotonin in a subject. Described herein are compositions comprising viable or non-viable serotonin modulating bacteria, conditioned medium(s) of serotonin-modulating bacteria, cell pellet(s) of serotonin modulating bacteria, and/or metabolites and/or proteins derived from serotonin-modulating bacteria. Also described herein are methods of treating serotonin-related disease or disorders.

### CROSS-REFERENCE TO RELATED APPLICATIONS

This application claims benefit under 35 U.S.C. § 119(e) of U.S. Provisional Application No. 62/867,592 filed Jun. 27, 2019, the contents of which are incorporated herein by reference in their entirety.

### SEQUENCE LISTING

The instant application contains a Sequence Listing which has been submitted in ASCII format via EFS-Web and is hereby incorporated by reference in its entirety. Said ASCII copy, created on Jun. 26, 2020, is named 083103-095640WOPT_SL.txt and is 607,528 bytes in size.

### TECHNICAL FIELD

The technology described herein relates to compositions and methods for modulating serotonin.

### BACKGROUND

Recent work has connected the human microbiome—the trillions of bacteria that reside on or inside the body—to many components of health and disease. Of particular importance is the gut microbiome, the complex bacterial community located in the gastrointestinal tract. Incredibly, not only has the gut microbiome been found to be essential for maintaining metabolic and immune health, there is also amassing evidence that the gut microbiome interacts with the enteric and central nervous systems (ENS and CNS, respectively) via communication along the “gut-brain-axis”. Mechanistically, these interactions generally appear to be driven by direct production of metabolites that interact with these systems (e.g. neurotransmitters like GABA, or short-chain fatty acids like butyrate and acetate), modulation of dietary inputs which feed into these pathways (e.g. removal/alteration of the fate of tryptophan, which is an input for serotonin biosynthesis), or direct protein-protein or ligand-protein interactions (e.g. the sensing of microbial lipopolysaccharide (LPS) by toll-like receptors, which can induce inflammatory or anti-inflammatory cellular responses, depending on the LPS source organism). Of relevance is the link between serotonin (e.g., 5-hydroxytryptamine, 5-HT) and the microbiome.

More than 90% of the body's 5-HT is produced in the gastrointestinal (GI) tract. In many instances said 5-HT is produced by host gut cells under the influence of the gut microbiota, not produced by the microbiota itself. In the GI tract, 5-HT activates as many as 14 different 5-HT receptor subtypes, including those found on immune cells, enterocytes, and enteric nerves. In addition, circulating platelets sequester 5-HT from the GI tract, releasing it to promote hemostasis and distributing it to various body sites.

Serotonin generally has been shown to be involved in numerous physiological systems and disorders. This includes, as examples, intestinal movements, platelet activation/aggregation, stimulation of myenteric neurons and gut mobility, mood, appetite, sleep, some cognitive functions such as memory and learning, bone metabolism and remodeling, reward seeking behavior, regulation of vascular tone, primary hemostasis, hemopoiesis, cell-mediated immune responses, tumor growth, angiogenesis, cancer cell differentiation, and cardiac functions. An abnormal level of serotonin can cause pathological conditions including, but not limited to, depression, anxiety, obsessive-compulsive disorder, irritable bowel syndrome, cardiovascular disease, osteoporosis, abnormal gastrointestinal motility, fibrosis, abnormal platelet aggregation, abnormal platelet activation, metabolic disease, and an abnormal immune response.

The microbiome plays a major role in influencing serotonergic neurotransmission. In germ free animals, there is a significant reduction of serotonin in the blood and colon of mice compared to controls.

Modulation of the gut microbiota, e.g., through microbiota-based therapeutics, has been shown to be an effective treatment for a number of diseases in mouse models, including, but not limited to obesity, colitis, colon cancer, and Clostridium difficile infection, giving proof of principle for microbiota-based therapeutics. These therapeutics could be in the form of live bacteria, dead bacteria, microbial metabolites or proteins, bacteria engineered to perform specific functions or produce certain metabolites/proteins, or means to alter the microbiome—e.g. diet, prebiotics, antibiotics, sorbents, or inhibitors of specific microbial/host functions.

### SUMMARY

The present disclosure provides compositions and methods for decreasing at least one symptom of a serotonin-related disease or disorder in a subject in need thereof by altering the serotonin-modulating gut microbiota, serotonin-modulating gut-microbiota-derived metabolome, or serotonin-modulating gut-microbiota-derived proteome of the subject. The compositions can comprise one or more live serotonin-modulating bacteria, one or more dead-serotonin modulating bacteria, one or more conditioned medium(s) of serotonin-modulating bacteria, one or more cell pellet(s) of serotonin-modulating bacteria, and/or one or more 5-HT agonists, metabolites and/or proteins (derived from serotonin-modulating bacteria), and therapeutic compositions comprising the same. The method can comprise administering combinations of one or more live serotonin-modulating bacteria, one or more dead-serotonin modulating bacteria, one or more conditioned medium(s) of serotonin-modulating bacteria, one or more cell pellet(s) of serotonin-modulating bacteria, and/or one or more 5-HT agonists, metabolites and/or proteins (derived from serotonin-modulating bacteria).

The present technology has the advantage of alleviating the symptoms of serotonin-related disease or disorders without the aid of synthetic medications (e.g., serotonin-reuptake inhibitors), which can have unwanted side-effects, or in combination with existing medications. Additionally, the present technology can have the advantage of further improving other aspects of health of the subject, as the bacteria can perform multiple mechanisms (e.g. alter serotonin signaling but also alter the immune system). Additional features and advantages of the present technology will be apparent to one of skill in the art.

In one aspect described herein is a therapeutic composition for increasing serotonin level in a mammalian subject in need thereof, the composition comprising an amount of a live isolated serotonin-increasing bacterial species, dead isolated serotonin-increasing bacterial species, conditioned medium from an isolated, cultured serotonin-increasing bacterial species, cell pellet of an isolated serotonin-increasing bacterial species, a purified metabolite produced by an isolated serotonin-increasing bacterial species, a purified protein produced by an isolated serotonin-increasing bacterial species, or a combination thereof sufficient to increase serotonin level in the subject, and an excipient or carrier suitable for delivery to the gut.

In some embodiments of any of the aspects, the isolated serotonin-increasing bacterial species increases serotonin in at least one of the following ways: production of serotonin; production of secreted metabolites or secreted proteins that induce serotonin production; production of ligands that induce serotonin production; or production of an agonist of a serotonin receptor or the trace amine-associated receptor (TAAR).

In some embodiments of any of the aspects, the isolated serotonin-increasing bacterial species is a serotonin-producing bacterial species.

In some embodiments of any of the aspects, the serotonin-producing bacterial species comprises one or more species selected from Enterococcus durans, Clostridium lavalense, Clostridium asparagiforme, Ruminococcus gnavus.

In some embodiments of any of the aspects, the serotonin-producing bacterial species comprises one or more species selected from Enterococcus durans HB-48, Clostridium lavalense HB-452c, Ruminococcus gnavus HB-40, Ruminococcus gnavus HB-516.

In some embodiments of any of the aspects, the serotonin-producing bacterial species comprises a 16S sequence at least 95% identical to a 16S sequence selected from SEQ ID NOs: 1-4.

In some embodiments of any of the aspects, the serotonin-producing bacterial species produces serotonin under conditions found in the mammalian gut.

In some embodiments of any of the aspects, the mammalian subject is a human subject.

In some embodiments of any of the aspects, the isolated serotonin-producing bacterial species encodes and expresses a decarboxylase enzyme that catalyzes the production of tryptamine from tryptophan.

In some embodiments of any of the aspects, the decarboxylase enzyme is a tryptophan decarboxylase.

In some embodiments of any of the aspects, the decarboxylase enzyme belongs to the EC number 4.1.1.105.

In some embodiments of any of the aspects, the decarboxylase enzyme is at least 50% identical to an enzyme comprising an amino acid sequence selected from SEQ ID Nos: 115-119.

In some embodiments of any of the aspects, the isolated serotonin-producing bacterial species encodes and expresses an enzyme that hydroxylates tryptamine to produce serotonin.

In some embodiments of any of the aspects, the enzyme that hydroxylates tryptamine is a tryptamine 5-hydroxylase.

In some embodiments of any of the aspects, the enzyme that hydroxylates tryptamine is at least 50% identical to the enzyme of SEQ ID NO: 134.

In some embodiments of any of the aspects, the enzyme that hydroxylates tryptamine is an anaerobic hydroxylase.

In some embodiments of any of the aspects, the isolated serotonin-producing bacterial species encodes and expresses a decarboxylase enzyme that catalyzes the production of tryptamine from tryptophan and an enzyme that hydroxylates tryptamine to produce serotonin.

In some embodiments of any of the aspects, the live isolated serotonin-producing bacterial species, dead isolated serotonin producing bacterial species, conditioned medium from an isolated, cultured serotonin-producing bacterial species or cell pellet of an isolated serotonin-producing bacterial species comprises a first bacterial species that encodes and expresses a decarboxylase enzyme that catalyzes the production of tryptamine from tryptophan and a second bacterial species that encodes and expresses an enzyme that hydroxylates tryptamine to produce serotonin.

In some embodiments of any of the aspects, the decarboxylase is a lysine decarboxylase family enzyme.

In some embodiments of any of the aspects, the live isolated serotonin-producing bacterial species, dead isolated serotonin producing bacterial species, conditioned medium from an isolated, cultured serotonin-producing bacterial species or cell pellet of an isolated serotonin-producing bacterial species comprises one or more bacterial species that encode and express an enzyme that converts tryptophan to 5-hydroxy-L-tryptophan (5-HTP).

In some embodiments of any of the aspects, the enzyme that converts tryptophan to 5-hydroxy-L-tryptophan (5-HTP) has an amino acid sequence at least 50% identical to a sequence selected from SEQ ID Nos: 120-126.

In some embodiments of any of the aspects, the enzyme that converts tryptophan to 5-hydroxy-L-tryptophan (5-HTP) belongs to the EC number 1.14.16.4.

In some embodiments of any of the aspects, the live isolated serotonin-producing bacterial species, dead isolated serotonin producing bacterial species, conditioned medium from an isolated, cultured serotonin-producing bacterial species or cell pellet of an isolated serotonin-producing bacterial species comprises a bacterial species that encodes and expresses an enzyme that converts 5-hydroxy-L-tryptophan to serotonin.

In some embodiments of any of the aspects, the enzyme that converts 5-hydroxy-L-tryptophan to serotonin is an aromatic L-amino acid decarboxylase.

In some embodiments of any of the aspects, the enzyme that catalyzes the conversion of 5-hydroxy-L-tryptophan to serotonin belongs to the EC number 4.1.1.28.

In some embodiments of any of the aspects, the enzyme that converts 5-hydroxy-L-tryptophan to serotonin is at least 50% identical to a sequence selected from SEQ ID NOs: 127-133.

In some embodiments of any of the aspects, the isolated serotonin-producing bacterial species further encodes and expresses an enzyme that converts 5-hydroxy-L-tryptophan to serotonin.

In some embodiments of any of the aspects, the enzyme that converts 5-hydroxy-L-tryptophan to serotonin is an aromatic L-amino acid decarboxylase.

In some embodiments of any of the aspects, the enzyme that converts 5-hydroxy-L-tryptophan to serotonin is at least 50% identical to a sequence selected from SEQ ID NOs: 127-133.

In some embodiments of any of the aspects, the live isolated serotonin-producing bacterial species, dead isolated serotonin producing bacterial species, conditioned medium from an isolated, cultured serotonin-producing bacterial species or cell pellet of an isolated serotonin-producing bacterial species comprises a first bacterial species that encodes and expresses an enzyme that catalyzes the conversion of tryptophan to 5-hydroxy-L-tryptophan and a second bacterial species that encodes and expresses an enzyme that converts 5-hydroxy-L-tryptophan to serotonin.

In some embodiments of any of the aspects, the enzyme that catalyzes the conversion of tryptophan to 5-hydroxy-L-tryptophan is a tryptophan hydroxylase.

In some embodiments of any of the aspects, the enzyme that catalyzes the conversion of tryptophan to 5-hydroxy-L-tryptophan has an amino acid sequence at least 50% identical to a sequence selected from SEQ ID Nos: 120-126, and the enzyme that converts 5-hydroxy-L-tryptophan to serotonin is at least 50% identical to a sequence selected from SEQ ID NOs: 127-133.

In some embodiments of any of the aspects, the enzyme that catalyzes the conversion of tryptophan to 5-hydroxy-L-tryptophan is a phenylalanine hydroxylase.

In some embodiments of any of the aspects, the phenylalanine hydroxylase comprises an amino acid sequence comprising one or more of phenylalanine at the position corresponding to W192, isoleucine or leucine at the position corresponding to F197, and cysteine at the position corresponding to E219 of the phenylalanine hydroxylase of Cupriavidus taiwanensis (SEQ ID NO: 227).

In one aspect described herein is a pharmaceutical composition comprising a therapeutic composition as described herein and a pharmaceutically acceptable carrier.

In one aspect described herein is a method of increasing serotonin level in a mammalian subject in need thereof, the method comprising administering a composition as described herein to the subject, whereby a serotonin level is increased.

In some embodiments of any of the aspects, the administering is to the gut of the subject.

In some embodiments of any of the aspects, the level of serotonin in the gut is increased.

In some embodiments of any of the aspects, the level of serotonin in circulation is increased.

In one aspect described herein is a method of treating a disease or disorder involving or characterized by low serotonin in a subject in need thereof, the method comprising administering a composition as described herein to the subject, whereby the disease or disorder is treated.

In some embodiments of any of the aspects, the administering is to the gut of the subject.

In some embodiments of any of the aspects, the level of serotonin in the gut is increased.

In some embodiments of any of the aspects, the level of serotonin in circulation is increased.

In some embodiments of any of the aspects, the disease or disorder is selected from the group consisting of constipation, IBS-C, depression, anxiety, addiction, neurodegenerative disorders, autism spectrum disorder, sleep disorders, attention deficit hyperactivity disorder (ADHD), memory loss (e.g. dementia), learning difficulties, osteoporosis, heartburn, dermatological conditions (eczema and itch), GERD, and pain disorders.

In some embodiments of any of the aspects, the disease or disorder is not a gut disease or disorder.

In some embodiments of any of the aspects, the disease or disorder is selected from the group consisting of depression, anxiety, addiction, neurodegenerative disorders, autism spectrum disorder, sleep disorders, attention deficit hyperactivity disorder (ADHD), memory loss (e.g. dementia), learning difficulties, osteoporosis, dermatological conditions (eczema and itch), and pain disorders.

In some embodiments of any of the aspects, the composition comprising an amount of a live isolated serotonin-increasing bacterial species, dead isolated serotonin increasing bacterial species, conditioned medium from an isolated, cultured serotonin-increasing bacterial species, cell pellet of an isolated serotonin-increasing bacterial species, a purified metabolite produced by an isolated serotonin-increasing bacterial species, a purified protein produced by an isolated serotonin-increasing bacterial species, or a combination thereof sufficient to increase serotonin level in the subject, and an excipient or carrier suitable for delivery to the gut promotes production of serotonin by cells of a subject in which the composition is delivered to their gut.

In some embodiments of any of the aspects, the composition promotes expression of tryptophan hydroxylase 1 in cells of the subject.

In some embodiments of any of the aspects, the isolated serotonin-increasing bacterial species increases serotonin in at least one of the following ways: production of secreted metabolites or secreted proteins that induce serotonin production; production of ligands that induce serotonin production; or production of an agonist of a serotonin receptor or the trace amine-associated receptor (TAAR).

In some embodiments of any of the aspects, the isolated serotonin-increasing bacterial species increases serotonin through production of secreted metabolites or secreted proteins that induce serotonin production.

In some embodiments of any of the aspects, the isolated serotonin-increasing bacterial species comprises one or more species selected from the group consisting of: Acidaminococcus intestini, Agathobacter rectalis, Alistipes onderdonkii, Alistipes putredinis, Anaerotruncus colihominis, Bacillus cereus, Bacteroides caccae, Bacteroides cellulosilyticus, Bacteroides dorei, Bacteroides inegoldii, Bacteroides fragilis, Bacteroides koreensis, Bacteroides ovatus, Bacteroides plebeius, Bacteroides salyersiae, Bacteroides stercoris, Bacteroides uniformis, Bacteroides vulgatus, Bacteroides xylanisolvens, Bifidobacterium adolescentis, Bifidobacterium breve, Bifidobacterium faecale, Bifidobacterium longum, Bilophila wadsworthia, Butyricimonas paravirosa, Clostridium aldenese, Clostridium bolteae, Clostridium clostridioforme, Clostridium hathewayi, Clostridium innoculum, Clostridium paraputrificum, Clostridium saudiense, Clostridium scindens, Clostridium tyrobutyricum, Clostridium hylemonae HB-73, Collinsella aerofaciens, Coprococcus comes, Coprococcus eutactus, Dysosmobacter welbionis, Eisenbergiella tayi, Enterococcus faecium, Erysipelatoclostridium ramosum, Eubacterium eligens, Faecalitalea cylindroides, Flavonifractor plautii, Flintibacter butyricus, Gemmiger formicilis, Gordonibacter pamelaeae, Hungatella effluvii, Hungatella hathewayi, Intestinimonas butyriciproducens, Lactobacillus brevis, Mediterraneibacter faecis, Oscillibacter sp., Parabacteroides distasonis, Parabacteroides johnsonii, Parabacteroides merdae, Parasutterella excrementihominis, Ruminococcus bicirculans, Ruminococcus gnavus, Streptococcus gordonii, and Turicibacter sanguinis.

In some embodiments of any of the aspects, the isolated serotonin-increasing bacterial species comprises one or more species selected from the group consisting of Acidaminococcus intestini HB-95, Agathobacter rectalis HB-257, Alistipes onderdonkii HB-311, Alistipes putredinis HB-324, Anaerotruncus colihominis HB-474, Anaerotruncus colihominis HB-83, Bacillus cereus HB-25, Bacteroides caccae HB-11, Bacteroides cellulosilyticus HB-227, Bacteroides dorei HB-12, Bacteroides finegoldii HB-31, Bacteroides fragilis HB-58, Bacteroides koreensis HB-385, Bacteroides ovatus HB-70, Bacteroides plebeius HB-237, Bacteroides salyersiae HB-32, Bacteroides stercoris HB-33, Bacteroides uniformis HB-13, Bacteroides vulgatus HB-10, Bacteroides xylanisolvens HB-35, Bifidobacterium adolescentis HB-179, Bifidobacterium breve HB-90, Bifidobacterium faecale HB-159, Bifidobacterium longum HB-71, Bilophila wadsworthia HB-693, Butyricimonas paravirosa HB-453, Clostridium aldenese HB-440, Clostridium bolteae HB-442, Clostridium clostridioforme HB-642, Clostridium hathewayi HB-152, Clostridium innoculum HB-82, Clostridium paraputrificum HB-27, Clostridium saudiense HB-142, Clostridium scindens HB-444, Clostridium tyrobutyricum HB-469, Clostridium hylemonae HB-73, Collinsella aerofaciens HB-274, Coprococcus comes HB-376, Coprococcus eutactus HB-155, Dysosmobacter welbionis HB-45, Eisenbergiella tayi HB-437, Eisenbergiella tayi HB-612, Enterococcus faecium HB-640, Enterococcus faecium HB-85, Erysipelatoclostridium ramosum HB-24, Eubacterium eligens HB-252, Faecalitalea cylindroides HB-664, Flavonifractor plautii HB-472, Flintibacter butyricus HB-344, Gemmiger formicilis HB-325, Gordonibacter pamelaeae HB-15, Hungatella effluvii HB-02, Hungatella hathewayi HB-01, Intestinimonas butyriciproducens HB-478, Lactobacillus brevis HB-87, Mediterraneibacter faecis HB-364, Oscillibacter sp. HB-28, Parabacteroides distasonis HB-20, Parabacteroides johnsonii HB-03, Parabacteroides merdae HB-63, Parasutterella excrementihominis HB-330, Ruminococcus bicirculans HB-268, Ruminococcus gnavus HB-40, Ruminococcus gnavus HB-516, Streptococcus gordonii HB-62, Streptococcus gordonii HB-98, and Turicibacter sanguinis HB-147.

In some embodiments of any of the aspects, the isolated serotonin-increasing bacterial species comprises a 16S sequence at least 95% identical to a sequence selected from SEQ ID Nos 1, 2, and 5-69.

In some embodiments of any of the aspects, the isolated serotonin-increasing bacterial species increases serotonin through production of an agonist of a serotonin receptor or the trace amine-associated receptor (TAAR).

In some embodiments of any of the aspects, the isolated serotonin-increasing bacterial species encodes and expresses enzymes sufficient for the production of an agonist of a serotonin receptor or the trace amine-associated receptor (TAAR).

In some embodiments of any of the aspects, the isolated bacterial species comprises one or more species selected from the group consisting of: Akkermansia muciniphila, Adlercreutzia equolifaciens, Clostridium sporogenes, Clostridium lavalense, Clostridium asparagiforme, Coprococcus eutactus, Coprococcus comes, Enterococcus durans, Enterorhabdus muris, Enterorhabdus caecimuris, Mycolicibacterium smegmatis, Peptostreptococcus russelli, and Ruminococcus gnavus.

In some embodiments of any of the aspects, the isolated bacterial species comprises one or more species selected from the group consisting of: Enterococcus durans HB-48, Clostridium lavalense HB-452c, Ruminococcus gnavus HB-40, Ruminococcus gnavus HB-516. In some embodiments, the 5-HT agonist-producing bacteria are greater than 95% similar by 16S sequencing to Enterococcus durans HB-48, Clostridium lavalense HB-452c, Ruminococcus gnavus HB-40, Ruminococcus gnavus HB-516, Clostridium sporogenes JCM 7836, Akkermansia muciniphila BAA-835, Clostridium sporogenes McClung 2004, Peptostreptococcus russelli RT-10B, Mycolicibacterium smegmatis ATCC 19420, Enterorhabdus muris WCA-131-CoC-2, Adlercreutzia equolifaciens FJC-B9, Enterorhabdus caecimuris B7, Coprococcus eutactus ATCC 27759, and Coprococcus comes ATCC 27758.

In some embodiments of any of the aspects, the isolated bacterial species comprises a 16S sequence at least 95% identical to a sequence selected from SEQ ID Nos 1-4 and 105-114.

In some embodiments of any of the aspects, the agonist of the serotonin receptor or TAAR is selected from the group consisting of N-methyltryptamine, N,N-dimethyltryptamine, N-methylserotonin, and N,N-dimethylserotonin.

In some embodiments of any of the aspects, the isolated serotonin-increasing bacterial species encodes and expresses one or more enzymes that catalyze the methylation of tryptamine by a mechanism corresponding to that of human indolethylamine N-methyltransferase, or one or more enzymes at least 50% identical to the radical S-adenosyl-L-methionine-dependent, ergothioneine biosynthetic enzyme egtD, or one or more phosphatidylethanolamine N-methyltransferase enzymes (e.g., SEQ ID NOs: 228 or 229).

In some embodiments of any of the aspects, culture supernatant of the isolated bacterial species increases expression of tryptophan hydroxylase 1 (TPH-1) in cells of the host.

In some embodiments of any of the aspects, the isolated bacterial species comprises one or more species selected from the group consisting of Enterococcus durans, Clostridium lavalense, Lactobacillus brevis, Bifidobacterium faecale, Anaerotruncus colihominis, and Clostridium ramosum.

In some embodiments of any of the aspects, the isolated bacterial species comprises one or more species selected from the group consisting of Enterococcus durans HB-48, Clostridium lavalense HB-452c, Lactobacillus brevis HB-87, Bifidobacterium faecale HB-159, Anaerotruncus colihominis HB-83, and Clostridium ramosum HB-24 or a combination thereof.

In some embodiments of any of the aspects, the isolated bacterial species comprises a 16S sequence at least 95% identical to a sequence selected from the group consisting of SEQ ID Nos: 3, 4, 11, 28, 30 and 39.

In some embodiments of any of the aspects, the isolated bacterial species comprises one or more species selected from the group consisting of Clostridium scindens, Bifidobacterium faecale, Enterococcus durans, Clostridium lavalense, Anaerotruncus colihominis, and Erysipelatoclostridium ramosum.

In some embodiments of any of the aspects, the isolated bacterial species comprises one or more species selected from the group consisting of Clostridium scindens HB-444, Bifidobacterium faecale HB-159, Enterococcus durans HB-48, Clostridium lavalense HB-452c, Anaerotruncus colihominis HB-83, and Erysipelatoclostridium ramosum HB-24.

In some embodiments of any of the aspects, the isolated bacterial species comprises a 16S sequence that is at least 95% identical to one of SEQ ID NOs: 3, 4, 11, 23, 28 and 39.

In some embodiments of any of the aspects, the isolated serotonin-increasing bacterial species increases serotonin through production of ligands that induce serotonin production.

In some embodiments of any of the aspects, a cell pellet from the isolated serotonin increasing bacterial species modulates serotonin when administered to a subject.

In some embodiments of any of the aspects, the isolated bacterial species comprises one or more species selected from the group consisting of Anaerotruncus colihominis, Bacteroides caccae, Bacteroides clarus, Bacteroides dorei, Bacteroides finegoldii, Bacteroides ovatus, Bacteroides salyersiae, Bacteroides thetaiotaomicron, Bacteroides xylanisolvens, Bifidobacterium adolescentis, Bifidobacterium faecale, Bittarella massiliensis, Blautia wexlerae, Clostridium aldenese, Clostridium bolteae, Clostridium hathewayi, Clostridium saudiense, Clostridium scindens, Clostridium tyrobutyricum, Dialister invisus, Eisenbergiella tayi, Enterococcus durans, Enterococcus faecium, Eubacterium eligens, Gemmiger formicilis, Gordonibacter pamelaeae, Hungatella effluvii, Lactobacillus brevis, Longibaculum muris, Mediterraneibacter faecis, Parabacteroides distasonis, Parabacteroides merdae, Parasutterella excrementihominis, Prevotella copri, Prevotella sp, Prevotella sp., Romboutsia lituseburensis, Ruminococcus sp., Ruminococcus gnavus, Sellimonas intestinalis, and Sutterella wadsworthensis.

In some embodiments of any of the aspects, the isolated bacterial species comprises one or more species selected from the group consisting of Anaerotruncus colihominis HB-83, Bacteroides caccae HB-11, Bacteroides clarus HB-30, Bacteroides dorei HB-12, Bacteroides inegoldii HB-31, Bacteroides ovatus HB-70, Bacteroides salyersiae HB-32, Bacteroides thetaiotaomicron HB-34, Bacteroides xylanisolvens HB-35, Bifidobacterium adolescentis HB-179, Bifidobacterium faecale HB-159, Bittarella massiliensis HB-477, Blautia wexlerae HB-16, Clostridium aldenese HB-440, Clostridium bolteae HB-442, Clostridium hathewayi HB-152, Clostridium saudiense HB-142, Clostridium scindens HB-444, Clostridium tyrobutyricum HB-469, Dialister invisus HB-387, Eisenbergiella tayi HB-612, Enterococcus durans HB-48, Enterococcus faecium HB-85, Eubacterium eligens HB-252, Gemmiger formicilis HB-325, Gordonibacter pamelaeae HB-15, Hungatella effluvii HB-02, Lactobacillus brevis HB-87, Longibaculum muris HB-79, Mediterraneibacter faecis HB-364, Parabacteroides distasonis HB-20, Parabacteroides merdae HB-63, Parasutterella excrementihominis HB-330, Prevotella copri HB-373, Prevotella sp HB-649, Prevotella sp. HB-333, Romboutsia lituseburensis HB-102, Ruminococcus sp. HB-626, Ruminococcus gnavus HB-40, Ruminococcus gnavus HB-516, Sellimonas intestinalis HB-443, and Sutterella wadsworthensis HB-259.

In some embodiments of any of the aspects, the isolated bacterial species comprises a 16S sequence at least 95% identical to a sequence selected from the group consisting of SEQ ID Nos 1-3, 5-30 and 70-82.

In some embodiments of any of the aspects, the cell pellet increases expression of TPH-1 in cells of the subject.

In some embodiments of any of the aspects, the isolated bacterial species comprises one or more species selected from Clostridium lavalense, Lactobacillus brevis, Bifidobacterium faecale, Anaerotruncus colihominis, and Clostridium ramosum.

In some embodiments of any of the aspects, the isolated bacterial species comprises one or more species selected from Clostridium lavalense HB-452c, Lactobacillus brevis HB-87, Bifidobacterium faecale HB-159, Anaerotruncus colihominis HB-83, and Clostridium ramosum HB-24.

In some embodiments of any of the aspects, the isolated bacterial species comprises one or more species that comprise a 16S sequence at least 95% identical to a sequence selected from SEQ ID Nos 4, 11, 28, 30, and 39.

In some embodiments of any of the aspects, the isolated bacterial species comprises one or more species selected from Clostridium scindens, Bifidobacterium faecale, Enterococcus durans, Clostridium lavalense, Anaerotruncus colihominis, and Erysipelatoclostridium ramosum.

In some embodiments of any of the aspects, the isolated bacterial species comprises one or more species selected from Clostridium scindens HB-444, Bifidobacterium faecale HB-159, Enterococcus durans HB-48, Clostridium lavalense HB-452c, Anaerotruncus colihominis HB-83, and Erysipelatoclostridium ramosum HB-24.

In some embodiments of any of the aspects, the isolated bacterial species comprises one or more species that comprise a 16S sequence at least 95% identical to a sequence selected from SEQ ID Nos 3, 4, 11, 23, 28 and 39.

In some embodiments of any of the aspects, the isolated bacterial species are grown in medium containing one or more nutrients selected from the group consisting of conditioned medium from other bacteria, N-Acetyl-D-Galactosamine, N-Acetyl-D-Glucosamine, N-Acetyl-β-D-Mannosamine, Adonitol, Amygdalin, D-Arabitol, Arbutin, D-Cellobiose, α-Cyclodextrin, β-Cyclodextrin, Dextrin, Dulcitol, i-Erythritol, D-Fructose, L-Fucose, D-Galactose, D-Galacturonic Acid, Gentiobiose, D-Gluconic Acid, D-Glucosaminic Acid, α-D-Glucose, α-D-Glucose 1-Phosphate, D-Glucose6-Phosphate, Glycerol, D,L-α-Glycerol Phosphate, m-Inositol, α-D-Lactose, Lactulose, Maltose, Maltotriose, D-Mannitol, D-Mannose, D-Melezitose, D-Melibiose, 3-Methyl-DGlucose, α-Methyl-DGalactoside, β-Methyl-D-Galactoside, α-Methyl-D-Glucoside, β-Methyl-D-Glucoside, Mucin, Palatinose, D-Raffinose, L-Rhamnose, Salicin, D-Sorbitol, Stachyose, Sucrose, D-Trehalose, Turanose, Acetic Acid, Formic Acid, Fumaric Acid, Glyoxylic Acid, α-Hydroxybutyric Acid, β-Hydroxybutyric Acid, Itaconic Acid, α-Ketobutyric Acid, α-Ketovaleric Acid, D,L-Lactic Acid, L-Lactic Acid, D-Lactic Acid Methyl Ester, D-Malic Acid, L-Malic Acid, Propionic Acid, Pyruvic Acid, Pyruvic Acid Methyl Ester, D-Saccharic Acid, Succinamic Acid, Succinic Acid, Succinic Acid Mono-Methyl Ester, m-Tartaric Acid, Urocanic Acid, Alaninamide, L-Alanine, L-Alanyl-LGlutamine, L-Alanyl-LHistidine, L-Alanyl-LThreonine, L-Asparagine, L-Glutamic Acid, L-Glutamine, Glycyl-LAspartic Acid, Glycyl-LGlutamine, Glycyl-LMethionine, Glycyl-LProline, L-Methionine, L-Phenylalanine, L-Serine, L-Threonine, L-Valine, L-Valine plus L-Aspartic Acid, 2′-Deoxy Adenosine, Inosine, Thymidine, Uridine, Thymidine-5′-Mono-phosphate, and Uridine-5′-Monophosphate.

In some embodiments of any of the aspects, an isolated serotonin-increasing bacterial species in the composition produces tryptophan.

In some embodiments of any of the aspects, an isolated serotonin-increasing bacterial species in the composition encodes or expresses at least one enzyme involved in tryptophan production.

In some embodiments of any of the aspects, the enzyme involved in tryptophan production is selected from the group consisting of: Tryptophan synthase; Indole-3-glycerol phosphate synthase; Anthranilate phosphoribosyltransferase; Anthranilate synthase; and N-(5′-phosphoribosyl)anthranilate isomerase; 1-(5-phosphoribosyl)-5-[(5-phosphoribosylamino)methylideneamino] imidazole-4-carboxamide isomerase.

In some embodiments of any of the aspects, the enzyme involved in tryptophan production belongs to an EC number selected from the group consisting of: EC 4.2.1.20, EC 4.1.1.48, EC 2.4.2.18, EC 4.1.3.27, EC 5.3.1.24, and EC 5.3.1.36.

In some embodiments of any of the aspects, the enzyme involved in tryptophan production has an amino acid sequence at least 50% identical to a sequence selected from SEQ ID Nos 135-163.

In some embodiments of any of the aspects, an isolated serotonin-increasing bacterial species in the composition produces a metabolite of phenylalanine selected from phenethylamine, tyramine or N-methylated derivatives thereof that activate the TAAR system.

In some embodiments of any of the aspects, the isolated serotonin-increasing bacterial species in the composition produces one or more indole-3-carboxylic acid derivatives of tryptophan.

In some embodiments of any of the aspects, the indole-3-carboxylic acid derivative of tryptophan is one or more of indole-3-propionic acid, indole-3-acrylic acid, indole-3-lactic acid, indole-3-pyruvic acid, or indole-3-acetic acid.

In some embodiments of any of the aspects, the isolated bacterial species comprises and expresses genes of the fldAIBC gene cluster.

In some embodiments of any of the aspects, the isolated bacterial species encodes and expresses acyl-CoA dehydrogenase.

In some embodiments of any of the aspects, the acyl-CoA dehydrogenase belongs to EC 1.3.99.3; EC 1.3.8.7; EC 1.3.8.8; or EC 1.3.8.9.

In some embodiments of any of the aspects, the acyl-CoA dehydrogenase has an amino acid sequence at least 50% identical to a sequence selected from SEQ ID Nos 164-171.

In some embodiments of any of the aspects, the isolated bacterial species encodes and expresses an enzyme with a sequence at least 50% identical to an enzyme having an amino acid sequence of any one of SEQ ID Nos 172-184.

In some embodiments of any of the aspects, the isolated bacterial species encodes and expresses an enzyme belonging to an EC group selected from: EC 4.1.99.1; EC 2.8.3.17; EC 4.2.1.175; EC 5.6.1.9; and EC 2.1.1.

In one aspect described herein is a pharmaceutical composition comprising the therapeutic composition as described herein, and a pharmaceutically acceptable carrier.

In one aspect described herein is a method of increasing serotonin level in a mammalian subject in need thereof, the method comprising administering a composition as described herein to the subject, whereby a serotonin level is increased.

In some embodiments of any of the aspects, the administering is to the gut of the subject.

In some embodiments of any of the aspects, the level of serotonin in the gut is increased.

In some embodiments of any of the aspects, the level of serotonin in circulation is increased.

In one aspect described herein is a method of treating a disease or disorder involving or characterized by low serotonin in a subject in need thereof, the method comprising administering a composition as described herein to the subject, whereby the disease or disorder is treated.

In some embodiments of any of the aspects, the administering is to the gut of the subject.

In some embodiments of any of the aspects, the level of serotonin in the gut is increased.

In some embodiments of any of the aspects, the level of serotonin in circulation is increased.

In some embodiments of any of the aspects, the disease or disorder is selected from the group consisting of constipation, IBS-C, depression, anxiety, addiction, neurodegenerative disorders, autism spectrum disorder, sleep disorders, attention deficit hyperactivity disorder (ADHD), memory loss (e.g. dementia), learning difficulties, osteoporosis, heartburn, dermatological conditions (eczema and itch), GERD, and pain disorders.

In some embodiments of any of the aspects, the disease or disorder is not a gut disease or disorder.

In some embodiments of any of the aspects, the disease or disorder is selected from the group consisting of depression, anxiety, addiction, neurodegenerative disorders, autism spectrum disorder, sleep disorders, attention deficit hyperactivity disorder (ADHD), memory loss (e.g. dementia), learning difficulties, osteoporosis, dermatological conditions (eczema and itch), and pain disorders.

In one aspect described herein is a therapeutic composition for decreasing serotonin level in a mammalian subject in need thereof, the composition comprising an amount of a live isolated bacterial species, dead isolated bacterial species, conditioned medium from an isolated, cultured bacterial species, cell pellet of an isolated bacterial species, a purified metabolite produced by an isolated bacterial species, a purified protein produced by an isolated bacterial species, or a combination thereof sufficient to decrease serotonin level in the subject, and an excipient or carrier suitable for delivery to the gut.

In some embodiments of any of the aspects, bacterial species consumes serotonin and/or reduces host biosynthesis of serotonin.

In some embodiments of any of the aspects, the bacterial species is selected from one or more of Bifidobacterium longum, Blautia coccoides, Blautia obeum, Clostridium butyricum, Coprococcus comes, Dorea longicatena, Eubacterium rectale, Lachnoclostridium sp., and Slackia isoflavoniconvertens.

In some embodiments of any of the aspects, the bacterial species is selected from one or more of: Bifidobacterium longum HB-234, Blautia coccoides HB-23, Blautia obeum HB-14, Clostridium butyricum HB-88, Coprococcus comes HB-80, Dorea longicatena HB-17, Eubacterium rectale HB-22, Lachnoclostridium sp. HB-698, and Slackia isoflavoniconvertens HB-326.

In some embodiments of any of the aspects, the isolated bacterial species comprises a 16S sequence at least 95% identical to a sequence selected from SEQ ID Nos 96-104.

In one aspect described herein is a method of decreasing serotonin in a mammalian subject in need thereof, the method comprising administering a composition as described herein to the subject, whereby a serotonin level is decreased.

In some embodiments of any of the aspects, the administering is to the gut of the subject.

In some embodiments of any of the aspects, the level of serotonin in the gut is decreased.

In some embodiments of any of the aspects, the level of serotonin in circulation is decreased.

In one aspect described herein is a method of treating a disease or disorder involving or characterized by high or elevated serotonin in a subject in need thereof, the method comprising administering a composition as described herein to the subject, whereby the disease or disorder is treated.

In some embodiments of any of the aspects, the administering is to the gut of the subject.

In some embodiments of any of the aspects, the level of serotonin in the gut is decreased.

In some embodiments of any of the aspects, the level of serotonin in circulation is decreased.

In some embodiments of any of the aspects, the disease or disorder is selected from the group consisting of diarrhea, IBS-D, inflammatory bowel disease, anxiety, social phobia, sleep disorders, schizophrenia, cancer, obesity, diabetes, coronary artery disease, heart valve disease, congenital heart disease, cardiomyopathies, pericarditis, or platelet disorders (e.g. essential thrombocytosis).

In some embodiments of any of the aspects, the disease or disorder is not a gut disease or disorder.

In some embodiments of any of the aspects, the disease or disorder is selected from the group consisting of anxiety, social phobia, sleep disorders, schizophrenia, cancer, obesity, diabetes, coronary artery disease, heart valve disease, congenital heart disease, cardiomyopathies, pericarditis, or platelet disorders (e.g. essential thrombocytosis).

In one aspect described herein is a therapeutic composition comprising one or more live isolated serotonin-modulating bacteria, dead isolated serotonin modulating bacteria, conditioned medium(s) from an isolated, cultured serotonin-modulating bacteria, cell pellet(s) of isolated serotonin-modulating bacteria, purified metabolite(s) produced by isolated serotonin-modulating bacteria, purified protein(s) produced by an isolated serotonin-modulating bacteria, or a combination thereof, which alter serotonin signaling or biosynthesis in a subject in need thereof.

In some embodiments of any of the aspects, the at least one isolated serotonin-modulating bacteria belongs to a genus selected from the group consisting of: Acidaminococcus, Agathobacter, Adlercreutzia, Akkermansia, Alistipes, Anaerotruncus, Bacillus, Bacteroides, Bifidobacterium, Bilophila, Bittarella, Blautia, Blautia, Butyricimonas, Clostridium, Clostridium, Collinsella, Coprococcus, Dialister, Dorea, Dysosmobacter, Eisenbergiella, Enterococcus, Enterorhabdus, Erysipelatoclostridium, Escherichia, Eubacterium, Faecalitalea, Flavonifractor, Flintibacter, Gemmiger, Gordonibacter, Hungatella, Intestinimonas, Lachnoclostridium, Lactobacillus, Lawsonibacter, Longibaculum, Mediterraneibacter, Mycolicibacterium, Oscillibacter, Parabacteroides, Parasutterella, Peptostreptococcus, Prevotella, Romboutsia, Ruminococcus, Sellimonas, Slackia, Streptococcus, Sutterella, Turicibacter, and Veillonella.

In some embodiments of any of the aspects, the at least one isolated serotonin-modulating bacteria are species selected from the group consisting of: Acidaminococcus intestini, Agathobacter rectalis, Akkermansia muciniphila, Adlercreutzia equolifaciens, Alistipes onderdonkii, Alistipes putredinis, Anaerotruncus colihominis, Bacillus cereus, Bacteroides caccae, Bacteroides cellulosilyticus, Bacteroides clarus, Bacteroides dorei, Bacteroides finegoldii, Bacteroides fragilis, Bacteroides koreensis, Bacteroides ovatus, Bacteroides plebeius, Bacteroides salyersiae, Bacteroides stercoris, Bacteroides thetaiotaomicron, Bacteroides unformis, Bacteroides vulgatus, Bacteroides xylanisolvens, Bifidobacterium adolescentis, Bifidobacterium breve, Bifidobacterium faecale, Bifidobacterium longum, Bilophila wadsworthia, Bittarella massiliensis, Blautia coccoides, Blautia obeum, Blautia wexlerae, Butyricimonas paravirosa, Clostridium asparagiforme, Clostridium aldenese, Clostridium bolteae, Clostridium butyricum, Clostridium clostridioforme, Clostridium hathewayi, Clostridium innoculum, Clostridium lavalense, Clostridium paraputrificum, Clostridium saudiense, Clostridium scindens, Clostridium sp., Clostridium sporogenes, Clostridium sphenoides, Clostridium symbiosum, Clostridium tyrobutyricum, Clostridium hylemonae, Collinsella aerofaciens, Coprococcus comes, Coprococcus eutactus, Dialister invisus, Dorea longicatena, Dysosmobacter welbionis, Eisenbergiella tayi, Enterococcus durans, Enterococcus faecium, Enterorhabdus caecimuris, Enterorhabdus muris, Erysipelatoclostridium ramosum, Escherichia coli, Eubacterium callanderi, Eubacterium eligens, Eubacterium rectale, Faecalitalea cylindroides, Flavonifractorplautii, Flintibacter butyricus, Gemmiger formicilis, Gemmiger sp., Gordonibacter pamelaeae, Hungatella effluvii, Hungatella hathewayi, Intestinimonas butyriciproducens, Intestinimonas massiliensis, Lachnoclostridium sp., Lactobacillus brevis, Lawsonibacter asaccharolyticus, Longibaculum muris, Longibaculum sp., Mediterraneibacter faecis, Mycolicibacterium smegmatis, Oscillibacter sp., Parabacteroides distasonis, Parabacteroides goldsteinii, Parabacteroides johnsonii, Parabacteroides merdae, Parasutterella excrementihominis, Peptostreptococcus russellii, Prevotella copri, Prevotella sp, Prevotella sp., Romboutsia lituseburensis, Ruminococcus bicirculans, Ruminococcus gnavus, Ruminococcus sp., Sellimonas intestinalis, Slackia isoflavoniconvertens, Streptococcus gordonii, Sutterella wadsworthensis, Turicibacter sanguinis, and Veillonella atypica.

In some embodiments of any of the aspects, the one or more serotonin-modulating bacteria include a strain selected from the group consisting of: Acidaminococcus intestini HB-95, Agathobacter rectalis HB-257, Akkermansia muciniphila BAA-835, Adlercreutzia equolifaciens FJC-B9, Alistipes onderdonkii HB-311, Alistipes putredinis HB-324, Anaerotruncus colihominis HB-474, Anaerotruncus colihominis HB-83, Bacillus cereus HB-25, Bacteroides caccae HB-11, Bacteroides cellulosilyticus HB-227, Bacteroides clarus HB-30, Bacteroides dorei HB-12, Bacteroides finegoldii HB-31, Bacteroides fragilis HB-58, Bacteroides koreensis HB-385, Bacteroides ovatus HB-70, Bacteroides plebeius HB-237, Bacteroides salyersiae HB-32, Bacteroides stercoris HB-33, Bacteroides thetaiotaomicron HB-34, Bacteroides uniformis HB-13, Bacteroides vulgatus HB-10, Bacteroides xylanisolvens HB-35, Bifidobacterium adolescentis HB-179, Bifidobacterium breve HB-90, Bifidobacterium faecale HB-159, Bifidobacterium longum HB-234, Bifidobacterium longum HB-71, Bilophila wadsworthia HB-693, Bittarella massiliensis HB-477, Blautia coccoides HB-23, Blautia obeum HB-14, Blautia wexlerae HB-16, Butyricimonas paravirosa HB-453, Clostridium aldenese HB-440, Clostridium bolteae HB-442, Clostridium butyricum HB-88, Clostridium clostridioforme HB-642, Clostridium hathewayi HB-152, Clostridium innoculum HB-82, Clostridium lavalense HB-452c, Clostridium paraputrificum HB-27, Clostridium saudiense HB-142, Clostridium scindens HB-444, Clostridium sp. HB-358, Clostridium sphenoides HB-470, Clostridium sporogenes JCM 7836, Clostridium sporogenes McClung 2004, Clostridium symbiosum HB-67, Clostridium tyrobutyricum HB-469, Clostridium hylemonae HB-73, Collinsella aerofaciens HB-274, Coprococcus comes HB-376, Coprococcus comes HB-80, Coprococcus comes ATCC 27758, Coprococcus eutactus HB-155, Coprococcus eutactus ATCC 27759, Dialister invisus HB-387, Dorea longicatena HB-17, Dysosmobacter welbionis HB-45, Eisenbergiella tayi HB-437, Eisenbergiella tayi HB-612, Enterococcus durans HB-48, Enterococcus faecium HB-640, Enterococcus faecium HB-85, Enterorhabdus caecimuris B7, Enterorhabdus muris WCA-131-CoC-2, Erysipelatoclostridium ramosum HB-24, Escherichia coli HB-490, Eubacterium callanderi HB-59, Eubacterium eligens HB-252, Eubacterium rectale HB-22, Faecalitalea cylindroides HB-664, Flavonifractor plautii HB-472, Flintibacter butyricus HB-344, Gemmiger formicilis HB-325, Gemmiger sp. HB-567, Gordonibacter pamelaeae HB-15, Hungatella effluvii HB-02, Hungatella hathewayi HB-01, Intestinimonas butyriciproducens HB-478, Intestinimonas massiliensis HB-651, Lachnoclostridium sp. HB-698, Lactobacillus brevis HB-87, Lawsonibacter asaccharolyticus HB-521, Longibaculum muris HB-79, Longibaculum sp. HB-681, Mediterraneibacter faecis HB-364, Mycolicibacterium smegmatis ATCC 19420, Oscillibacter sp. HB-28, Parabacteroides distasonis HB-20, Parabacteroides distasonis HB-214, Parabacteroides goldsteinii HB-44, Parabacteroides johnsonii HB-03, Parabacteroides merdae HB-63, Parasutterella excrementihominis HB-330, Peptostreptococcus russeliii RT-10B, Prevotella copri HB-373, Prevotella sp HB-649, Prevotella sp. HB-333, Romboutsia lituseburensis HB-102, Ruminococcus bicirculans HB-105, Ruminococcus bicirculans HB-268, Ruminococcus gnavus HB-40, Ruminococcus gnavus HB-516, Ruminococcus sp. HB-626, Sellimonas intestinalis HB-443, Slackia isoflavoniconvertens HB-326, Streptococcus gordonii HB-62, Streptococcus gordonii HB-98, Sutterella wadsworthensis HB-259, Turicibacter sanguinis HB-147, and Veillonella atypica HB-251.

In some embodiments of any of the aspects, the one or more serotonin-modulating bacteria consists of a bacteria comprising a 16S rDNA sequence at least about 95% identical to a 16S rDNA sequence selected from one of SEQ ID NOs: 1-114.

In some embodiments of any of the aspects, the serotonin-modulating bacteria encode genes in their genome, which when expressed, result in the production of one or more metabolites or proteins that influence subject serotonin signaling/biosynthesis.

In some embodiments of any of the aspects, the encoded genes are expressed at physiologically relevant conditions of the human gastrointestinal tract, resulting in the production of metabolites or proteins that influence subject serotonin signaling/biosynthesis.

In some embodiments of any of the aspects, the composition is in the form of a probiotic, prebiotic, a capsule, a tablet, a caplet, a pill, a troche, a lozenge, a powder, a granule, a medical food, a supplement, or a combination thereof.

In some embodiments of any of the aspects, the composition is formulated to be administered orally, intravenously, intramuscularly, intrathecally, subcutaneously, sublingually, buccally, rectally, vaginally, by the ocular route, by the otic route, nasally, via inhalation, by nebulization, cutaneously, transdermally, or a combination thereof.

In one aspect described herein is a pharmaceutical composition comprising the therapeutic composition as described herein, and a pharmaceutically acceptable carrier.

In one aspect described herein is a method of treating a disease or disorder in a subject in need thereof, the method comprising administering to the subject an effective amount of a therapeutic composition comprising one or more live isolated serotonin-modulating bacteria, dead isolated serotonin modulating bacteria, conditioned medium(s) derived from an isolated serotonin-modulating bacteria, cell pellet(s) of isolated serotonin-modulating bacteria, purified metabolite(s) produced by isolated serotonin-modulating bacteria, purified protein(s) produced by isolated serotonin-modulating bacteria, or a combination thereof, thereby altering serotonin signaling or biosynthesis in the subject to treat the disease or disorder.

In some embodiments of any of the aspects, the disease or disorder is a serotonin-related disease or disorder.

In some embodiments of any of the aspects, the serotonin-related disease or disorder is selected from the group consisting of intestinal motility disorders, irritable bowel syndrome, inflammatory bowel disease, depression (e.g. major depressive disorder, treatment resistant depression, post-partum depression), anxiety disorders, addiction, social phobia, neurodegenerative disorders, autism spectrum disorder, sleep disorders, attention deficit hyperactivity disorder (ADHD), memory loss (e.g. dementia), learning difficulties, sleep disorders, schizophrenia, bone disease (e.g. osteoporosis), cancer (e.g. polycythemia vera or myelosclerosis), metabolic disease (e.g. obesity or diabetes), a dysregulated immune system, cardiac disease (e.g. coronary artery disease, heart valve disease, congenital heart disease, cardiomyopathies, pericarditis, or aorta disease), heartburn, dermatological conditions (e.g. eczema and itch), GERD, platelet disorders (e.g. essential thrombocytosis), and pain disorders.

In some embodiments of any of the aspects, the disease or disorder is caused by high serotonin levels and is selected from the group: diarrhea, IBS-D, inflammatory bowel disease, anxiety, social phobia, sleep disorders, schizophrenia, cancer, obesity, diabetes, coronary artery disease, heart valve disease, congenital heart disease, cardiomyopathies, pericarditis, or platelet disorders (e.g. essential thrombocytosis).

In some embodiments of any of the aspects, the disease or disorder is caused by low serotonin levels and is selected from the group: constipation, IBS-C, depression, anxiety, addiction, neurodegenerative disorders, autism spectrum disorder, sleep disorders, attention deficit hyperactivity disorder (ADHD), memory loss (e.g. dementia), learning difficulties, osteoporosis, heartburn, dermatological conditions (eczema and itch), GERD, or pain disorders.

In some embodiments of any of the aspects, treating a disease or disorder comprises decreasing at least one symptom of the disease or disorder, selected from: fatigue, insomnia, stress, persistent anxiety, persistent sadness, social withdrawal, substance withdrawal, irritability, thoughts of suicide, thoughts of self-harm, restlessness, low sex drive, lack of focus, loss of appetite, high blood pressure, low blood pressure, high heart rate, low heart rate, constipation, diarrhea, chronic pain, heartburn, fatigue, trouble breathing, stomach aches, nosebleeds, gum, stomach bleeding, headaches, weight gain, burning of the skin, altered inflammatory markers, neurodevelopmental deficits, and/or seizures.

In some embodiments of any of the aspects, the at least one isolated serotonin-modulating bacteria belongs to a genus selected from the group consisting of: Acidaminococcus, Agathobacter, Adlercreutzia, Akkermansia, Alistipes, Anaerotruncus, Bacillus, Bacteroides, Bifidobacterium, Bilophila, Bittarella, Blautia, Blautia, Butyricimonas, Clostridium, Clostridium, Collinsella, Coprococcus, Dialister, Dorea, Dysosmobacter, Eisenbergiella, Enterococcus, Enterorhabdus, Erysipelatoclostridium, Escherichia, Eubacterium, Faecalitalea, Flavonifractor, Flintibacter, Gemmiger, Gordonibacter, Hungatella, Intestinimonas, Lachnoclostridium, Lactobacillus, Lawsonibacter, Longibaculum, Mediterraneibacter, Mycolicibacterium, Oscillibacter, Parabacteroides, Parasutterella, Peptostreptococcus, Prevotella, Romboutsia, Ruminococcus, Sellimonas, Slackia, Streptococcus, Sutterella, Turicibacter, and Veillonella.

In some embodiments of any of the aspects, the at least one isolated serotonin-modulating bacteria are species selected from the group consisting of: Acidaminococcus intestini, Agathobacter rectalis, Akkermansia muciniphila, Adlercreutzia equolifaciens, Alistipes onderdonkii, Alistipes putredinis, Anaerotruncus colihominis, Bacillus cereus, Bacteroides caccae, Bacteroides cellulosilyticus, Bacteroides clarus, Bacteroides dorei, Bacteroides finegoldii, Bacteroides fragilis, Bacteroides koreensis, Bacteroides ovatus, Bacteroides plebeius, Bacteroides salyersiae, Bacteroides stercoris, Bacteroides thetaiotaomicron, Bacteroides unformis, Bacteroides vulgatus, Bacteroides xylanisolvens, Bifidobacterium adolescentis, Bifidobacterium breve, Bifidobacterium faecale, Bifidobacterium longum, Bilophila wadsworthia, Bittarella massiliensis, Blautia coccoides, Blautia obeum, Blautia wexlerae, Butyricimonas paravirosa, Clostridium asparagiforme, Clostridium aldenese, Clostridium bolteae, Clostridium butyricum, Clostridium clostridioforme, Clostridium hathewayi, Clostridium innoculum, Clostridium lavalense, Clostridium paraputrificum, Clostridium saudiense, Clostridium scindens, Clostridium sp., Clostridium sporogenes, Clostridium sphenoides, Clostridium symbiosum, Clostridium tyrobutyricum, Clostridium hylemonae, Collinsella aerofaciens, Coprococcus comes, Coprococcus eutactus, Dialister invisus, Dorea longicatena, Dysosmobacter welbionis, Eisenbergiella tayi, Enterococcus durans, Enterococcus faecium, Enterorhabdus caecimuris, Enterorhabdus muris, Erysipelatoclostridium ramosum, Escherichia coli, Eubacterium callanderi, Eubacterium eligens, Eubacterium rectale, Faecalitalea cylindroides, Flavonifractorplautii, Flintibacter butyricus, Gemmiger formicilis, Gemmiger sp., Gordonibacter pamelaeae, Hungatella effluvii, Hungatella hathewayi, Intestinimonas butyriciproducens, Intestinimonas massiliensis, Lachnoclostridium sp., Lactobacillus brevis, Lawsonibacter asaccharolyticus, Longibaculum muris, Longibaculum sp., Mediterraneibacter faecis, Mycolicibacterium smegmatis, Oscillibacter sp., Parabacteroides distasonis, Parabacteroides goldsteinii, Parabacteroides johnsonii, Parabacteroides merdae, Parasutterella excrementihominis, Peptostreptococcus russellii, Prevotella copri, Prevotella sp, Prevotella sp., Romboutsia lituseburensis, Ruminococcus bicirculans, Ruminococcus gnavus, Ruminococcus sp., Sellimonas intestinalis, Slackia isoflavoniconvertens, Streptococcus gordonii, Sutterella wadsworthensis, Turicibacter sanguinis, and Veillonella atypica.

In some embodiments of any of the aspects, the one or more serotonin-modulating bacteria include a strain selected from the group consisting of: Acidaminococcus intestini HB-95, Agathobacter rectalis HB-257, Akkermansia muciniphila BAA-835, Adlercreutzia equolifaciens FJC-B9, Alistipes onderdonkii HB-311, Alistipes putredinis HB-324, Anaerotruncus colihominis HB-474, Anaerotruncus colihominis HB-83, Bacillus cereus HB-25, Bacteroides caccae HB-11, Bacteroides cellulosilyticus HB-227, Bacteroides clarus HB-30, Bacteroides dorei HB-12, Bacteroides finegoldii HB-31, Bacteroides fragilis HB-58, Bacteroides koreensis HB-385, Bacteroides ovatus HB-70, Bacteroides plebeius HB-237, Bacteroides salyersiae HB-32, Bacteroides stercoris HB-33, Bacteroides thetaiotaomicron HB-34, Bacteroides uniformis HB-13, Bacteroides vulgatus HB-10, Bacteroides xylanisolvens HB-35, Bifidobacterium adolescentis HB-179, Bifidobacterium breve HB-90, Bifidobacterium faecale HB-159, Bifidobacterium longum HB-234, Bifidobacterium longum HB-71, Bilophila wadsworthia HB-693, Bittarella massiliensis HB-477, Blautia coccoides HB-23, Blautia obeum HB-14, Blautia wexlerae HB-16, Butyricimonas paravirosa HB-453, Clostridium aldenese HB-440, Clostridium bolteae HB-442, Clostridium butyricum HB-88, Clostridium clostridioforme HB-642, Clostridium hathewayi HB-152, Clostridium innoculum HB-82, Clostridium lavalense HB-452c, Clostridium paraputrificum HB-27, Clostridium saudiense HB-142, Clostridium scindens HB-444, Clostridium sp. HB-358, Clostridium sphenoides HB-470, Clostridium sporogenes JCM 7836, Clostridium sporogenes McClung 2004, Clostridium symbiosum HB-67, Clostridium tyrobutyricum HB-469, Clostridium hylemonae HB-73, Collinsella aerofaciens HB-274, Coprococcus comes HB-376, Coprococcus comes HB-80, Coprococcus comes ATCC 27758, Coprococcus eutactus HB-155, Coprococcus eutactus ATCC 27759, Dialister invisus HB-387, Dorea longicatena HB-17, Dysosmobacter welbionis HB-45, Eisenbergiella tayi HB-437, Eisenbergiella tayi HB-612, Enterococcus durans HB-48, Enterococcus faecium HB-640, Enterococcus faecium HB-85, Enterorhabdus caecimuris B7, Enterorhabdus muris WCA-131-CoC-2, Erysipelatoclostridium ramosum HB-24, Escherichia coli HB-490, Eubacterium callanderi HB-59, Eubacterium eligens HB-252, Eubacterium rectale HB-22, Faecalitalea cylindroides HB-664, Flavonifractor plautii HB-472, Flintibacter butyricus HB-344, Gemmiger formicilis HB-325, Gemmiger sp. HB-567, Gordonibacter pamelaeae HB-15, Hungatella effluvii HB-02, Hungatella hathewayi HB-01, Intestinimonas butyriciproducens HB-478, Intestinimonas massiliensis HB-651, Lachnoclostridium sp. HB-698, Lactobacillus brevis HB-87, Lawsonibacter asaccharolyticus HB-521, Longibaculum muris HB-79, Longibaculum sp. HB-681, Mediterraneibacter faecis HB-364, Mycolicibacterium smegmatis ATCC 19420, Oscillibacter sp. HB-28, Parabacteroides distasonis HB-20, Parabacteroides distasonis HB-214, Parabacteroides goldsteinii HB-44, Parabacteroides johnsonii HB-03, Parabacteroides merdae HB-63, Parasutterella excrementihominis HB-330, Peptostreptococcus russeliii RT-10B, Prevotella copri HB-373, Prevotella sp HB-649, Prevotella sp. HB-333, Romboutsia lituseburensis HB-102, Ruminococcus bicirculans HB-105, Ruminococcus bicirculans HB-268, Ruminococcus gnavus HB-40, Ruminococcus gnavus HB-516, Ruminococcus sp. HB-626, Sellimonas intestinalis HB-443, Slackia isoflavoniconvertens HB-326, Streptococcus gordonii HB-62, Streptococcus gordonii HB-98, Sutterella wadsworthensis HB-259, Turicibacter sanguinis HB-147, and Veillonella atypica HB-251.

In some embodiments of any of the aspects, the one or more serotonin-modulating bacteria consists of a bacteria comprising a 16S rDNA sequence at least about 95% identical to a 16S rDNA sequence selected from one of SEQ ID NOs: 1-114.

In some embodiments of any of the aspects, the serotonin-modulating bacteria encode genes in their genome, which when expressed, result in the production of one or more metabolites or proteins that influence subject serotonin signaling/biosynthesis.

In some embodiments of any of the aspects, the encoded genes are expressed at physiologically relevant conditions of the human gastrointestinal tract, resulting in the production of metabolites or proteins that influence subject serotonin signaling/biosynthesis.

In some embodiments of any of the aspects, the composition is in the form of a probiotic, prebiotic, a capsule, a tablet, a caplet, a pill, a troche, a lozenge, a powders, a granule, a medical food, supplement or a combination thereof.

In some embodiments of any of the aspects, the composition is administered orally, intravenously, intramuscularly, intrathecally, subcutaneously, sublingually, buccally, rectally, vaginally, by the ocular route, by the otic route, nasally, via inhalation, by nebulization, cutaneously, transdermally, or a combination thereof

In some embodiments of any of the aspects, the method further comprises identifying a subject in need of treatment by measuring a serotonin level in a sample from the subject, and comparing the level to a reference level.

In some embodiments of any of the aspects, the serotonin level is measured in stool, blood, or tissue of the subject.

In some embodiments of any of the aspects, a serotonin level less than the reference level identifies a subject in need of treatment.

In some embodiments of any of the aspects, the levels of serotonin in the stool, blood, or tissue of the subject are altered relative to their initial quantitated amounts, after administering the therapeutic composition.

In some embodiments of any of the aspects, the method further comprises identifying a subject in need of treatment by measuring levels of fecal serotonin modulating bacteria.

In some embodiments of any of the aspects, the level of fecal serotonin-modulating bacteria is measured by fecal 16S rDNA sequencing, fecal shotgun metagenomic sequencing, measurement of fecal genes involved in the production of microbiota-derived serotonin modulating metabolites, measurement of proteins by sequencing or proteomics or comparable methods, or levels of fecal, blood, or tissue serotonin-modulating metabolites via LC/MS or comparable methods.

In some embodiments of any of the aspects, the levels of serotonin modulating bacteria, genes involved in the production of microbiota-derived serotonin modulating metabolites or proteins, or levels of serotonin-modulating metabolites are altered relative to their initial quantitated amounts after administering the therapeutic composition.

### BRIEF DESCRIPTION OF THE DRAWINGS

FIG. 1 is a schematic showing a phylogenetic tree generated to identify microbial enzymes and the microbes that produce them that can carry out the tryptophan decarboxylase function that generates tryptamine from tryptophan (see e.g., SEQ ID NOs: 185-226).

FIG. 2A-2B are a series of bar graphs showing that supernatants and cell pellets from 5-HT modulating bacterial strains increase mammalian Tryptophan Hydroxylase 1 (TPH-1) expression. (FIG. 2A) Supernatant and (FIG. 2B) cell pellets from 48-hour cultures of 5-HT modulating bacteria were introduced into cultures of RIN14B pancreas cells. Expression of Tryptophan Hydroxylase 1, a rate limiting step of 5-HT biosynthesis, was measured via qPCR. HM2 indicates the bacterial medium.

FIG. 3 is a bar graph showing that 5-HT modulating bacteria elevate 5-HT signaling in a human gut simulator complex background using bacterial supernatants. Human fecal samples were spiked into an in-house human gut simulator, creating a mock microbiome community. This community normalized for 48 hours, and then 10\hat{ }8 CFU of 5-HT modulating bacteria alone or in combination were spiked into the system. The supernatants of this system were then collected after 48 hours, spiked into RIN14B rat pancreas cell culture at 50% final volume, incubated at 37° C. in a CO₂ incubator for an hour, and 5-HT is measured via ELISA. HD-3=donor microbiome background spiked only with vehicle, buffer=HBSS, ionomycin=positive control, Media 10% and 50% are basal mediums used in the gut simulator.

FIG. 4 is a bar graph showing that 5-HT modulating bacteria elevate 5-HT signaling in a human gut simulator complex background using bacterial cell pellets. Human fecal samples were loaded into an in-house human gut simulator, creating a mock microbiome community. This community normalized for 48 hours, and then 10\hat{ }8 CFU of 5-HT modulating bacteria alone or in combination were spiked into the system. The cell pellets of this system were then collected after 48 hours, spiked into RIN14B rat pancreas cell culture at 100 ng/mL, incubated at 37° C. in a CO₂ incubator for an hour, and 5-HT was measured via ELISA. HD-3=donor microbiome background spiked only with vehicle, buffer=HBSS, ionomycin=positive control, Media 10% and 50% are basal mediums used in the gut simulator.

### DETAILED DESCRIPTION

Embodiments of the technology described herein provide compositions and methods for decreasing at least one symptom of a serotonin-related disease or disorder in a subject in need thereof by altering the serotonin-modulating gut microbiota, serotonin-modulating gut-microbiota-derived metabolome, or serotonin-modulating gut-microbiota-derived proteome of the subject. The compositions can comprise one or more live serotonin-modulating bacteria, one or more dead-serotonin modulating bacteria, one or more conditioned medium(s) of serotonin-modulating bacteria, one or more cell pellet(s) of serotonin-modulating bacteria, and/or one or more 5-HT agonists, metabolites and/or proteins (derived from serotonin-modulating bacteria), or therapeutic or pharmaceutical compositions comprising the same. The method can comprise administering combinations of one or more live serotonin-modulating bacteria, one or more dead-serotonin modulating bacteria, one or more conditioned medium(s) of serotonin-modulating bacteria, one or more cell pellet(s) of serotonin-modulating bacteria, and/or one or more 5-HT agonists, metabolites and/or proteins (derived from serotonin-modulating bacteria).

The present technology has the advantage of alleviating the symptoms of serotonin-related disease or disorders without the aid of synthetic medications (e.g., serotonin-reuptake inhibitors), which can have unwanted side-effects, or in combination with existing medications. Additionally, the present technology can have the advantage of further improving other aspects of health of the subject, as the bacteria can perform multiple mechanisms (e.g. alter serotonin signaling but also alter the immune system). Additional features and advantages of the present technology will be apparent to one of ordinary skill in the art.

### Serotonin

The term “Serotonin” should be understood as referring to 5-hydroxytryptamine, which has the below chemical structure:

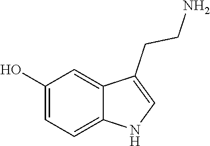 [SMILES: NCCc1c[nH]c2ccc(O)cc12]

Serotonin is a monoamine neurotransmitter involved in a wide range of physiological processes, including mood, anxiety, sleep, appetite, temperature, eating behavior, sexual behavior, movements and gastrointestinal motility. Serotonin is synthesized from the amino acid L-tryptophan by two enzymes—tryptophan hydroxylase (TPH) and aromatic amino acid decarboxylase (DDC), which can be expressed by the host.

More than 90% of the body's 5-HT is produced in the gastrointestinal (GI) tract. In many instances said 5-HT is produced by host gut cells under the influence of the gut microbiota, not produced by the microbiota itself. In the GI tract, 5-HT activates as many as 14 different 5-HT receptor subtypes, including those found on immune cells, enterocytes, and enteric nerves. In addition, circulating platelets sequester 5-HT from the GI tract, releasing it to promote hemostasis and distributing it to various body sites. As such, gut-derived 5-HT regulates diverse functions, including the immune response and intestinal motility. Perhaps not surprising due to its function, a disrupted serotonergic system appears to be strongly associated with symptomologies of IBS, particularly for IBS-C.

### Serotonin and the Microbiome

The microbiome plays a major role in influencing serotonergic neurotransmission. In germ free animals, there is a significant reduction of serotonin in the blood and colon of mice compared to controls, a feature which is associated with reduced intestinal motility. Both serotonin levels and normal GI transit motility can be restored via recolonization with a consortium of “spore-forming” bacteria. These general findings were repeated using a single species of bacteria, Clostridium ramosum, which when introduced into germ-free mice restored serotonin levels in the cecum and serum. Further supporting this microbiome-serotonin connection is a study wherein it was found that the disrupted intestinal motility and damaged GI barrier phenotype of chronic constipation could be transplanted from humans to rodents via fecal microbiome transplant (FMT). No effect was observed when the microbiome from healthy humans was transferred. This phenotype was associated with reduced serum levels of serotonin as well as increased expression of the serotonin transporter SERT.

The present disclosure provides compositions and methods for decreasing at least one symptom of a serotonin-related disease or disorder in a subject in need thereof by altering the serotonin-modulating gut microbiota, serotonin-modulating gut-microbiota-derived metabolome, or serotonin-modulating gut-microbiota-derived proteome of the subject. For more information concerning the microbiome and/or serotonin, see e.g., Mayer et al. Neuroscience 34, 15490-15496 (2014); Lynch & Pedersen, N Engl J Med 375, 2369-2379 (2016); Fung et al. Nat Neurosci 20, 145-155 (2017). Strandwitz, Brain Res 1693, 128-133 (2018); Agus et al., Cell host & microbe 23, 716-724 (2018); d'Hennezelv et al., mSystems 2, e00046-17 (2017); Skelly et al., Nat Rev Immunol, 2019 May, 19(5):305-323; Gershon & Tack, Gastroenterology 132, 397-414 (2007); Amireault et al., ACS Chem Neurosci 4, 64-71 (2013); Matthes & Bader, Trends Pharmacol Sci 39, 560-572 (2018); Spohn & Mawe, Nat Rev Gastroenterol Hepatol 14, 412-420 (2017); Wikoff et al. Proc Natl Acad Sci USA 106, 3698-3703, (2009); Tumbaugh et al., Nature 444, 1027-1031 (2006); Rooks et al., ISME J 8, 1403-1417 (2014); Zackular et al., MBio 4, e00692-00613 (2013); Petrof et al., Microbiome 1, 3 (2013); Baganz & Blakely, ACS Chem Neurosci 4, 48-63 (2013); Sikander et al., Clin Chim Acta 403, 47-55 (2009); Yano et al., Cell 161, 264-276 (2015); Mandic et al., Sci Rep 9, 1177 (2019); Cao et al. Sci Rep 7, 10322 (2017); the contents of each of which are incorporated by reference herein in their entireties.

### Serotonin Modulation by Bacteria

The microbiota can modulate a subject's serotonin signaling and/or biosynthesis by producing specific metabolites which are sensed by a subject's cells resulting in alterations in host gene expression, protein activity, or metabolic output/requirements, influencing serotonin biosynthesis and salvage pathways. In some embodiments, a bacterial species can produce a co-factor or nutrient that is used in these pathways, stimulating activity of host serotonin biosynthesis. In other embodiments, the microbiota can alternatively remove nutrients from the host, via consumption or secretion of secondary metabolites that bind to nutrients (e.g. siderophores, which bind soluble iron, an essential nutrient), preventing or reducing biosynthesis of serotonin.

The microbiota can also produce proteins including agonist or other proteins that interact with host receptors that sense microbial components. In some embodiments, these proteins are sensed by Toll-like receptors (TLRs), which signal to the host to produce or restrict production of serotonin.

The microbiota can also modulate pharmacological agents, which are intended to interact with a subject's serotonin signaling/biosynthesis pathways. In some embodiments, these can be agonists or antagonists of Tph1 and Tph2, SERT, 5-HT_{1A}, 5-HT_{IB}, 5-HT_{1D}, 5-HT_{1E}, 5-HT_{1F}, 5-HT_{2A}, 5-HT_{2B}, 5-HT_{2C}, 5-HT₃, 5-HT₄, 5-HT_{5A}, 5-HT_{5B}, 5-HT₆, 5-HT₇, among others. In some embodiments, compositions, described below, can be employed to alter these biotransformations.

### Serotonin-Modulating Bacterial Compositions

In some embodiments, described herein are therapeutic compositions comprising live serotonin-modulating bacteria, dead or inactivated serotonin-modulating bacteria, conditioned medium(s) of cultured serotonin-modulating bacteria, cell pellet(s) of serotonin-modulating bacteria, and/or metabolites and/or proteins derived from serotonin-modulating bacteria, that are delivered to the gastrointestinal tract of the subject to modulate serotonin signaling and/or biosynthesis of the subject, either directly or by altering native microbial (e.g., bacterial, archaeal, fungal, protist, or viral) community composition or gene expression, resulting in increased or reduced levels of serotonin-modulating bacteria, or alterations in the microbiota-derived metabolome and/or microbiota-derived proteome to a more serotonin-stimulating or serotonin-inhibitory state.

In some embodiments, the serotonin-modulating gut microbiota, serotonin-modulating gut-microbiota-derived metabolome, or serotonin-modulating gut-microbiota-derived proteome of the subject is altered by increasing/decreasing the number of at least one serotonin-modulating bacteria by administering an effective amount of a microbiota modulator selected from: serotonin-modulating bacteria, probiotics, antimicrobials, species-specific antimicrobials, prebiotics, bacteriophages, genetic elements (e.g. CRISPR) or any combination thereof.

In some embodiments, the serotonin-modulating bacteria encode one or more genes in their genome, which when expressed, result in the production of one or more metabolites or proteins that influence subject serotonin signaling/biosynthesis. In some embodiments, these genes are expressed at physiologically relevant conditions of the human gastrointestinal tract. Non-limiting examples of such enzymes, and the species that encode and express them, are described further herein and specifically indicated in the sequence listing.

In some embodiments, the serotonin-modulating bacterial species are serotonin-increasing bacterial species. In some embodiments, the serotonin-increasing bacterial species increases serotonin in at least one of the following ways: production of serotonin; production of secreted metabolites or secreted proteins that induce serotonin production; production of ligands that induce serotonin production; production of an agonist (e.g., of a serotonin receptor or the trace amine-associated receptor (TAAR)); or any combination thereof.

### Bacterial Compositions

In some embodiments, described herein are therapeutic compositions comprising one or more bacteria (e.g. purified bacteria) that are capable of increasing or decreasing subject serotonin levels in a subject in need thereof, when delivered to the intestinal tract. The bacteria can be capable of producing metabolites or proteins at physiologically relevant conditions, such as those conditions found in the human gut, that modulate subject serotonin signaling/biosynthesis in the body (e.g. the gastrointestinal tract, the circulatory system, or the brain). The bacteria can also be capable of altering native microbial (e.g., bacterial, archaeal, fungal, protist, or viral) community composition or gene expression, resulting in increased or reduced levels of serotonin-modulating bacteria, or alterations in the microbiota-derived metabolome and/or microbiota-derived proteome to a more serotonin-stimulating or serotonin-inhibitory state. In some embodiments, the one or more bacteria are not viable, or the one or more bacteria comprise a combination of viable and non-viable bacteria. In some embodiments, the bacterial composition comprises at least 1, 2, 3, 4, 5, 6, 7, 8, 9, or 10, or more, types of isolated bacteria. Where non-viable bacteria or a component thereof can modulate serotonin, it is likely that a factor, e.g., a protein or other factor comprised by the bacteria, or a collection of such factors is involved in the serotonin-inducing effects, rather than simply a metabolite produced by the bacteria.

In some embodiments, the composition comprises a consortium of serotonin-modulating bacteria. As a non-limiting example, the consortium can comprise at least 2 species or strains, at least 3 species or strains, at least 4 species or strains, at least 5 species or strains, at least 6 species or strains, at least 7 species or strains, at least 8 species or strains, at least 9 species or strains, at least 10 species or strains, at least 11 species or strains, at least 12 species or strains, at least 13 species or strains, at least 14 species or strains, at least 15 species or strains, at least 16 species or strains, at least 17 species or strains, at least 18 species or strains, at least 19 species or strains, or at least 20 species or strains of serotonin-modulating bacteria. In some embodiments, a consortium comprises fewer than 50 species or strains of serotonin-modulating bacteria, fewer than 40 species or strains, fewer than 30 species or strains, fewer than 25 species or strains, fewer than 20 species or strains, fewer than 19 species or strains, fewer than 18 species or strains, fewer than 17 species or strains, fewer than 16 species or strains, fewer than 15 species or strains, fewer than 14 species or strains, fewer than 13 species or strains, fewer than 12 species or strains, fewer than 11 species or strains, fewer than 10 species or strains, fewer than 9 species or strains, fewer than 8 species or strains, fewer than 7 species or strains, fewer than 6 species or strains, fewer than 5 species or strains, fewer than 4 species or strains, or fewer than 3 species or strains of serotonin-modulating bacteria. In one embodiment, a consortium comprises species that modulate serotonin production via different mechanisms. Such consortia can provide additive or synergistic effects on serotonin levels. As a non-limiting example, in one embodiment, a consortium comprises a species that encodes and expresses one or more enzymes that generate serotonin from one or more biosynthetic precursor substrates and a species that stimulates host serotonin production. As another non-limiting example, in one embodiment, a consortium comprises a first species that encodes and expresses one or more enzymes for the production of a biosynthetic serotonin precursor, and a second species that encodes and expresses one or more enzymes that convert the biosynthetic serotonin precursor to serotonin.

In some embodiments, combinations of bacteria are selected for therapeutics, food, medical foods, or any other product for synergistic effects on host 5-HT signaling. In a non-limiting example, combinations of bacteria can be selected to capture multiple mechanisms involved in modulating host 5-HT signaling. As a non-limiting example, a strain with only the ability to elevate 5-HT signaling via its cell pellet can be combined with a separate bacterium, wherein said second bacterium has a supernatant that elicits an effect, produces 5-HT, and/or or produces 5-HT agonists. In some embodiments, by combining mechanisms or adding redundancy of any mechanism (e.g., multiple organisms with the capability to produce 5-HT) within a product, one can to elicit a stronger or more consistent effect on host 5-HT signaling, and thus a more favorable impact in the target indications and/or symptoms. In a non-limiting example, Clostridium lavalense HB-452C, which produces 5-HT and the 5-HT agonist tryptamine, can be combined with Bifidobacterium adolescentis HB-179, which has a strong cell pellet and supernatant induction phenotype.

In some embodiments, the one or more serotonin-modulating bacteria belong to a genus selected from the group consisting of: Acidaminococcus, Agathobacter, Adlercreutzia, Akkermansia, Alistipes, Anaerotruncus, Bacillus, Bacteroides, Bifidobacterium, Bilophila, Bittarella, Blautia, Blautia, Butyricimonas, Clostridium, Clostridium, Collinsella, Coprococcus, Dialister, Dorea, Dysosmobacter, Eisenbergiella, Enterococcus, Enterorhabdus, Erysipelatoclostridium, Escherichia, Eubacterium, Faecalitalea, Flavonifractor, Flintibacter, Gemmiger, Gordonibacter, Hungatella, Intestinimonas, Lachnoclostridium, Lactobacillus, Lawsonibacter, Longibaculum, Mediterraneibacter, Mycolicibacterium, Oscillibacter, Parabacteroides, Parasutterella, Peptostreptococcus, Prevotella, Romboutsia, Ruminococcus, Sellimonas, Slackia, Streptococcus, Sutterella, Turicibacter, and Veillonella.

In some embodiments, the one or more serotonin-modulating bacteria are species selected from the group consisting of: Acidaminococcus intestini, Agathobacter rectalis, Akkermansia muciniphila, Adlercreutzia equolifaciens, Alistipes onderdonkii, Alistipes putredinis, Anaerotruncus colihominis, Bacillus cereus, Bacteroides caccae, Bacteroides cellulosilyticus, Bacteroides clarus, Bacteroides dorei, Bacteroides finegoldii, Bacteroides fragilis, Bacteroides koreensis, Bacteroides ovatus, Bacteroides plebeius, Bacteroides salyersiae, Bacteroides stercoris, Bacteroides thetaiotaomicron, Bacteroides unformis, Bacteroides vulgatus, Bacteroides xylanisolvens, Bifidobacterium adolescentis, Bifidobacterium breve, Bifidobacterium faecale, Bifidobacterium longum, Bilophila wadsworthia, Bittarella massiliensis, Blautia coccoides, Blautia obeum, Blautia wexlerae, Butyricimonas paravirosa, Clostridium asparagiforme, Clostridium aldenese, Clostridium bolteae, Clostridium butyricum, Clostridium clostridioforme, Clostridium hathewayi, Clostridium innoculum, Clostridium lavalense, Clostridium paraputrificum, Clostridium saudiense, Clostridium scindens, Clostridium sp., Clostridium sporogenes, Clostridium sphenoides, Clostridium symbiosum, Clostridium tyrobutyricum, Clostridium hylemonae, Collinsella aerofaciens, Coprococcus comes, Coprococcus eutactus, Dialister invisus, Dorea longicatena, Dysosmobacter welbionis, Eisenbergiella tayi, Enterococcus durans, Enterococcus faecium, Enterorhabdus caecimuris, Enterorhabdus muris, Erysipelatoclostridium ramosum, Escherichia coli, Eubacterium callanderi, Eubacterium eligens, Eubacterium rectale, Faecalitalea cylindroides, Flavonifractor plautii, Flintibacter butyricus, Gemmiger formicilis, Gemmiger sp., Gordonibacter pamelaeae, Hungatella effluvii, Hungatella hathewayi, Intestinimonas butyriciproducens, Intestinimonas massiliensis, Lachnoclostridium sp., Lactobacillus brevis, Lawsonibacter asaccharolyticus, Longibaculum muris, Longibaculum sp., Mediterraneibacter faecis, Mycolicibacterium smegmatis, Oscillibacter sp., Parabacteroides distasonis, Parabacteroides goldsteinii, Parabacteroides johnsonii, Parabacteroides merdae, Parasutterella excrementihominis, Peptostreptococcus russellii, Prevotella copri, Prevotella sp, Prevotella sp., Romboutsia lituseburensis, Ruminococcus bicirculans, Ruminococcus gnavus, Ruminococcus sp., Sellimonas intestinalis, Slackia isoflavoniconvertens, Streptococcus gordonii, Sutterella wadsworthensis, Turicibacter sanguinis, and Veillonella atypica.

In some embodiments, the one or more serotonin-modulating bacteria include a strain selected from the group consisting of: Acidaminococcus intestini HB-95, Agathobacter rectalis HB-257, Akkermansia muciniphila BAA-835, Adlercreutzia equolifaciens FJC-B9, Alistipes onderdonkii HB-311, Alistipes putredinis HB-324, Anaerotruncus colihominis HB-474, Anaerotruncus colihominis HB-83, Bacillus cereus HB-25, Bacteroides caccae HB-11, Bacteroides cellulosilyticus HB-227, Bacteroides clarus HB-30, Bacteroides dorei HB-12, Bacteroides finegoldii HB-31, Bacteroides fragilis HB-58, Bacteroides koreensis HB-385, Bacteroides ovatus HB-70, Bacteroides plebeius HB-237, Bacteroides salyersiae HB-32, Bacteroides stercoris HB-33, Bacteroides thetaiotaomicron HB-34, Bacteroides uniformis HB-13, Bacteroides vulgatus HB-10, Bacteroides xylanisolvens HB-35, Bifidobacterium adolescentis HB-179, Bifidobacterium breve HB-90, Bifidobacterium faecale HB-159, Bifidobacterium longum HB-234, Bifidobacterium longum HB-71, Bilophila wadsworthia HB-693, Bittarella massiliensis HB-477, Blautia coccoides HB-23, Blautia obeum HB-14, Blautia wexlerae HB-16, Butyricimonas paravirosa HB-453, Clostridium aldenese HB-440, Clostridium bolteae HB-442, Clostridium butyricum HB-88, Clostridium clostridioforme HB-642, Clostridium hathewayi HB-152, Clostridium innoculum HB-82, Clostridium lavalense HB-452c, Clostridium paraputrificum HB-27, Clostridium saudiense HB-142, Clostridium scindens HB-444, Clostridium sp. HB-358, Clostridium sphenoides HB-470, Clostridium sporogenes JCM 7836, Clostridium sporogenes McClung 2004, Clostridium symbiosum HB-67, Clostridium tyrobutyricum HB-469, Clostridium hylemonae HB-73, Collinsella aerofaciens HB-274, Coprococcus comes HB-376, Coprococcus comes HB-80, Coprococcus comes ATCC 27758, Coprococcus eutactus HB-155, Coprococcus eutactus ATCC 27759, Dialister invisus HB-387, Dorea longicatena HB-17, Dysosmobacter welbionis HB-45, Eisenbergiella tayi HB-437, Eisenbergiella tayi HB-612, Enterococcus durans HB-48, Enterococcus faecium HB-640, Enterococcus faecium HB-85, Enterorhabdus caecimuris B7, Enterorhabdus muris WCA-131-CoC-2, Erysipelatoclostridium ramosum HB-24, Escherichia coli HB-490, Eubacterium callanderi HB-59, Eubacterium eligens HB-252, Eubacterium rectale HB-22, Faecalitalea cylindroides HB-664, Flavonifractor plautii HB-472, Flintibacter butyricus HB-344, Gemmiger formicilis HB-325, Gemmiger sp. HB-567, Gordonibacter pamelaeae HB-15, Hungatella effluvii HB-02, Hungatella hathewayi HB-01, Intestinimonas butyriciproducens HB-478, Intestinimonas massiliensis HB-651, Lachnoclostridium sp. HB-698, Lactobacillus brevis HB-87, Lawsonibacter asaccharolyticus HB-521, Longibaculum muris HB-79, Longibaculum sp. HB-681, Mediterraneibacter faecis HB-364, Mycolicibacterium smegmatis ATCC 19420, Oscillibacter sp. HB-28, Parabacteroides distasonis HB-20, Parabacteroides distasonis HB-214, Parabacteroides goldsteinii HB-44, Parabacteroides johnsonii HB-03, Parabacteroides merdae HB-63, Parasutterella excrementihominis HB-330, Peptostreptococcus russelli RT-10B, Prevotella copri HB-373, Prevotella sp HB-649, Prevotella sp. HB-333, Romboutsia lituseburensis HB-102, Ruminococcus bicirculans HB-105, Ruminococcus bicirculans HB-268, Ruminococcus gnavus HB-40, Ruminococcus gnavus HB-516, Ruminococcus sp. HB-626, Sellimonas intestinalis HB-443, Slackia isoflavoniconvertens HB-326, Streptococcus gordonii HB-62, Streptococcus gordonii HB-98, Sutterella wadsworthensis HB-259, Turicibacter sanguinis HB-147, and Veillonella atypica HB-251.

In some embodiments, the one or more serotonin-modulating bacteria comprises a 16S rDNA sequence that is at least about 95% identical to a 16S rDNA sequence selected from one of SEQ ID NOs: 1-114. As a non-limiting example, a serotonin-modulating bacteria comprises a 16S rDNA sequence with at least 95% identity, at least 96% identity, at least 97% identity, at least 98% identity, at least 99% identity, at least 99.5% identity, at least 99.5% identity or 100% identity to a 16S rDNA sequence described herein (e.g., SEQ ID NOs: 1-114).

In some embodiments, a live bacteria, which directly produces metabolites or proteins which interact with the host, or modulates the native serotonin-modulating microbiota, serotonin-modulating metabolome, or serotonin-modulating proteome, can be superior to pharmacological interventions, as neurotransmission follows circadian rhythms, which are difficult to appropriately capture therapeutically with small molecules (while a serotonin-modulating bacteria or the native microbiota, which also has circadian rhythms, can capture this capability).

In some embodiments, a dead bacteria, which can serve as a source of metabolites or proteins to interact with the host, or modulates the native serotonin-modulating microbiota, serotonin-modulating metabolome, or serotonin-modulating proteome, can be superior to pharmacological interventions, as neurotransmission follows circadian rhythms, which are difficult to appropriately capture therapeutically with small molecules (while altering the native microbiota, which also has circadian rhythms, can capture this capability).

In some embodiments, a live or dead bacteria can be superior to pharmacological interventions, as they can capture multiple therapeutic mechanisms (e.g. the live or dead bacteria alter serotonin via small molecules as well as proteins).

In some embodiments, synergistic effects of serotonin modulation can be observed when using more than one live or dead bacteria, or by combining live or dead bacteria, or when combining single strains with the native microbiota.

In some embodiments, the serotonin-modulating bacteria described herein are exemplary in their serotonin-modulating characteristics, as compared to other bacterial strains.

In some embodiments, the serotonin-modulating bacteria produce serotonin-modulating metabolites or express genes found to elicit host serotonin release, at a physiologically relevant condition of the human gastrointestinal tract.

In some embodiments, the serotonin-modulating bacteria are engineered to produce serotonin-modulating metabolites and/or produce serotonin-modulating proteins. In some embodiments, these engineered serotonin-modulating bacteria produce the recombinant serotonin-modulating metabolites or proteins under physiologically relevant conditions of the human gastrointestinal tract.

In some embodiments, the serotonin-modulating bacteria can be delivered to the gastrointestinal tract in the form of a capsule, a tablet, a caplet, a pill, a troche, a lozenge, a powders, a granule, a medical food, supplement or a combination thereof. In some embodiments, the serotonin-modulating bacteria is administered as a fecal transplant or suppository.

In some embodiments, the viable serotonin-modulating bacteria is encapsulated, lyophilized, formulated in a food item, or is formulated in a liquid, gel, fluid-gel, or nanoparticles in a liquid. In some embodiments of any of the aspects, the composition further comprises a pre-biotic composition.

In some embodiments, serotonin-modulating bacteria described herein can be associated with negative health conditions. Without being bound by theory, commensal variants of these bacteria and/or therapeutics comprising bacterial products are free of virulence factors (e.g. exotoxins and endotoxins) and/or antimicrobial resistance. In some embodiments, commensal, non-pathogenic variants of Bacteroides vulgatus, Bilophila wadsworthia, Clostridium aldenese, Clostridium bolteae, Clostridium clostridioforme, Clostridium hathewayi, Clostridium innoculum, Eisenbergiella tayi, Enterococcus faecium, Hungatella effluvii, Hungatella hathewayi, and/or Parabacteroides distasonis are used.

### 5-HT Producers

In some embodiments, described herein are therapeutic compositions comprising one or more bacteria (e.g. purified bacteria) that produce 5-HT. These bacteria were identified to produce 5-HT via a cell culture screen and then validated with liquid chromatography-mass spectrometry (LC/MS) (see e.g., Tables 1A-1D “5-HT producer”). In some embodiments, the 5-HT producing bacteria belong to the order Lactobacillales or Clostridia. In some embodiments, the 5-HT producing bacteria belong to the genus Lactobacillus, Enterococcus, Clostridium, or Ruminococcus. In some embodiments, the 5-HT producing bacteria are the species Enterococcus durans, Clostridium lavalense, Clostridium asparagiforme, or Ruminococcus gnavus. In some embodiments, the 5-HT producing bacteria are the strains Enterococcus durans HB-48, Clostridium lavalense HB-452c, Ruminococcus gnavus HB-40, or Ruminococcus gnavus HB-516. In some embodiments, the 16S sequence of the 5-HT producing bacteria is at least 95% identical (e.g., at least 96%, at least 97%, at least 98%, at least 99%, at least 99.5%, at least 99.9% identical, or 100% identical) to a 16S sequence of a bacterial strain selected from the group consisting of: Enterococcus durans HB-48, Clostridium lavalense HB-452c, Ruminococcus gnavus HB-40, and Ruminococcus gnavus HB-516. Thus, in some embodiments, bacteria that have a 16S sequence at least 95%, at least 96%, at least 97%, at least 98%, at least 99%, at least 99.5% or at least 99.9% identical to the 16S sequence of a bacterial strain selected from Enterococcus durans HB-48, Clostridium lavalense HB-452c, Ruminococcus gnavus HB-40, and Ruminococcus gnavus HB-516 are identified as candidate 5-HT-producing bacteria. Such candidates can be confirmed as 5-HT producers via LC/MS analysis as described herein or as known in the art. In some embodiments, the 16S sequence of the 5-HT producing bacteria is at least 95% identical (e.g., at least 96%, at least 97%, at least 98%, at least 99%, at least 99.5%, at least 99.9% identical, or 100% identical) to one of SEQ ID NOs: 1-4. In some embodiments, 5-HT producing bacteria produce 5-HT at physiologically relevant conditions of the human gastrointestinal tract.

In some embodiments, the 5-HT producing bacteria have encoded in their genomes and express under the conditions in the human gastrointestinal tract: (1) a decarboxylase that catalyzes the production of tryptamine from tryptophan (e.g., a tryptophan decarboxylase); and/or (2) an enzyme to perform hydroxylation to form 5-HT from tryptamine. Tryptamine is a precursor to 5-HT, as well as a 5-HT agonist. In some embodiments, the decarboxylase that catalyzes the production of tryptamine from tryptophan is annotated under the Enzyme Commission Number (EC Number) of 4.1.1.105. In some embodiments, the decarboxylase that catalyzes the production of tryptamine from tryptophan is referred to as L-tryptophan decarboxylase or tryptophan decarboxylase. Given an EC number, one can readily identify bacterial species that encode such enzymes, thereby identifying candidate serotonin-modulating bacteria and, through their genomic sequences, structural information regarding the encoded enzymes themselves.

In some embodiments, the amino acid sequence of the decarboxylase that catalyzes the production of tryptamine from tryptophan is at least 50% similar (e.g., at least 50%, at least 55%, at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, at least 99%, at least 99.5%, at least 99.9%, or 100% similar) to one of SEQ ID NOs: 115-119. In some embodiments, the amino acid sequence of the decarboxylase that catalyzes the production of tryptamine from tryptophan is at least 50% identical (e.g., at least 50%, at least 55%, at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, at least 99%, at least 99.5%, at least 99.9%, or 100% identical) to one of SEQ ID NOs: 115-119.

In some embodiments, the decarboxylase is not annotated as a tryptophan decarboxylase, but is or can be identified by: (1) performing a neighbor-joining alignment of all decarboxylases identified in a given genome; (2) aligning those decarboxylases to positive control enzymes from other bacteria; and (3) identifying genes which cluster with those positive controls. As a non-limiting example, a decarboxylase capable of converting tryptophan to tryptamine was predicted by: (1) sequencing the complete genome of Enterococcus durans HB-48; (2) annotating all decarboxylases found within the genome using PROKKA or a similar tool); and (3) creating a phylogenetic alignment using MEGA5 (statistical method=maximum likelihood; model=Jones-Taylor Thornton; all other settings default) or a similar tool (see e.g., Seemann, Bioinformatics. 2014 Jul. 15; 30(14):2068-9; Tamura et al., Mol Biol Evol. 2011 October; 28(10):2731-9; the contents of each of which are incorporated herein by reference in their entireties). As a non-limiting example, “all decarboxylases found within the genome” can include decarboxylases annotated to the EC number 4.1.1. As a non-limiting example, “all decarboxylases” can include decarboxylases annotated to the following EC numbers: 1.1.1; 1.1.1.40; 4.1.1; 4.1.1.18; 4.1.1.17; 4.1.1.19; 4.1.1.20; 4.1.1.23; 4.1.1.3; 4.1.1.36; 4.1.1.41; 4.1.1.44; 4.1.1.65; 4.1.1.81; 4.1.1.96; 6.3.2.5; or 6.4.1.2.

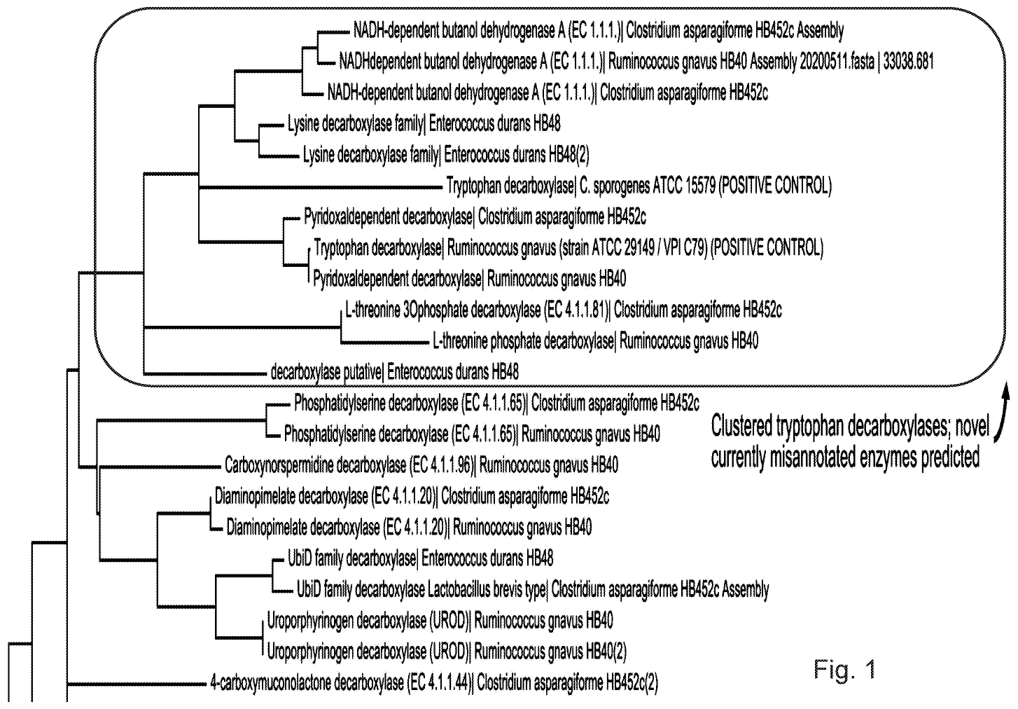

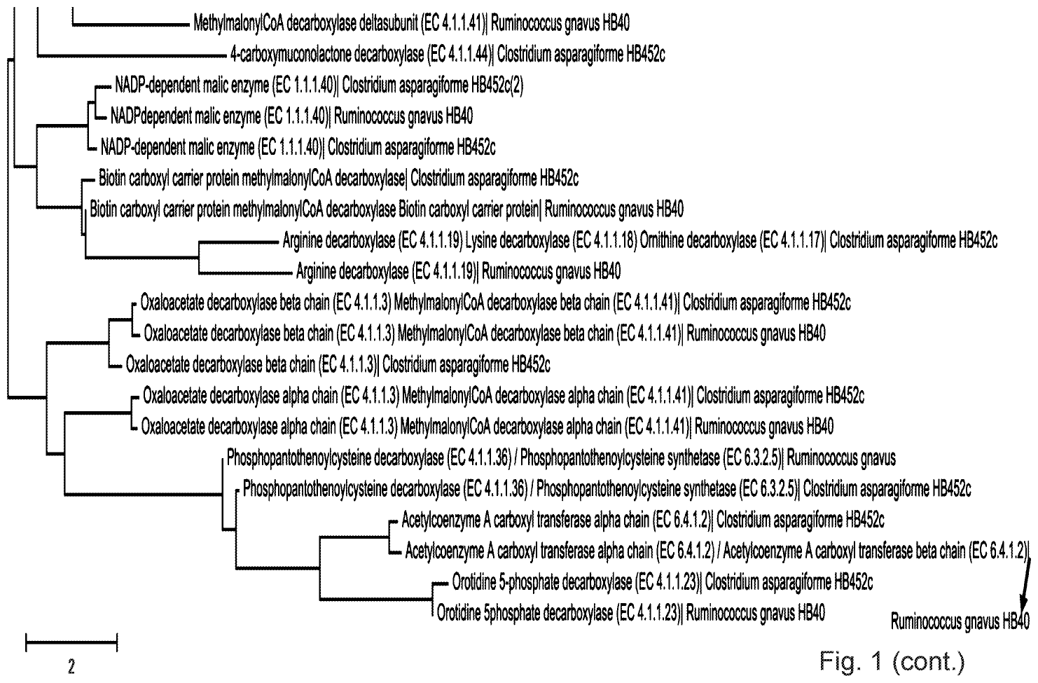

In this non-limiting example, an enzyme previously identified as a “Lysine decarboxylase family” enzyme (e.g., EC 4.1.1.18) from Enterococcus durans HB48 was clustered with positive control tryptophan decarboxylase enzymes (see e.g., FIG. 1, SEQ ID NOs: 119, 188 and 226). Bacteria encoding and expressing this lysine decarboxylase family enzyme are thus identified as candidate 5-HT producing bacteria. LC/MS performed as described herein can demonstrate 5-HT production by such bacteria. A similar approach can be applied to identify and confirm bacterial enzymes, and thereby bacteria encoding such enzymes, that catalyze additional reactions that generate serotonin from one or more precursor substrates. In particular, this approach can be applied to the enzymes described in the following.

In some embodiments, the amino acid sequence of the decarboxylase that catalyzes the production of tryptamine from tryptophan (e.g., despite being annotated as a lysine decarboxylase) is at least 50% similar (e.g., at least 50%, at least 55%, at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, at least 99%, at least 99.5%, at least 99.9%, or 100% similar) to one of SEQ ID NOs: 119, 188 or 226. In some embodiments, the amino acid sequence of the decarboxylase that catalyzes the production of tryptamine from tryptophan (e.g., despite being annotated as a lysine decarboxylase) is at least 50% identical (e.g., at least 50%, at least 55%, at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, at least 99%, at least 99.5%, at least 99.9%, or 100% identical) to one of SEQ ID NOs: 119, 188 or 226.

In some embodiments, the enzyme capable of converting tryptamine to 5-HT is tryptamine 5-hydroxylase. A similar approach to that described above is applicable to identify additional bacterial enzymes, and thereby additional candidate bacteria encoding such enzymes, that catalyze the conversion of tryptamine to 5-HT. In some embodiments, the enzyme capable of converting tryptamine to 5-HT belongs to the EC Number 1.14 or 1.14.14. In some embodiments, the enzyme capable of converting tryptamine to 5-HT is an O₂-independent hydroxylating enzyme (e.g., a molybdoenzyme capable of performing an O₂-independent hydroxylation; e.g., SEQ ID NO: 134) as described further herein. In some embodiments, multiple enzymes convert tryptamine to 5-HT in a multi-step process.

In some embodiments, the 5-HT producing bacteria have encoded in their genomes and express under the conditions in the human gastrointestinal tract: (1) an enzyme to convert tryptophan to 5-hydroxy-L-tryptophan (5-HTP) (e.g., tryptophan hydroxylase); and/or (2) an enzyme to convert 5-hydroxy-L-tryptophan to serotonin (e.g., aromatic L-amino acid decarboxylase). In some embodiments, the hydroxylase capable of converting tryptophan to 5-hydroxy-L-tryptophan is annotated under the EC Number 1.14.16.4. In some embodiments, the hydroxylase capable of converting tryptophan to 5-hydroxy-L-tryptophan is referred to as: tryptophan 5-monooxygenase; L-tryptophan hydroxylase; indoleacetic acid-5-hydroxylase; tryptophan 5-hydroxylase; and/or tryptophan hydroxylase.

In some embodiments, the amino acid sequence of the hydroxylase capable of converting tryptophan to 5-hydroxy-L-tryptophan is at least 50% similar (e.g., at least 50%, at least 55%, at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, at least 99%, at least 99.5%, at least 99.9%, or 100% similar) to one of SEQ ID NOs: 120-126. In some embodiments, the amino acid sequence of the hydroxylase capable of converting tryptophan to 5-hydroxy-L-tryptophan is at least 50% identical (e.g., at least 50%, at least 55%, at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, at least 99%, at least 99.5%, at least 99.9%, or 100% identical) to one of SEQ ID NOs: 120-126. Bacteria that encode and express both an enzyme to convert tryptophan to 5-hydroxy-L-tryptophan (5-HTP) (e.g., tryptophan hydroxylase) and an enzyme to convert 5-hydroxy-L-tryptophan to serotonin (e.g., aromatic L-amino acid decarboxylase) can produce serotonin from tryptophan. It is specifically contemplated that one or more bacterial species that encode and express an enzyme that converts tryptophan to 5-hydroxy-L-tryptophan (5-HTP) (e.g., tryptophan hydroxylase) could be paired with one or more bacterial species that encode and express an enzyme that converts 5-hydroxy-L-tryptophan to serotonin (e.g., aromatic L-amino acid decarboxylase) such that together, the paired bacterial species can generate serotonin from tryptophan.

In some embodiments, the decarboxylase capable of converting 5-hydroxy-L-tryptophan to serotonin is annotated under the EC number 4.1.1.28. In some embodiments, the decarboxylase capable of converting 5-hydroxy-L-tryptophan to serotonin is referred to as: aromatic-L-amino-acid decarboxylase; DOPA decarboxylase; tryptophan decarboxylase; hydroxytryptophan decarboxylase; L-DOPA decarboxylase; aromatic amino acid decarboxylase; and/or 5-hydroxytryptophan decarboxylase. In some embodiments, the amino acid sequence of the decarboxylase capable of converting 5-hydroxy-L-tryptophan to serotonin is at least 50% similar (e.g., at least 50%, at least 55%, at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, at least 99%, at least 99.5%, at least 99.9%, or 100% similar) to one of SEQ ID NOs: 127-133. In some embodiments, the amino acid sequence of the decarboxylase capable of converting 5-hydroxy-L-tryptophan to serotonin is at least 50% identical (e.g., at least 50%, at least 55%, at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, at least 99%, at least 99.5%, at least 99.9%, or 100% identical) to one of SEQ ID NOs: 127-133.

In some embodiments, the enzyme capable of converting tryptophan to 5-hydroxy-L-tryptophan is annotated as a phenylalanine hydroxylase. In a non-limiting example, a single point mutation in a phenylalanine hydroxylase (e.g., W192F, F197I, F197L, E219C, or any combination thereof in a phenylalanine hydroxylase from Cupriavidus taiwanensis (CtAAAH); see e.g., SEQ ID NO: 227 NCBI Reference Sequence: WP_012354318.1; e.g., EC 1.14.16.1) permits conversion of tryptophan to 5-HTP (see e.g., Mora-Villalobos and Zeng, J Biol Eng. 2018 Mar. 15; 12:3, the content of which is incorporated herein by reference in its entirety).

In some embodiments, a tryptamine 5-hydroxylase and/or tryptophan hydroxylase is not expected to function because the known enzymes require oxygen and the mammalian gastrointestinal tract environment is anaerobic. In some embodiments, 5-HT producing bacteria can circumvent the oxygen requirements of these enzymes via activity of a molybdoenzyme capable of performing an O₂-independent hydroxylation. A non-limiting example of an O₂-independent hydroxylating enzyme can be observed in Sterolibacterium denitrificans (see e.g., Demer and Fuchs, J Biol Chem. 2012 Oct. 26; 287(44):36905-16, the content of which is incorporated herein by reference in its entirety).

In some embodiments, the amino acid sequence of the O₂-independent hydroxylating enzyme is at least 50% similar (e.g., at least 50%, at least 55%, at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, at least 99%, at least 99.5%, at least 99.9%, or 100% similar) to SEQ ID NO: 134. In some embodiments, the amino acid sequence of the O₂-independent hydroxylating enzyme is at least 50% identical (e.g., at least 50%, at least 55%, at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, at least 99%, at least 99.5%, at least 99.9%, or 100% identical) to SEQ ID NO: 134.

Without wishing to be bound by theory, such anaerobic hydrolases are thought to exist in the human gut microbiome and in 5-HT producing bacteria, permitting hydroxylation of tryptamine and/or 5-hydroxy-L-tryptophan. In some embodiments, bacteria can have one or more of the enzymes involved in 5-HT production, resulting in synergistic production of 5-HT. In a non-limiting example, a bacteria (native or exogenous) can possess (i.e., encode and express) a functional tryptophan decarboxylase that produces tryptamine, and another bacteria in the same environment (native or exogenous) encodes and expresses a hydroxylase that effectively converts tryptamine to 5-HT.

Bacteria with a particularly strong result in the RIN14B model were profiled for serotonin and tryptamine production via LC/MS. Using this method, four strains were identified as tryptamine and 5-HT producers—Enterococcus durans HB-48, Clostridium lavalense HB-452c, Ruminococcus gnavus HB-40, Ruminococcus gnavus HB-516 (see e.g., Tables 1A-1D; see e.g., SEQ ID NOs: 1-4).

In some embodiments, the genes involved in 5-HT production (e.g., from tryptamine or 5-HTP) can be identified by performing a genome similarity assessment. Without being bound by theory, closely related genomes of bacteria found to be 5-HT producing bacteria and non-5-HT producing bacteria can be compared to identify sequences found only in the 5-HT producers. Identification of such genes permits directed gene-deletion strategies, using methods like CRISPR-Cas, to determine which enzymes are responsible for 5-HT production. In some embodiments, keystone 5-HT producing bacteria can be identified by searching for expression of 5-HT producing genes, as identified herein, in human fecal and cecal transcriptomic cohorts.

### Conditioned Medium

In some embodiments, described herein are therapeutic compositions comprising one or more conditioned medium or media derived from serotonin-modulating bacteria, that are delivered to the gastrointestinal tract of the subject to alter serotonin signaling/biosynthesis, either directly or by altering native microbial (e.g., bacterial, archaeal, fungal, protist, or viral) community composition or gene expression, resulting in increased or reduced levels of serotonin-modulating bacteria, or alterations in the microbiota-derived metabolome and/or microbiota-derived proteome to a more serotonin-stimulating or serotonin-inhibitory state. In some embodiments, the composition of conditioned medium or media are derived from at least 1, 2, 3, 4, 5, 6, 7, 8, 9, or 10, or more, types of isolated serotonin-modulating bacteria.

Described herein are organisms of which their supernatant modulates host 5-HT biosynthesis (see e.g., Tables 1A-1D “5-HT positive Supernatant”). In some embodiments, the 5-HT modulating bacteria can produce metabolites or proteins (e.g., secreted metabolites or secreted proteins in the supernatant) that influence host 5-HT biosynthesis. In some embodiments, the 5-HT modulating bacterial species that can produce metabolites that influence host 5-HT biosynthesis belongs to a genus selected from the group consisting of: Acidaminococcus, Agathobacter, Alistipes, Anaerotruncus, Bacillus, Bacteroides, Bifidobacterium, Bilophila, Butyricimonas, Clostridium, Clostridium hylemonae, Collinsella, Coprococcus, Dysosmobacter, Eisenbergiella, Enterococcus, Erysipelatoclostridium, Eubacterium, Faecalitalea, Flavonifractor, Flintibacter, Gemmiger, Gordonibacter, Hungatella, Intestinimonas, Lactobacillus, Mediterraneibacter, Oscillibacter, Parabacteroides, Parasutterella, Ruminococcus, Streptococcus, and Turicibacter.

In some embodiments, the 5-HT modulating bacterial species that can produce metabolites or proteins (e.g., secreted metabolites or secreted proteins in the supernatant) that influence host 5-HT biosynthesis is a species selected from the group consisting of: Acidaminococcus intestini, Agathobacter rectalis, Alistipes onderdonkii, Alistipes putredinis, Anaerotruncus colihominis, Bacillus cereus, Bacteroides caccae, Bacteroides cellulosilyticus, Bacteroides dorei, Bacteroides finegoldii, Bacteroides fragilis, Bacteroides koreensis, Bacteroides ovatus, Bacteroides plebeius, Bacteroides salyersiae, Bacteroides stercoris, Bacteroides uniformis, Bacteroides vulgatus, Bacteroides xylanisolvens, Bifidobacterium adolescentis, Bifidobacterium breve, Bifidobacterium faecale, Bifidobacterium longum, Bilophila wadsworthia, Butyricimonas paravirosa, Clostridium aldenese, Clostridium bolteae, Clostridium clostridioforme, Clostridium hathewayi, Clostridium innoculum, Clostridium paraputrificum, Clostridium saudiense, Clostridium scindens, Clostridium tyrobutyricum, Clostridium hylemonae HB-73, Collinsella aerofaciens, Coprococcus comes, Coprococcus eutactus, Dysosmobacter welbionis, Eisenbergiella tayi, Enterococcus faecium, Erysipelatoclostridium ramosum, Eubacterium eligens, Faecalitalea cylindroides, Flavonifractor plautii, Flintibacter butyricus, Gemmiger formicilis, Gordonibacter pamelaeae, Hungatella effluvii, Hungatella hathewayi, Intestinimonas butyriciproducens, Lactobacillus brevis, Mediterraneibacter faecis, Oscillibacter sp., Parabacteroides distasonis, Parabacteroides johnsonii, Parabacteroides merdae, Parasutterella excrementihominis, Ruminococcus bicirculans, Ruminococcus gnavus, Streptococcus gordonii, and Turicibacter sanguinis.

In some embodiments, the 5-HT modulating bacteria that can produce metabolites or proteins (e.g., secreted metabolites or secreted proteins in the supernatant) that influence host 5-HT biosynthesis is a strain selected from the group consisting of: Acidaminococcus intestini HB-95, Agathobacter rectalis HB-257, Alistipes onderdonkii HB-311, Alistipes putredinis HB-324, Anaerotruncus colihominis HB-474, Anaerotruncus colihominis HB-83, Bacillus cereus HB-25, Bacteroides caccae HB-11, Bacteroides cellulosilyticus HB-227, Bacteroides dorei HB-12, Bacteroides inegoldii HB-31, Bacteroides fragilis HB-58, Bacteroides koreensis HB-385, Bacteroides ovatus HB-70, Bacteroides plebeius HB-237, Bacteroides salyersiae HB-32, Bacteroides stercoris HB-33, Bacteroides uniformis HB-13, Bacteroides vulgatus HB-10, Bacteroides xylanisolvens HB-35, Bifidobacterium adolescentis HB-179, Bifidobacterium breve HB-90, Bifidobacterium faecale HB-159, Bifidobacterium longum HB-71, Bilophila wadsworthia HB-693, Butyricimonas paravirosa HB-453, Clostridium aldenese HB-440, Clostridium bolteae HB-442, Clostridium clostridioforme HB-642, Clostridium hathewayi HB-152, Clostridium innoculum HB-82, Clostridium paraputrificum HB-27, Clostridium saudiense HB-142, Clostridium scindens HB-444, Clostridium tyrobutyricum HB-469, Clostridium hylemonae HB-73, Collinsella aerofaciens HB-274, Coprococcus comes HB-376, Coprococcus eutactus HB-155, Dysosmobacter welbionis HB-45, Eisenbergiella tayi HB-437, Eisenbergiella tayi HB-612, Enterococcus faecium HB-640, Enterococcus faecium HB-85, Erysipelatoclostridium ramosum HB-24, Eubacterium eligens HB-252, Faecalitalea cylindroides HB-664, Flavonifractor plautii HB-472, Flintibacter butyricus HB-344, Gemmiger formicilis HB-325, Gordonibacter pamelaeae HB-15, Hungatella effluvii HB-02, Hungatella hathewayi HB-01, Intestinimonas butyriciproducens HB-478, Lactobacillus brevis HB-87, Mediterraneibacter faecis HB-364, Oscillibacter sp. HB-28, Parabacteroides distasonis HB-20, Parabacteroides johnsonii HB-03, Parabacteroides merdae HB-63, Parasutterella excrementihominis HB-330, Ruminococcus bicirculans HB-268, Ruminococcus gnavus HB-40, Ruminococcus gnavus HB-516, Streptococcus gordonii HB-62, Streptococcus gordonii HB-98, and Turicibacter sanguinis HB-147.

In some embodiments, the 5-HT modulating bacteria that can produce metabolites or proteins (e.g., secreted metabolites or secreted proteins in the supernatant) that influence host 5-HT biosynthesis comprise a 16S sequence that is at 95% identical (e.g., at least 96%, at least 97%, at least 98%, at least 99%, at least 99.5%, at least 99.9% identical) to one of SEQ ID NOs: 1-2 and 5-69. In some embodiments, the 5-HT modulating bacteria that can produce metabolites or proteins (e.g., secreted metabolites or secreted proteins in the supernatant) that influence host 5-HT biosynthesis do so at physiologically relevant conditions of the human gastrointestinal tract.

In some embodiments, the metabolites or proteins (e.g., secreted metabolites or secreted proteins in the supernatant) that influence host 5-HT biosynthesis can be identified by leveraging metabolomics. Without being bound by theory, untargeted or targeted metabolomics can be performed on supernatant from bacteria identified to elevate host 5-HT biosynthesis, as well as supernatant from bacteria with supernatant with no effect. By comparing metabolites from these two pools of organisms, candidate 5-HT modulating metabolites can be identified. Similarly, as described above, a genome exclusion method can be applied. Here the genomes of bacteria with supernatant that elevate host 5-HT biosynthesis can be compared to those where their supernatant has no effect, to identify candidate genetic functions associated with altered 5-HT biosynthesis. Once genetic elements are identified, transcriptomics of human fecal and cecal cohorts can be leveraged to identify bacteria in humans that express these genes in the human gastrointestinal tract.

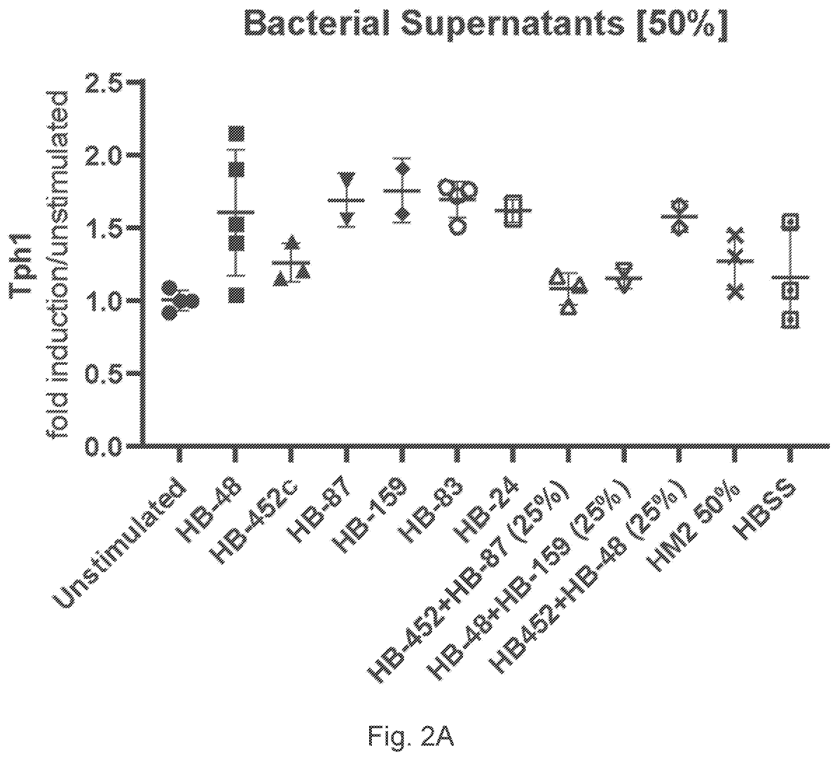

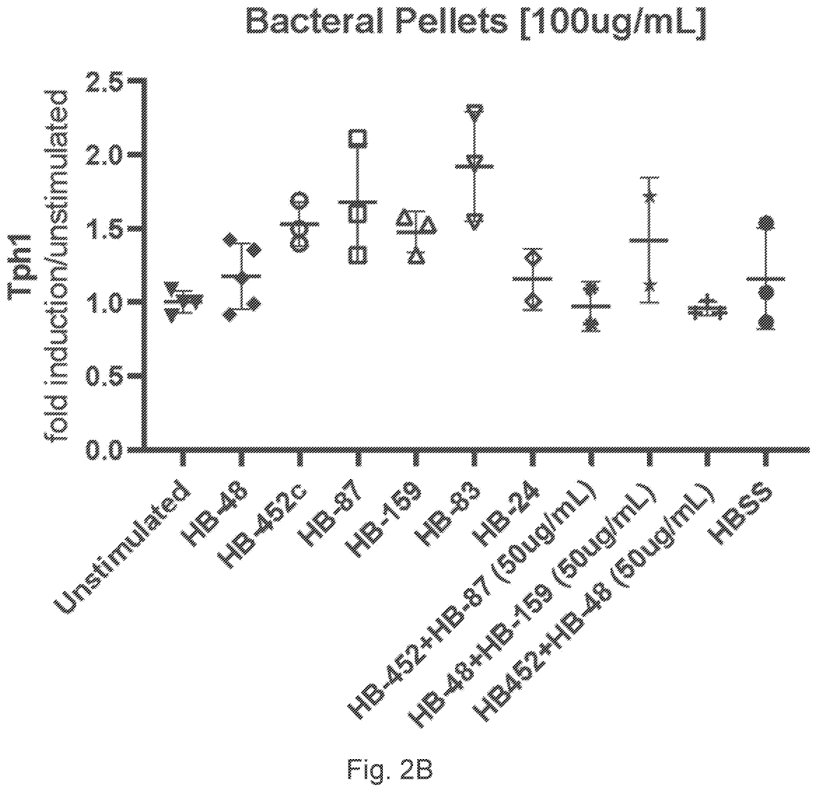

Non-limiting examples of bacterial supernatants that result in increased expression of host Tryptophan Hydroxylase 1 (TPH-1) are provided in FIG. 2A. In some embodiments, the 5-HT modulating bacterial species that can produce metabolites or proteins (e.g., secreted metabolites or secreted proteins in the supernatant) that influence host THP-1 expression belongs to a genus selected from the group consisting of: Enterococcus, Clostridium, Lactobacillus, Bifidobacterium, and Anaerotruncus. In some embodiments, the 5-HT modulating bacterial species that can produce metabolites or proteins (e.g., secreted metabolites or secreted proteins in the supernatant) that influence host THP-1 expression is a species selected from the group consisting of: Enterococcus durans, Clostridium lavalense, Lactobacillus brevis, Bifidobacterium faecale, Anaerotruncus colihominis, and Clostridium ramosum. In some embodiments, the 5-HT modulating bacterial species that can produce metabolites or proteins (e.g., secreted metabolites or secreted proteins in the supernatant) that influence host THP-1 expression is a strain selected from the group consisting of: Enterococcus durans HB-48, Clostridium lavalense HB-452c, Lactobacillus brevis HB-87, Bifidobacterium faecale HB-159, Anaerotruncus colihominis HB-83, and Clostridium ramosum HB-24. In some embodiments, the 5-HT modulating bacteria that can produce metabolites or proteins (e.g., secreted metabolites or secreted proteins in the supernatant) that influence host THP-1 expression comprises a 16S sequence that is at 95% identical (e.g., at least 96%, at least 97%, at least 98%, at least 99%, at least 99.5%, at least 99.9% identical) to one of SEQ ID NOs: 3, 4, 11, 28, 30, and 39.

In some embodiments, a composition comprises at least two 5-HT modulating bacteria that can produce metabolites or proteins (e.g., secreted metabolites or secreted proteins in the supernatant) that influence host THP-1 expression. Non-limiting examples of such combinations include: a bacterium belonging to the genus Clostridium and a bacterium belonging to the genus Lactobacillus; a bacterium belonging to the genus Enterococcus and a bacterium belonging to the genus Bifidobacterium; a bacterium belonging to the genus Clostridium and a bacterium belonging to the genus Enterococcus; Clostridium lavalense and Lactobacillus brevis; Enterococcus durans and Bifidobacterium faecale; Clostridium lavalense and Enterococcus durans; Clostridium lavalense HB-452c and Lactobacillus brevis HB-87; Enterococcus durans HB-48 and Bifidobacterium faecale HB-159; and Clostridium lavalense HB-452c and Enterococcus durans HB-48.

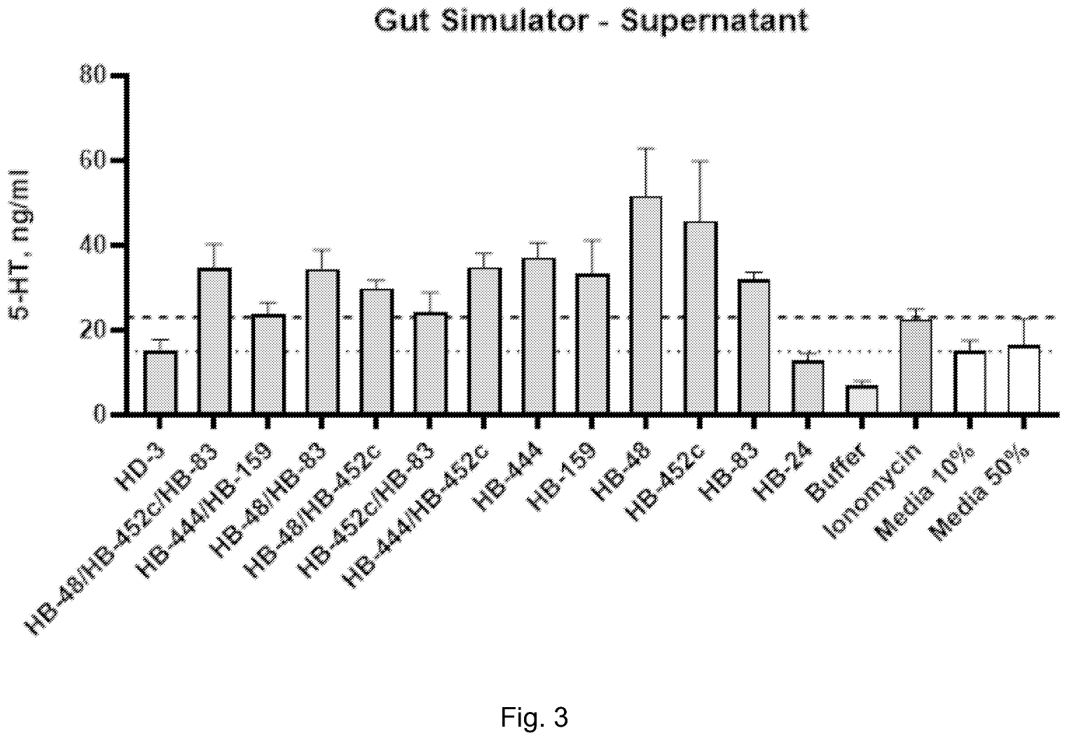

Non-limiting examples of bacterial supernatants that result in increased expression of host 5-HT in a gut simulator are provided in FIG. 3. In some embodiments, the 5-HT modulating bacterial species that can produce metabolites or proteins (e.g., secreted metabolites or secreted proteins in the supernatant) that influence host 5-HT expression belongs to a genus selected from the group consisting of: Clostridium, Bifidobacterium, Enterococcus, Anaerotruncus, and Erysipelatoclostridium. In some embodiments, the 5-HT modulating bacterial species that can produce metabolites or proteins (e.g., secreted metabolites or secreted proteins in the supernatant) that influence host 5-HT expression is a species selected from the group consisting of: Clostridium scindens, Bifidobacterium faecale, Enterococcus durans, Clostridium lavalense, Anaerotruncus colihominis, and Erysipelatoclostridium ramosum. In some embodiments, the 5-HT modulating bacterial species that can produce metabolites or proteins (e.g., secreted metabolites or secreted proteins in the supernatant) that influence host 5-HT expression is a strain selected from the group consisting of: Clostridium scindens HB-444, Bifidobacterium faecale HB-159, Enterococcus durans HB-48, Clostridium lavalense HB-452c, Anaerotruncus colihominis HB-83, and Erysipelatoclostridium ramosum HB-24. In some embodiments, the 5-HT modulating bacterial species that can produce metabolites or proteins (e.g., secreted metabolites or secreted proteins in the supernatant) that influence host 5-HT expression comprises a 16S sequence that is at 95% identical (e.g., at least 96%, at least 97%, at least 98%, at least 99%, at least 99.5%, at least 99.9% identical) to one of SEQ ID NOs: 3, 4, 11, 23, 28 and 39.

In some embodiments, a composition comprises at least two 5-HT modulating bacterial species that can produce metabolites or proteins (e.g., secreted metabolites or secreted proteins in the supernatant) that influence host 5-HT expression. Non-limiting examples of such combinations include: a bacterial species belonging to the genus Enterococcus, a bacterial species belonging to the genus Clostridium, and a bacterial species belonging to the genus Anaerotruncus; a bacterial species belonging to the genus Clostridium and a bacterial species belonging to the genus Bifidobacterium; a bacterial species belonging to the genus Enterococcus and a bacterial species belonging to the genus Anaerotruncus; a bacterial species belonging to the genus Enterococcus and a bacterial species belonging to the genus Clostridium; a bacterial species belonging to the genus Clostridium and a bacterial species belonging to the genus Anaerotruncus; two bacterial species belonging to the genus Clostridium; Enterococcus durans, Clostridium lavalense, and Anaerotruncus colihominis; Clostridium scindens and Bifidobacterium faecale; Enterococcus durans and Anaerotruncus colihominis; Enterococcus durans and Clostridium lavalense; Clostridium lavalense and Anaerotruncus colihominis; Clostridium scindens and Clostridium lavalense; Enterococcus durans HB-48, Clostridium lavalense HB-452c, and Anaerotruncus colihominis HB-83; Clostridium scindens HB-444 and Bifidobacterium faecale HB-159; Enterococcus durans HB-48 and Anaerotruncus colihominis HB-83; Enterococcus durans HB-48 and Clostridium lavalense HB-452c; Clostridium lavalense HB-452c and Anaerotruncus colihominis HB-83; and Clostridium scindens HB-444 and Clostridium lavalense HB-452c.

In some embodiments the conditioned medium is prepared by growing bacteria for a time period, ranging between 1 minute to 480 hours in medium containing one or more of the following nutrients: conditioned medium from other bacteria, N-Acetyl-D-Galactosamine, N-Acetyl-D-Glucosamine, N-Acetyl-β-D-Mannosamine, Adonitol, Amygdalin, D-Arabitol, Arbutin, D-Cellobiose, α-Cyclodextrin, β-Cyclodextrin, Dextrin, Dulcitol, i-Erythritol, D-Fructose, L-Fucose, D-Galactose, D-Galacturonic Acid, Gentiobiose, D-Gluconic Acid, D-Glucosaminic Acid, α-D-Glucose, α-D-Glucose1-Phosphate, D-Glucose6-Phosphate, Glycerol, D,L-α-Glycerol Phosphate, m-Inositol, α-D-Lactose, Lactulose, Maltose, Maltotriose, D-Mannitol, D-Mannose, D-Melezitose, D-Melibiose, β-Methyl-DGlucose, α-Methyl-DGalactoside, β-Methyl-D-Galactoside, α-Methyl-D-Glucoside, β-Methyl-D-Glucoside, Mucin, Palatinose, D-Raffinose, L-Rhamnose, Salicin, D-Sorbitol, Stachyose, Sucrose, D-Trehalose, Turanose, Acetic Acid, Formic Acid, Fumaric Acid, Glyoxylic Acid, α-Hydroxybutyric Acid, β-Hydroxybutyric Acid, Itaconic Acid, α-Ketobutyric Acid, α-Ketovaleric Acid, D,L-Lactic Acid, L-Lactic Acid, D-Lactic Acid Methyl Ester, D-Malic Acid, L-Malic Acid, Propionic Acid, Pyruvic Acid, Pyruvic Acid Methyl Ester, D-Saccharic Acid, Succinamic Acid, Succinic Acid, Succinic Acid Mono-Methyl Ester, m-Tartaric Acid, Urocanic Acid, Alaninamide, L-Alanine, L-Alanyl-LGlutamine, L-Alanyl-LHistidine, L-Alanyl-LThreonine, L-Asparagine, L-Glutamic Acid, L-Glutamine, Glycyl-LAspartic Acid, Glycyl-LGlutamine, Glycyl-LMethionine, Glycyl-LProline, L-Methionine, L-Phenylalanine, L-Serine, L-Threonine, L-Valine, L-Valine plus L-Aspartic Acid, 2′-Deoxy Adenosine, Inosine, Thymidine, Uridine, Thymidine-5′-Mono-phosphate, or Uridine-5′-Monophosphate. In some embodiments of any of the aspects, the conditioned medium is prepared by growing bacteria in HM2 media.

In some embodiments, the conditioned medium is prepared by sterilize filtration. In some embodiments, the conditioned medium is prepared by centrifugation. In some embodiments, conditioned medium is also referred to herein as a supernatant.

In some embodiments, conditioned medium from serotonin-modulating bacteria are superior to live or dead bacteria, as they can be simpler to manufacture and formulate, as long-term viability of the source organism is not necessary.

In some embodiments, conditioned medium from serotonin-modulating bacteria are superior to live or dead bacteria, or purified metabolites or proteins from serotonin-modulating bacteria, as they can capture a broader range of mechanisms. In some embodiments, this broader range of mechanisms includes serving as a prebiotic source for the native microbiota.

In some embodiments, conditioned medium from serotonin-modulating bacteria can be superior to pharmacological interventions, as they can capture multiple therapeutic mechanisms (e.g. the conditioned medium alter serotonin via small molecules or metabolites as well as proteins).

In some embodiments, the conditioned medium from serotonin-modulating bacteria described herein are exemplary in their serotonin-modulating characteristics, as compared to other bacterial strains or as compared to conditioned medium from other bacterial strains.

In some embodiments, the conditioned medium from serotonin-modulating bacteria can be delivered to the gastrointestinal tract in the form of a capsule, a tablet, a caplet, a pill, a troche, a lozenge, a powder, a granule, a medical food, supplement or a combination thereof. In some embodiments, the conditioned medium is administered rectally or via suppository.

### Cell Pellets

In some embodiments, described herein are therapeutic compositions comprising one or more cell pellets derived from serotonin-modulating bacteria, that are delivered to the gastrointestinal tract of the subject to alter serotonin signaling/biosynthesis, either directly or by altering native microbial (e.g., bacterial, archaeal, fungal, protist, or viral) community composition or gene expression, resulting in increased or reduced levels of serotonin-modulating bacteria, or alterations in the microbiota-derived metabolome and/or microbiota-derived proteome to a more serotonin-stimulating or serotonin-inhibitory state. In some embodiments, the cell pellet composition is derived from at least 1, 2, 3, 4, 5, 6, 7, 8, 9, or 10, or more, types of isolated serotonin-modulating bacteria.

Described herein are organisms that modulate 5-HT signaling via production of ligands present in the cell pellet (see e.g., Tables 1A-1D “5-HT Positive Cell Pellet”). In some embodiments, the ligand in the pellet is a polypeptide. In some embodiments, bacteria and/or bacterial ligand modulate serotonin through a Toll-like receptor (TLR)-mediated mechanism. Non-limiting examples of TLRs related to bacterial sensing include TLR1, TLR2, TLR4, TLR5, TLR6, and/or TLR9. As a non-limiting example, microbial proteins can influence host 5-HT signaling via interactions with toll-like receptor 2 (TLR2). TLR2 is a key component of the host innate immune system for maintenance of intestinal homeostasis and detection of various microbial-associated molecular patterns. Both Gram-positive and Gram-negative bacteria possess TLR2 agonists, which are generally cell-wall components, such as lipoproteins, glycoproteins and lipids, peptidoglycan, and atypical LPS molecules. Together with TLR1 and TLR4, TLR2 is expressed in human and murine Enterochromaffin (EC) cells. TLR2 has been identified as a target for host 5-HT modulation. Microbiota-dependent TLR2 signaling was found necessary to sustain EC cell number and biology in the mouse intestine, to regulate TPH1 expression and 5-HT production, and restore EC cell function in germ-free and antibiotic-treated animals. See e.g., Wang et al., J Immunol. 2019 May 15; 202(10):3041-3052; Akira & Takeda, Nat Rev Immunol 4, 499-511, (2004); Bogunovic, et al. Enteroendocrine cells express functional Toll-like receptors. Am J Physiol Gastrointest Liver Physiol 292 (2007); the contents of each of which are incorporated herein by reference in their entireties.

In some embodiments, the 5-HT modulating bacteria have cell pellets that can increase host 5-HT biosynthesis. In some embodiments, the 5-HT modulating bacteria that have cell pellets that can increase host 5-HT biosynthesis belong to a genus selected from the group consisting of: Anaerotruncus, Bacteroides, Bifidobacterium, Bittarella, Blautia, Clostridium, Dialister, Eisenbergiella, Enterococcus, Eubacterium, Gemmiger, Gordonibacter, Hungatella, Lactobacillus, Longibaculum, Mediterraneibacter, Parabacteroides, Parasutterella, Prevotella, Romboutsia, Ruminococcus, Sellimonas, and Sutterella.

In some embodiments, the 5-HT modulating bacteria that have cell pellets that can increase host 5-HT biosynthesis are a species selected from the group consisting of: Anaerotruncus colihominis, Bacteroides caccae, Bacteroides clarus, Bacteroides dorei, Bacteroides finegoldii, Bacteroides ovatus, Bacteroides salyersiae, Bacteroides thetaiotaomicron, Bacteroides xylanisolvens, Bifidobacterium adolescentis, Bifidobacterium faecale, Bittarella massiliensis, Blautia wexlerae, Clostridium aldenese, Clostridium bolteae, Clostridium hathewayi, Clostridium saudiense, Clostridium scindens, Clostridium tyrobutyricum, Dialister invisus, Eisenbergiella tayi, Enterococcus durans, Enterococcus faecium, Eubacterium eligens, Gemmiger formicilis, Gordonibacter pamelaeae, Hungatella effluvii, Lactobacillus brevis, Longibaculum muris, Mediterraneibacter faecis, Parabacteroides distasonis, Parabacteroides merdae, Parasutterella excrementihominis, Prevotella copri, Prevotella sp, Prevotella sp., Romboutsia lituseburensis, Ruminococcus sp., Ruminococcus gnavus, Sellimonas intestinalis, and Sutterella wadsworthensis.

In some embodiments, the 5-HT modulating bacterial species that have cell pellets that can increase host 5-HT biosynthesis include a strain selected from the group consisting of: Anaerotruncus colihominis HB-83, Bacteroides caccae HB-11, Bacteroides clarus HB-30, Bacteroides dorei HB-12, Bacteroides finegoldii HB-31, Bacteroides ovatus HB-70, Bacteroides salyersiae HB-32, Bacteroides thetaiotaomicron HB-34, Bacteroides xylanisolvens HB-35, Bifidobacterium adolescentis HB-179, Bifidobacterium faecale HB-159, Bittarella massiliensis HB-477, Blautia wexlerae HB-16, Clostridium aldenese HB-440, Clostridium bolteae HB-442, Clostridium hathewayi HB-152, Clostridium saudiense HB-142, Clostridium scindens HB-444, Clostridium tyrobutyricum HB-469, Dialister invisus HB-387, Eisenbergiella tayi HB-612, Enterococcus durans HB-48, Enterococcus faecium HB-85, Eubacterium eligens HB-252, Gemmiger formicilis HB-325, Gordonibacter pamelaeae HB-15, Hungatella effluvii HB-02, Lactobacillus brevis HB-87, Longibaculum muris HB-79, Mediterraneibacter faecis HB-364, Parabacteroides distasonis HB-20, Parabacteroides merdae HB-63, Parasutterella excrementihominis HB-330, Prevotella copri HB-373, Prevotella sp HB-649, Prevotella sp. HB-333, Romboutsia lituseburensis HB-102, Ruminococcus sp. HB-626, Ruminococcus gnavus HB-40, Ruminococcus gnavus HB-516, Sellimonas intestinalis HB-443, and Sutterella wadsworthensis HB-259.

In some embodiments, the 5-HT modulating bacterial species that have cell pellets that can increase host 5-HT biosynthesis comprise a 16S sequences that is at least 95% identical (e.g., at least 96%, at least 97%, at least 98%, at least 99%, at least 99.5%, at least 99.9% identical) to one of SEQ ID NOs: 1-3, 5-30, and 70-82. In some embodiments, the 5-HT modulating bacterial species that have cell pellets that can increase host 5-HT biosynthesis do so at physiologically relevant conditions of the human gastrointestinal tract. In some embodiments, the proteins or cell pellet ligands that influence host 5-HT biosynthesis can be identified by leveraging proteomics. Without being bound by theory, proteomics can be performed on cell pellets from bacteria identified to elevate host 5-HT biosynthesis, as well as cell pellets from bacteria with no effect. By comparing proteins from these two pools of organisms, candidate 5-HT modulating proteins or ligands can be identified. Similarly, as described above, a genome exclusion method can be applied. Here the genomes of bacteria with cell pellets that elevate host 5-HT biosynthesis can be compared to those where their cell pellet has no effect, to identify candidate genetic functions associated with altered 5-HT biosynthesis. Once genetic elements are identified, transcriptomics of human fecal and cecal cohorts can be leveraged to identify bacteria in humans that express these genes in the human gastrointestinal tract.

Non-limiting examples of bacterial pellets that result in increased expression of host Tryptophan Hydroxylase 1 (TPH-1) are provided in FIG. 2B. In some embodiments, the 5-HT modulating bacterial species that have cell pellets that influence host THP-1 expression belongs to a genus selected from the group consisting of: Clostridium, Lactobacillus, Bifidobacterium, and Anaerotruncus. In some embodiments, the 5-HT modulating bacterial species that have cell pellets that influence host THP-1 expression is a species selected from the group consisting of: Clostridium lavalense, Lactobacillus brevis, Bifidobacterium faecale, Anaerotruncus colihominis, and Clostridium ramosum. In some embodiments, the 5-HT modulating bacterial species that have cell pellets that influence host THP-1 expression is a strain selected from the group consisting of: Clostridium lavalense HB-452c, Lactobacillus brevis HB-87, Bifidobacterium faecale HB-159, Anaerotruncus colihominis HB-83, and Clostridium ramosum HB-24. In some embodiments, the 5-HT modulating bacterial species that have cell pellets that influence host THP-1 expression comprises a 16S sequence that is at 95% identical (e.g., at least 96%, at least 97%, at least 98%, at least 99%, at least 99.5%, at least 99.9% identical) to one of SEQ ID NOs: 4, 11, 28, 30, and 39.

In some embodiments, a composition comprises at least two 5-HT modulating bacteria that have cell pellets that influence host THP-1 expression. Non-limiting examples of such combinations include: a bacterial species belonging to the genus Clostridium and a bacterial species belonging to the genus Lactobacillus; a bacterial species belonging to the genus Enterococcus and a bacterial species belonging to the genus Bifidobacterium; a bacterial species belonging to the genus Clostridium and a bacterial species belonging to the genus Enterococcus; Clostridium lavalense and Lactobacillus brevis; Enterococcus durans and Bifidobacterium faecale; Clostridium lavalense and Enterococcus durans; Clostridium lavalense HB-452c and Lactobacillus brevis HB-87; Enterococcus durans HB-48 and Bifidobacterium faecale HB-159; and Clostridium lavalense HB-452c and Enterococcus durans HB-48.

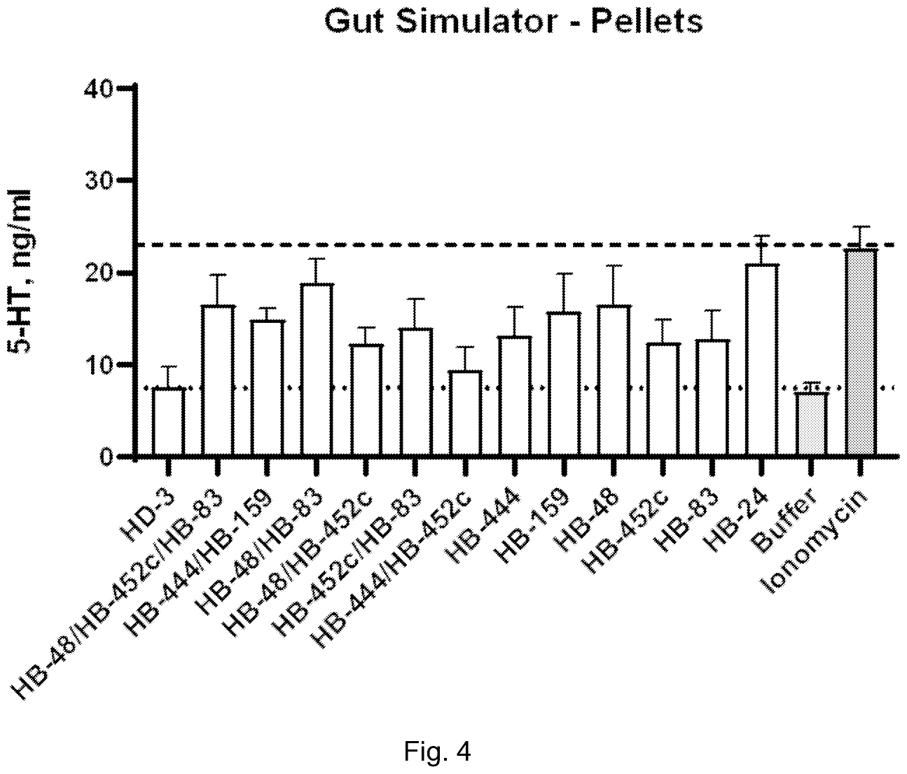

Non-limiting examples of bacterial pellets that result in increased expression of host 5-HT in a gut simulator are provided in FIG. 4. In some embodiments, the 5-HT modulating bacterial species that have cell pellets that influence host 5-HT expression belongs to a genus selected from the group consisting of: Clostridium, Bifidobacterium, Enterococcus, Anaerotruncus, and Erysipelatoclostridium. In some embodiments, the 5-HT modulating bacterial species that has a cell pellet that influences host 5-HT expression is a species selected from the group consisting of: Clostridium scindens, Bifidobacterium faecale, Enterococcus durans, Clostridium lavalense, Anaerotruncus colihominis, and Erysipelatoclostridium ramosum. In some embodiments, the 5-HT modulating bacterial species that has a cell pellet that influences host 5-HT expression is a strain selected from the group consisting of: Clostridium scindens HB-444, Bifidobacterium faecale HB-159, Enterococcus durans HB-48, Clostridium lavalense HB-452c, Anaerotruncus colihominis HB-83, and Erysipelatoclostridium ramosum HB-24. In some embodiments, the 5-HT modulating bacterial species that have cell pellets that influence host 5-HT expression comprises a 16S sequence that is at 95% identical (e.g., at least 96%, at least 97%, at least 98%, at least 99%, at least 99.5%, at least 99.9% identical) to one of SEQ ID NOs: 3, 4, 11, 23, 28 and 39.

In some embodiments, a composition comprises at least two 5-HT modulating bacteria that have cell pellets that influence host 5-HT expression. Non-limiting examples of such combinations include: a bacterial species belonging to the genus Enterococcus, a bacterial species belonging to the genus Clostridium, and a bacterial species belonging to the genus Anaerotruncus; a bacterial species belonging to the genus Clostridium and a bacterial species belonging to the genus Bifidobacterium; a bacterial species belonging to the genus Enterococcus and a bacterial species belonging to the genus Anaerotruncus; a bacterial species belonging to the genus Enterococcus and a bacterial species belonging to the genus Clostridium; a bacterial species belonging to the genus Clostridium and a bacterial species belonging to the genus Anaerotruncus; two bacterial species belonging to the genus Clostridium; Enterococcus durans, Clostridium lavalense, and Anaerotruncus colihominis; Clostridium scindens and Bifidobacterium faecale; Enterococcus durans and Anaerotruncus colihominis; Enterococcus durans and Clostridium lavalense; Clostridium lavalense and Anaerotruncus colihominis; Clostridium scindens and Clostridium lavalense; Enterococcus durans HB-48, Clostridium lavalense HB-452c, and Anaerotruncus colihominis HB-83; Clostridium scindens HB-444 and Bifidobacterium faecale HB-159; Enterococcus durans HB-48 and Anaerotruncus colihominis HB-83; Enterococcus durans HB-48 and Clostridium lavalense HB-452c; Clostridium lavalense HB-452c and Anaerotruncus colihominis HB-83; and Clostridium scindens HB-444 and Clostridium lavalense HB-452c.

In some embodiments the cell pellets are prepared by growing bacteria for a time period, ranging between 1 minute to 480 hours in medium containing one or more of the following nutrients: conditioned medium from other bacteria, N-Acetyl-D-Galactosamine, N-Acetyl-D-Glucosamine, N-Acetyl-β-D-Mannosamine, Adonitol, Amygdalin, D-Arabitol, Arbutin, D-Cellobiose, α-Cyclodextrin, (3-Cyclodextrin, Dextrin, Dulcitol, i-Erythritol, D-Fructose, L-Fucose, D-Galactose, D-Galacturonic Acid, Gentiobiose, D-Gluconic Acid, D-Glucosaminic Acid, α-D-Glucose, α-D-Glucose 1-Phosphate, D-Glucose6-Phosphate, Glycerol, D,L-α-Glycerol Phosphate, m-Inositol, α-D-Lactose, Lactulose, Maltose, Maltotriose, D-Mannitol, D-Mannose, D-Melezitose, D-Melibiose, 3-Methyl-DGlucose, α-Methyl-DGalactoside, β-Methyl-D-Galactoside, α-Methyl-D-Glucoside, β-Methyl-D-Glucoside, Mucin, Palatinose, D-Raffinose, L-Rhamnose, Salicin, D-Sorbitol, Stachyose, Sucrose, D-Trehalose, Turanose, Acetic Acid, Formic Acid, Fumaric Acid, Glyoxylic Acid, α-Hydroxybutyric Acid, β-Hydroxybutyric Acid, Itaconic Acid, α-Ketobutyric Acid, α-Ketovaleric Acid, D,L-Lactic Acid, L-Lactic Acid, D-Lactic Acid Methyl Ester, D-Malic Acid, L-Malic Acid, Propionic Acid, Pyruvic Acid, Pyruvic Acid Methyl Ester, D-Saccharic Acid, Succinamic Acid, Succinic Acid, Succinic Acid Mono-Methyl Ester, m-Tartaric Acid, Urocanic Acid, Alaninamide, L-Alanine, L-Alanyl-LGlutamine, L-Alanyl-LHistidine, L-Alanyl-LThreonine, L-Asparagine, L-Glutamic Acid, L-Glutamine, Glycyl-LAspartic Acid, Glycyl-LGlutamine, Glycyl-LMethionine, Glycyl-LProline, L-Methionine, L-Phenylalanine, L-Serine, L-Threonine, L-Valine, L-Valine plus L-Aspartic Acid, 2′-Deoxy Adenosine, Inosine, Thymidine, Uridine, Thymidine-5′-Mono-phosphate, or Uridine-5′-Monophosphate. In some embodiments of any of the aspects, the cell pellets are prepared by growing bacteria in HM2 media.

In some embodiments, the cell pellets are prepared by killing viable bacteria using high temperatures, freeze-thaw cycles, chloroform treatment, irradiation, or other appropriate means known to those skilled in the art, optionally followed by centrifugation (or other concentrating step) and/or at least one wash step using phosphate buffered saline or another liquid.

In some embodiments, cell pellets from serotonin-modulating bacteria are superior to live or dead bacteria, as they can be simpler to manufacture and formulate, as long-term viability of the source organism is not necessary.

In some embodiments, cell pellets from serotonin-modulating bacteria are superior to live or dead bacteria, or purified metabolites or proteins from serotonin-modulating bacteria, as they can capture a broader range of mechanisms. In some embodiments, this broader range of mechanisms includes serving as a prebiotic source for the native microbiota.

In some embodiments, cell pellets from serotonin-modulating bacteria can be superior to pharmacological interventions, as they can capture multiple therapeutic mechanisms (e.g. the cell pellets alter serotonin via small molecules as well as proteins).

In some embodiments, the cell pellets from serotonin-modulating bacteria described herein are exemplary in their serotonin-modulating characteristics, as compared to other bacterial strains or as compared to cell pellets from other bacterial strains.

In some embodiments, the cell pellets from serotonin-modulating bacteria can be delivered to the gastrointestinal tract in the form of a capsule, a tablet, a caplet, a pill, a troche, a lozenge, a powders, a granule, a medical food, supplement or a combination thereof. In some embodiments, the cell pellets are administered rectally or via suppository.

### 5-HT Agonists, Purified Metabolites or Proteins

In some embodiments, described herein are therapeutic compositions comprising one or more purified 5-HT agonists, metabolites and/or proteins, derived from serotonin-modulating bacteria, that are delivered to the gastrointestinal tract to alter subject serotonin signaling/biosynthesis, either directly or by altering native microbial (e.g., bacterial, archaeal, fungal, protist, or viral) community composition or gene expression, resulting in increased or reduced levels of serotonin-modulating bacteria, or alterations in the microbiota-derived metabolome and/or microbiota-derived proteome to a more serotonin-stimulating or serotonin-inhibitory state. In some embodiments, the composition comprises purified 5-HT agonists, metabolites and/or proteins derived from at least 1, 2, 3, 4, 5, 6, 7, 8, 9, or 10, or more, types of isolated serotonin-modulating bacteria.

In some embodiments, described herein are therapeutic compositions comprising one or more bacterial species (e.g. purified bacteria) that can modulate 5-HT signaling by producing agonists of 5-HT receptors (see e.g., Tables 1A-1D “Agonist Producer”). In some embodiments, described herein are therapeutic compositions comprising purified 5-HT agonists from serotonin-modulating bacteria described herein.

There are a range of serotonin receptors, each responsible for eliciting release of a range of neurotransmitters and hormones. Likely the most relevant for enteric 5-HT signaling is the 5-HT4 receptor, which is a known pro-kinetic pharmacological target. Interestingly, the 5-HT4 receptor is widely distributed throughout the gastrointestinal tract and has been shown to sense and respond to lumen-derived metabolites. A non-limiting example of a 5-HT agonist is the tryptophan metabolite tryptamine, which is sensed in the same manner as the receptor's natural agonist 5-HT. Of note, tryptamine is produced by bacterial decarboxylation of dietary tryptophan, and microbiota-derived tryptamine has been shown to accelerate GI transit in a mouse model. This indicates it is possible to elicit a 5-HT-like effect, mediated via TLR-4, without serotonin. Tryptamine also activates the human Trace Amine Associated Receptor (TAAR) system, modulating serotonin by promoting its release into the synaptic cleft. See e.g., Manabe et al. Expert Opin Investig Drugs 19, 765-775, (2010); Bhattarai et al., Cell host & microbe 23, 775-785 e775, (2018); the contents of which are incorporated herein by reference in their entireties.

In some embodiments, the 5-HT modulating bacterial species that produces a 5-HT agonist (e.g., tryptamine) is used to treat a serotonin-related disease or disorder or its symptom(s) caused by low serotonin levels that is not a gut disease or disorder. In some embodiments, the serotonin-related disease or disorder or its symptom(s) caused by low serotonin levels is selected from the group consisting of depression, anxiety, addiction, neurodegenerative disorders, autism spectrum disorder, sleep disorders, attention deficit hyperactivity disorder (ADHD), memory loss (e.g. dementia), learning difficulties, osteoporosis, dermatological conditions (eczema and itch), and pain disorders.

In some embodiments, the 5-HT modulating bacterial species can elicit an elevation of host 5-HT signaling via production of 5-HT agonists or TAAR agonists. In some embodiments, the 5-HT agonist-producing bacterial species belongs to a genus selected from the group consisting of: Adlercreutzia, Akkermansia, Clostridium, Coprococcus, Enterococcus, Enterorhabdus, Mycolicibacterium, Peptostreptococcus, and Ruminococcus. In some embodiments, the 5-HT agonist-producing bacterial species is a species selected from the group consisting of: Akkermansia muciniphila, Adlercreutzia equolifaciens, Clostridium sporogenes, Clostridium lavalense, Clostridium asparagiforme, Coprococcus eutactus, Coprococcus comes, Enterococcus durans, Enterorhabdus muris, Enterorhabdus caecimuris, Mycolicibacterium smegmatis, Peptostreptococcus russelli, and Ruminococcus gnavus. In some embodiments, the 5-HT agonist-producing bacterial species is a strain selected from the group consisting of: Enterococcus durans HB-48, Clostridium lavalense HB-452c, Ruminococcus gnavus HB-40, Ruminococcus gnavus HB-516. In some embodiments, the 5-HT agonist-producing bacteria are greater than 95% similar by 16S sequencing to Enterococcus durans HB-48, Clostridium lavalense HB-452c, Ruminococcus gnavus HB-40, Ruminococcus gnavus HB-516, Clostridium sporogenes JCM 7836, Akkermansia muciniphila BAA-835, Clostridium sporogenes McClung 2004, Peptostreptococcus russeliii RT-10B, Mycolicibacterium smegmatis ATCC 19420, Enterorhabdus muris WCA-131-CoC-2, Adlercreutzia equolifaciens FJC-B9, Enterorhabdus caecimuris B7, Coprococcus eutactus ATCC 27759, and Coprococcus comes ATCC 27758.

In some embodiments, the 5-HT agonist-producing bacterial species comprises a 16S sequence that is at least 95% identical (e.g., at least 96%, at least 97%, at least 98%, at least 99%, at least 99.5%, at least 99.9% identical) to one of SEQ ID NOs: 1-4 and 105-114. In some embodiments, the 5-HT agonist-producing bacterial species produces the 5-HT agonist at physiologically relevant conditions of the human gastrointestinal tract.

Metabolites and derivatives of tryptophan which are known to have agonist properties at various 5-HT receptors and TAAR include, but are not limited to, N-methyltryptamine, N,N-dimethyltryptamine, N-methylserotonin, and N,N-dimethylserotonin. These compounds can be synthesized either by methylation of tryptamine by bacterial enzymes functionally homologous to human indolethylamine N-methyltransferase (e.g., GenBank Reference No: AAF18306.1; e.g., EC 2.1.1.49), or by human (or microbial as described further herein) tryptophan decarboxylase and hydroxylase enzymes (e.g., EC 4.1.1.105, EC 1.14.16.4) acting upon bacterially synthesized N-methylated derivatives of tryptophan including, but not limited to, L-abrine, N,N-alpha-dimethyltryptophan, and hypaphorine (also known as tryptophan betaine). These N-methylated tryptophan derivatives can be biosynthesized by radical S-adenosyl-L-methionine (SAM)-dependent enzymes with greater than 50% sequence homology to the ergothioneine biosynthetic enzyme egtD (e.g., UniProt AOR5M8 or NCBI Reference Sequence: WP_058127191.1; e.g., EC 2.1.1.44; see e.g., SEQ ID NO: 228), or by phosphatidylethanolamine N-methyltransferase enzymes similar to those used to biosynthesize phosphatidylcholine (e.g., EC 2.1.1.- or EC 2.1.1.17; e.g., SEQ ID NO: 229). Bacteria, particularly those in the genera Akkermansia, Eubacterium, Bacteroides, Coprococcus, or Enterorhabdus, which produce these compounds and their metabolites can be delivered to exert 5-HT agonist effects, or to indirectly modulate 5-HT activity via inhibition of 5-HT reuptake by the serotonin uptake transporter (SERT) or the vesicular monoamine transporters (VMAT), or by inhibition of the monoamine oxidase enzyme.

In some embodiments, the amino acid sequence of an enzyme involved in N-methylated tryptophan derivative production is at least 50% similar (e.g., at least 50%, at least 55%, at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, at least 99%, at least 99.5%, at least 99.9%, or 100% similar) to one of SEQ ID NOs: 228-229. In some embodiments, the amino acid sequence of an enzyme involved in N-methylated tryptophan derivative production is at least 50% identical (e.g., at least 50%, at least 55%, at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, at least 99%, at least 99.5%, at least 99.9%, or 100% identical) to one of SEQ ID NOs: 228-229.

In some embodiments, the serotonin-modulating bacteria can produce tryptophan itself, which feeds into production pathways of 5-HT, 5-HT agonists, and TAAR agonists. In some embodiments, the serotonin-modulating bacterial species encodes or expresses at least one functional enzyme involved in tryptophan biosynthesis. In some embodiments, the enzyme involved in tryptophan production is selected from the group consisting of: Tryptophan synthase (SEQ ID NOs: 135-144; EC 4.2.1.20); Indole-3-glycerol phosphate synthase (SEQ ID NOs: 145-148; EC 4.1.1.48); Anthranilate phosphoribosyltransferase (e.g., SEQ ID NOs: 149-153; EC 2.4.2.18); Anthranilate synthase (e.g., SEQ ID NO: 154-158; EC 4.1.3.27); N-(5′-phosphoribosyl)anthranilate isomerase (e.g., SEQ ID NO: 159-162, EC 5.3.1.24); 1-(5-phosphoribosyl)-5-[(5-phosphoribosylamino)methylideneamino] imidazole-4-carboxamide isomerase (e.g., SEQ ID NO: 163; EC 5.3.1.36). In some embodiments, the enzyme involved in tryptophan production belongs to an EC number selected from the group consisting of: EC 4.2.1.20, EC 4.1.1.48, EC 2.4.2.18, EC 4.1.3.27, EC 5.3.1.24, and EC 5.3.1.36.

In some embodiments, the amino acid sequence of enzyme involved in tryptophan production is at least 50% similar (e.g., at least 50%, at least 55%, at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, at least 99%, at least 99.5%, at least 99.9%, or 100% similar) to one of SEQ ID NOs: 135-163. In some embodiments, the amino acid sequence of enzyme involved in tryptophan production is at least 50% identical (e.g., at least 50%, at least 55%, at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, at least 99%, at least 99.5%, at least 99.9%, or 100% identical) to one of SEQ ID NOs: 135-163.

Metabolites of phenylalanine including, but not limited to, phenethylamine, tyramine, and N-methylated derivatives thereof can also activate the TAAR system, modulating serotonin release and reuptake. These metabolites can be biosynthesized in a similar manner to those described above by administered bacteria, particularly those in the genera Akkermansia, Eubacterium, Bacteroides, Coprococcus, or Enterorhabdus.

Indole-3-carboxylic acid derivatives of tryptophan include indole-3-propionic acid (I3PA), synthesized by the fldAIBC gene cluster, and the related compounds, indole-3-acrylic acid (I3A), indole-3-lactic acid (I3LA), indole-3-pyruvic acid (I3PyA) and indole-3-acetic acid (I3Ac). In some embodiments, bacteria in the genera Clostridium or Peptostreptococcus that produce I3PA can be used alone or in combination with other bacteria and their metabolites to modulate serotonergic tone either via direct action by I3PA and related metabolites at 5-HT receptors (projected based on structural similarity of I3PA to tryptamine and 5-HT) or via transporter and reuptake inhibition (projected based on pharmacodynamic similarity of I3PA and the known monoamine reuptake inhibitor hyperforin at the pregnane X receptor).

In some embodiments, combinations of bacteria may be used to capture combined functional production of I3PA or other indole-3-carboxylic acid derivatives of tryptophan, whereas single strains do not have complete functional capabilities of producing I3PA. In some embodiments, a key enzyme in production of I3PA is acyl-CoA dehydrogenase, which is found is in the 5-HT modulating genera Acidaminococcus, Agathobacter, Alistipes, Anaerotruncus, Bacillus, Bacteroides, Bifidobacterium, Butyricimonas, Clostridium, Coprococcus, Eisenbergiella, Enterococcus, Erysipelatoclostridium, Eubacterium, Faecalitalea, Flavonifractor, Gemmiger, Gordonibacter, Hungatella, Lachnoclostridium, Lactobacillus, Oscillibacter, Parabacteroides, Ruminococcus, and/or Streptococcus. In some embodiments, acyl-CoA dehydrogenase is not found in members of the genera Bilophila, Collinsella, Intestinimonas, Parasutterella, and/or Turicibacter.

In some embodiments, the acyl-CoA dehydrogenase belongs to EC 1.3.99.3; EC 1.3.8.7; EC 1.3.8.8; or EC 1.3.8.9. In some embodiments, the amino acid sequence of the acyl-CoA dehydrogenase is at least 50% similar (e.g., at least 50%, at least 55%, at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, at least 99%, at least 99.5%, at least 99.9%, or 100% similar) to one of SEQ ID NOs: 164-171. In some embodiments, the amino acid sequence of the acyl-CoA dehydrogenase is at least 50% identical (e.g., at least 50%, at least 55%, at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, at least 99%, at least 99.5%, at least 99.9%, or 100% identical) to one of SEQ ID NOs: 164-171.

Indole-3-alcohol and -aldehyde derivatives of tryptophan include but are not limited to indole-3-carbinol and indole-3-carboxaldehyde, and can exert serotonergic effects by the mechanisms described above.

In some embodiments, the bacterial enzymes involved in production of metabolites capable of acting as 5-HT agonists or signaling modifiers are selected from the group consisting of: Tryptophanase (EC 4.1.99.1; e.g., SEQ ID NO: 172); Indole-3-propionate biosynthesis enzymes (e.g., SEQ ID NO: 173-180); Cinnamoyl-CoA:phenyllactate CoA-transferase (EC 2.8.3.17; e.g., SEQ ID NOs: 173 or 177); (R)-3-(aryl)lactoyl-CoA dehydratase, alpha or beta subunit (EC 4.2.1.175; e.g., SEQ ID NOs: 174, 175, 178, 179); archerase (EC 5.6.1.9; e.g., SEQ ID NOs: 176 or 180); Tryptophan/aromatic amino acid N-methyltransferase (EC 2.1.1.—e.g., SEQ ID NOs: 181-184); see e.g., Table 3.

TABLE 3
Exemplary bacterial enzymes involved in 5-HT agonist or modifier production

| | | SEQ ID | |
|---|---|---|---|
| Enzyme | EC | NO | Example Bacteria |
| Tryptophanase (indole production) | EC 4.1.99.1 | 172 | Enterocloster lavalensis HB-452c |
| Indole-3-propionate biosynthesis | EC 2.8.3.17, | 173-180 | Clostridium sporogenes ; |
| enzymes | EC 4.2.1.175, | | Peptostreptococcus russellii |
| | EC 5.6.1.9 | | |
| Tryptophan/aromatic amino acid | EC 2.1.1.— | 181-184 | Escherichia coli; |
| N-methyltransferase | | | Mycolicibacterium; smegmatis; |
| | | | Mycobacterium vaccae; |
| | | | Akkermansia muciniphila; |
| | | | Adlercreutzia equolifaciens; |
| | | | Enterorhabdus spp. |

In some embodiments, the amino acid sequences of the bacterial enzymes involved in production of metabolites capable of acting as 5-HT agonists or signaling modifiers, including those named above, are at least 50% similar (e.g., at least 50%, at least 55%, at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, at least 99%, at least 99.5%, at least 99.9%, or 100% similar) to one of SEQ ID NOs: 172-184. In some embodiments, the amino acid sequences of the bacterial enzymes involved in production of metabolites capable of acting as 5-HT agonists or signaling modifiers, including those named above, are at least 50% identical (e.g., at least 50%, at least 55%, at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, at least 99%, at least 99.5%, at least 99.9%, or 100% identical) to one of SEQ ID NOs: 172-184.

In some embodiments, one or more 5-HT agonists, purified metabolites and/or proteins, derived from serotonin-modulating bacteria, are superior to live or dead serotonin-modulating bacteria or conditioned medium or cell pellets of serotonin-modulating bacteria. In some embodiments, superiority is observed through increased potency of the one or more 5-HT agonists, purified metabolites and/or proteins. In some embodiments, superiority is observed through ease of manufacturing and development.

In some embodiments, combinations of purified 5-HT agonists, metabolites and/or proteins, derived from serotonin-modulating bacteria, are synergistic in their capacity to modulate a serotonin signaling or biosynthesis in a subject.

In some embodiments, the one or more purified 5-HT agonists, metabolites and/or proteins, derived from serotonin-modulating bacteria described herein, are exemplary in their serotonin-modulating characteristics, as compared to purified 5-HT agonists, metabolites and/or proteins derived from other bacterial strains.

In some embodiments, the purified 5-HT agonists, metabolites and/or proteins, derived from serotonin-modulating bacteria, can be delivered to the gastrointestinal tract in the form of a capsule, a tablet, a caplet, a pill, a troche, a lozenge, a powders, a granule, a medical food, supplement or a combination thereof. In some embodiments, the 5-HT agonists, purified metabolites and/or proteins are administered rectally or via suppository.

### 5-HT Reducers

In some embodiments, described herein are therapeutic compositions comprising one or more bacteria (e.g. purified bacteria) that consume 5-HT and/or reduce host biosynthesis of 5-HT (see e.g., Tables 1A-1D “5-HT Reducer”). In some embodiments, the composition of serotonin reducers are derived from at least 2, 3, 4, 5, 6, 7, 8, 9, or 10, or more, types of isolated serotonin-modulating bacteria.

In some embodiments, a bacterial negative modulator of 5-HT signaling belongs to a genus selected from the group consisting of: Bifidobacterium, Blautia, Clostridium, Coprococcus, Dorea, Eubacterium, Lachnoclostridium, and Slackia. In some embodiments a bacterial negative modulator of 5-HT signaling is a species selected from the group consisting of: Bifidobacterium longum, Blautia coccoides, Blautia obeum, Clostridium butyricum, Coprococcus comes, Dorea longicatena, Eubacterium rectale, Lachnoclostridium sp., and Slackia isoflavoniconvertens. In some embodiments, the bacterial negative modulator of 5-HT signaling is a strain selected from the group consisting of: Bifidobacterium longum HB-234, Blautia coccoides HB-23, Blautia obeum HB-14, Clostridium butyricum HB-88, Coprococcus comes HB-80, Dorea longicatena HB-17, Eubacterium rectale HB-22, Lachnoclostridium sp. HB-698, and Slackia isoflavoniconvertens HB-326.

In some embodiments, a bacterial negative modulator of 5-HT signaling comprises a 16S sequence that is at least 95% identical (e.g., at least 96%, at least 97%, at least 98%, at least 99%, at least 99.5%, at least 99.9% identical) to one of SEQ ID NOs: 96-104. In some embodiments, the bacterial negative modulators of 5-HT signaling consume tryptophan, preventing access from the host. In some embodiments, the bacterial negative modulators of 5-HT signaling consume 5-HT agonists or 5-HT potentiating metabolites. In some embodiments the bacterial negative modulators of 5-HT signaling out-compete positive 5-HT modulating bacteria in the human gastrointestinal tract.

### Minimal 5-HT Impact

In some embodiments, described herein are bacteria with little to no effect on 5-HT (see e.g., Tables 1A-1D “No or Low 5-HT Impact”). In some embodiments, the 5-HT modulating bacteria genus is not selected from the group consisting of: Clostridium, Escherichia, Eubacterium, Gemmiger, Intestinimonas, Lawsonibacter, Longibaculum, Parabacteroides, Ruminococcus, and Veillonella, wherein the bacteria does not encode or express one or more, up to all of the enzymes described herein for 5-HT production, or for 5-HT modulation.

In some embodiments, the 5-HT modulating bacteria species is not selected the group consisting of: Clostridium sp., Clostridium sphenoides, Clostridium symbiosum, Escherichia coli, Eubacterium callanderi, Gemmiger sp., Intestinimonas massiliensis, Lawsonibacter asaccharolyticus, Longibaculum sp., Parabacteroides distasonis, Parabacteroides goldsteinii, Ruminococcus bicirculans, and Veillonella atypica, wherein the bacteria does not encode or express one or more, up to all of the enzymes described herein for 5-HT production, or for 5-HT modulation.

In some embodiments, the 5-HT modulating bacteria strain is not a bacterium selected from the group consisting of: Clostridium sp. HB-358, Clostridium sphenoides HB-470, Clostridium symbiosum HB-67, Escherichia coli HB-490, Eubacterium callanderi HB-59, Gemmiger sp. HB-567, Intestinimonas massiliensis HB-651, Lawsonibacter asaccharolyticus HB-521, Longibaculum sp. HB-681, Parabacteroides distasonis HB-214, Parabacteroides goldsteinii HB-44, Ruminococcus bicirculans HB-105, and Veillonella atypica HB-251, wherein the bacteria does not encode or express one or more, up to all of the enzymes described herein for 5-HT production, or for 5-HT modulation.

### Combined Compositions

It is noted that the individual bacterial species or strain described herein can modulate serotonin as individual species or strain. It is also contemplated that consortia of these species, either with other members of the species or strains described herein, or with other species or strains, that, for example, express one or more genes, or produce one or more metabolites that modulate serotonin levels, can provide additional benefits regarding serotonin modulation.

In some embodiments, described herein are therapeutic compositions comprising combinations of one or more live serotonin-modulating bacteria, one or more dead or inactivated serotonin-modulating bacteria, one or more conditioned medium(s) of serotonin-modulating bacteria, one or more cell pellet(s) of serotonin-modulating bacteria, and/or one or more metabolites and/or proteins (derived from serotonin-modulating bacteria), that are delivered to the gastrointestinal tract of the subject to modulate serotonin signaling and/or biosynthesis of the subject, either directly or by altering native microbial (e.g., bacterial, archaeal, fungal, protist, or viral) community composition or gene expression, resulting in increased or reduced levels of serotonin-modulating bacteria, or alterations in the microbiota-derived metabolome and/or microbiota-derived proteome to a more serotonin-stimulating or serotonin-inhibitory state. In some embodiments, the composition of live bacteria, dead bacteria, conditioned medium(s), cell pellet(s), purified 5-HT agonists, metabolites, or proteins are derived from at least 2, 3, 4, 5, 6, 7, 8, 9, or 10, or more, types of isolated serotonin-modulating bacteria.

In some embodiments, combinations of one or more live serotonin-modulating bacteria, one or more dead-serotonin modulating bacteria, one or more conditioned medium(s) of serotonin-modulating bacteria, one or more cell pellet(s) of serotonin-modulating bacteria, and/or one or more metabolites and/or proteins (derived from serotonin-modulating bacteria) are superior to alternative formulations, through activation of multiple mechanisms. For example, metabolites produced by serotonin-modulating bacteria can elevate serotonin biosynthesis by the host via TPH-1, while proteins derived from serotonin-modulating bacteria can elevate serotonin biosynthesis by the host via TPH-2. In some embodiments, combinations of one or more live serotonin-modulating bacteria, one or more dead-serotonin modulating bacteria, one or more conditioned medium(s) of serotonin-modulating bacteria, one or more cell pellet(s) of serotonin-modulating bacteria, and/or one or more metabolites and/or proteins (derived from serotonin-modulating bacteria) are synergistic in their capacity to modulate a subject's serotonin signaling or biosynthesis.

In some embodiments, combinations of one or more live serotonin-modulating bacteria, one or more dead-serotonin modulating bacteria, one or more conditioned medium(s) of serotonin-modulating bacteria, one or more cell pellet(s) of serotonin-modulating bacteria, and/or one or more metabolites and/or proteins (derived from serotonin-modulating bacteria) are exemplary in their serotonin-modulating characteristics, as compared to purified metabolites and/or proteins derived from bacteria bacterial strains.

In some embodiments, the combinations of one or more live serotonin-modulating bacteria, one or more dead-serotonin modulating bacteria, one or more conditioned medium(s) of serotonin-modulating bacteria, one or more cell pellet(s) of serotonin-modulating bacteria, and/or one or more metabolites and/or proteins (derived from serotonin-modulating bacteria) can be delivered to the gastrointestinal tract in the form of a capsule, a tablet, a caplet, a pill, a troche, a lozenge, a powders, a granule, a medical food, supplement or a combination thereof. In some embodiments, the composition is administered as a fecal transplant (in embodiments comprising live bacteria), rectally or via suppository.

In some embodiments, combinations of bacteria are selected for therapeutics, food, medical foods, or any other product for synergistic effects on host 5-HT signaling. In a non-limiting example, combinations of bacteria can be selected to capture multiple mechanisms involved in modulating host 5-HT signaling. As a non-limiting example, a strain with only the ability to elevate 5-HT signaling via its cell pellet can be combined with a separate bacterium, wherein said second bacterium has a supernatant that elicits an effect, produces 5-HT, and/or or produces 5-HT agonists. In some embodiments, by combining mechanisms or adding redundancy of any mechanism (e.g., multiple organisms with the capability to produce 5-HT) within a product, one can to elicit a stronger or more consistent effect on host 5-HT signaling, and thus a more favorable impact in the target indications and/or symptoms. In a non-limiting example, Clostridium lavalense HB-452C, which produces 5-HT and the 5-HT agonist tryptamine, can be combined with Bifidobacterium adolescentis HB-179, which has a strong cell pellet and supernatant induction phenotype.

### Therapeutic Compositions

Bacteria, their components, proteins, metabolites, or conditioned medium-derived products can be formulated and/or used as therapeutic compositions, e.g., to alter serotonin levels in an individual in need thereof. Such therapeutic compositions can also be pharmaceutical compositions, formulated with a pharmaceutically acceptable carrier. Thus, as used herein, the term “pharmaceutical composition” refers to the active therapeutic agent in combination with a pharmaceutically acceptable carrier e.g. a carrier commonly used in the pharmaceutical industry. The phrase “pharmaceutically acceptable” is employed herein to refer to those compounds, materials, compositions, and/or dosage forms which are, within the scope of sound medical judgment, suitable for use in contact with the tissues of human beings and animals without excessive toxicity, irritation, allergic response, or other problem or complication, commensurate with a reasonable benefit/risk ratio. In some embodiments of any of the aspects, a pharmaceutically acceptable carrier can be a carrier other than water. In some embodiments of any of the aspects, a pharmaceutically acceptable carrier can be a cream, emulsion, gel, liposome, nanoparticle, and/or ointment. In some embodiments of any of the aspects, a pharmaceutically acceptable carrier can be an artificial or engineered carrier, e.g., a carrier that the active ingredient would not be found to occur in in nature.

Any of the serotonin-modulating bacteria described herein (e.g., live or dead bacteria, natural bacteria or engineered bacteria), conditioned medium(s) of serotonin-modulating bacteria, cell pellet(s) of serotonin-modulating bacteria, and/or metabolites and/or proteins (derived from serotonin-modulating bacteria) can be incorporated into a therapeutic composition. For instance, the therapeutic compositions can be administered to a patient in need thereof to treat or alleviate the symptom of a serotonin-related disease or disorder.

In some embodiments, bacteria, conditioned medium, cell pellets, proteins, and/or metabolites are purified prior to incorporation into a therapeutic composition. For instance, bacteria can be purified so that the population of bacteria is substantially free of other bacteria (e.g., contains at least 90%, at least 91%, at least 92%, at least 93%, at least 94%, at least 95%, at least 96%, at least 97%, or at least 98%, at least 99% of the specific bacterial strain or strains desired in the composition).

In some embodiments, the therapeutic composition is a probiotic or a medical food comprising at least one serotonin-modulating bacterial strain, conditioned medium, cell pellets, purified metabolites, and/or proteins from one or more serotonin-modulating bacteria, or any combinations thereof). The therapeutic composition can be administered, for instance, as a probiotic, a capsule, a tablet, a caplet, a pill, a troche, a lozenge, a powder, granules, or any combination thereof. The composition can also be formulated as a medical food. The composition can also be administered as a fecal transplant or suppository.

In some embodiments, the composition is formulated for oral administration. In some embodiments the composition is formulated for rectal or colorectal administration. In some embodiments, the composition is administered orally, intravenously, intramuscularly, intrathecally, subcutaneously, sublingually, buccally, rectally, vaginally, by the ocular route, by the otic route, nasally, via inhalation, by nebulization, cutaneously, or transdermally. In some embodiments, the composition is administered via multiple methods or routes to increase potency or take advantage of synergistic effects.

In some embodiments, the dose of the therapeutic can comprise e.g., at least 1×10³ CFUs, 1×10⁴ CFUs, 1×10⁵ CFUs, 1×10⁶ CFUs, 1×10⁷ CFUs, 1×10⁸ CFUs, 1×10⁹ CFUs, 1×10¹⁰ CFUs, 1×10¹¹ CFUs, 1×10¹² CFUs, or greater than 1×10² CFUs of the desired bacterial species. In some embodiments, wherein the desired bacterial species are inactivated or not viable, CFUs correspond to the CFUs of an equivalent preparation of live bacterial species, or to the CFUs of the preparation prior to killing or inactivation of the bacterial species. In some embodiments, the dose of the therapeutic can contain the conditioned medium or cell pellet from e.g., at least 1×10³ CFUs, 1×10⁴ CFUs, 1×10⁵ CFUs, 1×10⁶ CFUs, 1×10⁷ CFUs, 1×10⁸ CFUs, 1×10⁹ CFUs, 1×10¹⁰ CFUs, 1×10¹ CFUs, 1×10² CFUs, 1×10¹³ CFUs, 1×10¹⁴ CFUs, or greater than 1×10² CFUs of the desired bacterial species. In some embodiments, the purified metabolite or protein can be derived from e.g., at least 1×10³ CFUs, 1×10⁴ CFUs, 1×10⁵ CFUs, 1×10⁶ CFUs, 1×10⁷ CFUs, 1×10⁸ CFUs, 1×10⁹ CFUs, 1×10¹⁰ CFUs, 1×10¹¹ CFUs, 1×10¹² CFUs, or greater than 1×10¹² CFUs of the desired bacterial species.

In some embodiments, the therapeutic composition or dose unit comprises a pharmaceutically acceptable formulation, including an enteric coating or similar to survive the acidity of the stomach and permit delivery into the small or large intestine, a prebiotic (such as, but not limited to, amino acids (e.g., arginine, glutarate, and ornithine), biotin, fructooligosaccharide, galactooligosaccharides, hemi celluloses (e.g., arabinoxylan, xylan, xyloglucan, and glucomannan), inulin, chitin, lactulose, mannan oligosaccharides, oligofructose-enriched inulin, gums (e.g., guar gum, gum arabic and carrageenan), oligofructose, oligodextrose, tagatose, resistant maltodextrins (e.g., resistant starch), trans-galactooligosaccharide, pectins (e.g., xylogalactouronan, citrus pectin, apple pectin, and rhamnogalacturonan-I), dietary fibers (e.g., soy fiber, sugarbeet fiber, pea fiber, corn bran, and oat fiber) xylooligosaccharides, polyamines (such as but not limited to spermidine and putrescine), an effective amount of an anti-bacterial agent, anti-fungal agent, anti-viral agent, or anti-parasitic agent, or any combinations of the above. As a non-limiting example, the therapeutic composition can also be in the form of a yogurt containing one or more purified strains of serotonin-modulating bacteria, conditioned medium, cell pellet(s), and/or purified metabolite(s) and/or protein(s) from one or more serotonin-modulating bacteria.

### Serotonin-Related Diseases or Disorders

In one or more embodiments of any of the above aspects, a serotonin-related disease or disorder that can be treated by administration of a therapeutic composition described herein is selected from the group consisting of: intestinal motility disorders (e.g., diarrhea or constipation), irritable bowel syndrome (e.g., IBS-D, IBS-C, IBS-M), inflammatory bowel disease, depression (e.g., major depressive disorder, treatment resistant depression, post-partum depression), anxiety, anxiety disorders, addiction, social phobia, major depressive disorder (MDD), neurodegenerative disorders, autism spectrum disorder, sleep disorders, attention deficit hyperactivity disorder (ADHD), memory loss (e.g. dementia), learning difficulties, sleep disorders, schizophrenia, bone disease (e.g. osteoporosis), cancer (e.g. polycythemia vera or myelosclerosis), metabolic disease (e.g. obesity or diabetes), a dysregulated immune system, cardiac disease (e.g. coronary artery disease, heart valve disease, congenital heart disease, cardiomyopathies, pericarditis, or aorta disease), heartburn, dermatological conditions (e.g. eczema and itch), GERD, platelet disorders (e.g. essential thrombocytosis), and pain disorders.

In some embodiments, the serotonin-related disease or disorder or its symptom(s) are caused by high serotonin levels. In some embodiments, the serotonin-related disease or disorder or its symptoms caused by high serotonin levels is selected from the group consisting of: diarrhea, IBS-D, inflammatory bowel disease, anxiety, social phobia, sleep disorders, schizophrenia, cancer, obesity, diabetes, coronary artery disease, heart valve disease, congenital heart disease, cardiomyopathies, pericarditis, or platelet disorders (e.g. essential thrombocytosis).

In some embodiments, the serotonin-related disease or disorder or its symptom(s) caused by high serotonin levels is not a gut disease or disorder. In some embodiments, the serotonin-related disease or disorder or its symptom(s) caused by high serotonin levels is selected from the group consisting of anxiety, social phobia, sleep disorders, schizophrenia, cancer, obesity, diabetes, coronary artery disease, heart valve disease, congenital heart disease, cardiomyopathies, pericarditis, or platelet disorders (e.g. essential thrombocytosis).

In some embodiments, the serotonin-related disease or disorder or its symptom(s) are caused by low serotonin levels. In some embodiments, the serotonin-related disease or disorder or its symptom(s) caused by low serotonin levels is selected from the group consisting of: constipation, IBS-C, depression, anxiety, addiction, neurodegenerative disorders, autism spectrum disorder, sleep disorders, attention deficit hyperactivity disorder (ADHD), memory loss (e.g. dementia), learning difficulties, osteoporosis, heartburn, dermatological conditions (e.g., eczema and itch), gastroesophageal reflux disease (GERD), or pain disorders.

In some embodiments, the serotonin-related disease or disorder or its symptom(s) caused by low serotonin levels is not a gut disease or disorder. In some embodiments, the serotonin-related disease or disorder or its symptom(s) caused by low serotonin levels is selected from the group consisting of depression, anxiety, addiction, neurodegenerative disorders, autism spectrum disorder, sleep disorders, attention deficit hyperactivity disorder (ADHD), memory loss (e.g. dementia), learning difficulties, osteoporosis, dermatological conditions (eczema and itch), and pain disorders.

It is noteworthy that both high and low serotonin levels have been connected to certain anxiety and sleep disorders. In some instances, it is clear that a serotonin level, whether higher or lower, can influence health, thereby emphasizing the importance of serotonin regulation. Where the gut microbiota is centrally involved in maintaining serotonin levels, a dysbiosis that causes a deviation from normal levels can be treated by restoring a healthy gut microbiota or altering the dysbiosis.

In some embodiments, the method further comprises decreasing at least one symptom of a serotonin-related disease or disorder in the subject selected from the group consisting of: fatigue, insomnia, stress, persistent anxiety, persistent sadness, social withdrawal, substance withdrawal, irritability, thoughts of suicide, thoughts of self-harm, restlessness, low sex drive, lack of focus, loss of appetite, high blood pressure, low blood pressure, high heart rate, low heart rate, constipation, diarrhea, chronic pain, heartburn, fatigue, trouble breathing, stomach aches, nosebleeds, gum, or stomach bleeding, headaches, weight gain, and burning of the skin, altered inflammatory markers, neurodevelopment, or seizures.

In some embodiments, the process of identifying a subject with a serotonin-related disease or disorder can be carried out by a trained psychologist, psychiatrist, gastroenterologist, cardiologist, neurologist, or otherwise appropriate medical provider. For instance, a psychiatrist, psychologist, or neurologist can diagnose a subject with a serotonin-related disease or disorder of the central nervous system by evaluating the subject's behavior for symptoms of serotonin-related disease or disorder. One of skill in the art will understand that mental illness can also be identified in a subject with the aid of the Diagnostic and Statistical Manual of Mental Disorders (DSM-5) (American Psychiatric Association).

In one or more embodiments, the process of identifying a subject with a serotonin-related disease or disorder can comprise diagnosing the subject with a serotonin-related disease or disorder. In some embodiments, the serotonin-related disease or disorder is identified or diagnosed using functional magnetic resonance imaging (fMRI). In some embodiments, the serotonin-related disease or disorder can be identified with standard psychological and neurological surveys, or in other methods known to experts in the field. In some embodiments, a serotonin-related disease or disorder can be diagnosed using gastrointestinal related methods, such as a colonoscopy, fecal consistency test, or fecal swab.

In some embodiments, a subject in need of treatment with a therapeutic composition described herein can be identified by identifying low levels of serotonin in the subject's blood, serum, stool, or other bodily fluid. The amount of serotonin can be measured by LC/MS or another technique known in the art. In some embodiments, the amount of serotonin in the brain can be measured using proton magnetic resonance (PMR), or another similar technique.

In some embodiments, a subject in need of treatment with a therapeutic composition described herein can be identified by identifying low or high levels of serotonin-modulating bacteria in the subject's stool or cecum, using such methods as 16S rDNA next-generation sequencing (NGS; e.g., Illumina™) or quantitative PCR. In some embodiments, the percentage of serotonin-modulating bacteria in the subject's gut represents more than 9%, about 9%, about 8%, about 7%, about 6%, about 5%, about 4%, about 3%, about 2%, about 1%, or less than about 1% of the total 16S sequences measured in the subject's stool or cecal sample.

Accordingly, the present disclosure provides for the treatment of one or more serotonin-related disease or disorders by administering to the subject one or combinations of one or more live serotonin-modulating bacteria, one or more dead-serotonin modulating bacteria, one or more conditioned medium(s) of one or more serotonin-modulating bacteria, one or more cell pellet(s) of serotonin-modulating bacteria, and/or one or more metabolites and/or proteins (derived from serotonin-modulating bacteria). The present disclosure provides for the treatment of one or more serotonin-related disease or disorder by treatment with a prebiotic.

### Treatment Methods

The therapeutic compositions described herein can be administered to a patient in need thereof, for instance for the treatment of a serotonin-related disease or disorder. In some embodiments, the method of treatment can comprise first diagnosing a patient who can benefit from treatment by a therapeutic composition described herein. In some embodiments, the method further comprises administering to the patient a therapeutic composition described herein.

As used herein, the terms “treat,” “treatment,” “treating,” or “amelioration” refer to therapeutic treatments, wherein the object is to reverse, alleviate, ameliorate, inhibit, slow down or stop the progression or severity of a condition associated with a disease or disorder, e.g. a serotonin-related disease or disorder. The term “treating” includes reducing or alleviating at least one adverse effect or symptom of a condition, disease or disorder associated with a serotonin-related disease or disorder. Treatment is generally “effective” if one or more symptoms or clinical markers are reduced. Alternatively, treatment is “effective” if the progression of a disease is reduced or halted. That is, “treatment” includes not just the improvement of symptoms or markers, but also a cessation of, or at least slowing of, progress or worsening of symptoms compared to what would be expected in the absence of treatment. Beneficial or desired clinical results include, but are not limited to, alleviation of one or more symptom(s), diminishment of extent of disease, stabilized (i.e., not worsening) state of disease, delay or slowing of disease progression, amelioration or palliation of the disease state, remission (whether partial or total), and/or decreased mortality, whether detectable or undetectable. The term “treatment” of a disease also includes providing relief from the symptoms or side-effects of the disease (including palliative treatment).

As described herein, levels of serotonin can be increased or decreased in a serotonin-related disease or disorder and/or in subjects with a serotonin-related disease or disorder, that is, that level of serotonin can deviate from a normal level.

In some embodiments of any of the aspects, the level of serotonin can be decreased in a serotonin-related disease or disorder and/or in subjects with a serotonin-related disease or disorder. Accordingly, in one aspect of any of the embodiments, described herein is a method of treating a serotonin-related disease or disorder in a subject in need thereof, the method comprising administering a composition comprising at least one serotonin modulating bacteria (e.g., a serotonin-increasing bacteria) and/or product(s) thereof as described herein to a subject determined to have a level of serotonin that is decreased relative to a reference. In one aspect of any of the embodiments, described herein is a method of treating a serotonin-related disease or disorder in a subject in need thereof, the method comprising: a) determining the level of serotonin in a sample obtained from a subject; and b) administering a composition comprising at least one serotonin modulating bacteria and/or product(s) as described herein to the subject if the level of serotonin is decreased relative to a reference.

In some embodiments of any of the aspects, the method comprises administering composition comprising at least one serotonin modulating bacteria and/or product(s) as described herein to a subject previously determined to have a level of serotonin that is decreased relative to a reference. In some embodiments of any of the aspects, described herein is a method of treating a serotonin-related disease or disorder in a subject in need thereof, the method comprising: a) first determining the level of serotonin in a sample obtained from a subject; and b) then administering a composition comprising at least one serotonin modulating bacteria and/or product(s) as described herein to the subject if the level of serotonin is decreased relative to a reference.

In one aspect of any of the embodiments, described herein is a method of treating a serotonin-related disease or disorder in a subject in need thereof, the method comprising: a) determining if the subject has a decreased level of serotonin; and b) administering a composition comprising at least one serotonin modulating bacteria and/or product(s) as described herein to the subject if the level of serotonin is decreased relative to a reference. In some embodiments of any of the aspects, the step of determining if the subject has a decreased level of serotonin can comprise i) obtaining or having obtained a sample from the subject and ii) performing or having performed an assay on the sample obtained from the subject to determine/measure the level of serotonin in the subject. In some embodiments of any of the aspects, the step of determining if the subject has a decreased level of serotonin can comprise performing or having performed an assay on a sample obtained from the subject to determine/measure the level of serotonin in the subject. In some embodiments of any of the aspects, the step of determining if the subject has a decreased level of serotonin can comprise ordering or requesting an assay on a sample obtained from the subject to determine/measure the level of serotonin in the subject. In some embodiments of any of the aspects, the step of determining if the subject has a decreased level of serotonin can comprise receiving the results of an assay on a sample obtained from the subject to determine/measure the level of serotonin in the subject. In some embodiments of any of the aspects, the step of determining if the subject has a decreased level of serotonin can comprise receiving a report, results, or other means of identifying the subject as a subject with a decreased level of serotonin.

In one aspect of any of the embodiments, described herein is a method of treating a serotonin-related disease or disorder in a subject in need thereof, the method comprising: a) determining if the subject has a decreased level of serotonin; and b) instructing or directing that the subject be administered a composition comprising at least one serotonin modulating bacteria and/or product(s) as described herein if the level of serotonin is decreased relative to a reference. In some embodiments of any of the aspects, the step of determining if the subject has a decreased level of serotonin can comprise i) obtaining or having obtained a sample from the subject and ii) performing or having performed an assay on the sample obtained from the subject to determine/measure the level of serotonin in the subject. In some embodiments of any of the aspects, the step of determining if the subject has a decreased level of serotonin can comprise performing or having performed an assay on a sample obtained from the subject to determine/measure the level of serotonin in the subject. In some embodiments of any of the aspects, the step of determining if the subject has a decreased level of serotonin can comprise ordering or requesting an assay on a sample obtained from the subject to determine/measure the level of serotonin in the subject. In some embodiments of any of the aspects, the step of instructing or directing that the subject be administered a particular treatment can comprise providing a report of the assay results. In some embodiments of any of the aspects, the step of instructing or directing that the subject be administered a particular treatment can comprise providing a report of the assay results and/or treatment recommendations in view of the assay results.

In some embodiments of any of the aspects, the level of serotonin can be increased in a serotonin-related disease or disorder and/or in subjects with a serotonin-related disease or disorder. Accordingly, in one aspect of any of the embodiments, described herein is a method of treating a serotonin-related disease or disorder in a subject in need thereof, the method comprising administering a composition comprising at least one serotonin modulating bacteria (e.g., a serotonin-reducing bacteria) and/or product(s) thereof as described herein to a subject determined to have a level of serotonin that is increased relative to a reference. In one aspect of any of the embodiments, described herein is a method of treating a serotonin-related disease or disorder in a subject in need thereof, the method comprising: a) determining the level of serotonin in a sample obtained from a subject; and b) administering a composition comprising at least one serotonin modulating bacteria and/or product(s) as described herein to the subject if the level of serotonin is increased relative to a reference.

In some embodiments of any of the aspects, the method comprises administering composition comprising at least one serotonin modulating bacteria and/or product(s) as described herein to a subject previously determined to have a level of serotonin that is increased relative to a reference. In some embodiments of any of the aspects, described herein is a method of treating a serotonin-related disease or disorder in a subject in need thereof, the method comprising: a) first determining the level of serotonin in a sample obtained from a subject; and b) then administering a composition comprising at least one serotonin modulating bacteria and/or product(s) as described herein to the subject if the level of serotonin is increased relative to a reference.

In one aspect of any of the embodiments, described herein is a method of treating a serotonin-related disease or disorder in a subject in need thereof, the method comprising: a) determining if the subject has an increased level of serotonin; and b) administering a composition comprising at least one serotonin modulating bacteria and/or product(s) as described herein to the subject if the level of serotonin is increased relative to a reference. In some embodiments of any of the aspects, the step of determining if the subject has an increased level of serotonin can comprise i) obtaining or having obtained a sample from the subject and ii) performing or having performed an assay on the sample obtained from the subject to determine/measure the level of serotonin in the subject. In some embodiments of any of the aspects, the step of determining if the subject has an increased level of serotonin can comprise performing or having performed an assay on a sample obtained from the subject to determine/measure the level of serotonin in the subject. In some embodiments of any of the aspects, the step of determining if the subject has an increased level of serotonin can comprise ordering or requesting an assay on a sample obtained from the subject to determine/measure the level of serotonin in the subject. In some embodiments of any of the aspects, the step of determining if the subject has an increased level of serotonin can comprise receiving the results of an assay on a sample obtained from the subject to determine/measure the level of serotonin in the subject. In some embodiments of any of the aspects, the step of determining if the subject has an increased level of serotonin can comprise receiving a report, results, or other means of identifying the subject as a subject with an increased level of serotonin.

In one aspect of any of the embodiments, described herein is a method of treating a serotonin-related disease or disorder in a subject in need thereof, the method comprising: a) determining if the subject has an increased level of serotonin; and b) instructing or directing that the subject be administered a composition comprising at least one serotonin modulating bacteria and/or product(s) as described herein if the level of serotonin is increased relative to a reference. In some embodiments of any of the aspects, the step of determining if the subject has an increased level of serotonin can comprise i) obtaining or having obtained a sample from the subject and ii) performing or having performed an assay on the sample obtained from the subject to determine/measure the level of serotonin in the subject. In some embodiments of any of the aspects, the step of determining if the subject has an increased level of serotonin can comprise performing or having performed an assay on a sample obtained from the subject to determine/measure the level of serotonin in the subject. In some embodiments of any of the aspects, the step of determining if the subject has an increased level of serotonin can comprise ordering or requesting an assay on a sample obtained from the subject to determine/measure the level of serotonin in the subject. In some embodiments of any of the aspects, the step of instructing or directing that the subject be administered a particular treatment can comprise providing a report of the assay results. In some embodiments of any of the aspects, the step of instructing or directing that the subject be administered a particular treatment can comprise providing a report of the assay results and/or treatment recommendations in view of the assay results.

In some embodiments, individuals that would benefit from an alteration of levels of the serotonin-modulating gut microbiota, serotonin-modulating gut-microbiota-derived metabolome, and/or or serotonin-modulating gut-microbiota-derived proteome of the subject are identified via next-generation DNA and/or RNA sequencing of microbial communities in that individual's stool or tissue samples; metabolomics of stool, urine, blood, or similar samples; or genome sequencing of that individual; or some combination thereof. In some embodiments, serotonin is measured in the stool, blood, or tissue of the subject. In some embodiments, levels of serotonin modulating bacteria are measured. In such embodiments, low levels of serotonin-modulating gut microbiota can indicate the need to introduce, promote, or select for increased serotonin-modulating bacteria. In some embodiments, levels of genes involved in the production of microbiota-derived serotonin modulating metabolites or proteins are measured. In some embodiments, levels of serotonin-modulating bacteria, serotonin-modulating metabolites, or serotonin-modulating proteins are altered relative to their initial quantitated amounts, after administering a therapeutic composition as described herein.

### Administration

Therapeutic compositions as described herein can be administered via any of a number of different routes or in different regimens. As used herein, the term “administering,” refers to the placement of a compound or bacteria as disclosed herein into a subject by a method or route which results in at least partial delivery of the agent at a desired site. Pharmaceutical compositions comprising the compounds or bacteria disclosed herein can be administered by any appropriate route which results in an effective treatment in the subject. In some embodiments, administration comprises physical human activity, e.g., an injection, act of ingestion, an act of application, and/or manipulation of a delivery device or machine. Such activity can be performed, e.g., by a medical professional and/or the subject being treated. The period of viability of the bacterial cells after administration to a subject can be as short as a few hours, e.g., twenty-four hours, to a few days, to as long as several years, i.e., long-term engraftment.

In other words, as used herein “administer” and “administration” encompasses embodiments in which one person directs another to consume, ingest, or otherwise take into the body a bacteria, bacterial composition, bacterial conditioned media, bacterial cell pellet, purified bacterial metabolites, purified bacterial proteins, or combinations thereof in a certain manner and/or for a certain purpose, and also situations in which a user uses any of these compositions in a certain manner and/or for a certain purpose independently of or in variance to any instructions received from a second person. Non-limiting examples of embodiments in which one person directs another to consume a composition described herein in a certain manner and/or for a certain purpose include when a physician prescribes a course of conduct and/or treatment to a patient, when a parent commands a minor user (such as a child) to consume a composition described herein, when a trainer advises a user (such as an athlete) to follow a particular course of conduct and/or treatment, and when a manufacturer, distributer, or marketer recommends conditions of use to an end user, for example through advertisements or labeling on packaging or on other materials provided in association with the sale or marketing of a product.

In some embodiments, the methods described herein relate to treating a subject having or diagnosed as having a serotonin-related disease or disorder with a composition comprising at least one serotonin modulating bacteria and/or product(s) as described herein. Subjects having a serotonin-related disease or disorder can be identified by a physician using current methods of diagnosing a serotonin-related disease or disorder. Symptoms and/or complications of a serotonin-related disease or disorder which characterize these conditions and aid in diagnosis are well known in the art, as described above. Tests that can aid in a diagnosis of, e.g. a serotonin-related disease or disorder are described above and can include, in addition to standard measurements of serotonin itself, detection or measurement of gut bacteria that modulate serotonin, detection or measurement of genetic sequences of such bacteria, including 16S sequences and/or genetic sequences encoding proteins that modulate serotonin, or detection or measurement of bacterial metabolites or proteins that modulate serotonin. A family history of a serotonin-related disease or disorder, or exposure to risk factors for a serotonin-related disease or disorder can also aid in determining if a subject is likely to have a serotonin-related disease or disorder or in making a diagnosis of a serotonin-related disease or disorder.

In some embodiments, the methods described herein comprise administering an effective amount of a composition or compositions described herein, e.g. a composition comprising at least one serotonin modulating bacteria and/or product(s) as described herein to a subject in order to alleviate a symptom of a serotonin-related disease or disorder. As used herein, “alleviating a symptom of a serotonin-related disease or disorder” is ameliorating any condition or symptom associated with the a serotonin-related disease or disorder. As compared with an equivalent untreated control, such amelioration comprises a reduction by at least 5%, 10%, 20%, 40%, 50%, 60%, 80%, 90%, 95%, 99% or more as measured by any standard technique. A variety of means for administering the compositions described herein to subjects are known to those of skill in the art. Such methods can include, but are not limited to oral, parenteral, intravenous, intramuscular, subcutaneous, transdermal, airway (aerosol), pulmonary, cutaneous, topical, injection, or intratumoral administration. Administration can be local or systemic.

The term “effective amount” as used herein refers to the amount of a composition comprising at least one serotonin modulating bacteria and/or product(s) as described herein needed to alleviate at least one or more symptom of the disease or disorder, and relates to a sufficient amount of pharmacological composition to provide the desired effect. The term “therapeutically effective amount” therefore refers to an amount of a composition comprising at least one serotonin modulating bacteria and/or product(s) as described herein that is sufficient to provide a particular anti-serotonin-related-disorder effect when administered to atypical subject. An effective amount as used herein, in various contexts, would also include an amount sufficient to delay the development of a symptom of the disease, alter the course of a symptom disease (for example but not limited to, slowing the progression of a symptom of the disease), or reverse a symptom of the disease. Thus, it is not generally practicable to specify an exact “effective amount”. However, for any given case, an appropriate “effective amount” can be determined by one of ordinary skill in the art using only routine experimentation.

Effective amounts, toxicity, and therapeutic efficacy can be determined by standard pharmaceutical procedures in cell cultures or experimental animals, e.g., for determining the LD50 (the dose lethal to 50% of the population) and the ED50 (the dose therapeutically effective in 50% of the population). The dosage can vary depending upon the dosage form employed and the route of administration utilized. The dose ratio between toxic and therapeutic effects is the therapeutic index and can be expressed as the ratio LD50/ED50. Compositions and methods that exhibit large therapeutic indices are preferred. A therapeutically effective dose can be estimated initially from cell culture assays. Also, where a therapeutic composition's active agent or ingredient comprises, consists essentially of, or consists of a metabolite or protein produced by a bacteria or bacterial composition as described herein, a dose can be formulated in animal models to achieve a concentration range in vivo that includes the IC50 (i.e., the concentration of a composition comprising at least one product of at least one serotonin modulating bacteria as described herein, which achieves a half-maximal inhibition of symptoms) as determined in cell culture, or in an appropriate animal model. Levels in biological samples can be measured, for example, by high performance liquid chromatography. The effects of any particular dosage can be monitored by a suitable bioassay, e.g., assay for serotonin, among others. The dosage can be determined by a physician and adjusted, as necessary, to suit observed effects of the treatment.

In some embodiments, the technology described herein relates to a pharmaceutical composition comprising a composition comprising at least one serotonin modulating bacteria and/or product(s) as described herein, and optionally a pharmaceutically acceptable carrier. In some embodiments, the active ingredients of the pharmaceutical composition comprise at least one serotonin modulating bacteria and/or product(s) as described herein. In some embodiments, the active ingredients of the pharmaceutical composition consist essentially of at least one serotonin modulating bacteria and/or product(s) as described herein. In some embodiments, the active ingredients of the pharmaceutical composition consist of at least one serotonin modulating bacteria and/or product(s) as described herein. Pharmaceutically acceptable carriers and diluents include saline, aqueous buffer solutions, solvents and/or dispersion media. The use of such carriers and diluents is well known in the art. Some non-limiting examples of materials which can serve as pharmaceutically-acceptable carriers include: (1) sugars, such as lactose, glucose and sucrose; (2) starches, such as corn starch and potato starch; (3) cellulose, and its derivatives, such as sodium carboxymethyl cellulose, methylcellulose, ethyl cellulose, microcrystalline cellulose and cellulose acetate; (4) powdered tragacanth; (5) malt; (6) gelatin; (7) lubricating agents, such as magnesium stearate, sodium lauryl sulfate and talc; (8) excipients, such as cocoa butter and suppository waxes; (9) oils, such as peanut oil, cottonseed oil, safflower oil, sesame oil, olive oil, corn oil and soybean oil; (10) glycols, such as propylene glycol; (11) polyols, such as glycerin, sorbitol, mannitol and polyethylene glycol (PEG); (12) esters, such as ethyl oleate and ethyl laurate; (13) agar; (14) buffering agents, such as magnesium hydroxide and aluminum hydroxide; (15) alginic acid; (16) pyrogen-free water; (17) isotonic saline; (18) Ringer's solution; (19) ethyl alcohol; (20) pH buffered solutions; (21) polyesters, polycarbonates and/or polyanhydrides; (22) bulking agents, such as polypeptides and amino acids (23) serum component, such as serum albumin, HDL and LDL; (24) C₂-C₁ₐ alcohols, such as ethanol; and (25) other non-toxic compatible substances employed in pharmaceutical formulations. Wetting agents, coloring agents, release agents, coating agents, sweetening agents, flavoring agents, perfuming agents, preservative and antioxidants can also be present in the formulation. The terms such as “excipient”, “carrier”, “pharmaceutically acceptable carrier” or the like are used interchangeably herein. In some embodiments, the carrier inhibits the degradation of the active agent, e.g. at least one serotonin modulating bacteria and/or product(s) as described herein.

In some embodiments, the pharmaceutical composition comprising at least one serotonin modulating bacteria and/or product(s) as described herein can be a parenteral dose form. Since administration of parenteral dosage forms typically bypasses the patient's natural defenses against contaminants, parenteral dosage forms are preferably sterile or capable of being sterilized prior to administration to a patient. Examples of parenteral dosage forms include, but are not limited to, solutions ready for injection, dry products ready to be dissolved or suspended in a pharmaceutically acceptable vehicle for injection, suspensions ready for injection, and emulsions. In addition, controlled-release parenteral dosage forms can be prepared for administration of a patient, including, but not limited to, DUROS®-type dosage forms and dose-dumping.

Suitable vehicles that can be used to provide parenteral dosage forms of at least one serotonin modulating bacteria and/or product(s) as disclosed within are well known to those skilled in the art. Examples include, without limitation: sterile water; water for injection USP; saline solution; glucose solution; aqueous vehicles such as but not limited to, sodium chloride injection, Ringer's injection, dextrose Injection, dextrose and sodium chloride injection, and lactated Ringer's injection; water-miscible vehicles such as, but not limited to, ethyl alcohol, polyethylene glycol, and propylene glycol; and non-aqueous vehicles such as, but not limited to, corn oil, cottonseed oil, peanut oil, sesame oil, ethyl oleate, isopropyl myristate, and benzyl benzoate. Compounds that alter or modify the solubility of a pharmaceutically acceptable salt of a serotonin modulating bacterial product as disclosed herein can also be incorporated into the parenteral dosage forms of the disclosure, including conventional and controlled-release parenteral dosage forms.

Pharmaceutical compositions comprising at least one serotonin modulating bacteria and/or product(s) can also be formulated to be suitable for oral administration, for example as discrete dosage forms, such as, but not limited to, tablets (including without limitation scored or coated tablets), pills, caplets, capsules, chewable tablets, powder packets, cachets, troches, wafers, aerosol sprays, or liquids, such as but not limited to, syrups, elixirs, solutions or suspensions in an aqueous liquid, a non-aqueous liquid, an oil-in-water emulsion, or a water-in-oil emulsion. Such compositions contain a predetermined amount of the pharmaceutically acceptable salt of the disclosed compounds, and can be prepared by methods of pharmacy well known to those skilled in the art. See generally, Remington: The Science and Practice of Pharmacy, 21st Ed., Lippincott, Williams, and Wilkins, Philadelphia Pa. (2005).

Conventional dosage forms generally provide rapid or immediate drug release from the formulation. Depending on the pharmacology and pharmacokinetics of the drug, use of conventional dosage forms can lead to wide fluctuations in the concentrations of the drug in a patient's blood and other tissues. These fluctuations can impact a number of parameters, such as dose frequency, onset of action, duration of efficacy, maintenance of therapeutic blood levels, toxicity, side effects, and the like. Advantageously, controlled-release formulations can be used to control a drug's onset of action, duration of action, plasma levels within the therapeutic window, and peak blood levels. In particular, controlled- or extended-release dosage forms or formulations can be used to ensure that the maximum effectiveness of a drug is achieved while minimizing potential adverse effects and safety concerns, which can occur both from under-dosing a drug (i.e., going below the minimum therapeutic levels) as well as exceeding the toxicity level for the drug. In some embodiments, the composition comprising at least one serotonin modulating bacteria and/or product(s) as described herein can be administered in a sustained release formulation.

Controlled-release pharmaceutical products have a common goal of improving drug therapy over that achieved by their non-controlled release counterparts. Ideally, the use of an optimally designed controlled-release preparation in medical treatment is characterized by a minimum of drug substance being employed to cure or control the condition in a minimum amount of time. Advantages of controlled-release formulations include: 1) extended activity of the drug; 2) reduced dosage frequency; 3) increased patient compliance; 4) usage of less total drug; 5) reduction in local or systemic side effects; 6) minimization of drug accumulation; 7) reduction in blood level fluctuations; 8) improvement in efficacy of treatment; 9) reduction of potentiation or loss of drug activity; and 10) improvement in speed of control of diseases or conditions. Kim, Cherng-ju, Controlled Release Dosage Form Design, 2 (Technomic Publishing, Lancaster, Pa.: 2000).

Most controlled-release formulations are designed to initially release an amount of drug (active ingredient) that promptly produces the desired therapeutic effect, and gradually and continually release other amounts of drug to maintain this level of therapeutic or prophylactic effect over an extended period of time. In order to maintain this constant level of drug in the body, the drug must be released from the dosage form at a rate that will replace the amount of drug being metabolized and excreted from the body. Controlled-release of an active ingredient can be stimulated by various conditions including, but not limited to, pH, ionic strength, osmotic pressure, temperature, enzymes, water, and other physiological conditions or compounds.

A variety of known controlled- or extended-release dosage forms, formulations, and devices can be adapted for use with the salts and compositions of the disclosure. Examples include, but are not limited to, those described in U.S. Pat. Nos. 3,845,770; 3,916,899; 3,536,809; 3,598,123; 4,008,719; 5,674,533; 5,059,595; 5,591,767; 5,120,548; 5,073,543; 5,639,476; 5,354,556; 5,733,566; and 6,365,185 B1; each of which is incorporated herein by reference. These dosage forms can be used to provide slow or controlled-release of one or more active ingredients using, for example, hydroxypropylmethyl cellulose, other polymer matrices, gels, permeable membranes, osmotic systems (such as OROS® (Alza Corporation, Mountain View, Calif. USA)), or a combination thereof to provide the desired release profile in varying proportions.

In some embodiments of any of the aspects, the composition comprising at least one serotonin modulating bacteria and/or product(s) as described herein described herein is administered as a monotherapy, e.g., another treatment for the serotonin-related disease or disorder is not administered to the subject.

In some embodiments of any of the aspects, the methods described herein can further comprise administering a second agent and/or treatment to the subject, e.g. as part of a therapy. The combination therapy, where employed, can be tailored to the particular indication. For example, where a serotonin-modulating bacteria or product(s) as described herein is administered to treat anxiety or depression, it can be administered in combination with an anti-anxiety or anti-depression drug or therapy as known in the art or approved for clinical treatment of anxiety or depression. Other indications can be similarly treated with serotonin modulating bacteria or their products as described herein in combination with agents known in the art or approved for the clinical treatment of those indications. As non-limiting examples, where the disease or serotonin-related disease or disorder is a metabolic disease, such as diabetes, one or more anti-diabetes drugs can be administered in combination with the compositions described herein. Alternatively, where the serotonin-related disease or disorder is cancer, the composition(s) described herein can be administered with one or more anti-cancer agents, e.g., chemotherapy agents or other anti-cancer agents known the art.

As a further non-limiting example, if a subject is to be treated for pain or inflammation related to or associated with a serotonin-related disease or disorder as described herein, the subject can also be administered a second agent and/or treatment known to be beneficial for subjects suffering from pain or inflammation. Examples of such agents and/or treatments include, but are not limited to non-steroidal anti-inflammatory drugs (NSAIDs—such as aspirin, ibuprofen, or naproxen); corticosteroids, including glucocorticoids (e.g. cortisol, prednisone, prednisolone, methylprednisolone, dexamethasone, betamethasone, triamcinolone, and beclometasone); methotrexate; sulfasalazine; leflunomide; anti-TNF medications; cyclophosphamide; pro-resolving drugs; mycophenolate; or opiates (e.g. endorphins, enkephalins, and dynorphin), steroids, analgesics, barbiturates, oxycodone, morphine, lidocaine, and the like.

In certain embodiments, an effective dose of a composition comprising at least one serotonin modulating bacteria and/or product(s) as described herein can be administered to a patient once. In certain embodiments, an effective dose of a composition comprising at least one serotonin modulating bacteria and/or product(s) as described herein can be administered to a patient repeatedly. For systemic administration, subjects can be administered a therapeutic amount of a composition comprising for example a metabolite or product of a serotonin-modulating bacteria as described herein, such as, e.g. 0.1 mg/kg, 0.5 mg/kg, 1.0 mg/kg, 2.0 mg/kg, 2.5 mg/kg, 5 mg/kg, 10 mg/kg, 15 mg/kg, 20 mg/kg, 25 mg/kg, 30 mg/kg, 40 mg/kg, 50 mg/kg, or more.

In some embodiments, after an initial treatment regimen, the treatments can be administered on a less frequent basis. For example, after treatment biweekly for three months, treatment can be repeated once per month, for six months or a year or longer. Depending upon the indication, treatment according to the methods described herein can increase levels of a marker (e.g., serotonin or other marker) or symptom of a condition, e.g. a serotonin-related disease or disorder by at least 10%, at least 15%, at least 20%, at least 25%, at least 30%, at least 40%, at least 50%, at least 60%, at least 70%, at least 80% or at least 90% or more. Alternatively, treatment according to the methods described herein can reduce levels of a marker (e.g., serotonin or other marker) or symptom of a condition, e.g. a serotonin-related disease or disorder by at least 10%, at least 15%, at least 20%, at least 25%, at least 30%, at least 40%, at least 50%, at least 60%, at least 70%, at least 80% or at least 90% or more.

The dosage of a composition as described herein can be determined by a physician and adjusted, as necessary, to suit observed effects of the treatment. With respect to duration and frequency of treatment, it is typical for skilled clinicians to monitor subjects in order to determine when the treatment is providing therapeutic benefit, and to determine whether to increase or decrease dosage, increase or decrease administration frequency, discontinue treatment, resume treatment, or make other alterations to the treatment regimen. The dosing schedule can vary from once a week to daily depending on a number of clinical factors, such as the subject's sensitivity to a composition comprising at least one serotonin modulating bacteria and/or product(s) as described herein. The desired dose or amount of activation can be administered at one time or divided into subdoses, e.g., 2-4 subdoses and administered over a period of time, e.g., at appropriate intervals through the day or other appropriate schedule. In some embodiments, administration can be chronic, e.g., one or more doses and/or treatments daily over a period of weeks or months. Examples of dosing and/or treatment schedules are administration daily, twice daily, three times daily or four or more times daily over a period of 1 week, 2 weeks, 3 weeks, 4 weeks, 1 month, 2 months, 3 months, 4 months, 5 months, or 6 months, or more. Alternative examples include dosing daily, every other day, twice weekly, every 10 days, every two weeks, once a month, every six weeks, every two months, or less frequently. A composition comprising at least one serotonin modulating bacteria and/or product(s) as described herein can be administered over a period of time, such as over a 5 minute, 10 minute, 15 minute, 20 minute, or 25 minute period.

The dosage ranges for the administration of a composition comprising at least one serotonin modulating bacteria and/or product(s) as described herein, according to the methods described herein depend upon, for example, the form of the composition, its potency, and the extent to which symptoms, markers, or indicators of a condition described herein are desired to be reduced, for example the percentage reduction or increase desired for serotonin. The dosage should not be so large as to cause adverse side effects. Generally, the dosage will vary with the age, condition, and sex of the patient and can be determined by one of skill in the art. The dosage can also be adjusted by the individual physician in the event of any complication.

The efficacy of a composition comprising at least one serotonin modulating bacteria and/or product(s) as described herein, e.g. the treatment of a condition described herein, or to induce a response as described herein (e.g. modulation of serotonin levels) can be determined by the skilled clinician. However, a treatment is considered “effective treatment,” as the term is used herein, if one or more of the signs or symptoms of a condition described herein are altered in a beneficial manner, other clinically accepted symptoms are improved, or even ameliorated, or a desired response is induced e.g., by at least 10% following treatment according to the methods described herein. Efficacy can be assessed, for example, by measuring a marker, indicator, symptom, and/or the incidence of a condition treated according to the methods described herein or any other measurable parameter appropriate. Efficacy can also be measured by a failure of an individual to worsen as assessed by hospitalization, or need for medical interventions (i.e., progression of the disease is halted). Methods of measuring these indicators are known to those of skill in the art and/or are described herein. Treatment includes any treatment of a disease in an individual or an animal (some non-limiting examples include a human or an animal) and includes: (1) inhibiting the disease, e.g., preventing a worsening of symptoms (e.g. pain or inflammation); or (2) relieving the severity of the disease, e.g., causing regression of symptoms. An effective amount for the treatment of a disease means that amount which, when administered to a subject in need thereof, is sufficient to result in effective treatment as that term is defined herein, for that disease. Efficacy of an agent can be determined by assessing physical indicators of a condition or desired response. It is well within the ability of one skilled in the art to monitor efficacy of administration and/or treatment by measuring any one of such parameters, or any combination of parameters. Efficacy can be assessed in animal models of a condition described herein, for example treatment of serotonin-related disease or disorder. When using an experimental animal model, efficacy of treatment is evidenced when a statistically significant change in a marker is observed, e.g. serotonin.

In vitro and animal model assays are provided herein which allow the assessment of a given dose of a composition comprising at least one serotonin modulating bacteria and/or product(s) as described herein. By way of non-limiting example, the effects of a dose of a composition comprising at least one serotonin modulating bacteria and/or product(s) as described herein can be assessed by a RIN14B cell culture model. A non-limiting example of a protocol for such an assay is described in Example 1.

The efficacy of a given dosage combination can also be assessed in an animal model, e.g. germ-free animal models or alternatively, in a specific pathogen-free (SPF) animal model, or in an animal model of a serotonin-related disease or disorder.

### Definitions

For convenience, the meaning of some terms and phrases used in the specification, examples, and appended claims, are provided below. Unless stated otherwise, or implicit from context, the following terms and phrases include the meanings provided below. The definitions are provided to aid in describing particular embodiments, and are not intended to limit the claimed invention, because the scope of the invention is limited only by the claims. Unless otherwise defined, all technical and scientific terms used herein have the same meaning as commonly understood by one of ordinary skill in the art to which this invention belongs. If there is an apparent discrepancy between the usage of a term in the art and its definition provided herein, the definition provided within the specification shall prevail.

For convenience, certain terms employed herein, in the specification, examples and appended claims are collected here.

The term ‘isolated’ encompasses a bacterium or other entity or substance that has been (1) separated from at least some of the components with which it was associated when initially produced (whether in nature, such as human stool, or in an experimental setting, such as a Petri plate consisting of artificial growth medium), and/or (2) produced, prepared, purified, and/or manufactured by the hand of man. Isolated bacteria can be separated from at least about 10%, about 20%, about 30%, about 40%, about 50%, about 60%, about 70%, about 80%, about 90%, or more of the other components with which they were initially associated. In some embodiments, isolated bacteria are more than about 80%, about 85%, about 90%, about 91%, about 92%, about 93%, about 94%, about 95%, about 96%, about 97%, about 98%, about 99%, or more than about 99% pure. As used herein, a substance is “pure’ if it is substantially free of other components (such as other bacterial species). Thus, a bacterial culture or preparation grown out of a single colony and lacking other species or strains is a pure culture.

The terms “purify,” “purifying” and “purified” refer to a bacterium or other material that has been separated from at least some of the components with which it was associated either when initially produced or generated (e.g., whether in nature or in an experimental setting), or during any time after its initial production, as recognized by those skilled in the art of bacterial cultivation. A bacterium or a bacterial population can be considered purified if it is isolated at or after production, such as from a material or environment containing the bacterium or bacterial population, and a purified bacterium or bacterial population can contain other materials up to about 10%, about 20%, about 30%, about 40%, about 50%, about 60%, about 70%, about 80%, about 90%, or above about 90% and still be considered “isolated.”

In some embodiments, purified bacteria and bacterial populations are more than about 80%, about 85%, about 90%, about 91%, about 92%, about 93%, about 94%, about 95%, about 96%, about 97%, about 98%, about 99%, or more than about 99% pure. In the instance of bacterial compositions provided herein, the one or more bacterial types present in the composition can be independently purified from one or more other bacteria produced and/or present in the material or environment containing the bacterial type. Bacterial compositions and the bacterial components thereof are generally purified from residual habitat products. In the instance of bacterial conditioned medium or cell pellets, these are considered pure if derived from an isolated bacteria, or combination of bacteria intentionally mixed (e.g. two serotonin modulating bacteria, which when mixed, result in the production of metabolites or proteins not produced or not produced efficiently in isolation). In the case of purified metabolites or proteins, these are considered to be “isolated” if they are free of about 10%, about 20%, about 30%, about 40%, about 50%, about 60%, about 70%, about 80%, about 90%, or above about 90% of other components of a bacterial conditioned medium or cell pellet, wherein the other components are not the target purified metabolite or protein.

As used herein, “probiotic” is understood to mean live microorganisms which when administered in adequate amounts confer a health benefit on the host.

As used herein, “prebiotic” is understood to mean an ingredient that allows or promotes specific changes, in the composition and/or activity of the gastrointestinal microbiota that may or may not confer benefits upon the host.

As used herein, “medical food” is understood to mean a food which is formulated to be consumed or administered enterally under the supervision of a physician and which is intended for the specific dietary management of a disease or condition for which distinctive nutritional requirements, based on recognized scientific principles, are established by medical evaluation.

As used herein, “supplement” (also referred to as a dietary supplement) is understood to mean a product taken orally that comprises one or more ingredients (e.g., vitamins, minerals, amino acids, an isolated microbe or product thereof as described herein) that are intended to supplement one's diet and are not considered food. As non-limiting examples, a supplement can be in the form of a capsule, a tablet, a caplet, a pill, a troche, a lozenge, a powder, or a granule.

As used herein “initial amount” is understood to mean the amount of a substance, e.g., serotonin, in an aliquot or sample, prior to administration of a therapeutic composition as described herein. Initial amount can be measured in terms of concentration. For instance, an initial amount can be measured in terms of micrograms of substance per milliliter of sample, e.g., micrograms of serotonin per milliliter of blood or serum g serotonin/mL blood or serum). The initial amount of serotonin can also be measured, for instance, as the amount of serotonin in regions of the brain, such as the prefrontal cortex prior to administration of a described composition. The amount of serotonin can be represented in terms of millimoles of serotonin per kg tissue (mmol serotonin per kg of brain tissue). The initial amount can also be measured, for instance, as the amount of serotonin in a subject's stool sample prior to administration of a described composition to the subject. The amount of serotonin can be represented in terms of micrograms of serotonin per gram of stool (ug serotonin/g stool). The initial amount can also be the level of expression of microbiota-derived serotonin modulating enzymes in the stool (log change of reads), as measured by qPCR or other appropriate method. The initial amount can also be the level(s) of microbiota-derived serotonin-modulating metabolite(s) (ug serotonin-metabolites/g stool) and/or protein(s) (ug serotonin-modulating protein/g stool). Unless otherwise defined herein, stool is weighed when wet or dry, i.e., without active drying, and within one hour of production of the stool. For instance, the stool can be weighed within 45 minutes, 30 minutes, 5 minutes, 10 minutes, or within 5 minutes of production of the stool.

As used herein, a “serotonergic response” means the response of a given organ (e.g., the subjects' cells, the brain, or vagus nerve) to differences in the concentrations of serotonin, serotonin-modulating bacteria (or their constituents—e.g. conditioned medium, cell pellets, purified metabolites, or purified proteins), or prebiotics to which it is exposed. A serotonergic response can include a change in concentrations of serotonin as well as expression levels and/or activity of different serotonin related genes/proteins, such as, but not limited to, tryptophan hydroxylase (Tph), Tph1 and Tph2, SERT, 5-HT_{1A}, 5-HT_{1B}, 5-HT_{1D}, 5-HT_{1E}, 5-HT_{1F}, 5-HT_{2A}, 5-HT_{2B}, 5-HT_{2C}, 5-HT₃, 5-HT₄, 5-HT_{5A}, 5-HT_{5B}, 5-HT₆, 5-HT₇

“Serotonin-modulating bacteria” is understood to mean bacteria that, when introduced to cell culture models or a host, can alter serotonin signaling and/or biosynthesis in a measurable way (e.g. LC/MS, ELISA, or other appropriate analytical assays) and by a statistically significant amount. In some embodiments, serotonin-modulating bacteria produce specific metabolites or proteins that interact with host cells to increase or reduce serotonin biosynthesis or signaling.

In some embodiments, “serotonin-modulating bacteria” produce these serotonin-modulating metabolites or proteins at physiological conditions of the human gut. In some embodiments, “serotonin-modulating bacteria” are naturally occurring. In some embodiments “serotonin-modulating bacteria” are engineered to produce metabolites or proteins, or combinations thereof, that interact with host cells to increase or reduce serotonin biosynthesis or signaling.

In some embodiments a “serotonin-modulating bacteria” is understood to mean a bacteria belonging to the genus Acidaminococcus, Agathobacter, Adlercreutzia, Akkermansia, Alistipes, Anaerotruncus, Bacillus, Bacteroides, Bifidobacterium, Bilophila, Bittarella, Blautia, Blautia, Butyricimonas, Clostridium, Clostridium, Collinsella, Coprococcus, Dialister, Dorea, Dysosmobacter, Eisenbergiella, Enterococcus, Enterorhabdus, Erysipelatoclostridium, Escherichia, Eubacterium, Faecalitalea, Flavonifractor, Flintibacter, Gemmiger, Gordonibacter, Hungatella, Intestinimonas, Lachnoclostridium, Lactobacillus, Lawsonibacter, Longibaculum, Mediterraneibacter, Mycolicibacterium, Oscillibacter, Parabacteroides, Parasutterella, Peptostreptococcus, Prevotella, Romboutsia, Ruminococcus, Sellimonas, Slackia, Streptococcus, Sutterella, Turicibacter, or Veillonella that modulates serotonin in a host

In some embodiments, a “serotonin-modulating bacteria” is understood to mean a bacteria belonging to the species Acidaminococcus intestini, Agathobacter rectalis, Akkermansia muciniphila, Adlercreutzia equolifaciens, Alistipes onderdonkii, Alistipes putredinis, Anaerotruncus colihominis, Bacillus cereus, Bacteroides caccae, Bacteroides cellulosilyticus, Bacteroides clarus, Bacteroides dorei, Bacteroides finegoldii, Bacteroides fragilis, Bacteroides koreensis, Bacteroides ovatus, Bacteroides plebeius, Bacteroides salyersiae, Bacteroides stercoris, Bacteroides thetaiotaomicron, Bacteroides uniformis, Bacteroides vulgatus, Bacteroides xylanisolvens, Bifidobacterium adolescentis, Bifidobacterium breve, Bifidobacterium faecale, Bifidobacterium longum, Bilophila wadsworthia, Bittarella massiliensis, Blautia coccoides, Blautia obeum, Blautia wexlerae, Butyricimonas paravirosa, Clostridium asparagiforme, Clostridium aldenese, Clostridium bolteae, Clostridium butyricum, Clostridium clostridioforme, Clostridium hathewayi, Clostridium innoculum, Clostridium lavalense, Clostridium paraputrificum, Clostridium saudiense, Clostridium scindens, Clostridium sp., Clostridium sporogenes, Clostridium sphenoides, Clostridium symbiosum, Clostridium tyrobutyricum, Clostridium hylemonae, Collinsella aerofaciens, Coprococcus comes, Coprococcus eutactus, Dialister invisus, Dorea longicatena, Dysosmobacter welbionis, Eisenbergiella tayi, Enterococcus durans, Enterococcus faecium, Enterorhabdus caecimuris, Enterorhabdus muris, Erysipelatoclostridium ramosum, Escherichia coli, Eubacterium callanderi, Eubacterium eligens, Eubacterium rectale, Faecalitalea cylindroides, Flavonifractor plautii, Flintibacter butyricus, Gemmiger formicilis, Gemmiger sp., Gordonibacter pamelaeae, Hungatella effluvii, Hungatella hathewayi, Intestinimonas butyriciproducens, Intestinimonas massiliensis, Lachnoclostridium sp., Lactobacillus brevis, Lawsonibacter asaccharolyticus, Longibaculum muris, Longibaculum sp., Mediterraneibacter faecis, Mycolicibacterium smegmatis, Oscillibacter sp., Parabacteroides distasonis, Parabacteroides goldsteinii, Parabacteroides johnsonii, Parabacteroides merdae, Parasutterella excrementihominis, Peptostreptococcus russellii, Prevotella copri, Prevotella sp, Prevotella sp., Romboutsia lituseburensis, Ruminococcus bicirculans, Ruminococcus gnavus, Ruminococcus sp., Sellimonas intestinalis, Slackia isoflavoniconvertens, Streptococcus gordonii, Sutterella wadsworthensis, Turicibacter sanguinis, or Veillonella atypica that modulates serotonin in a host.

In some embodiments, a serotonin-modulating bacteria is understood to mean the strains Acidaminococcus intestini HB-95, Agathobacter rectalis HB-257, Akkermansia muciniphila BAA-835, Adlercreutzia equolifaciens FJC-B9, Alistipes onderdonkii HB-311, Alistipes putredinis HB-324, Anaerotruncus colihominis HB-474, Anaerotruncus colihominis HB-83, Bacillus cereus HB-25, Bacteroides caccae HB-11, Bacteroides cellulosilyticus HB-227, Bacteroides clarus HB-30, Bacteroides dorei HB-12, Bacteroides finegoldii HB-31, Bacteroides fragilis HB-58, Bacteroides koreensis HB-385, Bacteroides ovatus HB-70, Bacteroides plebeius HB-237, Bacteroides salyersiae HB-32, Bacteroides stercoris HB-33, Bacteroides thetaiotaomicron HB-34, Bacteroides uniformis HB-13, Bacteroides vulgatus HB-10, Bacteroides xylanisolvens HB-35, Bifidobacterium adolescentis HB-179, Bifidobacterium breve HB-90, Bifidobacterium faecale HB-159, Bifidobacterium longum HB-234, Bifidobacterium longum HB-71, Bilophila wadsworthia HB-693, Bittarella massiliensis HB-477, Blautia coccoides HB-23, Blautia obeum HB-14, Blautia wexlerae HB-16, Butyricimonas paravirosa HB-453, Clostridium aldenese HB-440, Clostridium bolteae HB-442, Clostridium butyricum HB-88, Clostridium clostridioforme HB-642, Clostridium hathewayi HB-152, Clostridium innoculum HB-82, Clostridium lavalense HB-452c, Clostridium paraputrificum HB-27, Clostridium saudiense HB-142, Clostridium scindens HB-444, Clostridium sp. HB-358, Clostridium sphenoides HB-470, Clostridium sporogenes JCM 7836, Clostridium sporogenes McClung 2004, Clostridium symbiosum HB-67, Clostridium tyrobutyricum HB-469, Clostridium hylemonae HB-73, Collinsella aerofaciens HB-274, Coprococcus comes HB-376, Coprococcus comes HB-80, Coprococcus comes ATCC 27758, Coprococcus eutactus HB-155, Coprococcus eutactus ATCC 27759, Dialister invisus HB-387, Dorea longicatena HB-17, Dysosmobacter welbionis HB-45, Eisenbergiella tayi HB-437, Eisenbergiella tayi HB-612, Enterococcus durans HB-48, Enterococcus faecium HB-640, Enterococcus faecium HB-85, Enterorhabdus caecimuris B7, Enterorhabdus muris WCA-131-CoC-2, Erysipelatoclostridium ramosum HB-24, Escherichia coli HB-490, Eubacterium callanderi HB-59, Eubacterium eligens HB-252, Eubacterium rectale HB-22, Faecalitalea cylindroides HB-664, Flavonifractor plautii HB-472, Flintibacter butyricus HB-344, Gemmiger formicilis HB-325, Gemmiger sp. HB-567, Gordonibacter pamelaeae HB-15, Hungatella effluvii HB-02, Hungatella hathewayi HB-01, Intestinimonas butyriciproducens HB-478, Intestinimonas massiliensis HB-651, Lachnoclostridium sp. HB-698, Lactobacillus brevis HB-87, Lawsonibacter asaccharolyticus HB-521, Longibaculum muris HB-79, Longibaculum sp. HB-681, Mediterraneibacter faecis HB-364, Mycolicibacterium smegmatis ATCC 19420, Oscillibacter sp. HB-28, Parabacteroides distasonis HB-20, Parabacteroides distasonis HB-214, Parabacteroides goldsteinii HB-44, Parabacteroides johnsonii HB-03, Parabacteroides merdae HB-63, Parasutterella excrementihominis HB-330, Peptostreptococcus russeliii RT-10B, Prevotella copri HB-373, Prevotella sp HB-649, Prevotella sp. HB-333, Romboutsia lituseburensis HB-102, Ruminococcus bicirculans HB-105, Ruminococcus bicirculans HB-268, Ruminococcus gnavus HB-40, Ruminococcus gnavus HB-516, Ruminococcus sp. HB-626, Sellimonas intestinalis HB-443, Slackia isoflavoniconvertens HB-326, Streptococcus gordonii HB-62, Streptococcus gordonii HB-98, Sutterella wadsworthensis HB-259, Turicibacter sanguinis HB-147, or Veillonella atypica HB-251.

In some embodiments, a serotonin-modulating bacteria is understood to mean a bacteria comprising a 16S rDNA sequence at least about 95% identical to a 16S rDNA sequence selected from one of SEQ ID NOs: 1-114. As a non-limiting example, a serotonin-modulating bacteria comprises a 16S rDNA sequence with at least 95% identity, at least 96% identity, at least 97% identity, at least 98% identity, at least 99% identity, or 100% identity to a 16S rDNA sequence described herein (e.g., SEQ ID NOs: 1-114).

“Physiologically relevant condition” of the human intestinal tract is understood to mean conditions found in the human gastrointestinal tract or relevant portion thereof (e.g., small intestine, colon, etc.). For example, a pH range of about 4.5-7.5. It can also mean conditions such as levels of nutrients or other bacteria and/or their metabolites/proteins as found in the human gut.

The term “gut” is understood to refer to the human gastrointestinal tract, also known as the alimentary canal. The gut includes the mouth, pharynx, esophagus, stomach, small intestine (duodenum, jejunum, ileum), large intestines (cecum and colon) and rectum.

As used herein, “bacterium” is understood as a single bacterial cell of a given species.

The term “treating” with regard to a subject, refers to improving at least one symptom of the subject's disorder. Treating includes curing, improving, or at least partially ameliorating the disorder, and can, but need not necessarily encompass curing the disorder.

The terms “decrease”, “reduced”, “reduction”, or “inhibit” are all used herein to mean a decrease by a statistically significant amount. In some embodiments, “reduce,” “reduction” or “decrease” or “inhibit” typically means a decrease by at least 10% as compared to a reference level (e.g. the absence of a given treatment or agent) and can include, for example, a decrease by at least about 10%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 95%, at least about 98%, at least about 99%, or more. As used herein, “reduction” or “inhibition” does not encompass a complete inhibition or reduction as compared to a reference level. “Complete inhibition” is a 100% inhibition as compared to a reference level. A decrease can be preferably down to a level accepted as within the range of normal for an individual without a given disorder.

The terms “increased”, “increase”, “enhance”, or “activate” are all used herein to mean an increase by a statically significant amount. In some embodiments, the terms “increased”, “increase”, “enhance”, or “activate” can mean an increase of at least 10% as compared to a reference level, for example an increase of at least about 20%, or at least about 30%, or at least about 40%, or at least about 50%, or at least about 60%, or at least about 70%, or at least about 80%, or at least about 90% or up to and including a 100% increase or any increase between 10-100% as compared to a reference level, or at least about a 2-fold, or at least about a 3-fold, or at least about a 4-fold, or at least about a 5-fold or at least about a 10-fold increase, or any increase between 2-fold and 10-fold or greater as compared to a reference level. In the context of a marker or symptom, a “increase” is a statistically significant increase in such level.

The term “alter” or “modulate” as used herein in reference to a value or parameter means an increase or decrease in the parameter as those terms are defined herein.

As used herein, a “subject” means a human or animal. Usually the animal is a vertebrate such as a primate, rodent, domestic animal or game animal. Primates include chimpanzees, cynomologous monkeys, spider monkeys, and macaques, e.g., Rhesus. Rodents include mice, rats, woodchucks, ferrets, rabbits and hamsters. Domestic and game animals include cows, horses, pigs, deer, bison, buffalo, feline species, e.g., domestic cat, canine species, e.g., dog, fox, wolf, avian species, e.g., chicken, emu, ostrich, and fish, e.g., trout, catfish and salmon. In some embodiments, the subject is a mammal, e.g., a primate, e.g., a human. The terms, “individual,” “patient”, “host,” and “subject” are used interchangeably herein.

Preferably, the subject is a mammal. The mammal can be a human, non-human primate, mouse, rat, dog, cat, horse, or cow, but is not limited to these examples. Mammals other than humans can be advantageously used as subjects that represent animal models of a serotonin-related disease or disorder. A subject can be male or female.

A subject can be one who has been previously diagnosed with or identified as suffering from or having a condition in need of treatment (e.g. a serotonin-related disease or disorder) or one or more complications related to such a condition, and optionally, has already undergone treatment for a serotonin-related disease or disorder or the one or more complications related to a serotonin-related disease or disorder. Alternatively, a subject can also be one who has not been previously diagnosed as having a serotonin-related disease or disorder or one or more complications related to a serotonin-related disease or disorder. For example, a subject can be one who exhibits one or more risk factors for a serotonin-related disease or disorder or one or more complications related to a serotonin-related disease or disorder or a subject who does not exhibit risk factors.

A “subject in need” of treatment for a particular condition can be a subject having that condition, diagnosed as having that condition, or at risk of developing that condition.

As used herein, the terms “protein” and “polypeptide” are used interchangeably herein to designate a series of amino acid residues, connected to each other by peptide bonds between the alpha-amino and carboxy groups of adjacent residues. The terms “protein”, and “polypeptide” refer to a polymer of amino acids, including modified amino acids (e.g., phosphorylated, glycated, glycosylated, etc.) and amino acid analogs, regardless of its size or function. “Protein” and “polypeptide” are often used in reference to relatively large polypeptides, whereas the term “peptide” is often used in reference to small polypeptides, but usage of these terms in the art overlaps. The terms “protein” and “polypeptide” are used interchangeably herein when referring to a gene product and fragments thereof. Thus, exemplary polypeptides or proteins include gene products, naturally occurring proteins, homologs, orthologs, paralogs, fragments and other equivalents, variants, fragments, and analogs of the foregoing.

In the various embodiments described herein, it is further contemplated that variants (naturally occurring or otherwise), alleles, homologs, conservatively modified variants, and/or conservative substitution variants of any of the particular polypeptides described are encompassed. As to amino acid sequences, one of skill will recognize that individual substitutions, deletions or additions to a nucleic acid, peptide, polypeptide, or protein sequence which alters a single amino acid or a small percentage of amino acids in the encoded sequence is a “conservatively modified variant” where the alteration results in the substitution of an amino acid with a chemically similar amino acid and retains the desired activity of the polypeptide. Such conservatively modified variants are in addition to and do not exclude polymorphic variants, interspecies homologs, and alleles consistent with the disclosure.

A given amino acid can be replaced by a residue having similar physiochemical characteristics, e.g., substituting one aliphatic residue for another (such as Ile, Val, Leu, or Ala for one another), or substitution of one polar residue for another (such as between Lys and Arg; Glu and Asp; or Gln and Asn). Other such conservative substitutions, e.g., substitutions of entire regions having similar hydrophobicity characteristics, are well known. Polypeptides comprising conservative amino acid substitutions can be tested confirm that a desired activity, e.g. activity and/or specificity of a native or reference polypeptide is retained.

Amino acids can be grouped according to similarities in the properties of their side chains (in A. L. Lehninger, in Biochemistry, second ed., pp. 73-75, Worth Publishers, New York (1975)): (1) non-polar: Ala (A), Val (V), Leu (L), Ile (I), Pro (P), Phe (F), Trp (W), Met (M); (2) uncharged polar: Gly (G), Ser (S), Thr (T), Cys (C), Tyr (Y), Asn (N), Gln (Q); (3) acidic: Asp (D), Glu (E); (4) basic: Lys (K), Arg (R), His (H). Alternatively, naturally occurring residues can be divided into groups based on common side-chain properties: (1) hydrophobic: Norleucine, Met, Ala, Val, Leu, Ile; (2) neutral hydrophilic: Cys, Ser, Thr, Asn, Gln; (3) acidic: Asp, Glu; (4) basic: His, Lys, Arg; (5) residues that influence chain orientation: Gly, Pro; (6) aromatic: Trp, Tyr, Phe. Non-conservative substitutions will entail exchanging a member of one of these classes for another class. Particular conservative substitutions include, for example; Ala into Gly or into Ser; Arg into Lys; Asn into Gln or into His; Asp into Glu; Cys into Ser; Gln into Asn; Glu into Asp; Gly into Ala or into Pro; His into Asn or into Gln; Ile into Leu or into Val; Leu into Ile or into Val; Lys into Arg, into Gln or into Glu; Met into Leu, into Tyr or into Ile; Phe into Met, into Leu or into Tyr; Ser into Thr; Thr into Ser; Trp into Tyr; Tyr into Trp; and/or Phe into Val, into Ile or into Leu.

In some embodiments, the polypeptide described herein (or a nucleic acid encoding such a polypeptide) can be a functional fragment of one of the amino acid sequences described herein. As used herein, a “functional fragment” is a fragment or segment of a polypeptide which retains at least 50% of the wild-type reference polypeptide's activity. A functional fragment can comprise conservative substitutions of the sequences disclosed herein.

In some embodiments, the polypeptide described herein can be a variant of a sequence described herein. In some embodiments, the variant is a conservatively modified variant. Conservative substitution variants can be obtained by mutations of native nucleotide sequences, for example. A “variant,” as referred to herein, is a polypeptide substantially homologous to a native or reference polypeptide, but which has an amino acid sequence different from that of the native or reference polypeptide because of one or a plurality of deletions, insertions or substitutions. Variant polypeptide-encoding DNA sequences encompass sequences that comprise one or more additions, deletions, or substitutions of nucleotides when compared to a native or reference DNA sequence, but that encode a variant protein or fragment thereof that retains activity. A wide variety of PCR-based site-specific mutagenesis approaches are known in the art and can be applied by the ordinarily skilled artisan to generate and test artificial variants.

Alterations of the native amino acid sequence can be accomplished by any of a number of techniques known to one of skill in the art. Mutations can be introduced, for example, at particular loci by synthesizing oligonucleotides containing a mutant sequence, flanked by restriction sites enabling ligation to fragments of the native sequence. Following ligation, the resulting reconstructed sequence encodes an analog having the desired amino acid insertion, substitution, or deletion. Alternatively, oligonucleotide-directed site-specific mutagenesis procedures can be employed to provide an altered nucleotide sequence having particular codons altered according to the substitution, deletion, or insertion required. Techniques for making such alterations are very well established and include, for example, those disclosed by Walder et al. (Gene 42:133, 1986); Bauer et al. (Gene 37:73, 1985); Craik (BioTechniques, January 1985, 12-19); Smith et al. (Genetic Engineering: Principles and Methods, Plenum Press, 1981); and U.S. Pat. Nos. 4,518,584 and 4,737,462, which are herein incorporated by reference in their entireties. Any cysteine residue not involved in maintaining the proper conformation of the polypeptide also can be substituted, generally with serine, to improve the oxidative stability of the molecule and prevent aberrant crosslinking. Conversely, cysteine bond(s) can be added to the polypeptide to improve its stability or facilitate oligomerization.

Variant polypeptide-encoding DNA sequences encompass sequences that comprise one or more additions, deletions, or substitutions of nucleotides when compared to a native or reference DNA sequence, but that encode a variant protein or fragment thereof that retains activity. A wide variety of PCR-based site-specific mutagenesis approaches are known in the art and can be applied by the ordinarily skilled artisan.

A variant amino acid or nucleic acid sequence can be at least 50%, at least 60%, at least 70%, at least 80%, at least 90%, at least 91%, at least 92%, at least 93%, at least 94%, at least 95%, at least 96%, at least 97%, at least 98%, at least 99%, or more, identical to a native or reference sequence. The degree of homology (percent identity) between a native and a mutant sequence can be determined, for example, by comparing the two sequences using freely available computer programs commonly employed for this purpose on the world wide web (e.g. BLASTp or BLASTn with default settings).

A variant amino acid sequence can be at least 50%, at least 60%, at least 70%, at least 80%, at least 90%, at least 91%, at least 92%, at least 93%, at least 94%, at least 95%, at least 96%, at least 97%, at least 98%, at least 99%, or more, similar to a native or reference sequence. As used herein, “similarity” refers to an identical amino acid or a conservatively substituted amino acid, as described herein. Accordingly, the percentage of “sequence similarity” is the percentage of amino acids which is either identical or conservatively changed; e.g., “sequence similarity”=(% sequence identity)+(% conservative changes). It should be understood that a sequence that has a specified percent similarity to a reference sequence necessarily encompasses a sequence with the same specified percent identity to that reference sequence. The skilled person will be aware of several different computer programs, using different mathematical algorithms, that are available to determine the identity or similarity between two sequences. For instance, use can be made of a computer program employing the Needleman and Wunsch algorithm (Needleman et al. (1970)); the GAP program in the Accelrys GCG software package (Accelerys Inc., San Diego U.S.A.); the algorithm of E. Meyers and W. Miller (Meyers et al. (1989)) which has been incorporated into the ALIGN program (version 2.0); or more preferably the BLAST (Basic Local Alignment Tool using default parameters); see e.g., U.S. Pat. No. 10,023,890, the content of which is incorporated by reference herein in its entirety.

In some embodiments, sequencing comprises 16S rRNA gene sequencing, which can also be referred to as “16S ribosomal RNA sequencing”, “16S rDNA sequencing” or “16s rRNA sequencing”. Sequencing of the 16S rRNA gene can be used for genetic studies as it is highly conserved between different species of bacteria, but it is not present in eukaryotic species. In addition to highly conserved regions, the 16S rRNA gene also comprises nine hypervariable regions (V1-V9) that vary by species. 16S rRNA gene sequencing typically comprises using a plurality of universal primers that bind to conserved regions of the 16S rRNA gene, PCR amplifying the bacterial 16S rRNA gene regions (including hypervariable regions), and sequencing the amplified 16S rRNA genes with a next-generation sequencing technology as described herein (see also e.g., U.S. Pat. Nos. 5,654,418; 6,344,316; and 8,889,358; and US Patent Application Numbers US 2013/0157265 and US 2018/0195111, which are incorporated by reference in their entireties).

As used herein, the term “nucleic acid” or “nucleic acid sequence” refers to any molecule, preferably a polymeric molecule, incorporating units of ribonucleic acid, deoxyribonucleic acid or an analog thereof. The nucleic acid can be either single-stranded or double-stranded. A single-stranded nucleic acid can be one nucleic acid strand of a denatured double-stranded DNA. Alternatively, it can be a single-stranded nucleic acid not derived from any double-stranded DNA. In one aspect, the nucleic acid can be DNA. In another aspect, the nucleic acid can be RNA. Suitable DNA can include, e.g., genomic DNA or cDNA or bacterial DNA. Suitable RNA can include, e.g., mRNA or bacterial RNA.

The term “expression” refers to the cellular processes involved in producing RNA and proteins and as appropriate, secreting proteins, including where applicable, but not limited to, for example, transcription, transcript processing, translation and protein folding, modification and processing. Expression can refer to the transcription and stable accumulation of sense (mRNA) or antisense RNA derived from a nucleic acid fragment or fragments of the invention and/or to the translation of mRNA into a polypeptide.

In some embodiments, the expression of a biomarker(s), target(s), or gene/polypeptide described herein is/are tissue-specific. In some embodiments, the expression of a biomarker(s), target(s), or gene/polypeptide described herein is/are global. In some embodiments, the expression of a biomarker(s), target(s), or gene/polypeptide described herein is systemic.

“Expression products” include RNA transcribed from a gene, and polypeptides obtained by translation of mRNA transcribed from a gene. The term “gene” means the nucleic acid sequence which is transcribed (DNA) to RNA in vitro or in vivo when operably linked to appropriate regulatory sequences. The gene may or may not include regions preceding and following the coding region, e.g. 5′ untranslated (5′UTR) or “leader” sequences and 3′ UTR or “trailer” sequences, as well as intervening sequences (introns) between individual coding segments (exons).

“Marker” in the context of the present disclosure refers to an expression product, e.g., nucleic acid, polypeptide, or metabolite which is differentially present or differentially abundant in a sample taken from subjects having a serotonin-related disease or disorder, as compared to a comparable sample taken from control subjects (e.g., a healthy subject). The term “biomarker” is used interchangeably with the term “marker.”

In some embodiments, the methods described herein relate to measuring, detecting, or determining the level of at least one marker. As used herein, the term “detecting” or “measuring” refers to observing a signal from, e.g. a probe, label, or target molecule to indicate the presence of an analyte in a sample. Any method known in the art for detecting a particular label moiety can be used for detection. Exemplary detection methods include, but are not limited to, spectroscopic, fluorescent, photochemical, biochemical, immunochemical, electrical, optical or chemical methods. In some embodiments of any of the aspects, measuring can be a quantitative observation.

As used herein, a “reference” level refers to a level of, e.g., a marker as measured in or established for a sample representative of a known status. For example, a marker from a sample from an individual known not to have, or alternatively known to have, a given disease or disorder can be a reference. In one embodiment, a reference is the level in an individual without a given condition. In another embodiment, a reference is the level in an individual with a given condition.

In some embodiments of any of the aspects, a polypeptide, nucleic acid, or cell (e.g., a bacterial cell or a bacteria) as described herein can be engineered. As used herein, “engineered” refers to the aspect of having been manipulated by the hand of man. For example, a polypeptide is considered to be “engineered” when at least one aspect of the polypeptide, e.g., its sequence, has been manipulated by the hand of man to differ from the aspect as it exists in nature. As is common practice and is understood by those in the art, progeny of an engineered cell are typically still referred to as “engineered” even though the actual manipulation was performed on a prior entity.

As used herein, “contacting” refers to any suitable means for delivering, or exposing, an agent to at least one cell. Exemplary delivery methods include, but are not limited to, direct delivery to cell culture medium, perfusion, injection, or other delivery method well known to one skilled in the art. In some embodiments, contacting comprises physical human activity, e.g., an injection; an act of dispensing, mixing, and/or decanting; and/or manipulation of a delivery device or machine.

The term “statistically significant” or “significantly” refers to statistical significance and generally means a two standard deviation (2SD) or greater difference.

Other than in the operating examples, or where otherwise indicated, all numbers expressing quantities of ingredients or reaction conditions used herein should be understood as modified in all instances by the term “about.” The term “about” when used in connection with percentages can mean±1%.

As used herein, the term “comprising” means that other elements can also be present in addition to the defined elements presented. The use of “comprising” indicates inclusion rather than limitation.

The term “consisting of” refers to compositions, methods, and respective components thereof as described herein, which are exclusive of any element not recited in that description of the embodiment.

As used herein the term “consisting essentially of” refers to those elements required for a given embodiment. The term permits the presence of additional elements that do not materially affect the basic and novel or functional characteristic(s) of that embodiment of the invention.

The singular terms “a,” “an,” and “the” include plural referents unless context clearly indicates otherwise. Similarly, the word “or” is intended to include “and” unless the context clearly indicates otherwise. Although methods and materials similar or equivalent to those described herein can be used in the practice or testing of this disclosure, suitable methods and materials are described below. The abbreviation, “e.g.” is derived from the Latin exempli gratia, and is used herein to indicate a non-limiting example. Thus, the abbreviation “e.g.” is synonymous with the term “for example.”

Groupings of alternative elements or embodiments of the invention disclosed herein are not to be construed as limitations. Each group member can be referred to and claimed individually or in any combination with other members of the group or other elements found herein. One or more members of a group can be included in, or deleted from, a group for reasons of convenience and/or patentability. When any such inclusion or deletion occurs, the specification is herein deemed to contain the group as modified thus fulfilling the written description of all Markush groups used in the appended claims.

Unless otherwise defined herein, scientific and technical terms used in connection with the present application shall have the meanings that are commonly understood by those of ordinary skill in the art to which this disclosure belongs. It should be understood that this invention is not limited to the particular methodology, protocols, and reagents, etc., described herein and as such can vary. The terminology used herein is for the purpose of describing particular embodiments only, and is not intended to limit the scope of the present invention, which is defined solely by the claims. Definitions of common terms in immunology and molecular biology can be found in The Merck Manual of Diagnosis and Therapy, 20th Edition, published by Merck Sharp & Dohme Corp., 2018 (ISBN 0911910190, 978-0911910421); Robert S. Porter et al. (eds.), The Encyclopedia of Molecular Cell Biology and Molecular Medicine, published by Blackwell Science Ltd., 1999-2012 (ISBN 9783527600908); and Robert A. Meyers (ed.), Molecular Biology and Biotechnology: a Comprehensive Desk Reference, published by VCH Publishers, Inc., 1995 (ISBN 1-56081-569-8); Immunology by Werner Luttmann, published by Elsevier, 2006; Janeway's Immunobiology, Kenneth Murphy, Allan Mowat, Casey Weaver (eds.), W. W. Norton & Company, 2016 (ISBN 0815345054, 978-0815345053); Lewin's Genes XI, published by Jones & Bartlett Publishers, 2014 (ISBN-1449659055); Michael Richard Green and Joseph Sambrook, Molecular Cloning: A Laboratory Manual, 4th ed., Cold Spring Harbor Laboratory Press, Cold Spring Harbor, N.Y., USA (2012) (ISBN 1936113414); Davis et al., Basic Methods in Molecular Biology, Elsevier Science Publishing, Inc., New York, USA (2012) (ISBN 044460149X); Laboratory Methods in Enzymology: DNA, Jon Lorsch (ed.) Elsevier, 2013 (ISBN 0124199542); Current Protocols in Molecular Biology (CPMB), Frederick M. Ausubel (ed.), John Wiley and Sons, 2014 (ISBN 047150338X, 9780471503385), Current Protocols in Protein Science (CPPS), John E. Coligan (ed.), John Wiley and Sons, Inc., 2005; and Current Protocols in Immunology (CPI) (John E. Coligan, ADA M Kruisbeek, David H Margulies, Ethan M Shevach, Warren Strobe, (eds.) John Wiley and Sons, Inc., 2003 (ISBN 0471142735, 9780471142737), the contents of which are all incorporated by reference herein in their entireties.

Other terms are defined herein within the description of the various aspects of the invention.

All patents and other publications; including literature references, issued patents, published patent applications, and co-pending patent applications; cited throughout this application are expressly incorporated herein by reference for the purpose of describing and disclosing, for example, the methodologies described in such publications that might be used in connection with the technology described herein. These publications are provided solely for their disclosure prior to the filing date of the present application. Nothing in this regard should be construed as an admission that the inventors are not entitled to antedate such disclosure by virtue of prior invention or for any other reason. All statements as to the date or representation as to the contents of these documents is based on the information available to the applicants and does not constitute any admission as to the correctness of the dates or contents of these documents.

The description of embodiments of the disclosure is not intended to be exhaustive or to limit the disclosure to the precise form disclosed. While specific embodiments of, and examples for, the disclosure are described herein for illustrative purposes, various equivalent modifications are possible within the scope of the disclosure, as those skilled in the relevant art will recognize. For example, while method steps or functions are presented in a given order, alternative embodiments can perform functions in a different order, or functions can be performed substantially concurrently. The teachings of the disclosure provided herein can be applied to other procedures or methods as appropriate. The various embodiments described herein can be combined to provide further embodiments. Aspects of the disclosure can be modified, if necessary, to employ the compositions, functions and concepts of the above references and application to provide yet further embodiments of the disclosure. These and other changes can be made to the disclosure in light of the detailed description. All such modifications are intended to be included within the scope of the appended claims.

Specific elements of any of the foregoing embodiments can be combined or substituted for elements in other embodiments. Furthermore, while advantages associated with certain embodiments of the disclosure have been described in the context of these embodiments, other embodiments can also exhibit such advantages, and not all embodiments need necessarily exhibit such advantages to fall within the scope of the disclosure.

The technology described herein is further illustrated by the following examples which in no way should be construed as being further limiting.

Some embodiments of the technology described herein can be defined according to any of the following numbered paragraphs:
1. A therapeutic composition for increasing serotonin level in a mammalian subject in need thereof, the composition comprising an amount of a live isolated serotonin-increasing bacterial species, dead isolated serotonin-increasing bacterial species, conditioned medium from an isolated, cultured serotonin-increasing bacterial species, cell pellet of an isolated serotonin-increasing bacterial species, a purified metabolite produced by an isolated serotonin-increasing bacterial species, a purified protein produced by an isolated serotonin-increasing bacterial species, or a combination thereof sufficient to increase serotonin level in the subject, and an excipient or carrier suitable for delivery to the gut.
2. The therapeutic composition of paragraph 1, wherein the isolated serotonin-increasing bacterial species increases serotonin in at least one of the following ways: production of serotonin; production of secreted metabolites or secreted proteins that induce serotonin production; production of ligands that induce serotonin production; or production of an agonist of a serotonin receptor or the trace amine-associated receptor (TAAR).
3. The therapeutic composition of paragraph 1 or 2, wherein the isolated serotonin-increasing bacterial species is a serotonin-producing bacterial species.
4. The therapeutic composition of any one of paragraphs 1-3, wherein the serotonin-producing bacterial species comprises one or more species selected from Enterococcus durans, Clostridium lavalense, Clostridium asparagiforme, Ruminococcus gnavus.
5. The therapeutic composition of any one of paragraphs 1-4, wherein the serotonin-producing bacterial species comprises one or more species selected from Enterococcus durans HB-48, Clostridium lavalense HB-452c, Ruminococcus gnavus HB-40, Ruminococcus gnavus HB-516.
6. The therapeutic composition of any one of paragraphs 1-5, wherein the serotonin-producing bacterial species comprises a 16S sequence at least 95% identical to a 16S sequence selected from SEQ ID NOs: 1-4.
7. The therapeutic composition of any one of paragraphs 1-6, wherein the serotonin-producing bacterial species produces serotonin under conditions found in the mammalian gut.
8. The therapeutic composition of any one of paragraphs 1-7, wherein the mammalian subject is a human subject.
9. The therapeutic composition of paragraph 1, wherein the isolated serotonin-producing bacterial species encodes and expresses a decarboxylase enzyme that catalyzes the production of tryptamine from tryptophan.
10. The therapeutic composition of paragraph 9, wherein the decarboxylase enzyme is a tryptophan decarboxylase.
11. The therapeutic composition of paragraph 9 or 10, wherein the decarboxylase enzyme belongs to the EC number 4.1.1.105.
12. The therapeutic composition of any one of paragraphs 9-11, wherein the decarboxylase enzyme is at least 50% identical to an enzyme comprising an amino acid sequence selected from SEQ ID Nos: 115-119.
13. The therapeutic composition of paragraph 1, wherein the isolated serotonin-producing bacterial species encodes and expresses an enzyme that hydroxylates tryptamine to produce serotonin.
14. The therapeutic composition of paragraph 13, wherein the enzyme that hydroxylates tryptamine is a tryptamine 5-hydroxylase.
15. The therapeutic composition of paragraph 13 or 14, wherein the enzyme that hydroxylates tryptamine is at least 50% identical to the enzyme of SEQ ID NO: 134.
16. The therapeutic composition of any one of paragraphs 13-15, wherein the enzyme that hydroxylates tryptamine is an anaerobic hydroxylase.
17. The therapeutic composition of any one of paragraphs 1-16, wherein the isolated serotonin-producing bacterial species encodes and expresses a decarboxylase enzyme that catalyzes the production of tryptamine from tryptophan and an enzyme that hydroxylates tryptamine to produce serotonin.
18. The therapeutic composition of any one of paragraphs 1-17, wherein the live isolated serotonin-producing bacterial species, dead isolated serotonin producing bacterial species, conditioned medium from an isolated, cultured serotonin-producing bacterial species or cell pellet of an isolated serotonin-producing bacterial species comprises a first bacterial species that encodes and expresses a decarboxylase enzyme that catalyzes the production of tryptamine from tryptophan and a second bacterial species that encodes and expresses an enzyme that hydroxylates tryptamine to produce serotonin.
19. The therapeutic composition of any one of paragraphs 1-18, wherein the decarboxylase is a lysine decarboxylase family enzyme.
20. The therapeutic composition of paragraph 1, wherein the live isolated serotonin-producing bacterial species, dead isolated serotonin producing bacterial species, conditioned medium from an isolated, cultured serotonin-producing bacterial species or cell pellet of an isolated serotonin-producing bacterial species comprises one or more bacterial species that encode and express an enzyme that converts tryptophan to 5-hydroxy-L-tryptophan (5-HTP).
21. The therapeutic composition of paragraph 20, wherein the enzyme that converts tryptophan to 5-hydroxy-L-tryptophan (5-HTP) has an amino acid sequence at least 50% identical to a sequence selected from SEQ ID Nos: 120-126.
22. The therapeutic composition of paragraph 20 or 21, wherein the enzyme that converts tryptophan to 5-hydroxy-L-tryptophan (5-HTP) belongs to the EC number 1.14.16.4.
23. The therapeutic composition of paragraph 1, wherein the live isolated serotonin-producing bacterial species, dead isolated serotonin producing bacterial species, conditioned medium from an isolated, cultured serotonin-producing bacterial species or cell pellet of an isolated serotonin-producing bacterial species comprises a bacterial species that encodes and expresses an enzyme that converts 5-hydroxy-L-tryptophan to serotonin.
24. The therapeutic composition of paragraphs 23, wherein the enzyme that converts 5-hydroxy-L-tryptophan to serotonin is an aromatic L-amino acid decarboxylase.
25. The therapeutic composition of paragraph 23 or 24, wherein the enzyme that catalyzes the conversion of 5-hydroxy-L-tryptophan to serotonin belongs to the EC number 4.1.1.28.
26. The therapeutic composition of any one of paragraphs 23-25, wherein the enzyme that converts 5-hydroxy-L-tryptophan to serotonin is at least 50% identical to a sequence selected from SEQ ID NOs: 127-133.
27. The therapeutic composition of any one of paragraphs 20-22, wherein the isolated serotonin-producing bacterial species further encodes and expresses an enzyme that converts 5-hydroxy-L-tryptophan to serotonin.
28. The therapeutic composition of paragraph 27, wherein the enzyme that converts 5-hydroxy-L-tryptophan to serotonin is an aromatic L-amino acid decarboxylase.
29. The therapeutic composition of either of paragraphs 27 or 28, wherein the enzyme that converts 5-hydroxy-L-tryptophan to serotonin is at least 50% identical to a sequence selected from SEQ ID NOs: 127-133.
30. The therapeutic composition of paragraph 1, wherein the live isolated serotonin-producing bacterial species, dead isolated serotonin producing bacterial species, conditioned medium from an isolated, cultured serotonin-producing bacterial species or cell pellet of an isolated serotonin-producing bacterial species comprises a first bacterial species that encodes and expresses an enzyme that catalyzes the conversion of tryptophan to 5-hydroxy-L-tryptophan and a second bacterial species that encodes and expresses an enzyme that converts 5-hydroxy-L-tryptophan to serotonin.
31. The therapeutic composition of paragraph 30, wherein the enzyme that catalyzes the conversion of tryptophan to 5-hydroxy-L-tryptophan is a tryptophan hydroxylase.
32. The therapeutic composition of paragraph 30 or 31, wherein the enzyme that catalyzes the conversion of tryptophan to 5-hydroxy-L-tryptophan has an amino acid sequence at least 50% identical to a sequence selected from SEQ ID Nos: 120-126, and the enzyme that converts 5-hydroxy-L-tryptophan to serotonin is at least 50% identical to a sequence selected from SEQ ID NOs: 127-133.
33. The therapeutic composition of any one of paragraphs 20-22 or 30-32, wherein the enzyme that catalyzes the conversion of tryptophan to 5-hydroxy-L-tryptophan is a phenylalanine hydroxylase.
34. The therapeutic composition of paragraph 33, wherein the phenylalanine hydroxylase comprises an amino acid sequence comprising one or more of phenylalanine at the position corresponding to W192, isoleucine or leucine at the position corresponding to F197, and cysteine at the position corresponding to E219 of the phenylalanine hydroxylase of Cupriavidus taiwanensis (SEQ ID NO: 227).
35. A pharmaceutical composition comprising the therapeutic composition of any one of paragraphs 1-34, and a pharmaceutically acceptable carrier.
36. A method of increasing serotonin level in a mammalian subject in need thereof, the method comprising administering a composition of any one of paragraphs 1-35 to the subject, whereby a serotonin level is increased.
37. The method of paragraph 36, wherein the administering is to the gut of the subject.
38. The method of paragraph 36 or 37, wherein the level of serotonin in the gut is increased.
39. The method of any one of paragraphs 36-38, wherein the level of serotonin in circulation is increased.
40. A method of treating a disease or disorder involving or characterized by low serotonin in a subject in need thereof, the method comprising administering a composition of any one of paragraphs 1-35 to the subject, whereby the disease or disorder is treated.
41. The method of paragraph 40, wherein the administering is to the gut of the subject.
42. The method of paragraph 40 or 41, wherein the level of serotonin in the gut is increased.
43. The method of any one of paragraphs 40-42, wherein the level of serotonin in circulation is increased.
44. The method of any one of paragraphs 40-43, wherein the disease or disorder is selected from the group consisting of constipation, IBS-C, depression, anxiety, addiction, neurodegenerative disorders, autism spectrum disorder, sleep disorders, attention deficit hyperactivity disorder (ADHD), memory loss (e.g. dementia), learning difficulties, osteoporosis, heartburn, dermatological conditions (eczema and itch), GERD, and pain disorders.
45. The method of any one of paragraphs 40-44, wherein the disease or disorder is not a gut disease or disorder.
46. The method of any one of paragraphs 40-45, wherein the disease or disorder is selected from the group consisting of depression, anxiety, addiction, neurodegenerative disorders, autism spectrum disorder, sleep disorders, attention deficit hyperactivity disorder (ADHD), memory loss (e.g. dementia), learning difficulties, osteoporosis, dermatological conditions (eczema and itch), and pain disorders.
47. The therapeutic composition of paragraph 1, wherein the composition comprising an amount of a live isolated serotonin-increasing bacterial species, dead isolated serotonin increasing bacterial species, conditioned medium from an isolated, cultured serotonin-increasing bacterial species, cell pellet of an isolated serotonin-increasing bacterial species, a purified metabolite produced by an isolated serotonin-increasing bacterial species, a purified protein produced by an isolated serotonin-increasing bacterial species, or a combination thereof sufficient to increase serotonin level in the subject, and an excipient or carrier suitable for delivery to the gut promotes production of serotonin by cells of a subject in which the composition is delivered to their gut.
48. The therapeutic composition of paragraph 47, wherein the composition promotes expression of tryptophan hydroxylase 1 in cells of the subject.
49. The therapeutic composition of paragraph 47 or 48, wherein the isolated serotonin-increasing bacterial species increases serotonin in at least one of the following ways: production of secreted metabolites or secreted proteins that induce serotonin production; production of ligands that induce serotonin production; or production of an agonist of a serotonin receptor or the trace amine-associated receptor (TAAR).
50. The therapeutic composition of any one of paragraphs 47-49, wherein the isolated serotonin-increasing bacterial species increases serotonin through production of secreted metabolites or secreted proteins that induce serotonin production.
51. The therapeutic composition of any one of paragraphs 47-50, wherein the isolated serotonin-increasing bacterial species comprises one or more species selected from the group consisting of: Acidaminococcus intestini, Agathobacter rectalis, Alistipes onderdonkii, Alistipes putredinis, Anaerotruncus colihominis, Bacillus cereus, Bacteroides caccae, Bacteroides cellulosilyticus, Bacteroides dorei, Bacteroides finegoldii, Bacteroides fragilis, Bacteroides koreensis, Bacteroides ovatus, Bacteroides plebeius, Bacteroides salyersiae, Bacteroides stercoris, Bacteroides uniformis, Bacteroides vulgatus, Bacteroides xylanisolvens, Bifidobacterium adolescentis, Bifidobacterium breve, Bifidobacterium faecale, Bifidobacterium longum, Bilophila wadsworthia, Butyricimonas paravirosa, Clostridium aldenese, Clostridium bolteae, Clostridium clostridioforme, Clostridium hathewayi, Clostridium innoculum, Clostridium paraputrificum, Clostridium saudiense, Clostridium scindens, Clostridium tyrobutyricum, Clostridium hylemonae HB-73, Collinsella aerofaciens, Coprococcus comes, Coprococcus eutactus, Dysosmobacter welbionis, Eisenbergiella tayi, Enterococcus faecium, Erysipelatoclostridium ramosum, Eubacterium eligens, Faecalitalea cylindroides, Flavonifractor plautii, Flintibacter butyricus, Gemmiger formicilis, Gordonibacter pamelaeae, Hungatella effluvii, Hungatella hathewayi, Intestinimonas butyriciproducens, Lactobacillus brevis, Mediterraneibacter faecis, Oscillibacter sp., Parabacteroides distasonis, Parabacteroides johnsonii, Parabacteroides merdae, Parasutterella excrementihominis, Ruminococcus bicirculans, Ruminococcus gnavus, Streptococcus gordonii, and Turicibacter sanguinis.
52. The therapeutic composition of any one of paragraphs 47-51, wherein the isolated serotonin-increasing bacterial species comprises one or more species selected from the group consisting of Acidaminococcus intestini HB-95, Agathobacter rectalis HB-257, Alistipes onderdonkii HB-311, Alistipes putredinis HB-324, Anaerotruncus colihominis HB-474, Anaerotruncus colihominis HB-83, Bacillus cereus HB-25, Bacteroides caccae HB-11, Bacteroides cellulosilyticus HB-227, Bacteroides dorei HB-12, Bacteroides finegoldii HB-31, Bacteroides fragilis HB-58, Bacteroides koreensis HB-385, Bacteroides ovatus HB-70, Bacteroides plebeius HB-237, Bacteroides salyersiae HB-32, Bacteroides stercoris HB-33, Bacteroides uniformis HB-13, Bacteroides vulgatus HB-10, Bacteroides xylanisolvens HB-35, Bifidobacterium adolescentis HB-179, Bifidobacterium breve HB-90, Bifidobacterium faecale HB-159, Bifidobacterium longum HB-71, Bilophila wadsworthia HB-693, Butyricimonas paravirosa HB-453, Clostridium aldenese HB-440, Clostridium bolteae HB-442, Clostridium clostridioforme HB-642, Clostridium hathewayi HB-152, Clostridium innoculum HB-82, Clostridium paraputrificum HB-27, Clostridium saudiense HB-142, Clostridium scindens HB-444, Clostridium tyrobutyricum HB-469, Clostridium hylemonae HB-73, Collinsella aerofaciens HB-274, Coprococcus comes HB-376, Coprococcus eutactus HB-155, Dysosmobacter welbionis HB-45, Eisenbergiella tayi HB-437, Eisenbergiella tayi HB-612, Enterococcus faecium HB-640, Enterococcus faecium HB-85, Erysipelatoclostridium ramosum HB-24, Eubacterium eligens HB-252, Faecalitalea cylindroides HB-664, Flavonifractor plautii HB-472, Flintibacter butyricus HB-344, Gemmiger formicilis HB-325, Gordonibacter pamelaeae HB-15, Hungatella effluvii HB-02, Hungatella hathewayi HB-01, Intestinimonas butyriciproducens HB-478, Lactobacillus brevis HB-87, Mediterraneibacter faecis HB-364, Oscillibacter sp. HB-28, Parabacteroides distasonis HB-20, Parabacteroides johnsonii HB-03, Parabacteroides merdae HB-63, Parasutterella excrementihominis HB-330, Ruminococcus bicirculans HB-268, Ruminococcus gnavus HB-40, Ruminococcus gnavus HB-516, Streptococcus gordonii HB-62, Streptococcus gordonii HB-98, and Turicibacter sanguinis HB-147.
53. The therapeutic composition of any one of paragraphs 47-52, wherein the isolated serotonin-increasing bacterial species comprises a 16S sequence at least 95% identical to a sequence selected from SEQ ID Nos 1, 2, and 5-69.
54. The therapeutic composition of paragraph 47, wherein the isolated serotonin-increasing bacterial species increases serotonin through production of an agonist of a serotonin receptor or the trace amine-associated receptor (TAAR).
55. The therapeutic composition of paragraph 54, wherein the isolated serotonin-increasing bacterial species encodes and expresses enzymes sufficient for the production of an agonist of a serotonin receptor or the trace amine-associated receptor (TAAR).
56. The therapeutic composition of paragraph 54 or 55, wherein the isolated bacterial species comprises one or more species selected from the group consisting of: Akkermansia muciniphila, Adlercreutzia equolifaciens, Clostridium sporogenes, Clostridium lavalense, Clostridium asparagiforme, Coprococcus eutactus, Coprococcus comes, Enterococcus durans, Enterorhabdus muris, Enterorhabdus caecimuris, Mycolicibacterium smegmatis, Peptostreptococcus russelli, and Ruminococcus gnavus.
57. The therapeutic composition of any one of paragraphs 54-56, wherein the isolated bacterial species comprises one or more species selected from the group consisting of: Enterococcus durans HB-48, Clostridium lavalense HB-452c, Ruminococcus gnavus HB-40, Ruminococcus gnavus HB-516. In some embodiments, the 5-HT agonist-producing bacteria are greater than 95% similar by 16S sequencing to Enterococcus durans HB-48, Clostridium lavalense HB-452c, Ruminococcus gnavus HB-40, Ruminococcus gnavus HB-516, Clostridium sporogenes JCM 7836, Akkermansia muciniphila BAA-835, Clostridium sporogenes McClung 2004, Peptostreptococcus russeliii RT-10B, Mycolicibacterium smegmatis ATCC 19420, Enterorhabdus muris WCA-131-CoC-2, Adlercreutzia equolifaciens FJC-B9, Enterorhabdus caecimuris B7, Coprococcus eutactus ATCC 27759, and Coprococcus comes ATCC 27758.
58. The therapeutic composition of any one of paragraphs 54-57, wherein the isolated bacterial species comprises a 16S sequence at least 95% identical to a sequence selected from SEQ ID Nos 1-4 and 105-114.
59. The therapeutic composition of any one of paragraphs 54-58, wherein the agonist of the serotonin receptor or TAAR is selected from the group consisting of N-methyltryptamine, N,N-dimethyltryptamine, N-methylserotonin, and N,N-dimethylserotonin.
60. The therapeutic composition of either of any one of paragraphs 54-59, wherein the isolated serotonin-increasing bacterial species encodes and expresses one or more enzymes that catalyze the methylation of tryptamine by a mechanism corresponding to that of human indolethylamine N-methyltransferase, or one or more enzymes at least 50% identical to the radical S-adenosyl-L-methionine-dependent, ergothioneine biosynthetic enzyme egtD, or one or more phosphatidylethanolamine N-methyltransferase enzymes (e.g., SEQ ID NOs: 228 or 229).
61. The therapeutic composition of paragraph 47, wherein culture supernatant of the isolated bacterial species increases expression of tryptophan hydroxylase 1 (TPH-1) in cells of the host.
62. The therapeutic composition of paragraph 61, wherein the isolated bacterial species comprises one or more species selected from the group consisting of Enterococcus durans, Clostridium lavalense, Lactobacillus brevis, Bifidobacterium faecale, Anaerotruncus colihominis, and Clostridium ramosum.
63. The therapeutic composition of paragraph 61 or 62, wherein the isolated bacterial species comprises one or more species selected from the group consisting of Enterococcus durans HB-48, Clostridium lavalense HB-452c, Lactobacillus brevis HB-87, Bifidobacterium faecale HB-159, Anaerotruncus colihominis HB-83, and Clostridium ramosum HB-24 or a combination thereof.
64. The therapeutic composition of any one of paragraphs 61-63, wherein the isolated bacterial species comprises a 16S sequence at least 95% identical to a sequence selected from the group consisting of SEQ ID Nos: 3, 4, 11, 28, 30 and 39.
65. The therapeutic composition of paragraph 50, wherein the isolated bacterial species comprises one or more species selected from the group consisting of Clostridium scindens, Bifidobacterium faecale, Enterococcus durans, Clostridium lavalense, Anaerotruncus colihominis, and Erysipelatoclostridium ramosum.
66. The therapeutic composition of paragraph 65, wherein the isolated bacterial species comprises one or more species selected from the group consisting of Clostridium scindens HB-444, Bifidobacterium faecale HB-159, Enterococcus durans HB-48, Clostridium lavalense HB-452c, Anaerotruncus colihominis HB-83, and Erysipelatoclostridium ramosum HB-24.
67. The therapeutic composition of paragraph 65 or 66, wherein the isolated bacterial species comprises a 16S sequence that is at least 95% identical to one of SEQ ID NOs: 3, 4, 11, 23, 28 and 39.
68. The therapeutic composition of paragraph 47, wherein the isolated serotonin-increasing bacterial species increases serotonin through production of ligands that induce serotonin production.
69. The therapeutic composition of paragraph 68, wherein a cell pellet from the isolated serotonin increasing bacterial species modulates serotonin when administered to a subject.
70. The therapeutic composition of paragraph 68 or 69, wherein the isolated bacterial species comprises one or more species selected from the group consisting of Anaerotruncus colihominis, Bacteroides caccae, Bacteroides clarus, Bacteroides dorei, Bacteroides finegoldii, Bacteroides ovatus, Bacteroides salyersiae, Bacteroides thetaiotaomicron, Bacteroides xylanisolvens, Bifidobacterium adolescentis, Bifidobacterium faecale, Bittarella massiliensis, Blautia wexlerae, Clostridium aldenese, Clostridium bolteae, Clostridium hathewayi, Clostridium saudiense, Clostridium scindens, Clostridium tyrobutyricum, Dialister invisus, Eisenbergiella tayi, Enterococcus durans, Enterococcus faecium, Eubacterium eligens, Gemmiger formicilis, Gordonibacter pamelaeae, Hungatella effluvii, Lactobacillus brevis, Longibaculum muris, Mediterraneibacter faecis, Parabacteroides distasonis, Parabacteroides merdae, Parasutterella excrementihominis, Prevotella copri, Prevotella sp, Prevotella sp., Romboutsia lituseburensis, Ruminococcus sp., Ruminococcus gnavus, Sellimonas intestinalis, and Sutterella wadsworthensis.
71. The therapeutic composition of any one of paragraphs 68-70, wherein the isolated bacterial species comprises one or more species selected from the group consisting of Anaerotruncus colihominis HB-83, Bacteroides caccae HB-11, Bacteroides clarus HB-30, Bacteroides dorei HB-12, Bacteroides finegoldii HB-31, Bacteroides ovatus HB-70, Bacteroides salyersiae HB-32, Bacteroides thetaiotaomicron HB-34, Bacteroides xylanisolvens HB-35, Bifidobacterium adolescentis HB-179, Bifidobacterium faecale HB-159, Bittarella massiliensis HB-477, Blautia wexlerae HB-16, Clostridium aldenese HB-440, Clostridium bolteae HB-442, Clostridium hathewayi HB-152, Clostridium saudiense HB-142, Clostridium scindens HB-444, Clostridium tyrobutyricum HB-469, Dialister invisus HB-387, Eisenbergiella tayi HB-612, Enterococcus durans HB-48, Enterococcus faecium HB-85, Eubacterium eligens HB-252, Gemmiger formicilis HB-325, Gordonibacter pamelaeae HB-15, Hungatella effluvii HB-02, Lactobacillus brevis HB-87, Longibaculum muris HB-79, Mediterraneibacter faecis HB-364, Parabacteroides distasonis HB-20, Parabacteroides merdae HB-63, Parasutterella excrementihominis HB-330, Prevotella copri HB-373, Prevotella sp HB-649, Prevotella sp. HB-333, Romboutsia lituseburensis HB-102, Ruminococcus sp. HB-626, Ruminococcus gnavus HB-40, Ruminococcus gnavus HB-516, Sellimonas intestinalis HB-443, and Sutterella wadsworthensis HB-259.
72. The therapeutic composition of any one of paragraphs 68-71, wherein the isolated bacterial species comprises a 16S sequence at least 95% identical to a sequence selected from the group consisting of SEQ ID Nos 1-3, 5-30 and 70-82.
73. The therapeutic composition of any one of paragraphs 68-72, wherein the cell pellet increases expression of TPH-1 in cells of the subject.
74. The therapeutic composition of paragraph 73, wherein the isolated bacterial species comprises one or more species selected from Clostridium lavalense, Lactobacillus brevis, Bifidobacterium faecale, Anaerotruncus colihominis, and Clostridium ramosum.
75. The therapeutic composition of paragraph 73 or 74, wherein the isolated bacterial species comprises one or more species selected from Clostridium lavalense HB-452c, Lactobacillus brevis HB-87, Bifidobacterium faecale HB-159, Anaerotruncus colihominis HB-83, and Clostridium ramosum HB-24.
76. The therapeutic composition of any one of paragraphs 73-75, wherein the isolated bacterial species comprises one or more species that comprise a 16S sequence at least 95% identical to a sequence selected from SEQ ID Nos 4, 11, 28, 30, and 39.
77. The therapeutic composition of any one of paragraphs 73-76, wherein the isolated bacterial species comprises one or more species selected from Clostridium scindens, Bifidobacterium faecale, Enterococcus durans, Clostridium lavalense, Anaerotruncus colihominis, and Erysipelatoclostridium ramosum.
78. The therapeutic composition of any one of paragraphs 73-77, wherein the isolated bacterial species comprises one or more species selected from Clostridium scindens HB-444, Bifidobacterium faecale HB-159, Enterococcus durans HB-48, Clostridium lavalense HB-452c, Anaerotruncus colihominis HB-83, and Erysipelatoclostridium ramosum HB-24.
79. The therapeutic composition of any one of paragraphs 73-78, wherein the isolated bacterial species comprises one or more species that comprise a 16S sequence at least 95% identical to a sequence selected from SEQ ID Nos 3, 4, 11, 23, 28 and 39.
80. The therapeutic composition of any one of paragraphs 1-35 or 47-79, wherein the isolated bacterial species are grown in medium containing one or more nutrients selected from the group consisting of conditioned medium from other bacteria, N-Acetyl-D-Galactosamine, N-Acetyl-D-Glucosamine, N-Acetyl-β-D-Mannosamine, Adonitol, Amygdalin, D-Arabitol, Arbutin, D-Cellobiose, α-Cyclodextrin, β-Cyclodextrin, Dextrin, Dulcitol, i-Erythritol, D-Fructose, L-Fucose, D-Galactose, D-Galacturonic Acid, Gentiobiose, D-Gluconic Acid, D-Glucosaminic Acid, α-D-Glucose, α-D-Glucose 1-Phosphate, D-Glucose6-Phosphate, Glycerol, D,L-α-Glycerol Phosphate, m-Inositol, α-D-Lactose, Lactulose, Maltose, Maltotriose, D-Mannitol, D-Mannose, D-Melezitose, D-Melibiose, 3-Methyl-DGlucose, α-Methyl-DGalactoside, β-Methyl-D-Galactoside, α-Methyl-D-Glucoside, β-Methyl-D-Glucoside, Mucin, Palatinose, D-Raffinose, L-Rhamnose, Salicin, D-Sorbitol, Stachyose, Sucrose, D-Trehalose, Turanose, Acetic Acid, Formic Acid, Fumaric Acid, Glyoxylic Acid, α-Hydroxybutyric Acid, β-Hydroxybutyric Acid, Itaconic Acid, α-Ketobutyric Acid, α-Ketovaleric Acid, D,L-Lactic Acid, L-Lactic Acid, D-Lactic Acid Methyl Ester, D-Malic Acid, L-Malic Acid, Propionic Acid, Pyruvic Acid, Pyruvic Acid Methyl Ester, D-Saccharic Acid, Succinamic Acid, Succinic Acid, Succinic Acid Mono-Methyl Ester, m-Tartaric Acid, Urocanic Acid, Alaninamide, L-Alanine, L-Alanyl-LGlutamine, L-Alanyl-LHistidine, L-Alanyl-LThreonine, L-Asparagine, L-Glutamic Acid, L-Glutamine, Glycyl-LAspartic Acid, Glycyl-LGlutamine, Glycyl-LMethionine, Glycyl-LProline, L-Methionine, L-Phenylalanine, L-Serine, L-Threonine, L-Valine, L-Valine plus L-Aspartic Acid, 2′-Deoxy Adenosine, Inosine, Thymidine, Uridine, Thymidine-5′-Mono-phosphate, and Uridine-5′-Monophosphate.
81. The therapeutic composition of paragraph 47, wherein an isolated serotonin-increasing bacterial species in the composition produces tryptophan.
82. The therapeutic composition of paragraph 81, wherein an isolated serotonin-increasing bacterial species in the composition encodes or expresses at least one enzyme involved in tryptophan production.
83. The therapeutic composition of paragraph 81 or 82, wherein the enzyme involved in tryptophan production is selected from the group consisting of: Tryptophan synthase; Indole-3-glycerol phosphate synthase; Anthranilate phosphoribosyltransferase; Anthranilate synthase; and N-(5′-phosphoribosyl)anthranilate isomerase; 1-(5-phosphoribosyl)-5-[(5-phosphoribosylamino)methylideneamino] imidazole-4-carboxamide isomerase.
84. The therapeutic composition of any one of paragraphs 81-83, wherein the enzyme involved in tryptophan production belongs to an EC number selected from the group consisting of: EC 4.2.1.20, EC 4.1.1.48, EC 2.4.2.18, EC 4.1.3.27, EC 5.3.1.24, and EC 5.3.1.36.
85. The therapeutic composition of any one of paragraphs 81-84, wherein the enzyme involved in tryptophan production has an amino acid sequence at least 50% identical to a sequence selected from SEQ ID Nos 135-163.
86. The therapeutic composition of paragraph 47, wherein an isolated serotonin-increasing bacterial species in the composition produces a metabolite of phenylalanine selected from phenethylamine, tyramine or N-methylated derivatives thereof that activate the TAAR system.
87. The therapeutic composition of paragraph 47, wherein the isolated serotonin-increasing bacterial species in the composition produces one or more indole-3-carboxylic acid derivatives of tryptophan.
88. The therapeutic composition of paragraph 86 or 87, wherein the indole-3-carboxylic acid derivative of tryptophan is one or more of indole-3-propionic acid, indole-3-acrylic acid, indole-3-lactic acid, indole-3-pyruvic acid, or indole-3-acetic acid.
89. The therapeutic composition of paragraph 87 or 88, wherein the isolated bacterial species comprises and expresses genes of the fldAIBC gene cluster.
90. The therapeutic composition of any one of paragraphs 87-89, wherein the isolated bacterial species encodes and expresses acyl-CoA dehydrogenase.
91. The therapeutic composition of any one of paragraphs 87-90, wherein the acyl-CoA dehydrogenase belongs to EC 1.3.99.3; EC 1.3.8.7; EC 1.3.8.8; or EC 1.3.8.9.
92. The therapeutic composition of paragraph 91, wherein the acyl-CoA dehydrogenase has an amino acid sequence at least 50% identical to a sequence selected from SEQ ID Nos 164-171.
93. The therapeutic composition of paragraph 47, wherein the isolated bacterial species encodes and expresses an enzyme with a sequence at least 50% identical to an enzyme having an amino acid sequence of any one of SEQ ID Nos 172-184.
94. The therapeutic composition of paragraph 93, wherein the isolated bacterial species encodes and expresses an enzyme belonging to an EC group selected from: EC 4.1.99.1; EC 2.8.3.17; EC 4.2.1.175; EC 5.6.1.9; and EC 2.1.1.
95. A pharmaceutical composition comprising the therapeutic composition of any one of paragraphs 47-94, and a pharmaceutically acceptable carrier.
96. A method of increasing serotonin level in a mammalian subject in need thereof, the method comprising administering a composition of any one of paragraphs 47-95 to the subject, whereby a serotonin level is increased.
97. The method of paragraph 96, wherein the administering is to the gut of the subject.
98. The method of paragraph 96 or 97, wherein the level of serotonin in the gut is increased.
99. The method of any one of paragraphs 96-98, wherein the level of serotonin in circulation is increased.
100. A method of treating a disease or disorder involving or characterized by low serotonin in a subject in need thereof, the method comprising administering a composition of any one of paragraphs 47-95 to the subject, whereby the disease or disorder is treated.
101. The method of paragraph 100, wherein the administering is to the gut of the subject.
102. The method of paragraph 100 or 101, wherein the level of serotonin in the gut is increased.
103. The method of any one of paragraphs 100-102, wherein the level of serotonin in circulation is increased.
104. The method of any one of paragraphs 100-103, wherein the disease or disorder is selected from the group consisting of constipation, IBS-C, depression, anxiety, addiction, neurodegenerative disorders, autism spectrum disorder, sleep disorders, attention deficit hyperactivity disorder (ADHD), memory loss (e.g. dementia), learning difficulties, osteoporosis, heartburn, dermatological conditions (eczema and itch), GERD, and pain disorders.
105. The method of any one of paragraphs 108-104, wherein the disease or disorder is not a gut disease or disorder.
106. The method of any one of paragraphs 108-105, wherein the disease or disorder is selected from the group consisting of depression, anxiety, addiction, neurodegenerative disorders, autism spectrum disorder, sleep disorders, attention deficit hyperactivity disorder (ADHD), memory loss (e.g. dementia), learning difficulties, osteoporosis, dermatological conditions (eczema and itch), and pain disorders.
107. A therapeutic composition for decreasing serotonin level in a mammalian subject in need thereof, the composition comprising an amount of a live isolated bacterial species, dead isolated bacterial species, conditioned medium from an isolated, cultured bacterial species, cell pellet of an isolated bacterial species, a purified metabolite produced by an isolated bacterial species, a purified protein produced by an isolated bacterial species, or a combination thereof sufficient to decrease serotonin level in the subject, and an excipient or carrier suitable for delivery to the gut.
108. The therapeutic composition of paragraph 107, wherein bacterial species consumes serotonin and/or reduces host biosynthesis of serotonin.
109. The therapeutic composition of paragraph 107 or 108, wherein the bacterial species is selected from one or more of Bifidobacterium longum, Blautia coccoides, Blautia obeum, Clostridium butyricum, Coprococcus comes, Dorea longicatena, Eubacterium rectale, Lachnoclostridium sp., and Slackia isoflavoniconvertens.
110. The therapeutic composition of any one of paragraphs 107-109, wherein the bacterial species is selected from one or more of: Bifidobacterium longum HB-234, Blautia coccoides HB-23, Blautia obeum HB-14, Clostridium butyricum HB-88, Coprococcus comes HB-80, Dorea longicatena HB-17, Eubacterium rectale HB-22, Lachnoclostridium sp. HB-698, and Slackia isoflavoniconvertens HB-326.
111. The therapeutic composition of paragraph 107-110, wherein the isolated bacterial species comprises a 16S sequence at least 95% identical to a sequence selected from SEQ ID Nos 96-104.
112. A method of decreasing serotonin in a mammalian subject in need thereof, the method comprising administering a composition of any one of paragraphs 107-111 to the subject, whereby a serotonin level is decreased.
113. The method of paragraph 112, wherein the administering is to the gut of the subject.
114. The method of paragraph 112 or 113, wherein the level of serotonin in the gut is decreased.
115. The method of any one of paragraphs 112-114, wherein the level of serotonin in circulation is decreased.
116. A method of treating a disease or disorder involving or characterized by high or elevated serotonin in a subject in need thereof, the method comprising administering a composition of any one of paragraphs 107-111 to the subject, whereby the disease or disorder is treated.
117. The method of paragraph 116, wherein the administering is to the gut of the subject.
118. The method of paragraph 116 or 117, wherein the level of serotonin in the gut is decreased.
119. The method of any one of paragraphs 116-118, wherein the level of serotonin in circulation is decreased.
120. The method of any one of paragraphs 116-119, wherein the disease or disorder is selected from the group consisting of diarrhea, IBS-D, inflammatory bowel disease, anxiety, social phobia, sleep disorders, schizophrenia, cancer, obesity, diabetes, coronary artery disease, heart valve disease, congenital heart disease, cardiomyopathies, pericarditis, or platelet disorders (e.g. essential thrombocytosis).
121. The method of any one of paragraphs 116-120, wherein the disease or disorder is not a gut disease or disorder.
122. The method of any one of paragraphs 116-121, wherein the disease or disorder is selected from the group consisting of anxiety, social phobia, sleep disorders, schizophrenia, cancer, obesity, diabetes, coronary artery disease, heart valve disease, congenital heart disease, cardiomyopathies, pericarditis, or platelet disorders (e.g. essential thrombocytosis).
123. A therapeutic composition comprising one or more live isolated serotonin-modulating bacteria, dead isolated serotonin modulating bacteria, conditioned medium(s) from an isolated, cultured serotonin-modulating bacteria, cell pellet(s) of isolated serotonin-modulating bacteria, purified metabolite(s) produced by isolated serotonin-modulating bacteria, purified protein(s) produced by an isolated serotonin-modulating bacteria, or a combination thereof, which alter serotonin signaling or biosynthesis in a subject in need thereof.
124. The therapeutic composition of paragraph 123, wherein the at least one isolated serotonin-modulating bacteria belongs to a genus selected from the group consisting of: Acidaminococcus, Agathobacter, Adlercreutzia, Akkermansia, Alistipes, Anaerotruncus, Bacillus, Bacteroides, Bifidobacterium, Bilophila, Bittarella, Blautia, Blautia, Butyricimonas, Clostridium, Clostridium, Collinsella, Coprococcus, Dialister, Dorea, Dysosmobacter, Eisenbergiella, Enterococcus, Enterorhabdus, Erysipelatoclostridium, Escherichia, Eubacterium, Faecalitalea, Flavonifractor, Flintibacter, Gemmiger, Gordonibacter, Hungatella, Intestinimonas, Lachnoclostridium, Lactobacillus, Lawsonibacter, Longibaculum, Mediterraneibacter, Mycolicibacterium, Oscillibacter, Parabacteroides, Parasutterella, Peptostreptococcus, Prevotella, Romboutsia, Ruminococcus, Sellimonas, Slackia, Streptococcus, Sutterella, Turicibacter, and Veillonella.
125. The therapeutic composition of paragraph 123, wherein the at least one isolated serotonin-modulating bacteria are species selected from the group consisting of: Acidaminococcus intestini, Agathobacter rectalis, Akkermansia muciniphila, Adlercreutzia equolifaciens, Alistipes onderdonkii, Alistipes putredinis, Anaerotruncus colihominis, Bacillus cereus, Bacteroides caccae, Bacteroides cellulosilyticus, Bacteroides clarus, Bacteroides dorei, Bacteroides finegoldii, Bacteroides fragilis, Bacteroides koreensis, Bacteroides ovatus, Bacteroides plebeius, Bacteroides salyersiae, Bacteroides stercoris, Bacteroides thetaiotaomicron, Bacteroides unformis, Bacteroides vulgatus, Bacteroides xylanisolvens, Bifidobacterium adolescentis, Bifidobacterium breve, Bifidobacterium faecale, Bifidobacterium longum, Bilophila wadsworthia, Bittarella massiliensis, Blautia coccoides, Blautia obeum, Blautia wexlerae, Butyricimonas paravirosa, Clostridium asparagiforme, Clostridium aldenese, Clostridium bolteae, Clostridium butyricum, Clostridium clostridioforme, Clostridium hathewayi, Clostridium innoculum, Clostridium lavalense, Clostridium paraputrificum, Clostridium saudiense, Clostridium scindens, Clostridium sp., Clostridium sporogenes, Clostridium sphenoides, Clostridium symbiosum, Clostridium tyrobutyricum, Clostridium hylemonae, Collinsella aerofaciens, Coprococcus comes, Coprococcus eutactus, Dialister invisus, Dorea longicatena, Dysosmobacter welbionis, Eisenbergiella tayi, Enterococcus durans, Enterococcus faecium, Enterorhabdus caecimuris, Enterorhabdus muris, Erysipelatoclostridium ramosum, Escherichia coli, Eubacterium callanderi, Eubacterium eligens, Eubacterium rectale, Faecalitalea cylindroides, Flavonifractor plautii, Flintibacter butyricus, Gemmiger formicilis, Gemmiger sp., Gordonibacter pamelaeae, Hungatella effluvii, Hungatella hathewayi, Intestinimonas butyriciproducens, Intestinimonas massiliensis, Lachnoclostridium sp., Lactobacillus brevis, Lawsonibacter asaccharolyticus, Longibaculum muris, Longibaculum sp., Mediterraneibacter faecis, Mycolicibacterium smegmatis, Oscillibacter sp., Parabacteroides distasonis, Parabacteroides goldsteinii, Parabacteroides johnsonii, Parabacteroides merdae, Parasutterella excrementihominis, Peptostreptococcus russellii, Prevotella copri, Prevotella sp, Prevotella sp., Romboutsia lituseburensis, Ruminococcus bicirculans, Ruminococcus gnavus, Ruminococcus sp., Sellimonas intestinalis, Slackia isofavoniconvertens, Streptococcus gordonii, Sutterella wadsworthensis, Turicibacter sanguinis, and Veillonella atypica.
126. The therapeutic composition of paragraph 123, wherein the one or more serotonin-modulating bacteria include a strain selected from the group consisting of: Acidaminococcus intestini HB-95, Agathobacter rectalis HB-257, Akkermansia muciniphila BAA-835, Adlercreutzia equolifaciens FJC-B9, Alistipes onderdonkii HB-311, Alistipes putredinis HB-324, Anaerotruncus colihominis HB-474, Anaerotruncus colihominis HB-83, Bacillus cereus HB-25, Bacteroides caccae HB-11, Bacteroides cellulosilyticus HB-227, Bacteroides clarus HB-30, Bacteroides dorei HB-12, Bacteroides finegoldii HB-31, Bacteroides fragilis HB-58, Bacteroides koreensis HB-385, Bacteroides ovatus HB-70, Bacteroides plebeius HB-237, Bacteroides salyersiae HB-32, Bacteroides stercoris HB-33, Bacteroides thetaiotaomicron HB-34, Bacteroides uniformis HB-13, Bacteroides vulgatus HB-10, Bacteroides xylanisolvens HB-35, Bifidobacterium adolescentis HB-179, Bifidobacterium breve HB-90, Bifidobacterium faecale HB-159, Bifidobacterium longum HB-234, Bifidobacterium longum HB-71, Bilophila wadsworthia HB-693, Bittarella massiliensis HB-477, Blautia coccoides HB-23, Blautia obeum HB-14, Blautia wexlerae HB-16, Butyricimonas paravirosa HB-453, Clostridium aldenese HB-440, Clostridium bolteae HB-442, Clostridium butyricum HB-88, Clostridium clostridioforme HB-642, Clostridium hathewayi HB-152, Clostridium innoculum HB-82, Clostridium lavalense HB-452c, Clostridium paraputrificum HB-27, Clostridium saudiense HB-142, Clostridium scindens HB-444, Clostridium sp. HB-358, Clostridium sphenoides HB-470, Clostridium sporogenes JCM 7836, Clostridium sporogenes McClung 2004, Clostridium symbiosum HB-67, Clostridium tyrobutyricum HB-469, Clostridium hylemonae HB-73, Collinsella aerofaciens HB-274, Coprococcus comes HB-376, Coprococcus comes HB-80, Coprococcus comes ATCC 27758, Coprococcus eutactus HB-155, Coprococcus eutactus ATCC 27759, Dialister invisus HB-387, Dorea longicatena HB-17, Dysosmobacter welbionis HB-45, Eisenbergiella tayi HB-437, Eisenbergiella tayi HB-612, Enterococcus durans HB-48, Enterococcus faecium HB-640, Enterococcus faecium HB-85, Enterorhabdus caecimuris B7, Enterorhabdus muris WCA-131-CoC-2, Erysipelatoclostridium ramosum HB-24, Escherichia coli HB-490, Eubacterium callanderi HB-59, Eubacterium eligens HB-252, Eubacterium rectale HB-22, Faecalitalea cylindroides HB-664, Flavonifractor plautii HB-472, Flintibacter butyricus HB-344, Gemmiger formicilis HB-325, Gemmiger sp. HB-567, Gordonibacter pamelaeae HB-15, Hungatella effluvii HB-02, Hungatella hathewayi HB-01, Intestinimonas butyriciproducens HB-478, Intestinimonas massiliensis HB-651, Lachnoclostridium sp. HB-698, Lactobacillus brevis HB-87, Lawsonibacter asaccharolyticus HB-521, Longibaculum muris HB-79, Longibaculum sp. HB-681, Mediterraneibacter faecis HB-364, Mycolicibacterium smegmatis ATCC 19420, Oscillibacter sp. HB-28, Parabacteroides distasonis HB-20, Parabacteroides distasonis HB-214, Parabacteroides goldsteinii HB-44, Parabacteroides johnsonii HB-03, Parabacteroides merdae HB-63, Parasutterella excrementihominis HB-330, Peptostreptococcus russelli RT-10B, Prevotella copri HB-373, Prevotella sp HB-649, Prevotella sp. HB-333, Romboutsia lituseburensis HB-102, Ruminococcus bicirculans HB-105, Ruminococcus bicirculans HB-268, Ruminococcus gnavus HB-40, Ruminococcus gnavus HB-516, Ruminococcus sp. HB-626, Sellimonas intestinalis HB-443, Slackia isoflavoniconvertens HB-326, Streptococcus gordonii HB-62, Streptococcus gordonii HB-98, Sutterella wadsworthensis HB-259, Turicibacter sanguinis HB-147, and Veillonella atypica HB-251.
127. The therapeutic composition of paragraph 123, wherein the one or more serotonin-modulating bacteria consists of a bacteria comprising a 16S rDNA sequence at least about 95% identical to a 16S rDNA sequence selected from one of SEQ ID NOs: 1-114.
128. The therapeutic composition of paragraph 123, wherein the serotonin-modulating bacteria encode genes in their genome, which when expressed, result in the production of one or more metabolites or proteins that influence subject serotonin signaling/biosynthesis.
129. The therapeutic composition of paragraph 128, wherein the encoded genes are expressed at physiologically relevant conditions of the human gastrointestinal tract, resulting in the production of metabolites or proteins that influence subject serotonin signaling/biosynthesis.
130. The therapeutic composition of paragraph 123, wherein the composition is in the form of a probiotic, prebiotic, a capsule, a tablet, a caplet, a pill, a troche, a lozenge, a powder, a granule, a medical food, a supplement, or a combination thereof.
131. The therapeutic composition of paragraph 123, wherein the composition is formulated to be administered orally, intravenously, intramuscularly, intrathecally, subcutaneously, sublingually, buccally, rectally, vaginally, by the ocular route, by the otic route, nasally, via inhalation, by nebulization, cutaneously, transdermally, or a combination thereof.
132. A pharmaceutical composition comprising the therapeutic composition of any one of paragraphs 123-131, and a pharmaceutically acceptable carrier.
133. A method of treating a disease or disorder in a subject in need thereof, the method comprising administering to the subject an effective amount of a therapeutic composition comprising one or more live isolated serotonin-modulating bacteria, dead isolated serotonin modulating bacteria, conditioned medium(s) derived from an isolated serotonin-modulating bacteria, cell pellet(s) of isolated serotonin-modulating bacteria, purified metabolite(s) produced by isolated serotonin-modulating bacteria, purified protein(s) produced by isolated serotonin-modulating bacteria, or a combination thereof, thereby altering serotonin signaling or biosynthesis in the subject to treat the disease or disorder.
134. The method of paragraph 133, wherein the disease or disorder is a serotonin-related disease or disorder.
135. The method of paragraph 133, wherein the serotonin-related disease or disorder is selected from the group consisting of intestinal motility disorders, irritable bowel syndrome, inflammatory bowel disease, depression (e.g. major depressive disorder, treatment resistant depression, post-partum depression), anxiety disorders, addiction, social phobia, neurodegenerative disorders, autism spectrum disorder, sleep disorders, attention deficit hyperactivity disorder (ADHD), memory loss (e.g. dementia), learning difficulties, sleep disorders, schizophrenia, bone disease (e.g. osteoporosis), cancer (e.g. polycythemia vera or myelosclerosis), metabolic disease (e.g. obesity or diabetes), a dysregulated immune system, cardiac disease (e.g. coronary artery disease, heart valve disease, congenital heart disease, cardiomyopathies, pericarditis, or aorta disease), heartburn, dermatological conditions (e.g. eczema and itch), GERD, platelet disorders (e.g. essential thrombocytosis), and pain disorders.
136. The method of paragraph 133, wherein the disease or disorder is caused by high serotonin levels and is selected from the group: diarrhea, IBS-D, inflammatory bowel disease, anxiety, social phobia, sleep disorders, schizophrenia, cancer, obesity, diabetes, coronary artery disease, heart valve disease, congenital heart disease, cardiomyopathies, pericarditis, or platelet disorders (e.g. essential thrombocytosis).
137. The method of paragraph 133, wherein the disease or disorder is caused by low serotonin levels and is selected from the group: constipation, IBS-C, depression, anxiety, addiction, neurodegenerative disorders, autism spectrum disorder, sleep disorders, attention deficit hyperactivity disorder (ADHD), memory loss (e.g. dementia), learning difficulties, osteoporosis, heartburn, dermatological conditions (eczema and itch), GERD, or pain disorders.
138. The method of paragraph 133, wherein treating a disease or disorder comprises decreasing at least one symptom of the disease or disorder, selected from: fatigue, insomnia, stress, persistent anxiety, persistent sadness, social withdrawal, substance withdrawal, irritability, thoughts of suicide, thoughts of self-harm, restlessness, low sex drive, lack of focus, loss of appetite, high blood pressure, low blood pressure, high heart rate, low heart rate, constipation, diarrhea, chronic pain, heartburn, fatigue, trouble breathing, stomach aches, nosebleeds, gum, stomach bleeding, headaches, weight gain, burning of the skin, altered inflammatory markers, neurodevelopmental deficits, and/or seizures.
139. The method of paragraph 133, wherein the at least one isolated serotonin-modulating bacteria belongs to a genus selected from the group consisting of: Acidaminococcus, Agathobacter, Adlercreutzia, Akkermansia, Alistipes, Anaerotruncus, Bacillus, Bacteroides, Bifidobacterium, Bilophila, Bittarella, Blautia, Blautia, Butyricimonas, Clostridium, Clostridium, Collinsella, Coprococcus, Dialister, Dorea, Dysosmobacter, Eisenbergiella, Enterococcus, Enterorhabdus, Erysipelatoclostridium, Escherichia, Eubacterium, Faecalitalea, Flavonifractor, Flintibacter, Gemmiger, Gordonibacter, Hungatella, Intestinimonas, Lachnoclostridium, Lactobacillus, Lawsonibacter, Longibaculum, Mediterraneibacter, Mycolicibacterium, Oscillibacter, Parabacteroides, Parasutterella, Peptostreptococcus, Prevotella, Romboutsia, Ruminococcus, Sellimonas, Slackia, Streptococcus, Sutterella, Turicibacter, and Veillonella.
140. The method of paragraph 133, wherein the at least one isolated serotonin-modulating bacteria are species selected from the group consisting of: Acidaminococcus intestini, Agathobacter rectalis, Akkermansia muciniphila, Adlercreutzia equolifaciens, Alistipes onderdonkii, Alistipes putredinis, Anaerotruncus colihominis, Bacillus cereus, Bacteroides caccae, Bacteroides cellulosilyticus, Bacteroides clarus, Bacteroides dorei, Bacteroides finegoldii, Bacteroides fragilis, Bacteroides koreensis, Bacteroides ovatus, Bacteroides plebeius, Bacteroides salyersiae, Bacteroides stercoris, Bacteroides thetaiotaomicron, Bacteroides uniformis, Bacteroides vulgatus, Bacteroides xylanisolvens, Bifidobacterium adolescentis, Bifidobacterium breve, Bifidobacterium faecale, Bifidobacterium longum, Bilophila wadsworthia, Bittarella massiliensis, Blautia coccoides, Blautia obeum, Blautia wexlerae, Butyricimonas paravirosa, Clostridium asparagiforme, Clostridium aldenese, Clostridium bolteae, Clostridium butyricum, Clostridium clostridioforme, Clostridium hathewayi, Clostridium innoculum, Clostridium lavalense, Clostridium paraputrificum, Clostridium saudiense, Clostridium scindens, Clostridium sp., Clostridium sporogenes, Clostridium sphenoides, Clostridium symbiosum, Clostridium tyrobutyricum, Clostridium hylemonae, Collinsella aerofaciens, Coprococcus comes, Coprococcus eutactus, Dialister invisus, Dorea longicatena, Dysosmobacter welbionis, Eisenbergiella tayi, Enterococcus durans, Enterococcus faecium, Enterorhabdus caecimuris, Enterorhabdus muris, Erysipelatoclostridium ramosum, Escherichia coli, Eubacterium callanderi, Eubacterium eligens, Eubacterium rectale, Faecalitalea cylindroides, Flavonifractor plautii, Flintibacter butyricus, Gemmiger formicilis, Gemmiger sp., Gordonibacter pamelaeae, Hungatella effluvii, Hungatella hathewayi, Intestinimonas butyriciproducens, Intestinimonas massiliensis, Lachnoclostridium sp., Lactobacillus brevis, Lawsonibacter asaccharolyticus, Longibaculum muris, Longibaculum sp., Mediterraneibacter faecis, Mycolicibacterium smegmatis, Oscillibacter sp., Parabacteroides distasonis, Parabacteroides goldsteinii, Parabacteroides johnsonii, Parabacteroides merdae, Parasutterella excrementihominis, Peptostreptococcus russellii, Prevotella copri, Prevotella sp, Prevotella sp., Romboutsia lituseburensis, Ruminococcus bicirculans, Ruminococcus gnavus, Ruminococcus sp., Sellimonas intestinalis, Slackia isoflavoniconvertens, Streptococcus gordonii, Sutterella wadsworthensis, Turicibacter sanguinis, and Veillonella atypica.
141. The method of paragraph 133, wherein the one or more serotonin-modulating bacteria include a strain selected from the group consisting of: Acidaminococcus intestini HB-95, Agathobacter rectalis HB-257, Akkermansia muciniphila BAA-835, Adlercreutzia equolifaciens FJC-B9, Alistipes onderdonkii HB-311, Alistipes putredinis HB-324, Anaerotruncus colihominis HB-474, Anaerotruncus colihominis HB-83, Bacillus cereus HB-25, Bacteroides caccae HB-11, Bacteroides cellulosilyticus HB-227, Bacteroides clarus HB-30, Bacteroides dorei HB-12, Bacteroides finegoldii HB-31, Bacteroides fragilis HB-58, Bacteroides koreensis HB-385, Bacteroides ovatus HB-70, Bacteroides plebeius HB-237, Bacteroides salyersiae HB-32, Bacteroides stercoris HB-33, Bacteroides thetaiotaomicron HB-34, Bacteroides uniformis HB-13, Bacteroides vulgatus HB-10, Bacteroides xylanisolvens HB-35, Bifidobacterium adolescentis HB-179, Bifidobacterium breve HB-90, Bifidobacterium faecale HB-159, Bifidobacterium longum HB-234, Bifidobacterium longum HB-71, Bilophila wadsworthia HB-693, Bittarella massiliensis HB-477, Blautia coccoides HB-23, Blautia obeum HB-14, Blautia wexlerae HB-16, Butyricimonas paravirosa HB-453, Clostridium aldenese HB-440, Clostridium bolteae HB-442, Clostridium butyricum HB-88, Clostridium clostridioforme HB-642, Clostridium hathewayi HB-152, Clostridium innoculum HB-82, Clostridium lavalense HB-452c, Clostridium paraputrificum HB-27, Clostridium saudiense HB-142, Clostridium scindens HB-444, Clostridium sp. HB-358, Clostridium sphenoides HB-470, Clostridium sporogenes JCM 7836, Clostridium sporogenes McClung 2004, Clostridium symbiosum HB-67, Clostridium tyrobutyricum HB-469, Clostridium hylemonae HB-73, Collinsella aerofaciens HB-274, Coprococcus comes HB-376, Coprococcus comes HB-80, Coprococcus comes ATCC 27758, Coprococcus eutactus HB-155, Coprococcus eutactus ATCC 27759, Dialister invisus HB-387, Dorea longicatena HB-17, Dysosmobacter welbionis HB-45, Eisenbergiella tayi HB-437, Eisenbergiella tayi HB-612, Enterococcus durans HB-48, Enterococcus faecium HB-640, Enterococcus faecium HB-85, Enterorhabdus caecimuris B7, Enterorhabdus muris WCA-131-CoC-2, Erysipelatoclostridium ramosum HB-24, Escherichia coli HB-490, Eubacterium callanderi HB-59, Eubacterium eligens HB-252, Eubacterium rectale HB-22, Faecalitalea cylindroides HB-664, Flavonifractor plautii HB-472, Flintibacter butyricus HB-344, Gemmiger formicilis HB-325, Gemmiger sp. HB-567, Gordonibacter pamelaeae HB-15, Hungatella effluvii HB-02, Hungatella hathewayi HB-01, Intestinimonas butyriciproducens HB-478, Intestinimonas massiliensis HB-651, Lachnoclostridium sp. HB-698, Lactobacillus brevis HB-87, Lawsonibacter asaccharolyticus HB-521, Longibaculum muris HB-79, Longibaculum sp. HB-681, Mediterraneibacter faecis HB-364, Mycolicibacterium smegmatis ATCC 19420, Oscillibacter sp. HB-28, Parabacteroides distasonis HB-20, Parabacteroides distasonis HB-214, Parabacteroides goldsteinii HB-44, Parabacteroides johnsonii HB-03, Parabacteroides merdae HB-63, Parasutterella excrementihominis HB-330, Peptostreptococcus russelli RT-10B, Prevotella copri HB-373, Prevotella sp HB-649, Prevotella sp. HB-333, Romboutsia lituseburensis HB-102, Ruminococcus bicirculans HB-105, Ruminococcus bicirculans HB-268, Ruminococcus gnavus HB-40, Ruminococcus gnavus HB-516, Ruminococcus sp. HB-626, Sellimonas intestinalis HB-443, Slackia isoflavoniconvertens HB-326, Streptococcus gordonii HB-62, Streptococcus gordonii HB-98, Sutterella wadsworthensis HB-259, Turicibacter sanguinis HB-147, and Veillonella atypica HB-251.
142. The method of paragraph 133, wherein the one or more serotonin-modulating bacteria consists of a bacteria comprising a 16S rDNA sequence at least about 95% identical to a 16S rDNA sequence selected from one of SEQ ID NOs: 1-114.
143. The method of paragraph 133, wherein the serotonin-modulating bacteria encode genes in their genome, which when expressed, result in the production of one or more metabolites or proteins that influence subject serotonin signaling/biosynthesis.
144. The method of paragraph 143, wherein the encoded genes are expressed at physiologically relevant conditions of the human gastrointestinal tract, resulting in the production of metabolites or proteins that influence subject serotonin signaling/biosynthesis.
145. The method of paragraph 133, wherein the composition is in the form of a probiotic, prebiotic, a capsule, a tablet, a caplet, a pill, a troche, a lozenge, a powders, a granule, a medical food, supplement or a combination thereof.
146. The method of paragraph 133, wherein the composition is administered orally, intravenously, intramuscularly, intrathecally, subcutaneously, sublingually, buccally, rectally, vaginally, by the ocular route, by the otic route, nasally, via inhalation, by nebulization, cutaneously, transdermally, or a combination thereof
147. The method of paragraph 133, further comprising identifying a subject in need of treatment by measuring a serotonin level in a sample from the subject, and comparing the level to a reference level.
148. The method of paragraph 147, wherein the serotonin level is measured in stool, blood, or tissue of the subject.
149. The method of paragraph 147, wherein a serotonin level less than the reference level identifies a subject in need of treatment.
150. The method of paragraph 148, wherein the levels of serotonin in the stool, blood, or tissue of the subject are altered relative to their initial quantitated amounts, after administering the therapeutic composition.
151. The method of paragraph 133, further comprising identifying a subject in need of treatment by measuring levels of fecal serotonin modulating bacteria.
152. The method of paragraph 151, wherein the level of fecal serotonin-modulating bacteria is measured by fecal 16S rDNA sequencing, fecal shotgun metagenomic sequencing, measurement of fecal genes involved in the production of microbiota-derived serotonin modulating metabolites, measurement of proteins by sequencing or proteomics or comparable methods, or levels of fecal, blood, or tissue serotonin-modulating metabolites via LC/MS or comparable methods.
153. The method of paragraph 152, wherein the levels of serotonin modulating bacteria, genes involved in the production of microbiota-derived serotonin modulating metabolites or proteins, or levels of serotonin-modulating metabolites are altered relative to their initial quantitated amounts after administering the therapeutic composition.

Some embodiments of the technology described herein can be defined according to any of the following numbered paragraphs:
1. A therapeutic composition comprising one or more live isolated serotonin-modulating bacteria, dead isolated serotonin modulating bacteria, conditioned medium(s) from a isolated, cultured serotonin-modulating bacteria, cell pellet(s) of isolated serotonin-modulating bacteria, purified metabolite(s) produced by isolated serotonin-modulating bacteria, purified protein(s) produced by a isolated serotonin-modulating bacteria, or a combination thereof, which alter serotonin signaling or biosynthesis in a subject in need thereof.
2. The therapeutic composition of paragraph 1, wherein the at least one isolated serotonin-modulating bacteria belongs to the genera Acidaminococcus, Agathobacter, Alistipes, Anaerotruncus, Bacillus, Bacteroides, Bifidobacterium, Bilophila, Blautia, Butyricimonas, Clostridium, Collinsella, Coprococcus, Dialister, Dorea, Eisenbergiella, Enterococcus, Escherichia, Eubacterium, Faecalicatena, Flavonifractor, Flintibacter, Gemmiger, Gordonibacter, Hungatella, Intestinimonas, Lachnoclostridium, Lactobacillus, Lawsonibacter, Longibaculum, Mediterraneibacter, Oscillibacter, Parabacteroides, Parasutterella, Prevotella, Romboutsia, Ruminococcaceae, Ruminococcus, Sellimonas, Slackia, Streptococcus, Sutterella, Turicibacter, or Veillonella.
3. The therapeutic composition of paragraph 1, wherein the at least one isolated serotonin-modulating bacteria belongs to the species Acidaminococcus intestini, Agathobacter rectalis, Alistipes onderdonkii, Alistipes putredinis, Anaerotruncus colihominis, Bacillus cereus, Bacteroides vulgatus, Bacteroides plebeius, Bacteroides koreensis, Bacteroides cellulosilyticus, Bacteroides fragilis, Bacteroides xylanisolvens, Bacteroides uniformis, Bacteroides stercoris, Bacteroides dorei, Bacteroides caccae, Bacteroides thetaiotaomicron, Bacteroides salyersiae, Bacteroides ovatus, Bacteroides finegoldii, Bacteroides clarus, Bifidobacterium faecale, Bifidobacterium adolescentis, Bifidobacterium longum, Bifidobacterium brevis, Bilophila wadsworthia, Blautia producta, Blautia wexlerae, Blautia obeum, Butyricimonas paravirosa, Clostridium scindens, Clostridium inoculum, Clostridium bolteae, Clostridium aldenense, Clostridium saudiense, Clostridium lavalense, Clostridium amygdalium, Clostridium clostridioforme, Clostridium leptum, Clostridium tyrobutyricum, Clostridium ramosum, Clostridium paraputrificum, Clostridium sphenoides, Clostridium symbiosum, Clostridium sp., Clostridium butyricum, Collinsella aerofaciens, Coprococcus comes, Coprococcus eutactus, Coprococcus sp., Dialister invisus, Dorea longicatena, Eisenbergiella tayi, Eisenbergiella sp., Enterococcus lactis, Enterococcus hirae, Enterococcus durans, Escherichia coli, Eubacterium limosum, Eubacterium eligens, Eubacterium callanderi, Eubacterium rectale, Faecalicatena cylindroides, Flavonifractor plautii, Flintibacter sp., Gemmiger formicilis, Gemmiger sp., Genus Species, Gordonibacter pamelaeae, Hungatella hathewayi, Hungatella effluvii, Intestinimonas butyriciproducens, Intestinimonas massiliensis, Lachnoclostridium sp., Lactobacillus brevis, Lawsonibacter asaccharolyticus, Longibaculum muris, Longibaculum sp., Mediterraneibacter faecis, Oscillibacter rumenantium, Oscillibacter valericigenes, Parabacteroides distasonis, Parabacteroides johnsonii, Parabacteroides merdae, Parabacteroides goldsteinii, Parasutterella excrementihominis, Prevotella copri, Prevotella sp., Prevotella sp., Romboutsia lituseburensis, Ruminococcaceae sp., Ruminococcus gnavus, Ruminococcus bicirculans, Ruminococcus torques, Sellimonas intestinalis, Slackia isoflavoniconvertens, Streptococcus gordonii, Sutterella wadsworthensis, Turicibacter sanguinis, or Veillonella atypica.
4. The therapeutic composition of paragraph 1, wherein the one or more serotonin-modulating bacteria is/are selected from the strains Acidaminococcus intestini HB-95, Agathobacter rectalis HB-257, Alistipes onderdonkii HB-311, Alistipes putredinis HB-324, Anaerotruncus colihominis HB-474, Anaerotruncus colihominis HB-83, Bacillus cereus HB-25, Bacteroides caccae HB-11, Bacteroides cellulosilyticus HB-227, Bacteroides clarus HB-30, Bacteroides dorei HB-12, Bacteroides finegoldii HB-31, Bacteroides fragilis HB-58, Bacteroides koreensis HB-385, Bacteroides ovatus HB-70, Bacteroides plebeius HB-237, Bacteroides salyersiae HB-32, Bacteroides stercoris HB-33, Bacteroides thetaiotaomicron HB-34, Bacteroides uniformis HB-13, Bacteroides vulgatus HB-10, Bacteroides xylanisolvens HB-35, Bifidobacterium adolescentis HB-179, Bifidobacterium brevis HB-90, Bifidobacterium faecale HB-159, Bifidobacterium longum HB-234, Bifidobacterium longum HB-71, Bilophila wadsworthia HB-693, Blautia obeum HB-14, Blautia producta HB-23, Blautia wexlerae HB-16, Butyricimonas paravirosa HB-453, Clostridium aldenense HB-440, Clostridium amygdalium HB-152, Clostridium bolteae HB-442, Clostridium butyricum HB-88, Clostridium clostridioforme HB-642, Clostridium inoculum HB-82, Clostridium lavalense HB-452c, Clostridium leptum HB-73, Clostridium paraputrificum HB-27, Clostridium ramosum HB-24, Clostridium saudiense HB-142, Clostridium scindens HB-444, Clostridium sp. HB-358, Clostridium sphenoides HB-470, Clostridium symbiosum HB-67, Clostridium tyrobutyricum HB-469, Collinsella aerofaciens HB-04, Collinsella aerofaciens HB-274, Coprococcus comes HB-376, Coprococcus eutactus HB-155, Coprococcus sp. HB-80, Dialister invisus HB-387, Dorea longicatena HB-17, Eisenbergiella sp. HB-612, Eisenbergiella tayi HB-437, Enterococcus durans HB-85, Enterococcus hirae HB-48, Enterococcus lactis HB-640, Escherichia coli HB-490, Eubacterium callanderi HB-59, Eubacterium eligens HB-252, Eubacterium limosum HB-98, Eubacterium rectale HB-22, Faecalicatena cylindroides HB-664, Flavonifractor plautii HB-472, Flintibacter sp. HB-344, Gemmiger formicilis HB-325, Gemmiger sp. HB-567, Gordonibacter pamelaeae HB-15, Hungatella effluvii HB-02, Hungatella hathewayi HB-01, Intestinimonas butyriciproducens HB-478, Intestinimonas massiliensis HB-651, Lachnoclostridium sp. HB-698, Lactobacillus brevis HB-87, Lawsonibacter asaccharolyticus HB-521, Longibaculum muris HB-79, Longibaculum sp. HB-681, Mediterraneibacter faecis HB-364, Oscillibacter rumenantium HB-28, Oscillibacter valericigenes HB-45, Parabacteroides distasonis HB-20, Parabacteroides distasonis HB-214, Parabacteroides goldsteinii HB-44, Parabacteroides johnsonii HB-03, Parabacteroides merdae HB-63, Parasutterella excrementihominis HB-330, Prevotella copri HB-373, Prevotella sp. HB-333, Prevotella sp. HB-649, Romboutsia lituseburensis HB-102, Ruminococcaceae sp. HB-477, Ruminococcus bicirculans HB-105, Ruminococcus bicirculans HB-268, Ruminococcus gnavus HB-40, Ruminococcus gnavus HB-516, Ruminococcus torques HB-626, Sellimonas intestinalis HB-443, Slackia isoflavoniconvertens HB-326, Streptococcus gordonii HB-62, Sutterella wadsworthensis HB-259, Turicibacter sanguinis HB-147, and Veillonella atypica HB-251.
5. The therapeutic composition of paragraph 1, wherein the one or more serotonin-modulating bacteria consists of a bacteria comprising a 16S rDNA sequence at least about 95% identical to a 16S rDNA sequence selected from one of SEQ ID NOs: 1-105.
6. The therapeutic composition of paragraph 1, wherein the serotonin-modulating bacteria encode genes in their genome, which when expressed, result in the production of one or more metabolites or proteins that influence subject serotonin signaling/biosynthesis.
7. The therapeutic composition of paragraph 6, wherein the encoded genes are expressed at physiologically relevant conditions of the human gastrointestinal tract, resulting in the production of metabolites or proteins that influence subject serotonin signaling/biosynthesis.
8. The therapeutic composition of paragraph 1, wherein the composition is in the form of a probiotic, prebiotic, a capsule, a tablet, a caplet, a pill, a troche, a lozenge, a powder, a granule, a medical food, or a combination thereof.
9. The therapeutic composition of paragraph 1, wherein the composition is formulated to be administered orally, intravenously, intramuscularly, intrathecally, subcutaneously, sublingually, buccally, rectally, vaginally, by the ocular route, by the otic route, nasally, via inhalation, by nebulization, cutaneously, transdermally, or a combination thereof.
10. A method of treating a disease or disorder in a subject in need thereof, the method comprising administering to the subject an effective amount of a therapeutic composition comprising one or more live isolated serotonin-modulating bacteria, dead isolated serotonin modulating bacteria, conditioned medium(s) derived from a isolated serotonin-modulating bacteria, cell pellet(s) of isolated serotonin-modulating bacteria, purified metabolite(s) produced by isolated serotonin-modulating bacteria, purified protein(s) produced by isolated serotonin-modulating bacteria, or a combination thereof, thereby altering serotonin signaling or biosynthesis in the subject to treat the disease or disorder.
11. The method of paragraph 10, wherein the disease or disorder is a serotonin-related disease or disorder.
12. The method of paragraph 10, wherein the serotonin-related disease or disorder is selected from the group consisting of intestinal motility disorders, irritable bowel syndrome, inflammatory bowel disease, depression (e.g. major depressive disorder, treatment resistant depression, post-partum depression), anxiety disorders, addiction, social phobia, neurodegenerative disorders, autism spectrum disorder, sleep disorders, attention deficit hyperactivity disorder (ADHD), memory loss (e.g. dementia), learning difficulties, sleep disorders, schizophrenia, bone disease (e.g. osteoporosis), cancer (e.g. polycythemia vera or myelosclerosis), metabolic disease (e.g. obesity or diabetes), a dysregulated immune system, cardiac disease (e.g. coronary artery disease, heart valve disease, congenital heart disease, cardiomyopathies, pericarditis, or aorta disease), heartburn, dermatological conditions (e.g. eczema and itch), GERD, platelet disorders (e.g. essential thrombocytosis), and pain disorders.
13. The method of paragraph 10, wherein the disease or disorder is caused by high serotonin levels and is selected from the group: diarrhea, IBS-D, inflammatory bowel disease, anxiety, social phobia, sleep disorders, schizophrenia, cancer, obesity, diabetes, coronary artery disease, heart valve disease, congenital heart disease, cardiomyopathies, pericarditis, or platelet disorders (e.g. essential thrombocytosis).
14. The method of paragraph 10, wherein the disease or disorder is caused by low serotonin levels and is selected from the group: constipation, IBS-C, depression, anxiety, addiction, neurodegenerative disorders, autism spectrum disorder, sleep disorders, attention deficit hyperactivity disorder (ADHD), memory loss (e.g. dementia), learning difficulties, osteoporosis, heartburn, dermatological conditions (eczema and itch), GERD, or pain disorders.
15. The method of paragraph 10, wherein treating a disease or disorder comprises decreasing at least one symptom of the disease or disorder, selected from: fatigue, insomnia, stress, persistent anxiety, persistent sadness, social withdrawal, substance withdrawal, irritability, thoughts of suicide, thoughts of self-harm, restlessness, low sex drive, lack of focus, loss of appetite, high blood pressure, low blood pressure, high heart rate, low heart rate, constipation, diarrhea, chronic pain, heartburn, fatigue, trouble breathing, stomach aches, nosebleeds, gum, stomach bleeding, headaches, weight gain, burning of the skin, altered inflammatory markers, neurodevelopmental deficits, and/or seizures.
16. The method of paragraph 10, wherein the at least one isolated serotonin-modulating bacteria belongs to the genera Acidaminococcus, Agathobacter, Alistipes, Anaerotruncus, Bacillus, Bacteroides, Bifidobacterium, Bilophila, Blautia, Butyricimonas, Clostridium, Collinsella, Coprococcus, Dialister, Dorea, Eisenbergiella, Enterococcus, Escherichia, Eubacterium, Faecalicatena, Flavonifractor, Flintibacter, Gemmiger, Gordonibacter, Hungatella, Intestinimonas, Lachnoclostridium, Lactobacillus, Lawsonibacter, Longibaculum, Mediterraneibacter, Oscillibacter, Parabacteroides, Parasutterella, Prevotella, Romboutsia, Ruminococcaceae, Ruminococcus, Sellimonas, Slackia, Streptococcus, Sutterella, Turicibacter, or Veillonella.
17. The method of paragraph 10, wherein the at least one isolated serotonin-modulating bacteria belongs to the species Acidaminococcus intestini, Agathobacter rectalis, Alistipes onderdonkii, Alistipes putredinis, Anaerotruncus colihominis, Bacillus cereus, Bacteroides vulgatus, Bacteroides plebeius, Bacteroides koreensis, Bacteroides cellulosilyticus, Bacteroides fragilis, Bacteroides xylanisolvens, Bacteroides uniformis, Bacteroides stercoris, Bacteroides dorei, Bacteroides caccae, Bacteroides thetaiotaomicron, Bacteroides salyersiae, Bacteroides ovatus, Bacteroides finegoldii, Bacteroides clarus, Bifidobacterium faecale, Bifidobacterium adolescentis, Bifidobacterium longum, Bifidobacterium brevis, Bilophila wadsworthia, Blautia producta, Blautia wexlerae, Blautia obeum, Butyricimonas paravirosa, Clostridium scindens, Clostridium inoculum, Clostridium bolteae, Clostridium aldenense, Clostridium saudiense, Clostridium lavalense, Clostridium amygdalium, Clostridium clostridioforme, Clostridium leptum, Clostridium tyrobutyricum, Clostridium ramosum, Clostridium paraputrificum, Clostridium sphenoides, Clostridium symbiosum, Clostridium sp., Clostridium butyricum, Collinsella aerofaciens, Coprococcus comes, Coprococcus eutactus, Coprococcus sp., Dialister invisus, Dorea longicatena, Eisenbergiella tayi, Eisenbergiella sp., Enterococcus lactis, Enterococcus hirae, Enterococcus durans, Escherichia coli, Eubacterium limosum, Eubacterium eligens, Eubacterium callanderi, Eubacterium rectale, Faecalicatena cylindroides, Flavonifractor plautii, Flintibacter sp., Gemmiger formicilis, Gemmiger sp., Genus Species, Gordonibacter pamelaeae, Hungatella hathewayi, Hungatella effluvii, Intestinimonas butyriciproducens, Intestinimonas massiliensis, Lachnoclostridium sp., Lactobacillus brevis, Lawsonibacter asaccharolyticus, Longibaculum muris, Longibaculum sp., Mediterraneibacter faecis, Oscillibacter rumenantium, Oscillibacter valericigenes, Parabacteroides distasonis, Parabacteroides johnsonii, Parabacteroides merdae, Parabacteroides goldsteinii, Parasutterella excrementihominis, Prevotella copri, Prevotella sp., Prevotella sp., Romboutsia lituseburensis, Ruminococcaceae sp., Ruminococcus gnavus, Ruminococcus bicirculans, Ruminococcus torques, Sellimonas intestinalis, Slackia isoflavoniconvertens, Streptococcus gordonii, Sutterella wadsworthensis, Turicibacter sanguinis, or Veillonella atypica.
18. The method of paragraph 10, wherein the one or more serotonin-modulating bacteria is/are selected from the strains Acidaminococcus intestini HB-95, Agathobacter rectalis HB-257, Alistipes onderdonkii HB-311, Alistipes putredinis HB-324, Anaerotruncus colihominis HB-474, Anaerotruncus colihominis HB-83, Bacillus cereus HB-25, Bacteroides caccae HB-11, Bacteroides cellulosilyticus HB-227, Bacteroides clarus HB-30, Bacteroides dorei HB-12, Bacteroides finegoldii HB-31, Bacteroides fragilis HB-58, Bacteroides koreensis HB-385, Bacteroides ovatus HB-70, Bacteroides plebeius HB-237, Bacteroides salyersiae HB-32, Bacteroides stercoris HB-33, Bacteroides thetaiotaomicron HB-34, Bacteroides uniformis HB-13, Bacteroides vulgatus HB-10, Bacteroides xylanisolvens HB-35, Bifidobacterium adolescentis HB-179, Bifidobacterium brevis HB-90, Bifidobacterium faecale HB-159, Bifidobacterium longum HB-234, Bifidobacterium longum HB-71, Bilophila wadsworthia HB-693, Blautia obeum HB-14, Blautia producta HB-23, Blautia wexlerae HB-16, Butyricimonas paravirosa HB-453, Clostridium aldenense HB-440, Clostridium amygdalium HB-152, Clostridium bolteae HB-442, Clostridium butyricum HB-88, Clostridium clostridioforme HB-642, Clostridium inoculum HB-82, Clostridium lavalense HB-452c, Clostridium leptum HB-73, Clostridium paraputrificum HB-27, Clostridium ramosum HB-24, Clostridium saudiense HB-142, Clostridium scindens HB-444, Clostridium sp. HB-358, Clostridium sphenoides HB-470, Clostridium symbiosum HB-67, Clostridium tyrobutyricum HB-469, Collinsella aerofaciens HB-04, Collinsella aerofaciens HB-274, Coprococcus comes HB-376, Coprococcus eutactus HB-155, Coprococcus sp. HB-80, Dialister invisus HB-387, Dorea longicatena HB-17, Eisenbergiella sp. HB-612, Eisenbergiella tayi HB-437, Enterococcus durans HB-85, Enterococcus hirae HB-48, Enterococcus lactis HB-640, Escherichia coli HB-490, Eubacterium callanderi HB-59, Eubacterium eligens HB-252, Eubacterium limosum HB-98, Eubacterium rectale HB-22, Faecalicatena cylindroides HB-664, Flavonifractor plautii HB-472, Flintibacter sp. HB-344, Gemmiger formicilis HB-325, Gemmiger sp. HB-567, Gordonibacter pamelaeae HB-15, Hungatella effluvii HB-02, Hungatella hathewayi HB-01, Intestinimonas butyriciproducens HB-478, Intestinimonas massiliensis HB-651, Lachnoclostridium sp. HB-698, Lactobacillus brevis HB-87, Lawsonibacter asaccharolyticus HB-521, Longibaculum muris HB-79, Longibaculum sp. HB-681, Mediterraneibacter faecis HB-364, Oscillibacter rumenantium HB-28, Oscillibacter valericigenes HB-45, Parabacteroides distasonis HB-20, Parabacteroides distasonis HB-214, Parabacteroides goldsteinii HB-44, Parabacteroides johnsonii HB-03, Parabacteroides merdae HB-63, Parasutterella excrementihominis HB-330, Prevotella copri HB-373, Prevotella sp. HB-333, Prevotella sp. HB-649, Romboutsia lituseburensis HB-102, Ruminococcaceae sp. HB-477, Ruminococcus bicirculans HB-105, Ruminococcus bicirculans HB-268, Ruminococcus gnavus HB-40, Ruminococcus gnavus HB-516, Ruminococcus torques HB-626, Sellimonas intestinalis HB-443, Slackia isoflavoniconvertens HB-326, Streptococcus gordonii HB-62, Sutterella wadsworthensis HB-259, Turicibacter sanguinis HB-147, and Veillonella atypica HB-251.
19. The method of paragraph 10, wherein the one or more serotonin-modulating bacteria consists of a bacteria comprising a 16S rDNA sequence at least about 95% identical to a 16S rDNA sequence selected from one of SEQ ID NOs: 1-105.
20. The method of paragraph 10, wherein the serotonin-modulating bacteria encode genes in their genome, which when expressed, result in the production of one or more metabolites or proteins that influence subject serotonin signaling/biosynthesis.
21. The method of paragraph 20, wherein the encoded genes are expressed at physiologically relevant conditions of the human gastrointestinal tract, resulting in the production of metabolites or proteins that influence subject serotonin signaling/biosynthesis.
22. The method of paragraph 10, wherein the composition is in the form of a probiotic, prebiotic, a capsule, a tablet, a caplet, a pill, a troche, a lozenge, a powders, a granule, a medical food, or a combination thereof.
23. The method of paragraph 10, wherein the composition is administered orally, intravenously, intramuscularly, intrathecally, subcutaneously, sublingually, buccally, rectally, vaginally, by the ocular route, by the otic route, nasally, via inhalation, by nebulization, cutaneously, transdermally, or a combination thereof

24. The method of paragraph 10, further comprising identifying a subject in need of treatment by measuring a serotonin level in a sample from the subject, and comparing the level to a reference level.
25. The method of paragraph 24, wherein the serotonin level is measured in stool, blood, or tissue of the subject.
26. The method of paragraph 24, wherein a serotonin level less than the reference level identifies a subject in need of treatment.
27. The method of paragraph 22, wherein the levels of serotonin in the stool, blood, or tissue of the subject are altered relative to their initial quantitated amounts, after administering the therapeutic composition.
28. The method of paragraph 10, further comprising identifying a subject in need of treatment by measuring levels of fecal serotonin modulating bacteria.
29. The method of paragraph 28, wherein the level of fecal serotonin-modulating bacteria is measured by fecal 16S rDNA sequencing, fecal shotgun metagenomic sequencing, measurement of fecal genes involved in the production of microbiota-derived serotonin modulating metabolites, measurement of proteins by sequencing or proteomics or comparable methods, or levels of fecal, blood, or tissue serotonin-modulating metabolites via LC/MS or comparable methods.
30. The method of paragraph 22, wherein the levels of serotonin modulating bacteria, genes involved in the production of microbiota-derived serotonin modulating metabolites or proteins, or levels of serotonin-modulating metabolites are altered relative to their initial quantitated amounts after administering the therapeutic composition.

### EXAMPLES

### Example 1: Identification of Serotonin-Modulating Bacteria

Culturing Bacteria from Human Stool

Bacteria from human stool samples (leveraging microbiological methods to enrich and select for unique diversity—e.g. plating stool samples on multiple bacterial mediums with a variety of carbon and nitrogen sources, including those found in the human gastrointestinal tract and/or listed within this specification, at different concentrations; plating stool sample on bacterial mediums with antibiotics or bile acids, to selectively kill certain bacterial populations; treating stool samples prior to plating with oxygen, antibiotics, or chloroform), were tested for the ability to influence serotonin release in the RIN14B serotonin release assay.

### RIN14B Serotonin Release Assay

To run the RIN14B serotonin release assay, RIN14B cells (ATCC® CRL-2059™) were first grown following manufacturer's protocols (ATCC). RIN14B cells were then seeded in 24-well plates at 10⁵/cm² in 500 μL RPMI complete culture medium (GIBCO), and incubated at 37° C. in the CO₂ incubator for 48 hours. At this point, the stimuli (e.g., bacterial supernatant or cell pellets) were then prepared under aseptic conditions.

To prepare the stimuli for the RIN14B serotonin release assay, isolated bacteria were grown in vegetable-based bacteriological mediums and/or bacteriological mediums with mammalian components. In some embodiments, the mediums included at least one of the following: conditioned medium from other bacteria, N-Acetyl-D-Galactosamine, N-Acetyl-D-Glucosamine, N-Acetyl-β-D-Mannosamine, Adonitol, Amygdalin, D-Arabitol, Arbutin, D-Cellobiose, α-Cyclodextrin, O-Cyclodextrin, Dextrin, Dulcitol, i-Erythritol, D-Fructose, L-Fucose, D-Galactose, D-Galacturonic Acid, Gentiobiose, D-Gluconic Acid, D-Glucosaminic Acid, α-D-Glucose, α-D-Glucose 1-Phosphate, D-Glucose6-Phosphate, Glycerol, D,L-α-Glycerol Phosphate, m-Inositol, α-D-Lactose, Lactulose, Maltose, Maltotriose, D-Mannitol, D-Mannose, D-Melezitose, D-Melibiose, 3-Methyl-DGlucose, α-Methyl-DGalactoside, β-Methyl-D-Galactoside, α-Methyl-D-Glucoside, β-Methyl-D-Glucoside, Mucin, Palatinose, D-Raffinose, L-Rhamnose, Salicin, D-Sorbitol, Stachyose, Sucrose, D-Trehalose, Turanose, Acetic Acid, Formic Acid, Fumaric Acid, Glyoxylic Acid, α-Hydroxybutyric Acid, β-Hydroxybutyric Acid, Itaconic Acid, α-Ketobutyric Acid, α-Ketovaleric Acid, D,L-Lactic Acid, L-Lactic Acid, D-Lactic Acid Methyl Ester, D-Malic Acid, L-Malic Acid, Propionic Acid, Pyruvic Acid, Pyruvic Acid Methyl Ester, D-Saccharic Acid, Succinamic Acid, Succinic Acid, Succinic Acid Mono-Methyl Ester, m-Tartaric Acid, Urocanic Acid, Alaninamide, L-Alanine, L-Alanyl-LGlutamine, L-Alanyl-LHistidine, L-Alanyl-LThreonine, L-Asparagine, L-Glutamic Acid, L-Glutamine, Glycyl-LAspartic Acid, Glycyl-LGlutamine, Glycyl-LMethionine, Glycyl-LProline, L-Methionine, L-Phenylalanine, L-Serine, L-Threonine, L-Valine, L-Valine plus L-Aspartic Acid, 2′-Deoxy Adenosine, Inosine, Thymidine, Uridine, Thymidine-5′-Mono-phosphate, or Uridine-5′-Monophosphate. In some embodiments of any of the aspects, the isolated bacteria are grown in HM2 media. HM2 comprises: Vegitone infused Broth (Sigma™; e.g., 37 g/L); Yeast Extract (Fisher™; e.g., 5 g/L); MOPS 1M Buffer (Teknova™; e.g., 50 mL/L); Cysteine hydrochloride (10% stock) (Sigma™; e.g., 10 mL/L); and Hemin+Vitamin K (Remel™; e.g., 1 mL/L).

Samples of the bacterial cultures were collected at timepoints of 24 and 48 hours. In some embodiments, the strains were growth at atmospheric oxygen, microaerophilic conditions, or anaerobic conditions, at temperatures ranging from 20 to 50 degrees Celsius. At this point cultures underwent centrifugation to separate bacterial cell pellets and supernatant. Supernatant was then sterilized via passage through a 0.1 m or 0.22 m filter. Bacterial pellets were washed with Hanks' Balanced Salt Solution (HBSS) (GIBCO) buffer, and protein concentration using the Pierce™ Rapid Gold BCA Protein Assay Kit, per manufacturer's protocols (THERMO SCIENTIFIC).

Supernatant or washed bacterial cell pellet was then introduced into the RIN14B cell cultures. RIN14B culture supernatant was aspirated without disturbing the monolayer, and then washed with 250 uL of washing solution (HBSS supplemented with 0.1% Bovine Serum Albumin (SIGMA) and 2 uM Fluoxetine hydrochloride (SIGMA)). The washing solution was then aspirated, and 250 uL/well of stimuli was added to the cell cultures (base diluent=HBSS). The stimulate included at least one of the following: (A) Cell pellets tested at 100 μg/mL and 20 μg/mL, (B) Supernatants tested at 50% and 10% dilutions, (C) Heat Inactivated stimuli that were incubated at 96° C. for 15 min in a heating block, (D) Negative controls (e.g., HBSS, 1% DMSO, fresh bacterial culture media), or (E) Positive control (e.g., 15 μM Ionomycin, prepared in DMSO). Stimuli were incubated for one hour at 37° C. in the CO₂ incubator, and RIN14B supernatants were collected by centrifuging assay plates at 6000×g for 5 min, and supernatants were stored at 4° C. until use. Serotonin concentration in the RIN14B supernatant was quantitated by ELISA (EAGLE BIOSCIENCES) according to manufacturer's instructions.

After testing over 100 strains, many organisms were identified that had significant impact on serotonin release compared to positive controls, as well as organisms that had no impact (see e.g., FIG. 1-FIG. 4, Tables 1A-1D). Notably, effects were found with both conditioned mediums and cell pellets from these organisms, indicating multiple mechanisms (e.g., at least one metabolic in origin mechanism, and at least one protein-driven mechanism). In some embodiments, bacteria and/or bacterial metabolites modulate serotonin through a Toll-like receptor (TLR)-mediated mechanism. Non-limiting examples of TLRs related to bacterial sensing include TLR1, TLR2, TLR4, TLR5, TLR6, and/or TLR9.

Without wishing to be bound by theory, identification of the specific metabolites and/or proteins driving the alterations in serotonin in this model (e.g., by performing genome analysis, metagenomics, metabolomics and/or proteomics on strong inducers of serotonin vs. bacteria with no impact) can indicate the specific mechanisms. Alternatively, standard bio-assay purification techniques can be employed (e.g., fractionating active conditioned mediums using LC/MS or equivalent methods, eventually identifying a fraction with purified compounds that could be identified via NMR), or genetic screens of serotonin-modulating organisms can be employed (e.g. creating a transposon library of the active strains, and then screening for an inactive close, thus identifying genes eliciting the serotonin-modulating effects). These mechanisms can then be leveraged to identify additional bacteria with these characteristics (e.g. using genome analysis) to then deliver into therapeutic compositions. Furthermore, these data can be leveraged with human stool transcriptomic cohorts to identify not just bacteria which have the capability to perform these functions, but those bacteria that actively do so with a human under physiologically relevant conditions. These organisms will likely have superiority over others with the same genetic potential. Likewise, organisms can be engineered to perform these functions and then be delivered to the host.

### Human Gut Simulator

The serotonin-modulating strains described herein can influence 5-HT signaling in the presence of a complex human gut microbiome. In a non-limiting example, several 5-HT modulating strains were introduced into a human gut simulator, and the cell pellets and supernatants of the entire community were then introduced to RIN14B cells at multiple time points. Here, by testing the supernatant and cell pellet of the entire mock community (with and without the 5-HT modulating bacteria added), it was found that several of the 5-HT modulating strains positively impacted 5-HT signaling (see e.g., FIG. 3, FIG. 4). Briefly, this was done by inoculating a diluted human fecal sample into a gut simulator vessel loaded with pre-reduced Gifu Anaerobic Medium (GAM), diluted at 1:10 strength. After allowing the human-derived community to normalize for 48 hours, the 5-HT modulating bacteria were spiked into the gut simulator. Samples were collected from the gut simulator 48 hours later, and the impact of the collective cell pellet and supernatant of the community was tested in 5-HT release using the RIN14B cell culture assay.

The change in 5-HT signal of this mock community could be directly due to the ability of the introduced bacteria to produce metabolites and/or proteins that cause the effect. Alternatively, this could be due to a shift in the native microbiome, to amore 5-HT modulating state (e.g., increasing levels of native 5-HT modulating bacteria). This can also be a combination of these effects (e.g., activity of the introduced 5-HT modulating bacteria, as well as a shift to a more potent 5-HT modulating microbial community). Without being bound by theory, the signal of introduced 5-HT modulating bacteria can also be amplified by the presence of native 5-HT modulating bacteria, either through provision of nutrients essential for the introduced 5-HT modulating bacteria for growth or engraftment, or precursors of 5-HT modulating pathways (e.g., tryptophan).

### Strains

TABLE 1A
5-HT Modulating Potential of 114 Human-Derived Strains - Pathways of 5-HT Modulation

| | | Pathway of 5-HT Modulation | | | | | |
|---|---|---|---|---|---|---|---|
| | | | | 5-HT | No or | | |
| | | | 5-HT | Positive | Low | | |
| SEQ ID | | 5-HT | Positive | Cell | 5-HT | 5-HT | Agonist |
| NO | Strain | Producer | Supernantant | Pellet | Impact | Reducer | Producer |
| 1 | Ruminococcus gnavus HB-40 | X | X | X | | | X |
| 2 | Ruminococcus gnavus HB-516 | X | X | X | | | X |
| 3 | Enterococcus durans HB-48 | X | | X | | | X |
| 4 | Clostridium lavalense HB-452c | X | | | | | X |
| 5 | Hungatella effluvii HB-02 | | X | X | | | |
| 6 | Bacteroides caccae HB-11 | | X | X | | | |
| 7 | Bacteroides dorei HB-12 | | X | X | | | |
| 8 | Clostridium saudiense HB-142 | | X | X | | | |
| 9 | Gordonibacter pamelaeae HB-15 | | X | X | | | |
| 10 | Clostridium hathewayi HB-152 | | X | X | | | |
| 11 | Bifidobacterium faecale HB-159 | | X | X | | | |
| 12 | Bifidobacterium adolescentis HB-179 | | X | X | | | |
| 13 | Parabacteroides distasonis HB-20 | | X | X | | | |
| 14 | Eubacterium eligens HB-252 | | X | X | | | |
| 15 | Bacteroides fiinegoldii HB-31 | | X | X | | | |
| 16 | Bacteroides salyersiae HB-32 | | X | X | | | |
| 17 | Gemmiger formicilis HB-325 | | X | X | | | |
| 18 | Parasutterella excrementihominis HB-330 | | X | X | | | |
| 19 | Bacteroides xylanisolvens HB-35 | | X | X | | | |
| 20 | Mediterraneibacter faecis HB-364 | | X | X | | | |
| 21 | Clostridium aldenese HB-440 | | X | X | | | |
| 22 | Clostridium bolteae HB-442 | | X | X | | | |
| 23 | Clostridium scindens HB-444 | | X | X | | | |
| 24 | Clostridium tyrobutyricum HB-469 | | X | X | | | |
| 25 | Eisenbergiella tayi HB-612 | | X | X | | | |
| 26 | Parabacteroides merdae HB-63 | | X | X | | | |
| 27 | Bacteroides ovatus HB-70 | | X | X | | | |
| 28 | Anaerotruncus colihominis HB-83 | | X | X | | | |
| 29 | Enterococcus faecium HB-85 | | X | X | | | |
| 30 | Lactobacillus brevis HB-87 | | X | X | | | |
| 31 | Hungatella hathewayi HB-01 | | X | | | | |
| 32 | Parabacteroides johnsonii HB-03 | | X | | | | |
| 33 | Bacteroides vulgatus HB-10 | | X | | | | |
| 34 | Bacteroides uniformis HB-13 | | X | | | | |
| 35 | Turicibacter sanguinis HB-147 | | X | | | | |
| 36 | Coprococcus eutactus HB-155 | | X | | | | |
| 37 | Bacteroides cellulosilyticus HB-227 | | X | | | | |
| 38 | Bacteroides plebeius HB-237 | | X | | | | |
| 39 | Erysipelatoclostridium ramosum HB-24 | | X | | | | |
| 40 | Bacillus cereus HB-25 | | X | | | | |
| 41 | Agathobacter rectalis HB-257 | | X | | | | |
| 42 | Ruminococcus bicirculans HB-268 | | X | | | | |
| 43 | Clostridium paraputrificum HB-27 | | X | | | | |
| 44 | Collinsella aerofaciens HB-274 | | X | | | | |
| 45 | Oscillibacter sp. HB-28 | | X | | | | |
| 46 | Alistipes onderdonkii HB-311 | | X | | | | |
| 47 | Alistipes putredinis HB-324 | | X | | | | |
| 48 | Bacteroides stercoris HB-33 | | X | | | | |
| 49 | Flintibacter butyricus HB-344 | | X | | | | |
| 50 | Coprococcus comes HB-376 | | X | | | | |
| 51 | Bacteroides koreensis HB-385 | | X | | | | |
| 52 | Eisenbergiella tayi HB-437 | | X | | | | |
| 53 | Dysosmobacter welbionis HB-45 | | X | | | | |
| 54 | Butyricimonas paravirosa HB-453 | | X | | | | |
| 55 | Flavonifractor plautii HB-472 | | X | | | | |
| 56 | Anaerotruncus colihominis HB-474 | | X | | | | |
| 57 | Intestinimonas butyriciproducens HB-478 | | X | | | | |
| 58 | Bacteroides fragilis HB-58 | | X | | | | |
| 59 | Streptococcus gordonii HB-62 | | X | | | | |
| 60 | Enterococcus faecium HB-640 | | X | | | | |
| 61 | Clostridium clostridioforme HB-642 | | X | | | | |
| 62 | Faecalitalea cylindroides HB-664 | | X | | | | |
| 63 | Bilophila wadsworthia HB-693 | | X | | | | |
| 64 | Bifidobacterium longum HB-71 | | X | | | | |
| 65 | Clostridium hylemonae HB-73 | | X | | | | |
| 66 | Clostridium innoculum HB-82 | | X | | | | |
| 67 | Bifidobacterium breve HB-90 | | X | | | | |
| 68 | Acidaminococcus intestini HB-95 | | X | | | | |
| 69 | Streptococcus gordonii HB-98 | | X | | | | |
| 70 | Romboutsia lituseburensis HB-102 | | | X | | | |
| 71 | Blautia wexlerae HB-16 | | | X | | | |
| 72 | Sutterella wadsworthensis HB-259 | | | X | | | |
| 73 | Bacteroides clarus HB-30 | | | X | | | |
| 74 | Prevotella sp. HB-333 | | | X | | | |
| 75 | Bacteroides thetaiotaomicron HB-34 | | | X | | | |
| 76 | Prevotella copri HB-373 | | | X | | | |
| 77 | Dialister invisus HB-387 | | | X | | | |
| 78 | Sellimonas intestinalis HB-443 | | | X | | | |
| 79 | Bittarella massiliensis HB-477 | | | X | | | |
| 80 | Ruminococcus sp.HB-626 | | | X | | | |
| 81 | Prevotella sp HB-649 | | | X | | | |
| 82 | Longibaculum muris HB-79 | | | X | | | |
| 83 | Ruminococcus bicirculans HB-105 | | | | X | | |
| 84 | Parabacteroides distasonis HB-214 | | | | X | | |
| 85 | Veillonella atypica HB-251 | | | | X | | |
| 86 | Clostridium sp. HB-358 | | | | X | | |
| 87 | Parabacteroides goldsteinii HB-44 | | | | X | | |
| 88 | Clostridium sphenoides HB-470 | | | | X | | |
| 89 | Escherichia coli HB-490 | | | | X | | |
| 90 | Lawsonibacter asaccharolyticus HB-521 | | | | X | | |
| 91 | Gemmiger sp. HB-567 | | | | X | | |
| 92 | Eubacterium callanderi HB-59 | | | | X | | |
| 93 | Intestinimonas massiliensis HB-651 | | | | X | | |
| 94 | Clostridium symbiosum HB-67 | | | | X | | |
| 95 | Longibaculum sp. HB-681 | | | | X | | |
| 96 | Blautia obeum HB-14 | | | | | X | |
| 97 | Dorea longicatena HB-17 | | | | | X | |
| 98 | Eubacterium rectale HB-22 | | | | | X | |
| 99 | Blautia coccoides HB-23 | | | | | X | |
| 100 | Bifidobacterium longum HB-234 | | | | | X | |
| 101 | Slackia isofilavoniconvertens HB-326 | | | | | X | |
| 102 | Lachnoclostridium sp. HB-698 | | | | | X | |
| 103 | Coprococcus comes HB-80 | | | | | X | |
| 104 | Clostridium butyricum HB-88 | | | | | X | |
| 105 | Clostridium sporogenes JCM 7836 | | | | | | X |
| 106 | Akkermansia muciniphila BAA-835 | | | | | | X |
| 107 | Clostridium sporogenes McClung 2004 | | | | | | X |
| 108 | Peptostreptococcus russellii RT-10B | | | | | | X |
| 109 | Mycolicibacterium smegmatis ATCC 19420 | | | | | | X |
| 110 | Enterorhabdus muris WCA-131-CoC-2 | | | | | | X |
| 111 | Adlercreutzia equolifaciens FJC-B9 | | | | | | X |
| 112 | Enterorhabdus caecimuris B7 | | | | | | X |
| 113 | Coprococcus eutactus ATCC 27759 | | | | | | X |
| 114 | Coprococcus comes ATCC 27758 | | | | | | X |

TABLE 1B
5-HT Modulating Potential of 104 Human-Derived Strains - Serotonin Release in
ng/mL (HM2 media) Measured by ELISA (bolded number indicates a significant amount
of serotonin release over pre-determined threshold; see e.g., final row)

| | | Serotonin Release in ng/mL (HM2) | | | |
|---|---|---|---|---|---|
| | | Measured by ELISA | | | |
| SEQ ID | | RIN14B - Supernatant | | RIN14B - Cell Pellet | |
| NO | Strain | 50% | 10% | 100 ug/mL | 20 ug/mL |
| 1 | Ruminococcus gnavus HB-40 | 523.2 | — | 53.7 | 23.6 |
| 2 | Ruminococcus gnavus HB-516 | 378.0 | 151.9 | 46.2 | 56.8 |
| 3 | Enterococcus durans HB-48 | 86.8 | 49.9 | 12.4 | 49.3 |
| 4 | Clostridium lavalense HB-452c | 88.5 | 37.2 | 29.5 | 29.0 |
| 5 | Hungatella effluvii HB-02 | 57.9 | 51.2 | 46.7 | 66.5 |
| 6 | Bacteroides caccae HB-11 | 59.0 | 20.7 | 44.1 | 13.2 |
| 7 | Bacteroides dorei HB-12 | 64.9 | 17.0 | 45.4 | 10.3 |
| 8 | Clostridium saudiense HB-142 | 71.2 | 55.0 | 63.3 | 47.5 |
| 9 | Gordonibacter pamelaeae HB-15 | 42.7 | 53.7 | 43.0 | 13.3 |
| 10 | Clostridium hathewayi HB-152 | 62.5 | 63.1 | 62.7 | 40.7 |
| 11 | Bifidobacterium faecale HB-159 | 92.7 | 68.7 | 54.4 | 37.1 |
| 12 | Bifidobacterium adolescentis HB-179 | 23.7 | 46.3 | 27.2 | 46.8 |
| 13 | Parabacteroides distasonis HB-20 | 39.9 | 48.1 | 61.8 | 30.6 |
| 14 | Eubacterium eligens HB-252 | 55.1 | 12.3 | 59.8 | 21.9 |
| 15 | Bacteroides finegoldii HB-31 | 54.8 | 11.8 | 51.9 | 7.3 |
| 16 | Bacteroides salyersiae HB-32 | 27.4 | 48.7 | 43.2 | 45.5 |
| 17 | Gemmiger formicilis HB-325 | 67.0 | 13.2 | 57.8 | 22.4 |
| 18 | Parasutterella excrementihominis HB-330 | 64.7 | 11.2 | 61.5 | 38.9 |
| 19 | Bacteroides xylanisolvens HB-35 | 72.3 | 19.7 | 44.6 | 8.2 |
| 20 | Mediterraneibacter faecis HB-364 | 76.7 | 8.1 | 61.0 | 38.4 |
| 21 | Clostridium aldenese HB-440 | 78.0 | 57.4 | 76.3 | 48.7 |
| 22 | Clostridium bolteae HB-442 | 90.7 | 59.2 | 66.8 | 57.9 |
| 23 | Clostridium scindens HB-444 | 84.8 | 71.2 | 71.8 | 50.4 |
| 24 | Clostridium tyrobutyricum HB-469 | 56.2 | 26.1 | 40.2 | 8.6 |
| 25 | Eisenbergiella tayi HB-612 | 36.0 | 42.2 | 45.7 | 28.4 |
| 26 | Parabacteroides merdae HB-63 | 16.8 | 42.9 | 50.4 | 1.4 |
| 27 | Bacteroides ovatus HB-70 | 66.6 | 7.8 | 46.5 | 12.0 |
| 28 | Anaerotruncus colihominis HB-83 | 44.2 | 62.6 | 52.5 | 72.9 |
| 29 | Enterococcus faecium HB-85 | 72.5 | 37.9 | 5.3 | 49.1 |
| 30 | Lactobacillus brevis HB-87 | 95.5 | 39.0 | 50.7 | 16.1 |
| 31 | Hungatella hathewayi HB-01 | 132.7 | 37.0 | 13.4 | 11.2 |
| 32 | Parabacteroides johnsonii HB-03 | 25.6 | 43.4 | 22.4 | 13.2 |
| 33 | Bacteroides vulgatus HB-10 | 33.3 | 152.9 | 23.1 | 24.5 |
| 34 | Bacteroides uniformis HB-13 | 40.2 | 49.7 | 15.6 | 28.8 |
| 35 | Turicibacter sanguinis HB-147 | 39.9 | 58.8 | 9.2 | 1.1 |
| 36 | Coprococcus eutactus HB-155 | 59.4 | 8.9 | 32.7 | 11.7 |
| 37 | Bacteroides cellulosilyticus HB-227 | 61.8 | 57.1 | 20.8 | 27.4 |
| 38 | Bacteroides plebeius HB-237 | 79.4 | 51.6 | 35.5 | 39.4 |
| 39 | Erysipelatoclostridium ramosum HB-24 | 50.8 | 22.3 | 31.9 | 38.0 |
| 40 | Bacillus cereus HB-25 | 24.4 | 100.3 | 0.7 | 1.0 |
| 41 | Agathobacter rectalis HB-257 | 35.1 | 44.4 | 1.0 | 19.1 |
| 42 | Ruminococcus bicirculans HB-268 | 124.1 | 26.6 | 9.7 | 11.7 |
| 43 | Clostridium paraputrificum HB-27 | 22.5 | 42.9 | 4.2 | 23.7 |
| 44 | Collinsella aerofaciens HB-274 | 68.6 | 13.7 | 22.0 | 4.7 |
| 45 | Oscillibacter sp. HB-28 | 107.6 | 23.5 | 27.1 | 6.8 |
| 46 | Alistipes onderdonkii HB-311 | 59.6 | 78.5 | 31.6 | 34.9 |
| 47 | Alistipes putredinis HB-324 | 47.4 | 66.2 | 14.9 | 24.6 |
| 48 | Bacteroides stercoris HB-33 | 32.4 | 53.3 | 27.3 | 39.3 |
| 49 | Flintibacter butyricus HB-344 | 73.8 | 14.6 | 34.9 | 8.0 |
| 50 | Coprococcus comes HB-376 | 70.8 | 21.6 | 31.7 | 10.9 |
| 51 | Bacteroides koreensis HB-385 | 70.3 | 52.6 | 29.5 | 30.5 |
| 52 | Eisenbergiella tayi HB-437 | 41.1 | 54.1 | 2.4 | 14.7 |
| 53 | Dysosmobacter welbionis HB-45 | 82.0 | 35.5 | 25.2 | 11.0 |
| 54 | Butyricimonas paravirosa HB-453 | 54.4 | 32.7 | 23.2 | 29.0 |
| 55 | Flavonifractor plautii HB-472 | 53.6 | 34.4 | 17.4 | 27.5 |
| 56 | Anaerotruncus colihominis HB-474 | 42.2 | 44.8 | 1.0 | 5.9 |
| 57 | Intestinimonas butyriciproducens HB-478 | 22.0 | 42.7 | 8.4 | 38.7 |
| 58 | Bacteroides fragilis HB-58 | 48.2 | 47.0 | 17.7 | 26.5 |
| 59 | Streptococcus gordonii HB-62 | 284.4 | 23.3 | 22.1 | 4.2 |
| 60 | Enterococcus faecium HB-640 | 106.2 | 44.0 | 37.7 | 17.7 |
| 61 | Clostridium clostridioforme HB-642 | 52.8 | 56.8 | 5.7 | 9.3 |
| 62 | Faecalitalea cylindroides HB-664 | 42.3 | 47.9 | 11.3 | 20.1 |
| 63 | Bilophila wadsworthia HB-693 | 34.1 | 40.6 | 36.1 | 25.1 |
| 64 | Bifidobacterium longum HB-71 | 3.5 | 51.9 | 8.5 | 4.9 |
| 65 | Clostridium hylemonae HB-73 | 36.0 | 69.9 | 4.7 | 12.1 |
| 66 | Clostridium innoculum HB-82 | 42.2 | 109.9 | 1.0 | 7.7 |
| 67 | Bifidobacterium breve HB-90 | 3.9 | 44.1 | 1.0 | 1.0 |
| 68 | Acidaminococcus intestini HB-95 | 15.7 | 154.4 | 1.0 | 10.4 |
| 69 | Streptococcus gordonii HB-98 | 142.7 | 48.1 | 17.1 | 17.6 |
| 70 | Romboutsia lituseburensis HB-102 | 6.7 | 14.7 | 20.0 | 47.7 |
| 71 | Blautia wexlerae HB-16 | 19.9 | 9.2 | 20.2 | 45.2 |
| 72 | Sutterella wadsworthensis HB-259 | 2.3 | 9.8 | 5.0 | 50.7 |
| 73 | Bacteroides clarus HB-30 | 41.6 | 21.8 | 51.5 | 10.7 |
| 74 | Prevotella sp. HB-333 | 13.6 | 21.8 | 22.4 | 46.3 |
| 75 | Bacteroides thetaiotaomicron HB-34 | 45.8 | 33.6 | 30.8 | 48.0 |
| 76 | Prevotella copri HB-3 73 | 41.5 | 34.1 | 21.3 | 79.1 |
| 77 | Dialister invisus HB-387 | 39.2 | 37.5 | 56.1 | 49.9 |
| 78 | Sellimonas intestinalis HB-443 | 22.5 | 13.6 | 15.1 | 41.7 |
| 79 | Bittarella massiliensis HB-477 | 21.6 | 29.8 | 12.3 | 41.4 |
| 80 | Ruminococcus sp. HB-626 | 18.9 | 38.1 | 22.8 | 59.5 |
| 81 | Prevotella sp HB-649 | 10.9 | 1.0 | 9.7 | 44.5 |
| 82 | Longibaculum muris HB-79 | 49.3 | 37.6 | 45.7 | 16.4 |
| 83 | Ruminococcus bicirculans HB-105 | 45.7 | 30.8 | 28.1 | 19.8 |
| 84 | Parabacteroides distasonis HB-214 | 43.2 | 34.6 | 9.8 | 35.1 |
| 85 | Veillonella atypica HB-251 | 25.0 | 28.5 | 3.4 | 24.3 |
| 86 | Clostridium sp. HB-358 | 44.7 | 3.5 | 33.6 | 12.7 |
| 87 | Parabacteroides goldsteinii HB-44 | 31.3 | 27.7 | 6.9 | 19.9 |
| 88 | Clostridium sphenoides HB-470 | 36.5 | 25.8 | 39.9 | 33.0 |
| 89 | Escherichia coli HB-490 | 35.8 | 23.4 | 28.3 | 10.9 |
| 90 | Lawsonibacter asaccharolyticus HB-521 | 33.3 | 23.2 | 20.5 | 1.0 |
| 91 | Gemmiger sp. HB-567 | 36.9 | 27.4 | 24.0 | 1.0 |
| 92 | Eubacterium callanderi HB-59 | 36.1 | 27.4 | 25.7 | 27.4 |
| 93 | Intestinimonas massiliensis HB-651 | 29.2 | 21.7 | 1.0 | 38.0 |
| 94 | Clostridium symbiosum HB-67 | 32.5 | 26.4 | 1.9 | 30.1 |
| 95 | Longibaculum sp. HB-681 | 36.1 | 30.1 | 6.7 | 20.1 |
| 96 | Blautia obeum HB-14 | 9.0 | 6.4 | 6.6 | 30.4 |
| 97 | Dorea longicatena HB-17 | 22.8 | 3.5 | 5.8 | 18.8 |
| 98 | Eubacterium rectale HB-22 | 22.7 | 14.6 | 6.4 | 22.3 |
| 99 | Blautia coccoides HB-23 | 16.2 | 31.7 | 1.0 | 9.1 |
| 100 | Bifidobacterium longum HB-234 | 23.1 | 36.3 | 1.0 | 13.2 |
| 101 | Slackia isoflavoniconvertens HB-326 | 12.5 | 6.4 | 6.3 | 36.8 |
| 102 | Lachnoclostridium sp. HB-698 | 16.8 | 25.3 | 2.3 | 27.2 |
| 103 | Coprococcus comes HB-80 | 15.7 | 5.3 | 4.9 | 11.4 |
| 104 | Clostridium butyricum HB-88 | 25.7 | 18.4 | 1.0 | 9.3 |
| | Pre-determined threshold | >50.0 | >40.0 | >40.0 | >40.0 |

TABLE 1C
5-HT Modulating Potential of 104 Human-Derived Strains - Serotonin Release in
ng/mL (HM2) Measured by LC/MS (bolded number indicates a significant amount
of serotonin release over pre-determined threshold; see e.g., final row).

| | | Serotonin Release in ng/mL (HM2) | | | |
|---|---|---|---|---|---|
| | | Measured by LC/MS | | | |
| SEQ ID | | RIN14B - Supernatant | | Bacterial supernatant | |
| NO | Strain | 50% | 10% | 50% | 10% |
| 1 | Ruminococcus gnavus HB-40 | 331.3 | — | 380.6 | 89.5 |
| 2 | Ruminococcus gnavus HB-516 | 225.6 | 48.8 | 123.2 | 66.6 |
| 3 | Enterococcus durans HB-48 | 15.6 | 8.5 | 11.0 | 4.5 |
| 4 | Clostridium lavalense HB-452c | 22.0 | 7.8 | 10.6 | 4.2 |
| 5 | Hungatella effluvii HB-02 | — | — | — | — |
| 6 | Bacteroides caccae HB-11 | 7.3 | 4.1 | 0.3 | 1.1 |
| 7 | Bacteroides dorei HB-12 | 13.6 | 5.0 | 0.5 | 0.5 |
| 8 | Clostridium saudiense HB-142 | 5.4 | 3.0 | 0.3 | 0.5 |
| 9 | Gordonibacter pamelaeae HB-15 | 5.7 | 8.8 | 0.9 | 0.0 |
| 10 | Clostridium hathewayi HB-152 | — | — | — | — |
| 11 | Bifidobacterium faecale HB-159 | 9.1 | 5.0 | 1.0 | 0.5 |
| 12 | Bifidobacterium adolescentis HB-179 | — | — | — | — |
| 13 | Parabacteroides distasonis HB-20 | — | — | — | — |
| 14 | Eubacterium eligens HB-252 | 9.6 | 3.8 | 0.4 | 0.7 |
| 15 | Bacteroides finegoldii HB-31 | 9.1 | 3.8 | 0.9 | 0.8 |
| 16 | Bacteroides salyersiae HB-32 | 7.9 | 11.0 | 1.4 | 0.2 |
| 17 | Gemmiger formicilis HB-325 | — | — | — | — |
| 18 | Parasutterella excrementihominis HB-330 | — | — | — | — |
| 19 | Bacteroides xylanisolvens HB-35 | 9.1 | 4.6 | 0.5 | 0.0 |
| 20 | Mediterraneibacter faecis HB-364 | — | — | — | — |
| 21 | Clostridium aldenese HB-440 | — | — | — | — |
| 22 | Clostridium bolteae HB-442 | — | — | — | — |
| 23 | Clostridium scindens HB-444 | 9.9 | 9.9 | 0.5 | 0.7 |
| 24 | Clostridium tyrobutyricum HB-469 | 6.0 | 5.8 | 0.2 | 0.9 |
| 25 | Eisenbergiella tayi HB-612 | — | — | — | — |
| 26 | Parabacteroides merdae HB-63 | 13.8 | 7.7 | 1.1 | 0.1 |
| 27 | Bacteroides ovatus HB-70 | — | — | — | — |
| 28 | Anaerotruncus colihominis HB-83 | 8.4 | 11.1 | 0.6 | 0.0 |
| 29 | Enterococcus faecium HB-85 | — | — | — | — |
| 30 | Lactobacillus brevis HB-87 | 4.8 | 9.0 | 1.1 | 0.5 |
| 31 | Hungatella hathewayi HB-01 | — | — | — | — |
| 32 | Parabacteroides johnsonii HB-03 | — | — | — | — |
| 33 | Bacteroides vulgatus HB-10 | — | — | — | — |
| 34 | Bacteroides uniformis HB-13 | — | — | — | — |
| 35 | Turicibacter sanguinis HB-147 | 15.4 | 9.3 | 0.4 | 0.1 |
| 36 | Coprococcus eutactus HB-155 | 10.2 | 5.0 | 0.3 | 0.6 |
| 37 | Bacteroides cellulosilyticus HB-227 | — | — | — | — |
| 38 | Bacteroides plebeius HB-237 | — | — | — | — |
| 39 | Erysipelatoclostridium ramosum HB-24 | 27.8 | 5.5 | 0.4 | 0.0 |
| 40 | Bacillus cereus HB-25 | — | — | — | — |
| 41 | Agathobacter rectalis HB-257 | — | — | — | — |
| 42 | Ruminococcus bicirculans HB-268 | — | — | — | — |
| 43 | Clostridium paraputrificum HB-27 | — | — | — | — |
| 44 | Collinsella aerofaciens HB-274 | 8.4 | 6.3 | 0.5 | 0.7 |
| 45 | Oscillibacter sp. HB-28 | — | — | — | — |
| 46 | Alistipes onderdonkii HB-311 | 3.5 | 7.1 | 0.3 | 0.9 |
| 47 | Alistipes putredinis HB-324 | — | — | — | — |
| 48 | Bacteroides stercoris HB-33 | — | — | — | — |
| 49 | Flintibacter butyricus HB-344 | — | — | — | — |
| 50 | Coprococcus comes HB-376 | 7.0 | 9.1 | 0.6 | 0.8 |
| 51 | Bacteroides koreensis HB-385 | — | — | — | — |
| 52 | Eisenbergiella tayi HB-437 | — | — | — | — |
| 53 | Dysosmobacter welbionis HB-45 | — | — | — | — |
| 54 | Butyricimonas paravirosa HB-453 | 21.3 | 11 | 0.5 | — |
| 55 | Flavonifractor plautii HB-472 | — | — | — | — |
| 56 | Anaerotruncus colihominis HB-474 | — | — | — | — |
| 57 | Intestinimonas butyriciproducens HB-478 | — | — | — | — |
| 58 | Bacteroides fragilis HB-58 | — | — | — | — |
| 59 | Streptococcus gordonii HB-62 | — | — | — | — |
| 60 | Enterococcus faecium HB-640 | — | — | — | — |
| 61 | Clostridium clostridioforme HB-642 | — | — | — | — |
| 62 | Faecalitalea cylindroides HB-664 | 4.2 | 7.8 | 0.3 | 0.6 |
| 63 | Bilophila wadsworthia HB-693 | — | — | — | — |
| 64 | Bifidobacterium longum HB-71 | — | — | — | — |
| 65 | Clostridium hylemonae HB-73 | — | — | — | — |
| 66 | Clostridium innoculum HB-82 | — | — | — | — |
| 67 | Bifidobacterium breve HB-90 | — | — | — | — |
| 68 | Acidaminococcus intestini HB-95 | — | — | — | — |
| 69 | Streptococcus gordonii HB-98 | 5.7 | 10.8 | 0.6 | 0.7 |
| 70 | Romboutsia lituseburensis HB-102 | — | — | — | — |
| 71 | Blautia wexlerae HB-16 | — | — | — | — |
| 72 | Sutterella wadsworthensis HB-259 | — | — | — | — |
| 73 | Bacteroides clarus HB-30 | 6.6 | 4.1 | 0.2 | 0.8 |
| 74 | Prevotella sp. HB-333 | — | — | — | — |
| 75 | Bacteroides thetaiotaomicron HB-34 | — | — | — | — |
| 76 | Prevotella copri HB-373 | 10.1 | 2.9 | 0.9 | 0.8 |
| 77 | Dialister invisus HB-387 | — | — | — | — |
| 78 | Sellimonas intestinalis HB-443 | — | — | — | — |
| 79 | Bittarella massiliensis HB-477 | — | — | — | — |
| 80 | Ruminococcus sp. HB-626 | 4.7 | 7.1 | 0.8 | 0.0 |
| 81 | Prevotella sp HB-649 | — | — | — | — |
| 82 | Longibaculum muris HB-79 | — | — | — | — |
| 83 | Ruminococcus bicirculans HB-105 | — | — | — | — |
| 84 | Parabacteroides distasonis HB-214 | — | — | — | — |
| 85 | Veillonella atypica HB-251 | — | — | — | — |
| 86 | Clostridium sp. HB-358 | — | — | — | — |
| 87 | Parabacteroides goldsteinii HB-44 | — | — | — | — |
| 88 | Clostridium sphenoides HB-470 | — | — | — | — |
| 89 | Escherichia coli HB-490 | — | — | — | — |
| 90 | Lawsonibacter asaccharolyticus HB-521 | — | — | — | — |
| 91 | Gemmiger sp. HB-567 | — | — | — | — |
| 92 | Eubacterium callanderi HB-59 | — | — | — | — |
| 93 | Intestinimonas massiliensis HB-651 | — | — | — | — |
| 94 | Clostridium symbiosum HB-67 | — | — | — | — |
| 95 | Longibaculum sp. HB-681 | — | — | — | — |
| 96 | Blautia obeum HB-14 | — | — | — | — |
| 97 | Dorea longicatena HB-17 | — | — | — | — |
| 98 | Eubacterium rectale HB-22 | — | — | — | — |
| 99 | Blautia coccoides HB-23 | — | — | — | — |
| 100 | Bifidobacterium longum HB-234 | — | — | — | — |
| 101 | Slackia isoflavoniconvertens HB-326 | — | — | — | — |
| 102 | Lachnoclostridium sp. HB-698 | — | — | — | — |
| 103 | Coprococcus comes HB-80 | — | — | — | — |
| 104 | Clostridium butyricum HB-88 | — | — | — | — |
| | Pre-determined threshold | >9.1 | >7.0 | >2.0 | >2.0 |

TABLE 1D
5-HT Modulating Potential of 104 Human-Derived Strains - Serotonin Release in ng/mL
(BHI medium) Measured by LC/MS; and Tryptamine in 50% Bacterial Supernatant (ng/mL)
Measured by LC/MS (bolded number indicates a significant amount of serotonin or
tryptamine release over pre-determined threshold; see e.g., final row).

| | | Serotonin Release in | | | |
|---|---|---|---|---|---|
| | | ng/mL (BHI medium) | | Tryptamine in 50% | |
| | | Measured by LC/MS | | Bacterial Supernatant | |
| | | | Bacterial | (ng/mL) | |
| SEQ ID | | RIN-14B | Supernatant | Measured by LC/MS | |
| NO | Strain | 50% | 50% | HM2 | BHI |
| 1 | Ruminococcus gnavus HB-40 | 136.6 | 131.6 | 36529.2 | 20667.9 |
| 2 | Ruminococcus gnavus HB-516 | — | — | 21308.7 | — |
| 3 | Enterococcus durans HB-48 | 17.6 | 9 | 756.2 | 733.8 |
| 4 | Clostridium lavalense HB-452c | 15.4 | 6.6 | 729 | 488.7 |
| 5 | Hungatella effluvii HB-02 | — | — | — | — |
| 6 | Bacteroides caccae HB-11 | — | — | 45.3 | — |
| 7 | Bacteroides dorei HB-12 | 6.7 | 4.1 | 48.1 | 22.6 |
| 8 | Clostridium saudiense HB-142 | — | — | 40.9 | — |
| 9 | Gordonibacter pamelaeae HB-15 | — | — | 58.5 | — |
| 10 | Clostridium hathewayi HB-152 | — | — | — | — |
| 11 | Bifidobacterium faecale HB-159 | 10.1 | 2.4 | 47.3 | 21.5 |
| 12 | Bifidobacterium adolescentis HB-179 | — | — | — | — |
| 13 | Parabacteroides distasonis HB-20 | — | — | — | — |
| 14 | Eubacterium eligens HB-252 | — | — | 52.7 | — |
| 15 | Bacteroides finegoldii HB-31 | — | — | 47.4 | — |
| 16 | Bacteroides salyersiae HB-32 | — | — | 40.2 | — |
| 17 | Gemmiger formicilis HB-325 | — | — | — | — |
| 18 | Parasutterella excrementihominis HB-330 | — | — | — | — |
| 19 | Bacteroides xylanisolvens HB-35 | — | — | 38.3 | — |
| 20 | Mediterraneibacter faecis HB-364 | — | — | — | — |
| 21 | Clostridium aldenese HB-440 | — | — | — | — |
| 22 | Clostridium bolteae HB-442 | — | — | — | — |
| 23 | Clostridium scindens HB-444 | 10.1 | 2.3 | 60.7 | 22.6 |
| 24 | Clostridium tyrobutyricum HB-469 | — | — | 51.9 | — |
| 25 | Eisenbergiella tayi HB-612 | — | — | — | — |
| 26 | Parabacteroides merdae HB-63 | — | — | 41.5 | — |
| 27 | Bacteroides ovatus HB-70 | — | — | — | — |
| 28 | Anaerotruncus colihominis HB-83 | — | — | 48.4 | — |
| 29 | Enterococcus faecium HB-85 | — | — | — | — |
| 30 | Lactobacillus brevis HB-87 | 11.5 | 3.3 | 60.1 | 23.4 |
| 31 | Hungatella hathewayi HB-01 | — | — | — | — |
| 32 | Parabacteroides johnsonii HB-03 | — | — | — | — |
| 33 | Bacteroides vulgatus HB-10 | — | — | — | — |
| 34 | Bacteroides uniformis HB-13 | — | — | — | — |
| 35 | Turicibacter sanguinis HB-147 | 5.6 | 3.2 | 45.2 | 24.2 |
| 36 | Coprococcus eutactus HB-155 | 8.3 | 2.1 | 46.5 | 22.9 |
| 37 | Bacteroides cellulosilyticus HB-227 | — | — | — | — |
| 38 | Bacteroides plebeius HB-237 | — | — | — | — |
| 39 | Erysipelatoclostridium ramosum HB-24 | 10.5 | 3.1 | 50.5 | 21.5 |
| 40 | Bacillus cereus HB-25 | — | — | — | — |
| 41 | Agathobacter rectalis HB-257 | — | — | — | — |
| 42 | Ruminococcus bicirculans HB-268 | — | — | — | — |
| 43 | Clostridium paraputrificum HB-27 | — | — | — | — |
| 44 | Collinsella aerofaciens HB-274 | — | — | 43.6 | — |
| 45 | Oscillibacter sp. HB-28 | — | — | — | — |
| 46 | Alistipes onderdonkii HB-311 | — | — | 52.2 | — |
| 47 | Alistipes putredinis HB-324 | — | — | — | — |
| 48 | Bacteroides stercoris HB-33 | — | — | — | — |
| 49 | Flintibacter butyricus HB-344 | — | — | — | — |
| 50 | Coprococcus comes HB-376 | — | — | 50.6 | — |
| 51 | Bacteroides koreensis HB-385 | — | — | — | — |
| 52 | Eisenbergiella tayi HB-437 | — | — | — | — |
| 53 | Dysosmobacter welbionis HB-45 | — | — | — | — |
| 54 | Butyricimonas paravirosa HB-453 | 14.8 | 3.2 | 51.7 | 22.3 |
| 55 | Flavonifractor plautii HB-472 | — | — | — | — |
| 56 | Anaerotruncus colihominis HB-474 | — | — | — | — |
| 57 | Intestinimonas butyriciproducens HB-478 | — | — | — | — |
| 58 | Bacteroides fragilis HB-58 | — | — | — | — |
| 59 | Streptococcus gordonii HB-62 | — | — | — | — |
| 60 | Enterococcus faecium HB-640 | — | — | — | — |
| 61 | Clostridium clostridioforme HB-642 | — | — | — | — |
| 62 | Faecalitalea cylindroides HB-664 | 9.2 | 2.8 | 52.9 | 21.8 |
| 63 | Bilophila wadsworthia HB-693 | — | — | — | — |
| 64 | Bifidobacterium longum HB-71 | — | — | — | — |
| 65 | Clostridium hylemonae HB-73 | — | — | — | — |
| 66 | Clostridium innoculum HB-82 | — | — | — | — |
| 67 | Bifidobacterium breve HB-90 | — | — | — | — |
| 68 | Acidaminococcus intestini HB-95 | — | — | — | — |
| 69 | Streptococcus gordonii HB-98 | — | — | 42.5 | — |
| 70 | Romboutsia lituseburensis HB-102 | — | — | — | — |
| 71 | Blautia wexlerae HB-16 | — | — | — | — |
| 72 | Sutterella wadsworthensis HB-259 | — | — | — | — |
| 73 | Bacteroides clarus HB-30 | — | — | 42 | — |
| 74 | Prevotella sp. HB-333 | — | — | — | — |
| 75 | Bacteroides thetaiotaomicron HB-34 | — | — | — | — |
| 76 | Prevotella copri HB-373 | — | — | 49.7 | — |
| 77 | Dialister invisus HB-387 | — | — | — | — |
| 78 | Sellimonas intestinalis HB-443 | — | — | — | — |
| 79 | Bittarella massiliensis HB-477 | — | — | — | — |
| 80 | Ruminococcus sp. HB-626 | 9.9 | 2.7 | 42.5 | 20.3 |
| 81 | Prevotella HB-HB-649 | — | — | — | — |
| 82 | Longibaculum muris HB-79 | — | — | — | — |
| 83 | Ruminococcus bicirculans HB-105 | — | — | — | — |
| 84 | Parabacteroides distasonis HB-214 | — | — | — | — |
| 85 | Veillonella atypica HB-251 | — | — | — | — |
| 86 | Clostridium sp. HB-358 | — | — | — | — |
| 87 | Parabacteroides goldsteinii HB-44 | — | — | — | — |
| 88 | Clostridium sphenoides HB-470 | — | — | — | — |
| 89 | Escherichia coli HB-490 | — | — | — | — |
| 90 | Lawsonibacter asaccharolyticus HB-521 | — | — | — | — |
| 91 | Gemmiger sp. HB-567 | — | — | — | — |
| 92 | Eubacterium callanderi HB-59 | — | — | — | — |
| 93 | Intestinimonas massiliensis HB-651 | — | — | — | — |
| 94 | Clostridium symbiosum HB-67 | — | — | — | — |
| 95 | Longibaculum sp. HB-681 | — | — | — | — |
| 96 | Blautia obeum HB-14 | — | — | — | — |
| 97 | Dorea longicatena HB-17 | — | — | — | — |
| 98 | Eubacterium rectale HB-22 | — | — | — | — |
| 99 | Blautia coccoides HB-23 | — | — | — | — |
| 100 | Bifidobacterium longum HB-234 | — | — | — | — |
| 101 | Slackia isoflavoniconvertens HB-326 | — | — | — | — |
| 102 | Lachnoclostridium sp. HB-698 | — | — | — | — |
| 103 | Coprococcus comes HB-80 | — | — | — | — |
| 104 | Clostridium butyricum HB-88 | — | — | — | — |
| | Pre-determined threshold | >10.0 | >3.2 | >103.0 | >103.0 |

TABLE 2
Summary of SEQ ID NOs

| Sequence Category | SEQ ID NOs |
|---|---|
| 5-HT Producer 16S rDNA sequence | 1-4 |
| 5-HT Modulating Supernatant Producers 16S rDNA sequence | 1-2; 5-69 |
| 5-HT Modulating Pellet Producers 16S rDNA sequence | 1-3, 5-30; 70-82 |
| 5-HT Non- or Low Modulators 16S rDNA sequence | 83-95 |
| 5-HT Negative Modulators 16S rDNA sequence | 96-104 |
| 5-HT Agonist Producers 16S rDNA sequence | 1-4; 105-114 |
| Example Tryptophan Decarboxylases (e.g., EC 4.1.1.105) | 115-119 |
| Example Tryptophan Hydroxylases (e.g., EC 1.14.16.4) | 120-126 |
| Example Aromatic L-amino acid decarboxylases (e.g., EC 4.1.1.28) | 127-133 |
| Example Anaerobic Hydroxylase | 134 |
| Example Tryptophan Production Enzymes (EC 4.2.1.20, EC 4.1.1.48, | 135-163 |
| EC 2.4.2.18, EC 4.1.3.27, EC 5.3.1.24, EC 5.3.1.36) | |
| Example Acyl-CoA dehydrogenases (e.g., EC 1.3.99.3, EC 1.3.8.7; | 164-171 |
| EC 1.3.8.8; EC 1.3.8.9) | |
| Other bacterial enzymes involved in 5-HT agonist or modifier | 172-184 |
| production (e.g., EC 4.1.99.1; EC 2.8.3.17; EC 4.2.1.175; | |
| EC 5.6.1.9; EC 2.1.1.—) | |
| Example Decarboxylases (see e.g., FIG. 1) (e.g., EC 1.1.1; | 185-226 |
| EC 1.1.1.40; EC 4.1.1; EC 4.1 .1.18; EC 4.1.1.17; EC 4.1.1.19; | |
| EC 4.1.1.20; EC 4.1.1.23; EC 4.1.1.3; EC 4.1.1.36; EC 4.1.1.41; | |
| EC 4.1.1.44; EC 4.1.1.65; EC 4.1.1.81; EC 4.1.1.96; EC 6.3.2.5; | |
| or EC6.4.1.2) | |
| Example Phenylalanine Hydroxylase (e.g., EC 1.14.16.1) | 227 |
| Example N-methylated tryptophan derivative-producing enzymes | 228-229 |
| (e.g., EC 2.1.1.44; EC 2.1.1.— or EC 2.1.1.17) | |

1. A therapeutic composition for increasing serotonin level in a mammalian subject in need thereof, the composition comprising an amount of a live isolated serotonin-increasing bacterial species, dead isolated serotonin-increasing bacterial species, conditioned medium from an isolated, cultured serotonin-increasing bacterial species, cell pellet of an isolated serotonin-increasing bacterial species, a purified metabolite produced by an isolated serotonin-increasing bacterial species, a purified protein produced by an isolated serotonin-increasing bacterial species, or a combination thereof sufficient to increase serotonin level in the subject, and an excipient or carrier suitable for delivery to the gut.

2. (canceled)

3. The therapeutic composition of claim 1, wherein the isolated serotonin-increasing bacterial species comprises Bifidobacterium adolescentis.

4. The therapeutic composition of claim 1, wherein the serotonin increasing bacterial species comprises one or more species selected from Enterococcus durans, Clostridium lavalense, Clostridium asparagiforme, and Ruminococcus gnavus.

5. (canceled)

6. The therapeutic composition of claim 1, wherein the serotonin increasing bacterial species:
(a) comprises a 16S sequence at least 95% identical to a 16S sequence selected from SEQ ID NOs: 1-4;
(b) comprises a 16S sequence at least 95% identical to a 16S sequence comprising SEQ ID NO: 12; or
(c) increases serotonin under conditions found in the mammalian gut.

7. (canceled)

8. The therapeutic composition of claim 1, wherein the mammalian subject is a human subject.

9. The therapeutic composition of claim 1, wherein the serotonin-increasing bacterial species encodes and expresses:
(a) a decarboxylase enzyme that catalyzes the production of tryptamine from tryptophan; and/or
(b) an enzyme that hydroxylates tryptamine to produce serotonin.

10. The therapeutic composition of claim 9, wherein the decarboxylase enzyme:
(a) is a tryptophan decarboxylase;
(b) belongs to the EC number 4.1.1.105;
(c) is at least 50% identical to an enzyme comprising an amino acid sequence selected from SEQ ID Nos: 115-119; or
(d) is a lysine decarboxylase family enzyme.

11.-13. (canceled)

14. The therapeutic composition of claim 9, wherein the enzyme that hydroxylates tryptamine is:
(a) a tryptamine 5-hydroxylase;
(b) at least 50% identical to the enzyme of SEQ ID NO: 134; or
(c) an anaerobic hydroxylase.

15.-17. (canceled)

18. The therapeutic composition of claim 1, wherein the live isolated serotonin-increasing bacterial species, dead isolated serotonin-increasing bacterial species, conditioned medium from an isolated, cultured serotonin-increasing bacterial species or cell pellet of an isolated serotonin-increasing bacterial species comprises a bacterial species that encodes and expresses a decarboxylase enzyme that catalyzes the production of tryptamine from tryptophan.

19. (canceled)

20. The therapeutic composition of claim 1, wherein the serotonin-increasing bacterial species encodes and expresses:
(a) an enzyme that converts tryptophan to 5-hydroxy-L-tryptophan (5-HTP); and/or
(b) an enzyme that converts 5-hydroxy-L-tryptophan to serotonin.

21. The therapeutic composition of claim 20, wherein the enzyme that converts tryptophan to 5-hydroxy-L-tryptophan (5-HTP):
(a) has an amino acid sequence at least 50% identical to a sequence selected from SEQ ID Nos: 120-126;
(b) belongs to the EC number 1.14.16.4;
(c) is a tryptophan hydroxylase; or
(d) is a phenylalanine hydroxylase.

22.-23. (canceled)

24. The therapeutic composition of claim 20, wherein the enzyme that converts 5-hydroxy-L-tryptophan to serotonin:
(a) is an aromatic L-amino acid decarboxylase;
(b) belongs to the EC number 4.1.1.28; or
(c) is at least 50% identical to a sequence selected from SEQ ID NOs: 127-133.

25.-29. (canceled)

30. The therapeutic composition of claim 1, wherein the live isolated serotonin-increasing bacterial species, dead isolated serotonin-increasing bacterial species, conditioned medium from an isolated, cultured serotonin-increasing bacterial species or cell pellet of an isolated serotonin-increasing bacterial species comprises a bacterial species that encodes and expresses an enzyme that catalyzes the conversion of tryptophan to 5-hydroxy-L-tryptophan.

31.-33. (canceled)

34. The therapeutic composition of claim 21, wherein the phenylalanine hydroxylase comprises an amino acid sequence comprising one or more of phenylalanine at the position corresponding to W192, isoleucine or leucine at the position corresponding to F197, and cysteine at the position corresponding to E219 of the phenylalanine hydroxylase of Cupriavidus taiwanensis (SEQ ID NO: 227).

35. A pharmaceutical composition comprising the therapeutic composition of claim 1, and a pharmaceutically acceptable carrier.

36. A method of increasing serotonin level in a mammalian subject in need thereof, the method comprising administering a composition of claim 35 to the subject, whereby a serotonin level is increased.

37. The method of claim 36, wherein the administering is to the gut of the subject.

38. The method of claim 36, wherein the level of serotonin in the gut is increased.

39. The method of claim 36, wherein the level of serotonin in circulation is increased.

40. A method of treating a disease or disorder involving or characterized by low serotonin in a subject in need thereof, the method comprising administering a composition of claim 35 to the subject, whereby the disease or disorder is treated.

41.-153. (canceled)
